(19) United States
(12) Patent Application Publication
(10) Pub. No.: US 2016/0290989 A1
(43) Pub. Date: Oct. 6, 2016

(54) MARKER DETECTION FOR CHARACTERIZING THE RISK OF CARDIOVASCULAR DISEASE OR COMPLICATIONS THEREOF

(71) Applicant: The Cleveland Clinic Foundation, Cleveland, OH (US)

(72) Inventors: Stanley Hazen, Pepper Pike, OH (US); Yuping Wu, Beachwood, OH (US); Anupama Reddy, Boston, MA (US); Marie-Luise Brennan, Westlake Village, CA (US)

(21) Appl. No.: 15/088,917

(22) Filed: Apr. 1, 2016

Related U.S. Application Data
(63) Continuation of application No. 12/859,733, filed on Aug. 19, 2010, now abandoned.
(60) Provisional application No. 61/235,283, filed on Aug. 19, 2009.
(60) Provisional application No. 61/289,620, filed on Dec. 23, 2009.
(60) Provisional application No. 61/353,820, filed on Jun. 11, 2010.

(51) Int. Cl. G01N 33/49; G06F 19/24

(52) U.S. Cl. CPC G01N 33/49; G06F 19/24; G01N 2800/32; G01N 2800/50; G01N 2800/56

(57) ABSTRACT

The present invention provides methods, systems, devices, and software for determining values for one or more markers in order to characterize a subject's risk of developing cardiovascular disease or experiencing a complication thereof (e.g., within the ensuing one to three years). In certain embodiments, the markers are those derived from a blood sample using a hematology analyzer operably linked to a software application that is configured to compute a risk score for a subject based on the values for the markers detected in the blood sample.

This application is a Continuation of U.S. application Ser. No. 12/859,733 which claims priority to U.S. Provisional application 61/235,283, filed Aug. 19, 2009, U.S. Provisional application 61/289,620, filed Dec. 23, 2009, and U.S. Provisional application 61/353,820, filed Jun. 11, 2010, each of which is herein incorporated by reference in its entirety.

This invention was made with government support under Grant Nos. P01 HL076491-055328, P01 HL077107-050004, P01 HL087018-02000, awarded by the National Institutes of Health. The government has certain rights in the invention.

### FIELD OF THE INVENTION

The present invention relates to methods, systems, devices, and software for determining values for one or more markers in order to characterize a subject's risk of developing cardiovascular disease or experiencing a complication thereof (e.g., within the ensuing one to three years). In certain embodiments, the markers are those derived from a blood sample using a hematology analyzer operably linked to a software application that is configured to compute a risk score for a subject based on the values for the markers detected in the blood sample.

### BACKGROUND

Despite recent advances in both our understanding of the pathophysiology of cardiovascular disease and the ability to image atherosclerotic plaque, accurate determination of risk in stable cardiac patients remains a challenge. The clinically unidentified high-risk patient who does not undergo aggressive risk factor modification and experiences a major adverse cardiac event is of great concern (1, 2). Similarly, more accurate identification of low-risk subjects is needed to refocus finite health care resources to those who stand most to benefit. Most current clinical risk assessment tools involve algorithms developed from epidemiology based studies of untreated primary prevention populations and are limited in their application to a higher risk and medicated cardiology outpatient setting (3). An area of active investigation is the incorporation of combinations of novel biological markers, genetic polymorphisms, or noninvasive imaging approaches for additive prognostic value (4-7). Despite considerable interest, efforts to incorporate more holistic array-based phenotyping technologies (e.g., genomic, proteomic, metabolomic, expression array) for improved cardiac risk stratification remain in its infancy and have yet to be translated into efficient and robust platforms amenable to the high throughput demands of clinical practice.

Blood is a complex but integrated sensor of physiologic homeostasis. Perturbations in blood composition and blood cell function are seen in both acute and chronic inflammatory conditions. Elevated leukocyte count (both neutrophils and monocytes) has long been associated with cardiovascular morbidity and mortality (8, 9). Leukocyte adhesion, activation, degranulation and release of peroxidase containing granules are key steps in the inflammatory process and have been implicated in the development and progression of cardiovascular atheroma (10). Myeloperoxidase, an abundant leukocyte granule protein enriched within culprit lesions (11), is mechanistically linked with multiple stages of cardiovascular disease (12), including modification of lipoproteins (13-15), creation of pro-inflammatory lipid mediators (14,16), regulation of protease cascades (17, 18), and modulation of nitric oxide bioavailability and vascular tone (19-21).

Systemic myeloperoxidase levels are increased in patients presenting with chest pain (22) and suspected acute coronary syndromes (23) that subsequently experience near term adverse cardiovascular events, and alterations in leukocyte intracellular peroxidase activity are seen in patients with cardiovascular disease (24, 25). Similarly, erythrocytes are critical mediators of both oxygen delivery to tissues and regulation of nitric oxide delivery and bioavailability within the vascular compartment (26), and platelets are essential participants in atherothrombotic disease (27, 28). Thus, numerous mechanistic and epidemiological ties exist between various components and activities of circulating leukocytes, erythrocytes and platelets with processes critical to both vascular homeostasis and progression of cardiovascular disease (24, 25, 28-33).

### SUMMARY OF THE INVENTION

The present invention provides methods, systems, devices, and software for determining values for one or more markers in order to characterize a subject's risk of developing cardiovascular disease or experiencing a complication thereof (e.g., within the ensuing one to three years). In certain embodiments, the markers are those derived from a blood sample using a hematology analyzer operably linked to a software application that is configured to compute a risk score for a subject based on the values for the markers detected in the blood sample.

In some embodiments, the present invention provides methods of characterizing a subject's risk of developing cardiovascular disease or experiencing a complication of cardiovascular disease (or likelihood of having abnormal cardiac catheterization), comprising: a) determining the value of a first marker in a biological sample from the subject, wherein the first marker is selected from the group consisting of Markers 1-55 as defined in Table 50; and b) comparing the value of the first marker to a first threshold value (e.g., a value above or below which indicates a statistical likelihood of risk, such as high-risk or low risk) such that the subject's risk of developing cardiovascular disease or experiencing a complication of cardiovascular disease is at least partially characterized.

In certain embodiments, the first threshold value is a statistically generated threshold value. In some embodiments, the first threshold value is a control population or disease population generated threshold value. In particular embodiments, the comparing the value of the first marker to the first threshold value generates: i) a first high-risk indicator; ii) a non-high/low-risk indicator; or iii) a first low-risk indicator. In further embodiments, the first-risk indicator, the non-high/low-risk indicator, or the low-risk indicator is represented by a word, number, ratio, or character, all of which may be generated in a computer program. In certain embodiments, the first high-risk indicator is a word (e.g., “yes,” “no,” “plus,” “minus,” etc.), a number (e.g., 1, 10, 100, etc), a ratio, or character (“+” or “−” symbol)); ii) the non-high/low-risk indicator is a word (e.g., “no”), a number (e.g., 0), or a symbol (e.g., “−” symbol); and iii) the first low-risk indicator is a word (e.g., “yes”) a number (e.g., −1), or a symbol (e.g., “+” symbol). In certain embodiments, the abnormal cardiac catheterization is indicated by having one or more major coronary vessels with significant stenosis, or having an abnormal stress test, or having an abnormal myocardial perfusion study, etc.

In certain embodiments, the first high-risk indicator, the non-high/low-risk indicator, or the first low-risk indicator is employed to generate an overall risk score for the subject (e.g., a print out or electronic record that contains words, numbers, or characters that indicate the subject's risk (or at least partial risk) of developing cardiovascular disease or experiencing a complication of cardiovascular disease over a given time period, such as one to three years). In additional embodiments, the value of the first marker is greater than the first threshold value, and the subject's risk is at least partially characterized as high-risk. In other embodiments, the value of the first marker is less than the first threshold value, and the subject's risk is at least partially characterized as low-risk. In additional embodiments, the value of the first marker is greater than the first threshold value, and the subject's risk is at least partially characterized as low-risk. In additional embodiments, the value of the first marker is less than the first threshold value, and the subject's risk is at least partially characterized as high-risk.

In some embodiments, the methods further comprise: c) determining the value of a second marker (or third, fourth . . . tenth . . . twentieth . . . fifty-fifth marker) in the biological sample, wherein the second marked is selected from the group consisting Markers 1-75 as defined in Table 50; and d) comparing the value of the second marker to a second threshold (or a third, fourth . . . tenth . . . twentieth . . . fifty-fifth marker) value such that the subject's risk of developing cardiovascular disease or experiencing a complication of cardiovascular disease is further characterized. In certain embodiments, the cardiovascular disease or complication thereof is selected from: arteriosclerosis, atherosclerosis, myocardial infarction, acute coronary syndrome, angina, congestive heart failure, aortic aneurysm, aortic dissection, iliac or femoral aneurysm, pulmonary embolism, primary hypertension, atrial fibrillation, stroke, transient ischemic attack, systolic dysfunction, diastolic dysfunction, myocarditis, atrial tachycardia, ventricular fibrillation, endocarditis, arteriopathy, vasculitis, atherosclerotic plaque, vulnerable plaque, acute coronary syndrome, acute ischemic attack, sudden cardiac death, peripheral vascular disease, coronary artery disease (CAD), peripheral artery disease (PAD), and cerebrovascular disease.

In some embodiments, the present invention provides methods of characterizing a subject's risk of developing cardiovascular disease or experiencing a complication of cardiovascular disease, comprising: a) determining the value of a first marker in a biological sample from the subject, wherein the first marker is selected from the group consisting of: Markers 1-19, 47, and 54-55 as defined in Table 50, and b) comparing the value of the first marker to a first threshold value such that the subject's risk of developing cardiovascular disease or experiencing a complication of cardiovascular disease is at least partially characterized.

In certain embodiments, the present invention provides methods of characterizing a subject's risk of developing cardiovascular disease or experiencing a complication of cardiovascular disease, comprising: a) determining the value of a first marker in a biological sample from the subject, wherein the first marker is selected from the group consisting of: Markers 22, 24-26, 28, 30-31, 34-37, 39-45, 48, and 50-53 as defined in Table 50, and b) comparing the value of the first marker to a first threshold value such that the subject's risk of developing cardiovascular disease or experiencing a complication of cardiovascular disease is at least partially characterized.

In particular embodiments, the biological sample comprises blood or other biological fluid. In certain embodiments, the complication is one or more of the following: non-fatal myocardial infarction, stroke, angina pectoris, transient ischemic attacks, congestive heart failure, aortic aneurysm, aortic dissection, and death. In other embodiments, the risk is a risk of developing cardiovascular disease or experiencing a complication of cardiovascular disease within the ensuing one to three years. In certain embodiments, the method further comprises: c) determining the value of a second marker in the biological sample, wherein the second marker is different from the first marker and is selected from the group consisting Markers 1-75 as defined in Table 50; and d) comparing the value of the second marker to a second threshold value such that the subject's risk of developing cardiovascular disease or experiencing a complication of cardiovascular disease is further characterized. In additional embodiments, the method further comprises: c) determining the value of a third marker in the biological sample, wherein the third marker is different from the first and second markers and is selected from the group consisting Markers 1-75 as defined in Table 50; and d) comparing the value of the third marker to a third threshold value such that the subject's risk of developing cardiovascular disease or experiencing a complication of cardiovascular disease is further characterized. In other embodiments, the method further comprises: c) determining the value of a fourth marker in the biological sample, wherein the fourth marker is different from the first, second, and third markers and is selected from the group consisting Markers 1-75 as defined in Table 50; and d) comparing the value of the fourth marker to a fourth threshold value such that the subject's risk of developing cardiovascular disease or experiencing a complication of cardiovascular disease is further characterized.

In some embodiments, a hematology analyzer is employed to determine the value of the first marker. In further embodiments, the comparing is performed in at least partially automated fashion by computer software. In certain embodiments, the subject is a human, a dog, a horse, or a cat. In particular embodiments, the comparing the value of the first marker to the first threshold value generates a first high-risk indicator, a first non-high/low-risk indicator, or a first low-risk indicator. In other embodiments, the first high-risk indicator, the first non-high/low-risk indicator, or the first low-risk indicator is employed to generate an overall risk score for the subject.

In certain embodiments, the present invention provides methods of characterizing a subject's risk of developing cardiovascular disease or experiencing a complication of cardiovascular disease (or the likelihood of having abnormal cardiac catheterization), comprising: a) determining the value of a first marker and a second marker in a biological sample from the subject, wherein the first marker is selected from the group consisting of Markers 1-55 as defined in Table 50, and the second marker is different from the first marker and is selected from the group consisting of Markers 1-75; and b) comparing the value of the first marker to a first threshold value, and comparing the value of the second marker to a second threshold value, such that the subject's risk of developing cardiovascular disease or experiencing a complication of cardiovascular disease is at least partially characterized.

In some embodiments, the present invention provides methods of characterizing a subject's risk of developing cardiovascular disease or experiencing a complication of cardiovascular disease, comprising: a) determining the value of a first marker and a second marker in a biological sample from the subject, wherein the first marker is selected from the group consisting of Markers 1-19, 47, 54, and 55 as defined in Table 50, and the second marker is different from the first marker and is selected from the group consisting of Markers 1-75; and b) comparing the value of the first marker to a first threshold value, and comparing the value of the second marker to a second threshold value, such that the subject's risk of developing cardiovascular disease or experiencing a complication of cardiovascular disease is at least partially characterized.

In certain embodiments, the present invention provides methods of characterizing a subject's risk of developing cardiovascular disease or experiencing a complication of cardiovascular disease, comprising: a) determining the value of a first marker and a second marker in a biological sample from the subject, wherein the first marker is selected from the group consisting of Markers 20-46 and 48-53 as defined in Table 50, and the second marker is different from the first marker and is selected from the group consisting of Markers 1-75; and b) comparing the value of the first marker to a first threshold value, and comparing the value of the second marker to a second threshold value, such that the subject's risk of developing cardiovascular disease or experiencing a complication of cardiovascular disease is at least partially characterized.

In some embodiments, the comparing the value of the first marker to the first threshold value, and comparing the value of the second marker to the second threshold value, generates a first pattern high-risk indicator, a first pattern non-high/low-risk indicator, or a first pattern low-risk indicator. In other embodiments, the first pattern high-risk indicator, the first pattern non-high/low-risk indicator, or the first pattern low-risk indicator is employed to generate an overall risk score for the subject. In additional embodiments, the biological sample comprises blood or other suitable biological fluid. In some embodiments, the complication is one or more of the following: non-fatal myocardial infarction, stroke, angina pectoris, transient ischemic attacks, congestive heart failure, aortic aneurysm, aortic dissection, and death. In further embodiments, the risk is a risk of developing cardiovascular disease or experiencing a complication of cardiovascular disease within the ensuing one to three years.

In some embodiments, the methods further comprise: c) determining the value of a third marker in the biological sample, wherein the third (or fourth . . . twenty-fifth.) marker is different from the first and second markers and is selected from the group consisting Markers 1-75 as defined in Table 50; and d) comparing the value of the third marker to a third threshold value (or fourth . . . twenty fifth . . . ) such that the subject's risk of developing cardiovascular disease or experiencing a complication of cardiovascular disease is further characterized.

In particular embodiments, the methods further comprise: c) determining the value of a third marker and a fourth marker in the biological sample, wherein the third marker is different from the first and second markers and is selected from the group consisting Markers 1-75 as defined in Table 50, and wherein the fourth marker is different from the first, second, and third markers and is selected from the group consisting of Marker 1-75 as defined in Table 50; and d) comparing the value of the third marker to a third threshold value, and comparing the value of the fourth marker to a fourth threshold value, such that the subject's risk of developing cardiovascular disease or experiencing a complication of cardiovascular disease is further characterized. In certain embodiments, the comparing the value of the third marker to the third threshold value, and comparing the value of the fourth marker to the fourth threshold value, generates a second pattern high-risk indicator, a second pattern non-high/low-risk indicator, or a second pattern low-risk indicator. In further embodiments, the first pattern high-risk indicator or the first pattern low-risk indicator, and the second pattern high-risk indicator or the second pattern low-risk indicator, are employed to generate an overall risk score for the subject.

In additional embodiments, a hematology analyzer (e.g., one that employs peroxidase staining or one that does not) is employed to determine the values of the first and second markers. In further embodiments, the comparing is performed in at least partially automated fashion by computer software. In certain embodiments, the subject is a human (e.g., a male or a female). In further embodiments, the methods further comprise: c) determining the value of a fifth marker and a sixth marker (or further seventh and/or eighth markers; or ninth and/or tenth markers; or eleventh and/or twelfth markers; etc) in the biological sample, wherein the fifth marker is different from the first, second, third, and fourth markers and is selected from the group consisting Markers 1-75 as defined in Table 50, and wherein the sixth marker is different from the first, second, third, fourth, and fifth markers and is selected from the group consisting of Marker 1-75 as defined in Table 50; and d) comparing the value of the fifth marker to a fifth threshold value, and comparing the value of the sixth marker to a sixth threshold value, such that the subject's risk of developing cardiovascular disease or experiencing a complication of cardiovascular disease is further characterized. In particular embodiments, the comparing the value of the fifth marker to the fifth threshold value, and comparing the value of the sixth marker to the sixth threshold value, generates a third pattern high-risk indicator, a third pattern non-high/low-risk indicator, or a third pattern low-risk indicator. In additional embodiments, the first pattern high-risk indicator or the first pattern low-risk indicator, the second pattern high-risk indicator or the second pattern low-risk indicator, and the third pattern high-risk indicator or the third pattern low-risk indicator are employed to generate an overall risk score for the subject (e.g., which is displayed on a display panel or monitor, or which is printed on paper as words or a barcode; or which is emailed to a user such as a doctor, lab technician, a patient).

In certain embodiments, the present invention provides computer program products, comprising: a) a computer readable medium (e.g., hard disk, CD, DVD, flash drive, etc.); b) threshold value data on the computer readable medium comprising at least a first threshold value; and c) instructions (e.g., computer code) on the computer readable medium adapted to enable a computer processor to perform operations comprising: i) receiving subject data (e.g., over electrical wire, over the internet, etc.), wherein the subject data comprises the value of a first marker (e.g., as determined by a hematology analyzer) from a biological sample from the subject, wherein the first marker is selected from the group consisting of Markers 1-55 as defined in Table 50 (or wherein the first marker is selected from the group consisting of Markers 1-19, 22, 24-26, 28, 30-31, 34-37, 39-45, 47-48, and 50-55 as defined in Table 50; or Markers 1-19, 47, and 54-55 as defined in Table 50; or Markers 22, 24-26, 28, 30-31, 34-37, 39-45, 48, and 50-53 as defined in Table 50); ii) comparing the value of the first marker to the first threshold value; and iii) generating first high-risk indicator data, first non-high/low-risk indicator data, or first low-risk indicator data based on the comparing.

In some embodiments, the present invention provides computer program products, comprising: a) a computer readable medium; b) threshold value data on the computer readable medium comprising at least a first threshold value and a second threshold value; and c) instructions on the computer readable medium adapted to enable a computer processor to perform operations comprising: i) receiving subject data, wherein the subject data comprises the value of a first marker and the value of a second marker from a biological sample from the subject, wherein the first marker is selected from the group consisting of Markers 1-55 as defined in Table 50 (or wherein the first marker is selected from the group consisting of Markers 1-19, 47, 54, and 55 as defined in Table 50; or wherein the first marker is selected from the group consisting of Markers 20-46 and 48-53 as defined in Table 50), and the second marker is different from the first marker and is selected from the group consisting of Markers 1-75; ii) comparing the value of the first marker to the first threshold value, and comparing the value of the second marker to the second threshold value; and iii) generating first pattern high-risk indicator data, first pattern non-high/low risk indicator data, or first pattern low-risk indicator data based on the comparing.

In certain embodiments, the present invention provides systems comprising: a) a blood analyzer device; and b) a computer program component configured to: i) receiving subject data, wherein the subject data comprises the value of a first marker from a biological sample from the subject, wherein the first marker is selected from the group consisting of Markers 1-55 as defined in Table 50; and ii) calculate and display a risk profile of cardiovascular disease.

In other embodiments, the present invention provides systems comprising: a) a blood analyzer device; and b) a computer program component comprising: i) a computer readable medium; ii) threshold value data on the computer readable medium comprising at least a first threshold value; and iii) instructions on the computer readable medium adapted to enable a computer processor to perform operations comprising: A) receiving subject data, wherein the subject data comprises the value of a first marker from a biological sample from the subject, wherein the first marker is selected from the group consisting of Markers 1-55 as defined in Table 50 (or Markers 1-19, 47, and 54-55 as defined in Table 50; or Markers 22, 24-26, 28, 30-31, 34-37, 39-45, 48, and 50-53 as defined in Table 50); B) comparing the value of the first marker to the first threshold value; and C) generating first high-risk indicator data, first non-high/low risk indicator data, or first low-risk indicator data based on the comparing.

In further embodiments, the present invention provides systems comprising: a) a blood analyzer device; and b) a computer program component comprising: i) a computer readable medium; ii) threshold value data on the computer readable medium comprising at least a first threshold value and a second threshold value; and iii) instructions on the computer readable medium adapted to enable a computer processor to perform operations comprising: A) receiving subject data, wherein the subject data comprises the value of a first marker and the value of a second marker from a biological sample from the subject, wherein the first marker is selected from the group consisting of Markers 1-55 as defined in Table 50 (or wherein the first marker is selected from the group consisting of Markers 1-19, 47, 54, and 55 as defined in Table 50; or wherein the first marker is selected from the group consisting of Markers 20-46 and 48-53 as defined in Table 50), and the second marker is different from the first marker and is selected from the group consisting of Markers 1-75; B) comparing the value of the first marker to the first threshold value, and comparing the value of the second marker to the second threshold value; and C) generating first pattern high-risk indicator data, first pattern non-high/low-risk indicator data, or first pattern low-risk indicator data based on the comparing.

In some embodiments, the present invention provides systems comprising: a) a blood analyzer device; and b) a computer program component comprising: i) a computer readable medium; ii) threshold value data on the computer readable medium comprising at least a first threshold value; and iii) instructions on the computer readable medium adapted to enable a computer processor to perform operations comprising: A) receiving subject data, wherein the subject data comprises the value of a first marker from a biological sample from the subject, wherein the first marker is selected from the group consisting of Markers 1-19, 47, and 54-55 as defined in Table 50; B) comparing the value of the first marker to the first threshold value; and C) generating first high-risk indicator data, first non-high/low-risk indicator data, or first low-risk indicator data based on the comparing. In certain embodiments, the system further comprises a computer processor. In further embodiments, the blood analyzer device, the computer program component, and the computer process or operably connected (e.g., at least two of the components are connect via the internet or by wire, or are part of the same device).

In other embodiments, the present invention provides systems comprising: a) a blood analyzer device; and b) a computer program component comprising: i) a computer readable medium; ii) threshold value data on the computer readable medium comprising at least a first threshold value; and iii) instructions on the computer readable medium adapted to enable a computer processor to perform operations comprising: A) receiving subject data, wherein the subject data comprises the value of a first marker from a biological sample from the subject, wherein the first marker is selected from the group consisting of Markers 22, 24-26, 28, 30-31, 34-37, 39-45, 48, and 50-53 as defined in Table 50; B) comparing the value of the first marker to the first threshold value; and C) generating first high-risk indicator data, first non-high/low risk indicator data, or first low-risk indicator data based on the comparing.

In certain embodiments, the system further comprises a display component configured to display: i) the high-risk indicator data, first non-high/low risk indicator data, and/or first low-risk indicator data; and/or ii) a risk profile. In certain embodiments, the display component comprises an LCD screen, a t.v., or other type of readable screen. In some embodiments, the system further comprises a user interface (e.g., keyboard, mouse, touch screen, button pad, etc.). In further embodiments, the user interface allows a user to select which of the Markers are detected by the blood analyzer device, and/or which of the markers are employed in the comparing and generating steps. In further embodiments, the user interface allows a user to enter patient information, such as that related to Markers 56-75. In other embodiments, patient information, such as that in Markers 56-75 is imported (e.g., automatically) from a patient's medical records (e.g., via the internet). In other embodiments, the user interface allows a user to select the type or format of risk profile that is displayed on the display component.

In certain embodiments, the system further comprises the computer processor, and wherein the computer program component is operably linked to the computer processor, and wherein the computer processor is operably linked to the blood analyzer device. In further embodiments, the system further comprises a display component configured to display: i) the high-risk indicator data, first non-high/low risk indicator data, and/or first low-risk indicator data; and/or ii) a risk profile. In other embodiments, the system further comprises a user interface. In additional embodiments, at least a portion of the subject data is generated by the blood analyzer device. In some embodiments, the blood analyzer device comprises a hematology analyzer. In additional embodiments, the instruction are adapted to enable the computer processor to perform operations further comprising: iv) outputting the first high-risk indicator data, the first non-high/low risk indicator data, or the first low-risk indicator data. In further embodiments, the instruction are adapted to enable the computer processor to perform operations further comprising: generating an overall risk score for the subject based on the first high-risk indicator data, the non-high/low risk indicator data, or the first low-risk indicator data.

In particular embodiments, the instruction are adapted to enable the computer processor to perform operations further comprising: iv) outputting the overall risk score (e.g., such that it is readable on a display, or on paper, or as an email). In additional embodiments, the overall risk score at least partially characterizes the subject's risk of developing cardiovascular disease or experiencing a complication of cardiovascular disease based on the first high-risk indicator data, the first non-high/low-risk indicator data, or the first low-risk indicator data. In certain embodiments, the instruction are adapted to enable a computer processor to perform operations further comprising: outputting a result that at least partially characterizes the subject's risk of developing cardiovascular disease or experiencing a complication of cardiovascular disease based on the first high-risk indicator data or the first low-risk indicator data.

In some embodiments, the present invention provides systems comprising: a) a blood analyzer device; and b) a computer program component comprising: i) a computer readable medium; ii) threshold value data on the computer readable medium comprising at least a first threshold value and a second threshold value; and iii) instructions on the computer readable medium adapted to enable a computer processor to perform operations comprising: A) receiving subject data, wherein the subject data comprises the value of a first marker and the value of a second marker from a biological sample from the subject, wherein the first marker is selected from the group consisting of Markers 1-19, 47, 54, and 55 as defined in Table 50; and the second marker is different from the first marker and is selected from the group consisting of Markers 1-75; B) comparing the value of the first marker to the first threshold value, and comparing the value of the second marker to the second threshold value; and C) generating first pattern high-risk indicator data, first pattern non-high/low-risk indicator data, or first pattern low-risk indicator data based on the comparing.

In further embodiments, the present invention provides systems comprising: a) a blood analyzer device; and b) a computer program component comprising: i) a computer readable medium; ii) threshold value data on the computer readable medium comprising at least a first threshold value and a second threshold value; and iii) instructions on the computer readable medium adapted to enable a computer processor to perform operations comprising: A) receiving subject data, wherein the subject data comprises the value of a first marker and the value of a second marker from a biological sample from the subject, wherein the first marker is selected from the group consisting of Markers 20-46 and 48-53 as defined in Table 50; and the second marker is different from the first marker and is selected from the group consisting of Markers 1-75; B) comparing the value of the first marker to the first threshold value, and comparing the value of the second marker to the second threshold value; and C) generating first pattern high-risk indicator data, first pattern non-high/low risk indicator data, or first pattern low-risk indicator data based on the comparing.

In certain embodiments, the present invention provides devices comprising: a) a blood analyzer device; b) a computer processor; and c) a computer program component operably linked to said blood analyzer device and said computer processor, wherein said computer program component is configured for: i) receiving subject data, wherein the subject data comprises the value of a first marker from a biological sample from the subject, wherein the first marker is selected from the group consisting of Markers 1-55 as defined in Table 50; and ii) calculate and display a risk profile of cardiovascular disease. In further embodiments, the device further comprises a output display and/or a user interface.

In some embodiments, the present invention provides devices comprising: a) a blood analyzer component; b) a computer processor; and c) a computer program component operably linked to the blood analyzer component and the computer processor, wherein the computer program component comprises: i) a computer readable medium; ii) threshold value data on the computer readable medium comprising at least a first threshold value; and iii) instructions on the computer readable medium adapted to enable the computer processor to perform operations comprising: A) receiving subject data, wherein the subject data comprises the value of a first marker from a biological sample from the subject, wherein the first marker is selected from the group consisting of Markers 1-55 as defined in Table 50; B) comparing the value of the first marker to the first threshold value; and C) generating first high-risk indicator data, first non-high/low-risk indicator data, or first low-risk indicator data based on the comparing.

In further embodiments, the present invention provides devices comprising: a) a blood analyzer component; b) a computer processor; and c) a computer program component operably linked to the blood analyzer component and the computer processor, wherein the computer program component comprises: i) a computer readable medium; ii) threshold value data on the computer readable medium comprising at least a first threshold value and a second threshold value; and iii) instructions on the computer readable medium adapted to enable the computer processor to perform operations comprising: A) receiving subject data, wherein the subject data comprises the value of a first marker and the value of a second marker from a biological sample from the subject, wherein the first marker is selected from the group consisting of Markers 1-55 as defined in Table 50 (or wherein the first marker is selected from the group consisting of Markers 1-19, 47, 54, and 55 as defined in Table 50; or wherein the first marker is selected from the group consisting of Markers 20-46 and 48-53 as defined in Table 50), and the second marker is different from the first marker and is selected from the group consisting of Markers 1-75; B) comparing the value of the first marker to the first threshold value, and comparing the value of the second marker to the second threshold value; and C) generating first pattern high-risk indicator data, first pattern non-high/low-risk indicator data, or first pattern low-risk indicator data based on the comparing.

In certain embodiments, the present invention provides devices comprising: a) a blood analyzer component; b) a computer processor; and c) a computer program component operably linked to the blood analyzer component and the computer processor, wherein the computer program component comprises: i) a computer readable medium; ii) threshold value data on the computer readable medium comprising at least a first threshold value; and iii) instructions on the computer readable medium adapted to enable the computer processor to perform operations comprising: A) receiving subject data, wherein the subject data comprises the value of a first marker from a biological sample from the subject, wherein the first marker is selected from the group consisting of Markers 1-19, 47, and 54-55 as defined in Table 50; B) comparing the value of the first marker to the first threshold value; and C) generating first high-risk indicator data, first non-high/low-risk indicator data, or first low-risk indicator data based on the comparing.

In some embodiments, the present invention provides devices comprising: a) a blood analyzer component; b) a computer processor; and c) a computer program component operably linked to the blood analyzer component and the computer processor, wherein the computer program component comprises: i) a computer readable medium; ii) threshold value data on the computer readable medium comprising at least a first threshold value; and iii) instructions on the computer readable medium adapted to enable the computer processor to perform operations comprising: A) receiving subject data, wherein the subject data comprises the value of a first marker from a biological sample from the subject, wherein the first marker is selected from the group consisting of Markers 22, 24-26, 28, 30-31, 34-37, 39-45, 48, and 50-53 as defined in Table 50; B) comparing the value of the first marker to the first threshold value; and C) generating first high-risk indicator data, first non-high/low risk indicator data, or first low-risk indicator data based on the comparing.

In some embodiments, the present invention provides devices comprising: a) a blood analyzer component; b) a computer processor; and c) a computer program component operably linked to the blood analyzer component and the computer processor, wherein the computer program component comprises: i) a computer readable medium; ii) threshold value data on the computer readable medium comprising at least a first threshold value and a second threshold value; and iii) instructions on the computer readable medium adapted to enable the computer processor to perform operations comprising: A) receiving subject data, wherein the subject data comprises the value of a first marker and the value of a second marker from a biological sample from the subject, wherein the first marker is selected from the group consisting of Markers 1-19, 47, 54, and 55 as defined in Table 50; and the second marker is different from the first marker and is selected from the group consisting of Markers 1-75; B) comparing the value of the first marker to the first threshold value, and comparing the value of the second marker to the second threshold value; and C) generating first pattern high-risk indicator data, first pattern non-high/low-risk indicator data, or first pattern low-risk indicator data based on the comparing.

In certain embodiments, the present invention provides devices comprising: a) a blood analyzer component; b) a computer processor; and c) a computer program component operably linked to the blood analyzer component and the computer processor, wherein the computer program component comprises: i) a computer readable medium; ii) threshold value data on the computer readable medium comprising at least a first threshold value and a second threshold value; and iii) instructions on the computer readable medium adapted to enable the computer processor to perform operations comprising: A) receiving subject data, wherein the subject data comprises the value of a first marker and the value of a second marker from a biological sample from the subject, wherein the first marker is selected from the group consisting of Markers 20-46 and 48-53 as defined in Table 50; and the second marker is different from the first marker and is selected from the group consisting of Markers 1-75; B) comparing the value of the first marker to the first threshold value, and comparing the value of the second marker to the second threshold value; and C) generating first pattern high-risk indicator data, first pattern high/low-risk indicator data, or first pattern low-risk indicator data based on the comparing.

In certain embodiments, the blood analyzer component comprises a detecting unit for irradiating a blood sample with light and obtaining optical information which comprises at least scattered light information from each cell type contained in a blood sample. In further embodiments, the device further comprises a display component configured to display: i) the high-risk indicator data, first non-high/low risk indicator data, and/or first low-risk indicator data; and/or ii) a risk profile. In certain embodiments, the device further comprises a user interface. In particular embodiments, the blood analyzer component comprises a detecting unit for irradiating a blood sample with light and obtaining optical information which comprises at least scattered light information from each cell type contained in a blood sample.

In certain embodiments, the blood analyzer component comprises a detecting unit for irradiating a blood sample with light and obtaining optical information which comprises at least scattered light information from each cell type contained in a blood sample. In other embodiments, the system further comprises a display component configured to display: i) the high-risk indicator data, first non-high/low risk indicator data, and/or first low-risk indicator data; and/or ii) a risk profile. In additional embodiments, the system further comprises a user interface.

In other embodiments, the present invention provides methods of evaluating the efficacy of a therapeutic agent (or a therapeutic intervention such as lifestyle change (e.g., diet, exercise, use of a device, etc.)) in a subject with cardiovascular disease, comprising: a) determining the value of a first marker in a first biological sample from the subject prior to administration of the therapeutic agent, wherein the first marker is selected from the group consisting of Markers 1-55 as defined in Table 50; b) comparing the value of the first marker to a first threshold value, wherein the comparing the value of the first marker to the first threshold value generates a first high-risk indicator; c) administering the therapeutic agent to the subject; d) determining the value of the first marker in a second biological sample from the subject during or after administration of the therapeutic agent; and e) determining the therapeutic agent (or therapeutic intervention) to be efficacious in treating cardiovascular disease in the subject if the value of the first marker, when compared to the first threshold value, generates a non-high/low-risk indicator or a low-risk indicator.

In certain embodiments, the present invention provides methods of evaluating the efficacy of a therapeutic agent (or a therapeutic intervention such as lifestyle change (e.g., diet, exercise, use of a device, etc.)) in a subject with cardiovascular disease, comprising: a) determining the value of first and second markers in a first biological sample from the subject prior to administration of the therapeutic agent, wherein the first marker is selected from the group consisting of Markers 1-55 as defined in Table 50, and wherein the second marker is different from the first marker and is selected from the group consisting of Markers 1-75; b) comparing the value of the first marker to a first threshold value, and comparing the value of the second marker to a second threshold value, wherein the comparing generates a first pattern high-risk indicator; c) administering the therapeutic agent to the subject; d) determining the value of the first and second markers in a second biological sample from the subject during or after administration of the therapeutic agent; and e) determining the therapeutic agent (therapeutic intervention) to be efficacious in treating cardiovascular disease in the subject if the values of the first and second markers, when compared to the first and second threshold values, generates a non-high/low-risk indicator or low-risk indicator.

### BRIEF DESCRIPTION OF THE FIGURES

FIGS. 1A-F show Kaplan-Meier curves and composite risk for one-year outcomes based on tertiles of PEROX risk score in the Validation Cohort. Kaplan-Meier curves for cumulative probability of death (A), myocardial infarction (B), or either event (C) according to low, medium, and high tertiles of PEROX score. Spline curves (solid line) with 95% confidence intervals (dashed line) showing association between cumulative event (Y axis) for death (D), myocardial infarction (E), and death or myocardial infarction (F), for PEROX score (X axis) are shown. Also illustrated are the absolute event rates per decile of PEROX score within the Derivation (red filled circle) and Validation (blue filled circle) cohorts. Vertical dotted lines indicate the tertile cut-points separating low (<40), medium (≧40 to <48) and high (≧48) PEROX scores.

FIG. 2 shows a validation analysis of PEROX risk score. As described in Example 1, models were assessed for their association with one-year incident risk of myocardial infarction or death. Models were comprised of traditional risk factors alone (including age, gender, smoking, LDL cholesterol, HDL cholesterol, systolic blood pressure and history of diabetes) versus traditional risk factors plus PEROX score. Re-sampling (250 bootstrap samples from the Validation Cohort, n=1474) was performed. All data analyses, including ROC analyses and AUC determinations, were separately recalculated at each re-sampling for models with/without PEROX score. The AUCs calculated from the bootstrap samples are compared using side-by-side box plots where boxes represent inter quartile ranges (defined as the difference between the first quartile and the third quartile) and whiskers represent 5th and 95th percentile values.

FIG. 3 shows a comparison of classification accuracy for one-year death (A), myocardial infarction (B), and death or myocardial infarction (C), according to PEROX risk score, and alternative validated clinical risk scores in the Validation Cohort. Receiver operator characteristics curves plotting sensitivity (X axis) and 1-specificity (Y axis) are shown (within independent Validation Cohort subjects only, N=1,474) for PEROX (black line), ATP III (green line), Reynolds Risk (red line), and Duke Angiographic Risk (blue line) scores. Inset within each figure (death, myocardial infarction, and either outcome (Death/MI)) is the area under the curve (AUC, equivalent to accuracy) for each risk score. The p value for comparison of each risk score with the PEROX score is shown.

FIG. 4 shows a example, from Example 1, of a Cytogram (˜50,000 cells) as it appears on an analyzer screen. Cell types are distinguished based on differences in peroxidase staining and associated absorbance and scatter measurements. Clusters are in different colors and abbreviations are included to help in distinguishing cell types. Abbreviations: Neutrophils (Neut), Monocytes (Mono), Large unstained cells (LUC), Eosinophils (Eos), Lymphocytes and basophils (L/B), Platelet clumps (Pc) and Nucleated RBCs and Noise (NRBC/Noise).

FIG. 5 shows two examples of cytograms from different subjects from Example 1. Some of the hematology variables related to the neutrophil main cluster are shown. Subject A has a low PEROX risk score. Subject B has a high PEROX risk score. While visual inspection of the cytograms reveals clear differences, the ultimate assignment into “low” (e.g. bottom tertile) vs. “high” (top tertile) risk categories is not possible by visual inspection, since the final PEROX risk score is dependent upon the weighted presence of multiple binary pairs of low and high risk patterns derived from clinical data, laboratory data and hematological parameters from erythrocyte, leukocyte and platelet lineages. In general, cellular clusters (and subclusters) can be defined mathematically by an ellipse, with major and minor axes, distribution widths along major and minor axes, location and angles relative to the X and Y axes, etc.

FIG. 6, from Example 2, shows a comparison of classification of death or MI in 1 year according to CHRP risk score, and validated clinical risk scores on validation cohort. Receiver operator characteristics curves plotting sensitivity (X axis) and 1-specificity (Y axis) are shown for CHRP (N=1,474 patients), Framingham ATP III (N=1,474 patients), Reynolds Risk (N=1,403 patients), and Duke Angiographic Risk (n=1,129 patients) scores. Inset within the figure is the area under the curve (AUC) for each risk score.

FIGS. 7A-F, from Example 2, show Kaplan-Meier curves and composite risk for one-year death and MI based on tertiles of CHRP score in validation cohort. Kaplan-Meier curves for cumulative probability of death (A), myocardial infarction (B), or either event (C) according to low, medium, and high tertiles of CHRP risk score. Log-rank tests p-values show that the low, medium and high-risk tertiles have significantly different survival distributions. Spline curves (solid line) with 95% confidence intervals (dashed line) show association between cumulative event (Y axis) for death (D), myocardial infarction (E), and death or myocardial infarction (F), for CHRP risk score (X axis) are shown.

FIGS. 8A, B, and C, from Example 3, show a comparison of classification of death or MI in 1 year according to CHRP (PEROX) risk score, and validated clinical risk scores on validation cohort. Receiver operator characteristics curves plotting sensitivity (X axis) and 1-specificity (Y axis) are shown for CHRP (PEROX), Framingham ATP III, Reynolds Risk, and Duke Angiographic Risk scores. Inset within the figure is the area under the curve (AUC) for each risk score.

FIGS. 9A-F, from Example 3, show Kaplan-Meier curves and composite risk for one-year death and MI based on tertiles of CHRP (PEROX) score in validation cohort. Kaplan-Meier curves for cumulative probability of death (A), myocardial infarction (B), or either event (C) according to low, medium, and high tertiles of CHRP (PEROX) risk score. Log-rank tests p-values show that the low, medium and high-risk tertiles have significantly different survival distributions. Spline curves (solid line) with 95% confidence intervals (dashed line) showing association between cumulative event (Y axis) for death (D), myocardial infarction (E), and death or myocardial infarction (F), for CHRP (PEROX) risk score (X axis) are shown.

FIGS. 10A and B, from Example 4, illustrate that the methodology employed to develop embodiments of the PEROX risk score helps to define “stable” patterns. Hazard ratios (HRs) from 250 random bootstrap samples were determined with a sample size of 5,895 from the derivation cohort, along with their 2.5th, 5th, 25th, 50th, 75th, 95th and 97th percentile estimates.

### DEFINITIONS

As used herein, the terms “cardiovascular disease” (CVD) or “cardiovascular disorder” are terms used to classify numerous conditions affecting the heart, heart valves, and vasculature (e.g., veins and arteries) of the body and encompasses diseases and conditions including, but not limited to arteriosclerosis, atherosclerosis, myocardial infarction, acute coronary syndrome, angina, congestive heart failure, aortic aneurysm, aortic dissection, iliac or femoral aneurysm, pulmonary embolism, primary hypertension, atrial fibrillation, stroke, transient ischemic attack, systolic dysfunction, diastolic dysfunction, myocarditis, atrial tachycardia, ventricular fibrillation, endocarditis, arteriopathy, vasculitis, atherosclerotic plaque, vulnerable plaque, acute coronary syndrome, acute ischemic attack, sudden cardiac death, peripheral vascular disease, coronary artery disease (CAD), peripheral artery disease (PAD), and cerebrovascular disease.

As used herein, the term “atherosclerotic cardiovascular disease” or “disorder” refers to a subset of cardiovascular disease that include atherosclerosis as a component or precursor to the particular type of cardiovascular disease and includes, without limitation, CAD, PAD, cerebrovascular disease. Atherosclerosis is a chronic inflammatory response that occurs in the walls of arterial blood vessels. It involves the formation of atheromatous plaques that can lead to narrowing (“stenosis”) of the artery, and can eventually lead to partial or complete closure of the arterial opening and/or plaque ruptures. Thus atherosclerotic diseases or disorders include the consequences of atheromatous plaque formation and rupture including, without limitation, stenosis or narrowing of arteries, heart failure, aneurysm formation including aortic aneurysm, aortic dissection, and ischemic events such as myocardial infarction and stroke

A cardiovascular event, as used herein, refers to the manifestation of an adverse condition in a subject brought on by cardiovascular disease, such as sudden cardiac death or acute coronary syndromes including, but not limited to, myocardial infarction, unstable angina, aneurysm, or stroke. The term “cardiovascular event” can be used interchangeably herein with the term cardiovascular complication. While a cardiovascular event can be an acute condition, it can also represent the worsening of a previously detected condition to a point where it represents a significant threat to the health of the subject, such as the enlargement of a previously known aneurysm or the increase of hypertension to life threatening levels.

As used herein, the term “diagnosis” can encompass determining the nature of disease in a subject, as well as determining the severity and probable outcome of disease or episode of disease and/or prospect of recovery (prognosis). “Diagnosis” can also encompass diagnosis in the context of rational therapy, in which the diagnosis guides therapy, including initial selection of therapy, modification of therapy (e.g., adjustment of dose and/or dosage regimen or lifestyle change recommendations), and the like.

The terms “individual,” “host,” “subject,” and “patient” are used interchangeably herein, and generally refer to a mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos. In some embodiments, the subject is specifically a human subject. Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

It must be noted that as used herein and in the appended claims, the singular forms “a”, “and”, and “the” include plural referents unless the context clearly dictates otherwise. Thus, for example, reference to “a sample” includes a plurality of such samples and reference to a specific enzyme (e.g., arginase) includes reference to one or more arginase polypeptides and equivalents thereof known to those skilled in the art, and so forth.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values; however, inherently contain certain errors necessarily resulting from error found in their respective measurements.

TABLE 53
Definitions of Various Markers

| | Abbrs. | Definition |
|---|---|---|
| White Blood Cell Related | | |
| White blood cell count | WBC | White blood cell count using perox methodology |
| Neutrophil count | #NEUT | Neutrophil cell count from neutrophil region of perox cytogram |
| Lymphocyte count | #LYMPH | Lymphocyte cell count from lymphocyte region of perox cytogram |
| Monocyte count | #MONO | Monocyte cell count from monocyte region of perox cytogram |
| Eosinophil count | #EOS | Eosinophil cell count from eosinophil region of perox cytogram |
| Basophil count | #BASO | Basophil cell count from baso region of baso cytogram |
| Number of peroxidase saturated | # PERO SAT | Number of cells in last 3 channels of perox cytogram |
| cells | | |
| Neutrophil cluster mean X | NEUTX | Mean channel value of neutrophil cluster on X-axis |
| Neutrophil cluster mean Y | NEUTY | Mean channel value of neutrophil cluster on Y-axis |
| Ky | KY | Measure of fit; i.e. how well neutrophils and lymphocytes fit |
| | | predicted clusters |
| Peroxidase X sigma | PXXSIG | Distribution width of neutrophil cell cluster; Two standard deviations |
| | | from neutrophil X mean value |
| Peroxidase Y mean | PXY | Mean position of neutrophil cluster on Y axis; alternative measure |
| Peroxidase Y sigma | PXYSIG | Distribution width of neutrophil cell cluster; Two standard deviations |
| | | from neutrophil Y mean value |
| Lobularity index | LI | Measure of white blood cell maturity; ratio of mode channels of |
| | | polymorphonuclear cells per mononuclear cells |
| Lymphocyte/large unstained cell | LUC | Highest scatter value of lymphocytes from noise/lymphocyte valley |
| threshold | | |
| Perox d/D | PXDD | Measure of quality of distance between lymphocyte and noise clusters |
| Blasts | % BLASTS | Percent of cells in blast region of basophil cytogram |
| Polymorphonuclear ratio | | Ratio of neutrophils per eosinophils in basophil cytogram |
| Polymorphonuclear cluster x axis | PMNX | Mode of neutrophil cluster from basophil cytogram |
| mode | | |
| Mononuclear central x channel | MNX | Central X channel values from basophil cytogram |
| Mononuclear central y channel | | Central Y channel value from basophil cytogram |
| Mononuclear polymorphonuclear | MNPMN | Distance between mononuclear and polymorphonuclear clusters in |
| valley | | basophil cytogram |
| Large unstained cells count | #LUC | Number of large unstained cells (i.e., cells that do not have peroxidase |
| | | staining, which includes a variety of cell types). |
| Lymphocytic mode | LM | The most abundant value for lymphocytes in the lymphocyte region of |
| | | the cytogram. |
| Peroxidase y mean | PXY | The mean location of the neutrophil cluster on the Y-axis. |
| Blasts Count | #BLST | The absolute number of blasts. |
| Large unstained cells (%) | LUC % | The percentage of large unstained cells for the entire cytogram. |
| Red Blood Cell Related | | |
| RBC count | RBC | RBC counted in RBC/platelet cytogram |
| Hematocrit | HCT | Percent of blood consisting of RBCs; (RBC * MCV)/10 |
| Mean corpuscular volume | MCV | Mean channel of RBC volume histogram |
| Mean corpuscular hemoglobin | MCH | Mean hemoglobin; calculated as hemoglobin per RBC count |
| Mean corpuscular hemoglobin | MCHC | Mean hemoglobin concentration; Hemoglobin * 1000/RBC * MCV |
| concentration | | |
| RBC hemoglobin concentration | CHCM | Mean channel of RBC hemoglobin concentration channel |
| mean | | |
| RBC distribution width | RDW | Distribution width of RBC volumes; RBC volume standard |
| | | deviation/MCV * 100 |
| Hemoglobin distribution width | HDW | Distribution width of RBC hemoglobin concentration; Standard |
| | | deviation of hemoglobin concentration histogram |
| Hemoglobin content distribution | HCDW | Standard deviation of hemoglobin content histogram |
| width | | |
| Normochromic/Normocytic RBC | | RBCs normochromic (hemoglobin concentration between 28 to 41 g/dL) |
| count | | and normocytic (size between 20 to 120 fL) |
| Macrocytic RBC count | #MACRO | RBCs with volume greater than 120 fL |
| Hypochromic RBC count | #HYPO | RBCs with hemoglobin concentrations less than 28 g/dL |
| NRBC count | #NRBC | Nucleated red blood cell count. |
| Measured HGB | MHGB | Measured hemoglobin (e.g., per unit volume of blood). |
| Platelet Related | | |
| Plateletcrit | PCT | Percent of blood consisting of platelets; MPV * PLT |
| Mean-platelet | MPC | Mean platelet volume |
| volume | | |
| Platelet count | PLT | Platelet count |
| Mean-platelet | MPC | Mean of platelet component concentration |
| component | | |
| concentration | | |
| Platelet concentration | PCDW | Distribution width of platelet component concentration; two standard |
| distribution width | | deviations for platelet component concentration |
| Large platelets | #L-PLT | Percent of platelets that are between 20 to 30 fL |
| Platelet clumps | PLT CLU | Percent of platelet clumps in platelet cytogram |

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), flash drives, and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, flash drives, magnetic tape and servers for streaming media over networks.

As used herein, the terms “computer processor” and “central processing unit” or “CPU” are used interchangeably and refers to a device that is able to read a program from a computer memory (e.g., ROM or other computer memory) and perform a set of steps according to the program.

### DETAILED DESCRIPTION OF THE INVENTION

The present invention provides methods, systems, devices, and software for determining values for one or more markers in order to characterize a subject's risk of developing cardiovascular disease or experiencing a complication thereof (e.g., within the ensuing one to three years). In certain embodiments, the markers are those derived from a blood sample using a hematology analyzer operably linked to a software application that is configured to compute a risk score for a subject based on the values for the markers detected in the blood sample.

Work conducted during development of embodiments of the present invention has shown that that data derived from a common, high-throughput, hematology analyzer (including peroxidase-based hematology analyzer, which include leukocyte-, erythrocyte- and platelet-related parameters beyond standard complete blood count (CBC) and differential) can provide a broad spectrum of novel data for assessing and predicting cardiovascular disease risks.

### I. Exemplary Markers

Table 50 below provides fifty-five exemplary markers that can be tested for in a sample, such as blood sample, with an analyzer (e.g., hematology analyzer) in order to at least partially characterize a subject's risk of cardiovascular disease or experiencing a complication of cardiovascular disease. Markers 1-55 may be employed alone (i.e., without any of the other markers) to at least partially characterize the risks of cardio vascular disease or complications thereof. Single makers from Markers 1-55 may also be employed with one or more of the traditional markers shown as Markers 56-75. Also, as shown in Table 50, Markers 1-55 may be employed in a group consisting of, or comprising, one or more of the other markers in the table (i.e., in combination with any of Markers 1-75). Table 50 is presented below.

TABLE 50

| | Second | | | |
|---|---|---|---|---|
| | Marker | Third Marker | Fourth Marker | Fifth Marker |
| First Marker | Selected From: | Selected From: | Selected From: | Selected From: |
| Large unstained cells count = | Markers 2-75. | Markers 2-75, | Markers 2-75, | Markers 2-75, excluding |
| “Marker 1” | | excluding the | excluding the second | the second, third, and |
| Abbreviation: #LUC | | second marker. | and third markers. | fourth markers. |
| Ky = “Marker 2” | Markers 1 and 3- | Markers 1 and 3- | Markers 1 and 3-75, | Markers 1 and 3-75, |
| Abbreviation: KY | 75. | 75, excluding the | excluding the second | excluding the second, |
| | | second marker. | and third markers. | third, and fourth markers. |
| Number of peroxidase | Markers 1-2 and | Markers 1-2 and 4- | Markers 1-2 and 4-75, | Markers 1-2 and 4-75, |
| saturated cells = “Marker | 4-75. | 75, excluding the | excluding the second | excluding the second, |
| 3” | | second marker. | and third markers. | third, and fourth markers. |
| Abbreviation: #PERO SAT | | | | |
| Lymphocyte/large | Markers 1-3 and | Markers 1-3 and 5- | Markers 1-3 and 5-75, | Markers 1-3 and 5-75, |
| unstained cell threshold = | 5-75. | 75, excluding the | excluding the second | excluding the second, |
| “Marker 4” | | second marker. | and third markers. | third, and fourth markers. |
| Abbreviation: LUC | | | | |
| Lymphocytic mode = | Markers 1-4 and | Markers 1-4 and 6- | Markers 1-4 and 6-75, | Markers 1-4 and 6-75, |
| “Marker 5” | 6-75. | 75, excluding the | excluding the second | excluding the second and |
| Abbreviation: LM | | second marker. | and third markers. | third markers. |
| Perox d/D - “Marker 6” | Markers 1-5 and | Markers 1-5 and 7- | Markers 1-5 and 7-75, | Markers 1-5 and 7-75, |
| Abbreviation: PXDD | 7-75. | 75, excluding the | excluding the second | excluding the second, |
| | | second marker. | and third markers. | third, and fourth markers. |
| Peroxidase y sigma = | Markers 1-6 and | Markers 1-6 and 8- | Markers 1-6 and 8-75, | Markers 1-6 and 8-75, |
| “Marker 7” | 8-75. | 75, excluding the | excluding the second | excluding the second, |
| Abbreviation: PXYSIG | | second marker. | and third markers. | third, and fourth markers. |
| Peroxidase x sigma = | Markers 1-7 and | Markers 1-7 and 9- | Markers 1-7 and 9-75, | Markers 1-7 and 9-75, |
| “Marker 8” | 9-75. | 75, excluding the | excluding the second | excluding the second, |
| Abbreviation: PXXSIG | | second marker. | and third markers. | third, and fourth markers. |
| Peroxidase y mean = | Markers 1-8 and | Markers 1-8 and | Markers 1-8 and 10- | Markers 1-8 and 10-75, |
| “Marker 9” | 10-75. | 10-75, excluding | 75, excluding the | excluding the second, |
| Abbreviation: PXY | | the second marker. | second and third | third, and fourth markers. |
| | | | markers. | |
| Blasts (%) = “Marker 10” | Markers 1-9 and | Markers 1-9 and | Markers 1-9 and 11- | Markers 1-9 and 11-75, |
| Abbreviation: % BLASTS | 11-75. | 11-75, excluding | 75, excluding the | excluding the second, |
| | | the second marker. | second and third | third, and fourth markers. |
| | | | markers. | |
| Blasts count = “Marker 11” | Markers 1-10 and | Markers 1-10 and | Markers 1-10 and 12- | Markers 1-10 and 12-75, |
| Abbreviation: #BLST | 12-75. | 12-75, excluding | 75, excluding the | excluding the second, |
| | | the second marker. | second and third | third, and fourth markers. |
| | | | markers. | |
| Mononuclear central x | Markers 1-11 and | Markers 1-11 and | Markers 1-11 and 13- | Markers 1-11 and 13-75, |
| channel = “Marker 12” | 13-75. | 13-75, excluding | 75, excluding the | excluding the second, |
| Abbreviation: MNX | | the second marker. | second and third | third, and fourth markers. |
| | | | markers. | |
| Mononuclear central y | Markers 1-12 and | Markers 1-12 and | Markers 1-12 and 14- | Markers 1-12 and 14-75, |
| channel = “Marker 13” | 14-75. | 14-75, excluding | 75, excluding the | excluding the second, |
| Abbreviation: MNY | | the second marker. | second and third | third, and fourth markers. |
| | | | markers. | |
| Mononuclear | Markers 1-13 and | Markers 1-13 and | Markers 1-13 and 15- | Markers 1-13 and 15-75, |
| polymorphonuclear valley = | 15-75. | 15-75, excluding | 75, excluding the | excluding the second, |
| “Marker 14” | | the second marker. | second and third | third, and fourth markers. |
| Abbreviation: MNPMN | | | markers. | |
| Neutrophil cluster mean x = | Markers 1-14 and | Markers 1-14 and | Markers 1-14 and 16- | Markers 1-14 and 16-75, |
| “Marker 15” | 16-75. | 16-75, excluding | 75, excluding the | excluding the second, |
| Abbreviation: NEUTX | | the second marker. | second and third | third, and fourth markers. |
| | | | markers. | |
| Neutrophil cluster mean y = | Markers 1-15 and | Markers 1-15 and | Markers 1-15 and 17- | Markers 1-15 and 17-75, |
| “Marker 16” | 17-75. | 17-75, excluding | 75, excluding the | excluding the second, |
| Abbreviation: NEUTY | | the second marker. | second and third | third, and fourth markers. |
| | | | markers. | |
| Lobularity index = “Marker | Markers 1-16 and | Markers 1-16 and | Markers 1-16 and 18- | Markers 1-16 and 18-75, |
| 17” | 18-75. | 18-75, excluding | 75, excluding the | excluding the second, |
| Abbreviation: LI | | the second marker. | second and third | third, and fourth markers. |
| | | | markers. | |
| Polymorphonuclear ratio | Markers 1-17 and | Markers 1-17 and | Markers 1-17 and 19- | Markers 1-17 and 19-75, |
| (%) = “Marker 18” | 19-75. | 19-75, excluding | 75, excluding the | excluding the second, |
| Abbreviation: PMR | | the second marker. | second and third | third, and fourth markers. |
| | | | markers. | |
| Polymorphonuclear cluser | Markers 1-18 and | Markers 1-18 and | Markers 1-18 and 20- | Markers 1-18 and 20-75, |
| x axis mode = “Marker 19” | 20-75. | 20-75, excluding | 75, excluding the | excluding the second, |
| Abbreviation: PMNX | | the second marker. | second and third | third, and fourth markers. |
| | | | markers. | |
| White blood cell count = | Markers 1-19 and | Markers 1-19 and | Markers 1-19 and 21- | Markers 1-19 and 21-75, |
| “Marker 20” | 21-75. | 21-75, excluding | 75, excluding the | excluding the second, |
| Abbreviation: WBC | | the second marker. | second and third | third, and fourth markers. |
| | | | markers. | |
| Neutrophils (%) = “Marker | Markers 1-20 and | Markers 1-20 and | Markers 1-20 and 22- | Markers 1-20 and 22-75, |
| 21” | 22-75. | 22-75, excluding | 75, excluding the | excluding the second, |
| Abbreviation: NT % | | the second marker. | second and third | third, and fourth markers. |
| | | | markers. | |
| Lymphocytes (%) = | Markers 1-21 and | Markers 1-21 and | Markers 1-21 and 23- | Markers 1-21 and 23-75, |
| “Marker 22” | 23-75. | 23-75, excluding | 75, excluding the | excluding the second, |
| Abbreviation: LM % | | the second marker. | second and third | third, and fourth markers. |
| | | | markers. | |
| Monocytes (%) = “Marker | Markers 1-22 and | Markers 1-22 and | Markers 1-22 and 24- | Markers 1-22 and 24-75, |
| 23” | 24-75. | 24-75, excluding | 75, excluding the | excluding the second, |
| Abbreviation: MN % | | the second marker. | second and third | third, and fourth markers. |
| | | | markers. | |
| Eosinophils (%) = “Marker | Markers 1-23 and | Markers 1-23 and | Markers 1-23 and 25- | Markers 1-23 and 25-75, |
| 24” | 25-75. | 25-75, excluding | 75, excluding the | excluding the second, |
| Abbreviation: ES % | | the second marker. | second and third | third, and fourth markers. |
| | | | markers. | |
| Basophils (%) = “Marker | Markers 1-24 and | Markers 1-24 and | Markers 1-24 and 26- | Markers 1-24 and 26-75, |
| 25” | 26-75. | 26-75, excluding | 75, excluding the | excluding the second, |
| Abbreviation: BS % | | the second marker. | second and third | third, and fourth markers. |
| | | | markers. | |
| Large unstained cells (%) = | Markers 1-25 and | Markers 1-25 and | Markers 1-25 and 27- | Markers 1-25 and 27-75, |
| “Marker 26” | 27-75. | 27-75, excluding | 75, excluding the | excluding the second, |
| Abbreviation: LUC % | | the second marker. | second and third | third, and fourth markers. |
| | | | markers. | |
| Neutrophil count = | Markers 1-26 and | Markers 1-26 and | Markers 1-26 and 28- | Markers 1-26 and 28-75, |
| “Marker 27” | 28-75. | 28-75, excluding | 75, excluding the | excluding the second, |
| Abbreviation: #NEUT | | the second marker. | second and third | third, and fourth markers. |
| | | | markers. | |
| Lymphocyte count = | Markers 1-27 and | Markers 1-27 and | Markers 1-27 and 29- | Markers 1-27 and 29-75, |
| “Marker 28” | 29-75. | 29-75, excluding | 75, excluding the | excluding the second, |
| Abbreviation: #LYMPH | | the second marker. | second and third | third, and fourth markers. |
| | | | markers. | |
| Monocyte count = “Marker | Markers 1-28 and | Markers 1-28 and | Markers 1-28 and 30- | Markers 1-28 and 30-75, |
| 29” | 30-75. | 30-75, excluding | 75, excluding the | excluding the second, |
| Abbreviation: #MONO | | the second marker. | second and third | third, and fourth markers. |
| | | | markers. | |
| Eosinophil count = | Markers 1-29 and | Markers 1-29 and | Markers 1-29 and 31- | Markers 1-29 and 31-75, |
| “Marker 30” | 31-75. | 31-75, excluding | 75, excluding the | excluding the second, |
| Abbreviation: #EOS | | the second marker. | second and third | third, and fourth markers. |
| | | | markers. | |
| Basophil count = “Marker | Markers 1-30 and | Markers 1-30 and | Markers 1-30 and 32- | Markers 1-30 and 32-75, |
| 31” | 32-75. | 32-75, excluding | 75, excluding the | excluding the second, |
| Abbreviation: #BASO | | the second marker. | second and third | third, and fourth markers. |
| | | | markers. | |
| RBC count = “Marker 32” | Markers 1-31 and | Markers 1-31 and | Markers 1-31 and 33- | Markers 1-31 and 33-75, |
| Abbreviation: RBC | 33-75. | 33-75, excluding | 75, excluding the | excluding the second, |
| | | the second marker. | second and third | third, and fourth markers. |
| | | | markers. | |
| Hematocrit (%) = “Marker | Markers 1-32 and | Markers 1-32 and | Markers 1-32 and 34- | Markers 1-32 and 34-75, |
| 33” | 34-75. | 34-75, excluding | 75, excluding the | excluding the second, |
| Abbreviation: HCT | | the second marker. | second and third | third, and fourth markers. |
| | | | markers. | |
| Mean Corpuscular volume = | Markers 1-33 and | Markers 1-33 and | Markers 1-33 and 35- | Markers 1-33 and 35-75, |
| “Marker 34” | 35-75. | 35-75, excluding | 75, excluding the | excluding the second, |
| Abbreviation: MCV | | the second marker. | second and third | third, and fourth markers. |
| | | | markers. | |
| Mean corpuscular hgb = | Markers 1-34 and | Markers 1-34 and | Markers 1-34 and 36- | Markers 1-34 and 36-75, |
| “Marker 35” | 36-75. | 36-75, excluding | 75, excluding the | excluding the second, |
| Abbreviation: MCH | | the second marker. | second and third | third, and fourth markers. |
| | | | markers. | |
| Mean corpuscular hgb | Markers 1-35 and | Markers 1-35 and | Markers 1-35 and 37- | Markers 1-35 and 37-75, |
| concentration = Marker 36 | 37-75. | 37-75, excluding | 75, excluding the | excluding the second, |
| Abbreviation: MCHC | | the second marker. | second and third | third, and fourth markers. |
| | | | markers. | |
| RBC hgb concentration | Markers 1-36 and | Markers 1-36 and | Markers 1-36 and 38- | Markers 1-36 and 38-75, |
| mean = “Marker 37” | 38-75. | 38-75, excluding | 75, excluding the | excluding the second, |
| Abbreviation: CHCM | | the second marker. | second and third | third, and fourth markers. |
| | | | markers. | |
| RBC distribution width = | Markers 1-37 and | Markers 1-37 and | Markers 1-37 and 39- | Markers 1-37 and 39-75, |
| “Marker 38” | 39-75. | 39-75, excluding | 75, excluding the | excluding the second, |
| Abbreviation: RDW | | the second marker. | second and third | third, and fourth markers. |
| | | | markers. | |
| Hgb distribution width = | Markers 1-38 and | Markers 1-38 and | Markers 1-38 and 40- | Markers 1-38 and 40-75, |
| “Marker 39” | 40-75. | 40-75, excluding | 75, excluding the | excluding the second, |
| Abbreviation: HDW | | the second marker. | second and third | third, and fourth markers. |
| | | | markers. | |
| Hgb content distribution | Markers 1-39 and | Markers 1-39 and | Markers 1-39 and 41- | Markers 1-39 and 41-75, |
| width = “Marker 40” | 41-75. | 41-75, excluding | 75, excluding the | excluding the second, |
| Abbreviation: HCDW | | the second marker. | second and third | third, and fourth markers. |
| | | | markers. | |
| Macrocytic RBC count = | Markers 1-40 and | Markers 1-40 and | Markers 1-40 and 42- | Markers 1-40 and 42-75, |
| “Marker 41” | 42-75. | 42-75, excluding | 75, excluding the | excluding the second, |
| Abbreviation: #MACRO | | the second marker. | second and third | third, and fourth markers. |
| | | | markers. | |
| Hypochromic RBC count = | Markers 1-41 and | Markers 1-41 and | Markers 1-41 and 43- | Markers 1-41 and 43-75, |
| “Marker 42” | 43-75. | 43-75, excluding | 75, excluding the | excluding the second, |
| Abbreviation: #HYPO | | the second marker. | second and third | third, and fourth markers. |
| | | | markers. | |
| Hyperchromic RBC count = | Markers 1-42 and | Markers 1-42 and | Markers 1-42 and 44- | Markers 1-42 and 44-75, |
| “Marker 43” | 44-75. | 44-75, excluding | 75, excluding the | excluding the second, |
| Abbreviation: #HYPE | | the second marker. | second and third | third, and fourth markers. |
| | | | markers. | |
| Microcytic RBC count = | Markers 1-43 and | Markers 1-43 and | Markers 1-43 and 45- | Markers 1-43 and 45-75, |
| “Marker 44” | 45-75. | 45-75, excluding | 75, excluding the | excluding the second, |
| Abbreviation: #MRBC | | the second marker. | second and third | third, and fourth markers. |
| | | | markers. | |
| NRBC count = “Marker | Markers 1-44 and | Markers 1-44 and | Markers 1-44 and 46- | Markers 1-44 and 46-75, |
| 45” | 46-75. | 46-75, excluding | 75, excluding the | excluding the second, |
| Abbreviation: #NRBC | | the second marker. | second and third | third, and fourth markers. |
| | | | markers. | |
| Measured HGB = “Marker | Markers 1-45 and | Markers 1-45 and | Markers 1-45 and 47- | Markers 1-45 and 47-75, |
| 46” | 47-75. | 47-75, excluding | 75, excluding the | excluding the second, |
| Abbreviation: MHGB | | the second marker. | second and third | third, and fourth markers. |
| | | | markers. | |
| Normochromic/Normocytic | Markers 1-46 and | Markers 1-46 and | Markers 1-46 and 48- | Markers 1-46 and 48-75, |
| RBC count = “Marker 47” | 48-75. | 48-75, excluding | 75, excluding the | excluding the second, |
| Abbreviation: #NNRBC | | the second marker. | second and third | third, and fourth markers. |
| | | | markers. | |
| Platelet count = “Marker | Markers 1-47 and | Markers 1-47 and | Markers 1-47 and 49- | Markers 1-47 and 49-75, |
| 48” | 49-75. | 49-75, excluding | 75, excluding the | excluding the second, |
| Abbreviation: PLT | | the second marker. | second and third | third, and fourth markers. |
| | | | markers. | |
| Mean platelet volume = | Markers 1-48 and | Markers 1-48 and | Markers 1-48 and 50- | Markers 1-48 and 50-75, |
| “Marker 49” | 50-75. | 50-75, excluding | 75, excluding the | excluding the second, |
| Abbreviation: MPC | | the second marker. | second and third | third, and fourth markers. |
| | | | markers. | |
| Platelet distribution width = | Markers 1-49 and | Markers 1-49 and | Markers 1-49 and 51- | Markers 1-49 and 51-75, |
| “Marker 50” | 51-75. | 51-75, excluding | 75, excluding the | excluding the second, |
| Abbreviation: PDW | | the second marker. | second and third | third, and fourth markers. |
| | | | markers. | |
| Plateletcrit = “Marker 51” | Markers 1-50 and | Markers 1-50 and | Markers 1-50 and 52- | Markers 1-50 and 52-75, |
| Abbreviation: PCT | 52-75. | 52-75, excluding | 75, excluding the | excluding the second, |
| | | the second marker. | second and third | third, and fourth markers. |
| | | | markers. | |
| Mean platelet concentration = | Markers 1-51 and | Markers 1-51 and | Markers 1-51 and 53- | Markers 1-51 and 53-75, |
| “Marker 52” | 53-75. | 53-75, excluding | 75, excluding the | excluding the second, |
| Abbreviation: MPC | | the second marker. | second and third | third, and fourth markers. |
| | | | markers. | |
| Large platelets = “Marker | Markers 1-52 and | Markers 1-52 and | Markers 1-52 and 54- | Markers 1-52 and 54-75, |
| 53” | 54-75. | 54-75, excluding | 75, excluding the | excluding the second, |
| Abbreviation: #L-PLT | | the second marker. | second and third | third, and fourth markers. |
| | | | markers. | |
| Platelet clumps = “Marker | Markers 1-53 and | Markers 1-53 and | Markers 1-53 and 55- | Markers 1-53 and 55-75, |
| 54” | 55-75. | 55-75, excluding | 75, excluding the | excluding the second, |
| Abbreviation: PLT CLU | | the second marker. | second and third | third, and fourth markers. |
| | | | markers. | |
| Platelet conc. distribution | Markers 1-54 and | Markers 1-54 and | Markers 1-54 and 56- | Markers 1-54 and 56-75, |
| width = “Marker 55” | 56-75. | 56-75, excluding | 75, excluding the | excluding the second, |
| Abbreviation: PCDW | | the second marker. | second and third | third, and fourth markers. |
| | | | markers. | |
| Age = “Marker 56” | Markers 1-55. | Markers 1-55 and | Markers 1-55 and 57- | Markers 1-55 and 57-75, |
| | | 57-75, excluding | 75, excluding the | excluding the second, |
| | | the second marker. | second and third | third, and fourth markers. |
| | | | markers. | |
| Gender = “Marker 57” | Markers 1-55. | Markers 1-56 and | Markers 1-56 and 58- | Markers 1-56 and 58-75, |
| | | 58-75, excluding | 75, excluding the | excluding the second, |
| | | the second marker. | second and third | third, and fourth markers. |
| | | | markers. | |
| History of Hypertension = | Markers 1-55. | Markers 1-57 and | Markers 1-57 and 59- | Markers 1-57 and 59-75, |
| “Marker 58” | | 59-75, excluding | 75, excluding the | excluding the second, |
| | | the second marker. | second and third | third, and fourth markers. |
| | | | markers. | |
| Currently smoking = | Markers 1-55. | Markers 1-58 and | Markers 1-58 and 60- | Markers 1-58 and 60-75, |
| “Marker 59” | | 60-75, excluding | 75, excluding the | excluding the second, |
| | | the second marker. | second and third | third, and fourth markers. |
| | | | markers. | |
| History of smoking = | Markers 1-55. | Markers 1-59 and | Markers 1-59 and 61- | Markers 1-59 and 61-75, |
| “Marker 60” | | 61-75, excluding | 75, excluding the | excluding the second, |
| | | the second marker. | second and third | third, and fourth markers. |
| | | | markers. | |
| Diabetes mellitus status = | Markers 1-55. | Markers 1-60 and | Markers 1-60 and 62- | Markers 1-60 and 62-75, |
| “Marker 61” | | 62-75, excluding | 75, excluding the | excluding the second, |
| | | the second marker. | second and third | third, and fourth markers. |
| | | | markers. | |
| Fasting blood glucose level = | Markers 1-55. | Markers 1-61 and | Markers 1-61 and 63- | Markers 1-61 and 63-75, |
| “Marker 62” | | 63-75, excluding | 75, excluding the | excluding the second, |
| | | the second marker. | second and third | third, and fourth markers. |
| | | | markers. | |
| Creatinine level = “Marker | Markers 1-55. | Markers 1-62 and | Markers 1-62 and 64- | Markers 1-62 and 64-75, |
| 63” | | 64-75, excluding | 75, excluding the | excluding the second, |
| | | the second marker. | second and third | third, and fourth markers. |
| | | | markers. | |
| Potassium level = “Marker | Markers 1-55. | Markers 1-63 and | Markers 1-63 and 65- | Markers 1-63 and 65-75, |
| 64” | | 65-75, excluding | 75, excluding the | excluding the second, |
| | | the second marker. | second and third | third, and fourth markers. |
| | | | markers. | |
| C-reactive protein level = | Markers 1-55. | Markers 1-64 and | Markers 1-64 and 66- | Markers 1-64 and 66-75, |
| “Marker 65” | | 66-75, excluding | 75, excluding the | excluding the second, |
| | | the second marker. | second and third | third, and fourth markers. |
| | | | markers. | |
| Total cholesterol level = | Markers 1-55. | Markers 1-65 and | Markers 1-65 and 67- | Markers 1-65 and 67-75, |
| “Marker 66” | | 67-75, excluding | 75, excluding the | excluding the second, |
| | | the second marker. | second and third | third, and fourth markers. |
| | | | markers. | |
| LDL cholesterol level = | Markers 1-55. | Markers 1-66 and | Markers 1-66 and 68- | Markers 1-66 and 68-75, |
| “Marker 67” | | 68-75, excluding | 75, excluding the | excluding the second, |
| | | the second marker. | second and third | third, and fourth markers. |
| | | | markers. | |
| HDL cholesterol level = | Markers 1-55. | Markers 1-67 and | Markers 1-67 and 69- | Markers 1-67 and 69-75, |
| “Marker 68” | | 69-75, excluding | 75, excluding the | excluding the second, |
| | | the second marker. | second and third | third, and fourth markers. |
| | | | markers. | |
| Triglycerides level = | Markers 1-55. | Markers 1-68 and | Markers 1-68 and 70- | Markers 1-68 and 70-75, |
| “Marker 69” | | 70-75, excluding | 75, excluding the | excluding the second, |
| | | the second marker. | second and third | third, and fourth markers. |
| | | | markers. | |
| Systolic blood pressure = | Markers 1-55. | Markers 1-69 and | Markers 1-69 and 71- | Markers 1-69 and 71-75, |
| “Marker 70” | | 71-75, excluding | 75, excluding the | excluding the second, |
| | | the second marker. | second and third | third, and fourth markers. |
| | | | markers. | |
| Diastolic blood pressure = | Markers 1-55. | Markers 1-70 and | Markers 1-70 and 72- | Markers 1-70 and 72-75, |
| “Marker 71” | | 72-75, excluding | 75, excluding the | excluding the second, |
| | | the second marker. | second and third | third, and fourth markers. |
| | | | markers. | |
| Body mass index = | Markers 1-55. | Markers 1-71 and | Markers 1-71 and 73- | Markers 1-71 and 73-75, |
| “Marker 72” | | 73-75, excluding | 75, excluding the | excluding the second, |
| | | the second marker. | second and third | third, and fourth markers. |
| | | | markers. | |
| Aspirin use status = | Markers 1-55. | Markers 1-72 and | Markers 1-72 and 74- | Markers 1-72 and 74-75, |
| “Marker 73” | | 74-75, excluding | 75, excluding the | excluding the second, |
| | | the second marker. | second and third | third, and fourth markers. |
| | | | markers. | |
| Statin use status = “Marker | Markers 1-55. | Markers 1-73 and | Markers 1-73 and 75, | Markers 1-73 and 75, |
| 74” | | 75, excluding the | excluding the second | excluding the second, |
| | | second marker. | and third markers. | third, and fourth markers. |
| History of Cardiovascular | Markers 1-55. | Markers 1-74, | Markers 1-74, | Markers 1-74, excluding |
| Disease = “Marker 75” | | excluding the | excluding the second | the second, third, and |
| | | second marker. | and third markers. | fourth markers. |

Table 50 shows various combinations of Markers 1-55 with one or more markers 1-75, up to combinations of five markers. It is noted that the present invention is not limited to combinations of markers comprising or consisting of five markers. Instead, any and all combinations of markers from Table 50 may be made which include, for example, groups (comprising or consisting of) six markers, seven markers, eight markers, nine markers, ten markers . . . fifteen markers . . . twenty markers . . . thirty markers . . . fifty markers . . . and seventy five markers.

Examples of combinations of groups of two markers, provided in written out format, for every combination of two markers is shown below in Table 51. These combinations represent both groups that consist of these markers, as well as open-ended groups that comprise these sets of markers.

TABLE 51

| No | Marker 1 | Marker 2 |
|---|---|---|
| 1 | WBC | NT% |
| 2 | WBC | LM% |
| 3 | WBC | MN% |
| 4 | WBC | ES% |
| 5 | WBC | BS% |
| 6 | WBC | LUC% |
| 7 | WBC | #NEUT |
| 8 | WBC | #LYMPH |
| 9 | WBC | #MONO |
| 10 | WBC | #EOS |
| 11 | WBC | #BASO |
| 12 | WBC | #LUC |
| 13 | WBC | KY |
| 14 | WBC | #PERO SAT |
| 15 | WBC | LUC |
| 16 | WBC | LM |
| 17 | WBC | PXDD |
| 18 | WBC | PXYSIG |
| 19 | WBC | PXXSIG |
| 20 | WBC | PXY |
| 21 | WBC | %BLASTS |
| 22 | WBC | #BLST |
| 23 | WBC | MNX |
| 24 | WBC | MNY |
| 25 | WBC | MNPMN |
| 26 | WBC | NEUTX |
| 27 | WBC | NEUTY |
| 28 | WBC | LI |
| 29 | WBC | PMR |
| 30 | WBC | PMNX |
| 31 | WBC | RBC |
| 32 | WBC | HCT |
| 33 | WBC | MCV |
| 34 | WBC | MCH |
| 35 | WBC | MCHC |
| 36 | WBC | CHCM |
| 37 | WBC | RDW |
| 38 | WBC | HDW |
| 39 | WBC | HCDW |
| 40 | WBC | #MACRO |
| 41 | WBC | #HYPO |
| 42 | WBC | #HYPE |
| 43 | WBC | #MRBC |
| 44 | WBC | #NRBC |
| 45 | WBC | MHGB |
| 46 | WBC | #NNRBC |
| 47 | WBC | PLT |
| 48 | WBC | MPC |
| 49 | WBC | PDW |
| 50 | WBC | PCT |
| 51 | WBC | MPC |
| 52 | WBC | #L-PLT |
| 53 | WBC | PLT CLU |
| 54 | WBC | PCDW |
| 55 | NT% | LM% |
| 56 | NT% | MN% |
| 57 | NT% | ES% |
| 58 | NT% | BS% |
| 59 | NT% | LUC% |
| 60 | NT% | #NEUT |
| 61 | NT% | #LYMPH |
| 62 | NT% | #MONO |
| 63 | NT% | #EOS |
| 64 | NT% | #BASO |
| 65 | NT% | #LUC |
| 66 | NT% | KY |
| 67 | NT% | #PERO SAT |
| 68 | NT% | LUC |
| 69 | NT% | LM |
| 70 | NT% | PXDD |
| 71 | NT% | PXYSIG |
| 72 | NT% | PXXSIG |
| 73 | NT% | PXY |
| 74 | NT% | %BLASTS |
| 75 | NT% | #BLST |
| 76 | NT% | MNX |
| 77 | NT% | MNY |
| 78 | NT% | MNPMN |
| 79 | NT% | NEUTX |
| 80 | NT% | NEUTY |
| 81 | NT% | LI |
| 82 | NT% | PMR |
| 83 | NT% | PMNX |
| 84 | NT% | RBC |
| 85 | NT% | HCT |
| 86 | NT% | MCV |
| 87 | NT% | MCH |
| 88 | NT% | MCHC |
| 89 | NT% | CHCM |
| 90 | NT% | RDW |
| 91 | NT% | HDW |
| 92 | NT% | HCDW |
| 93 | NT% | #MACRO |
| 94 | NT% | #HYPO |
| 95 | NT% | #HYPE |
| 96 | NT% | #MRBC |
| 97 | NT% | #NRBC |
| 98 | NT% | MHGB |
| 99 | NT% | #NNRBC |
| 100 | NT% | PLT |
| 101 | NT% | MPC |
| 102 | NT% | PDW |
| 103 | NT% | PCT |
| 104 | NT% | MPC |
| 105 | NT% | #L-PLT |
| 106 | NT% | PLT CLU |
| 107 | NT% | PCDW |
| 108 | LM% | MN% |
| 109 | LM% | ES% |
| 110 | LM% | BS% |
| 111 | LM% | LUC% |
| 112 | LM% | #NEUT |
| 113 | LM% | #LYMPH |
| 114 | LM% | #MONO |
| 115 | LM% | #EOS |
| 116 | LM% | #BASO |
| 117 | LM% | #LUC |
| 118 | LM% | KY |
| 119 | LM% | #PERO SAT |
| 120 | LM% | LUC |
| 121 | LM% | LM |
| 122 | LM% | PXDD |
| 123 | LM% | PXYSIG |
| 124 | LM% | PXXSIG |
| 125 | LM% | PXY |
| 126 | LM% | %BLASTS |
| 127 | LM% | #BLST |
| 128 | LM% | MNX |
| 129 | LM% | MNY |
| 130 | LM% | MNPMN |
| 131 | LM% | NEUTX |
| 132 | LM% | NEUTY |
| 133 | LM% | LI |
| 134 | LM% | PMR |
| 135 | LM% | PMNX |
| 136 | LM% | RBC |
| 137 | LM% | HCT |
| 138 | LM% | MCV |
| 139 | LM% | MCH |
| 140 | LM% | MCHC |
| 141 | LM% | CHCM |
| 142 | LM% | RDW |
| 143 | LM% | HDW |
| 144 | LM% | HCDW |
| 145 | LM% | #MACRO |
| 146 | LM% | #HYPO |
| 147 | LM% | #HYPE |
| 148 | LM% | #MRBC |
| 149 | LM% | #NRBC |
| 150 | LM% | MHGB |
| 151 | LM% | #NNRBC |
| 152 | LM% | PLT |
| 153 | LM% | MPC |
| 154 | LM% | PDW |
| 155 | LM% | PCT |
| 156 | LM% | MPC |
| 157 | LM% | #L-PLT |
| 158 | LM% | PLT CLU |
| 159 | LM% | PCDW |
| 160 | MN% | ES% |
| 161 | MN% | BS% |
| 162 | MN% | LUC% |
| 163 | MN% | #NEUT |
| 164 | MN% | #LYMPH |
| 165 | MN% | #MONO |
| 166 | MN% | #EOS |
| 167 | MN% | #BASO |
| 168 | MN% | #LUC |
| 169 | MN% | KY |
| 170 | MN% | #PERO SAT |
| 171 | MN% | LUC |
| 172 | MN% | LM |
| 173 | MN% | PXDD |
| 174 | MN% | PXYSIG |
| 175 | MN% | PXXSIG |
| 176 | MN% | PXY |
| 177 | MN% | %BLASTS |
| 178 | MN% | #BLST |
| 179 | MN% | MNX |
| 180 | MN% | MNY |
| 181 | MN% | MNPMN |
| 182 | MN% | NEUTX |
| 183 | MN% | NEUTY |
| 184 | MN% | LI |
| 185 | MN% | PMR |
| 186 | MN% | PMNX |
| 187 | MN% | RBC |
| 188 | MN% | HCT |
| 189 | MN% | MCV |
| 190 | MN% | MCH |
| 191 | MN% | MCHC |
| 192 | MN% | CHCM |
| 193 | MN% | RDW |
| 194 | MN% | HDW |
| 195 | MN% | HCDW |
| 196 | MN% | #MACRO |
| 197 | MN% | #HYPO |
| 198 | MN% | #HYPE |
| 199 | MN% | #MRBC |
| 200 | MN% | #NRBC |
| 201 | MN% | MHGB |
| 202 | MN% | #NNRBC |
| 203 | MN% | PLT |
| 204 | MN% | MPC |
| 205 | MN% | PDW |
| 206 | MN% | PCT |
| 207 | MN% | MPC |
| 208 | MN% | #L-PLT |
| 209 | MN% | PLT CLU |
| 210 | MN% | PCDW |
| 211 | ES% | BS% |
| 212 | ES% | LUC% |
| 213 | ES% | #NEUT |
| 214 | ES% | #LYMPH |
| 215 | ES% | #MONO |
| 216 | ES% | #EOS |
| 217 | ES% | #BASO |
| 218 | ES% | #LUC |
| 219 | ES% | KY |
| 220 | ES% | #PERO SAT |
| 221 | ES% | LUC |
| 222 | ES% | LM |
| 223 | ES% | PXDD |
| 224 | ES% | PXYSIG |
| 225 | ES% | PXXSIG |
| 226 | ES% | PXY |
| 227 | ES% | %BLASTS |
| 228 | ES% | #BLST |
| 229 | ES% | MNX |
| 230 | ES% | MNY |
| 231 | ES% | MNPMN |
| 232 | ES% | NEUTX |
| 233 | ES% | NEUTY |
| 234 | ES% | LI |
| 235 | ES% | PMR |
| 236 | ES% | PMNX |
| 237 | ES% | RBC |
| 238 | ES% | HCT |
| 239 | ES% | MCV |
| 240 | ES% | MCH |
| 241 | ES% | MCHC |
| 242 | ES% | CHCM |
| 243 | ES% | RDW |
| 244 | ES% | HDW |
| 245 | ES% | HCDW |
| 246 | ES% | #MACRO |
| 247 | ES% | #HYPO |
| 248 | ES% | #HYPE |
| 249 | ES% | #MRBC |
| 250 | ES% | #NRBC |
| 251 | ES% | MHGB |
| 252 | ES% | #NNRBC |
| 253 | ES% | PLT |
| 254 | ES% | MPC |
| 255 | ES% | PDW |
| 256 | ES% | PCT |
| 257 | ES% | MPC |
| 258 | ES% | #L-PLT |
| 259 | ES% | PLT CLU |
| 260 | ES% | PCDW |
| 261 | BS% | LUC% |
| 262 | BS% | #NEUT |
| 263 | BS% | #LYMPH |
| 264 | BS% | #MONO |
| 265 | BS% | #EOS |
| 266 | BS% | #BASO |
| 267 | BS% | #LUC |
| 268 | BS% | KY |
| 269 | BS% | #PERO SAT |
| 270 | BS% | LUC |
| 271 | BS% | LM |
| 272 | BS% | PXDD |
| 273 | BS% | PXYSIG |
| 274 | BS% | PXXSIG |
| 275 | BS% | PXY |
| 276 | BS% | %BLASTS |
| 277 | BS% | #BLST |
| 278 | BS% | MNX |
| 279 | BS% | MNY |
| 280 | BS% | MNPMN |
| 281 | BS% | NEUTX |
| 282 | BS% | NEUTY |
| 283 | BS% | LI |
| 284 | BS% | PMR |
| 285 | BS% | PMNX |
| 286 | BS% | RBC |
| 287 | BS% | HCT |
| 288 | BS% | MCV |
| 289 | BS% | MCH |
| 290 | BS% | MCHC |
| 291 | BS% | CHCM |
| 292 | BS% | RDW |
| 293 | BS% | HDW |
| 294 | BS% | HCDW |
| 295 | BS% | #MACRO |
| 296 | BS% | #HYPO |
| 297 | BS% | #HYPE |
| 298 | BS% | #MRBC |
| 299 | BS% | #NRBC |
| 300 | BS% | MHGB |
| 301 | BS% | #NNRBC |
| 302 | BS% | PLT |
| 303 | BS% | MPC |
| 304 | BS% | PDW |
| 305 | BS% | PCT |
| 306 | BS% | MPC |
| 307 | BS% | #L-PLT |
| 308 | BS% | PLT CLU |
| 309 | BS% | PCDW |
| 310 | LUC% | #NEUT |
| 311 | LUC% | #LYMPH |
| 312 | LUC% | #MONO |
| 313 | LUC% | #EOS |
| 314 | LUC% | #BASO |
| 315 | LUC% | #LUC |
| 316 | LUC% | KY |
| 317 | LUC% | #PERO SAT |
| 318 | LUC% | LUC |
| 319 | LUC% | LM |
| 320 | LUC% | PXDD |
| 321 | LUC% | PXYSIG |
| 322 | LUC% | PXXSIG |
| 323 | LUC% | PXY |
| 324 | LUC% | %BLASTS |
| 325 | LUC% | #BLST |
| 326 | LUC% | MNX |
| 327 | LUC% | MNY |
| 328 | LUC% | MNPMN |
| 329 | LUC% | NEUTX |
| 330 | LUC% | NEUTY |
| 331 | LUC% | LI |
| 332 | LUC% | PMR |
| 333 | LUC% | PMNX |
| 334 | LUC% | RBC |
| 335 | LUC% | HCT |
| 336 | LUC% | MCV |
| 337 | LUC% | MCH |
| 338 | LUC% | MCHC |
| 339 | LUC% | CHCM |
| 340 | LUC% | RDW |
| 341 | LUC% | HDW |
| 342 | LUC% | HCDW |
| 343 | LUC% | #MACRO |
| 344 | LUC% | #HYPO |
| 345 | LUC% | #HYPE |
| 346 | LUC% | #MRBC |
| 347 | LUC% | #NRBC |
| 348 | LUC% | MHGB |
| 349 | LUC% | #NNRBC |
| 350 | LUC% | PLT |
| 351 | LUC% | MPC |
| 352 | LUC% | PDW |
| 353 | LUC% | PCT |
| 354 | LUC% | MPC |
| 355 | LUC% | #L-PLT |
| 356 | LUC% | PLT CLU |
| 357 | LUC% | PCDW |
| 358 | #NEUT | #LYMPH |
| 359 | #NEUT | #MONO |
| 360 | #NEUT | #EOS |
| 361 | #NEUT | #BASO |
| 362 | #NEUT | #LUC |
| 363 | #NEUT | KY |
| 364 | #NEUT | #PERO SAT |
| 365 | #NEUT | LUC |
| 366 | #NEUT | LM |
| 367 | #NEUT | PXDD |
| 368 | #NEUT | PXYSIG |
| 369 | #NEUT | PXXSIG |
| 370 | #NEUT | PXY |
| 371 | #NEUT | %BLASTS |
| 372 | #NEUT | #BLST |
| 373 | #NEUT | MNX |
| 374 | #NEUT | MNY |
| 375 | #NEUT | MNPMN |
| 376 | #NEUT | NEUTX |
| 377 | #NEUT | NEUTY |
| 378 | #NEUT | LI |
| 379 | #NEUT | PMR |
| 380 | #NEUT | PMNX |
| 381 | #NEUT | RBC |
| 382 | #NEUT | HCT |
| 383 | #NEUT | MCV |
| 384 | #NEUT | MCH |
| 385 | #NEUT | MCHC |
| 386 | #NEUT | CHCM |
| 387 | #NEUT | RDW |
| 388 | #NEUT | HDW |
| 389 | #NEUT | HCDW |
| 390 | #NEUT | #MACRO |
| 391 | #NEUT | #HYPO |
| 392 | #NEUT | #HYPE |
| 393 | #NEUT | #MRBC |
| 394 | #NEUT | #NRBC |
| 395 | #NEUT | MHGB |
| 396 | #NEUT | #NNRBC |
| 397 | #NEUT | PLT |
| 398 | #NEUT | MPC |
| 399 | #NEUT | PDW |
| 400 | #NEUT | PCT |
| 401 | #NEUT | MPC |
| 402 | #NEUT | #L-PLT |
| 403 | #NEUT | PLT CLU |
| 404 | #NEUT | PCDW |
| 405 | #LYMPH | #MONO |
| 406 | #LYMPH | #EOS |
| 407 | #LYMPH | #BASO |
| 408 | #LYMPH | #LUC |
| 409 | #LYMPH | KY |
| 410 | #LYMPH | #PERO SAT |
| 411 | #LYMPH | LUC |
| 412 | #LYMPH | LM |
| 413 | #LYMPH | PXDD |
| 414 | #LYMPH | PXYSIG |
| 415 | #LYMPH | PXXSIG |
| 416 | #LYMPH | PXY |
| 417 | #LYMPH | %BLASTS |
| 418 | #LYMPH | #BLST |
| 419 | #LYMPH | MNX |
| 420 | #LYMPH | MNY |
| 421 | #LYMPH | MNPMN |
| 422 | #LYMPH | NEUTX |
| 423 | #LYMPH | NEUTY |
| 424 | #LYMPH | LI |
| 425 | #LYMPH | PMR |
| 426 | #LYMPH | PMNX |
| 427 | #LYMPH | RBC |
| 428 | #LYMPH | HCT |
| 429 | #LYMPH | MCV |
| 430 | #LYMPH | MCH |
| 431 | #LYMPH | MCHC |
| 432 | #LYMPH | CHCM |
| 433 | #LYMPH | RDW |
| 434 | #LYMPH | HDW |
| 435 | #LYMPH | HCDW |
| 436 | #LYMPH | #MACRO |
| 437 | #LYMPH | #HYPO |
| 438 | #LYMPH | #HYPE |
| 439 | #LYMPH | #MRBC |
| 440 | #LYMPH | #NRBC |
| 441 | #LYMPH | MHGB |
| 442 | #LYMPH | #NNRBC |
| 443 | #LYMPH | PLT |
| 444 | #LYMPH | MPC |
| 445 | #LYMPH | PDW |
| 446 | #LYMPH | PCT |
| 447 | #LYMPH | MPC |
| 448 | #LYMPH | #L-PLT |
| 449 | #LYMPH | PLT CLU |
| 450 | #LYMPH | PCDW |
| 451 | #MONO | #EOS |
| 452 | #MONO | #BASO |
| 453 | #MONO | #LUC |
| 454 | #MONO | KY |
| 455 | #MONO | #PERO SAT |
| 456 | #MONO | LUC |
| 457 | #MONO | LM |
| 458 | #MONO | PXDD |
| 459 | #MONO | PXYSIG |
| 460 | #MONO | PXXSIG |
| 461 | #MONO | PXY |
| 462 | #MONO | %BLASTS |
| 463 | #MONO | #BLST |
| 464 | #MONO | MNX |
| 465 | #MONO | MNY |
| 466 | #MONO | MNPMN |
| 467 | #MONO | NEUTX |
| 468 | #MONO | NEUTY |
| 469 | #MONO | LI |
| 470 | #MONO | PMR |
| 471 | #MONO | PMNX |
| 472 | #MONO | RBC |
| 473 | #MONO | HCT |
| 474 | #MONO | MCV |
| 475 | #MONO | MCH |
| 476 | #MONO | MCHC |
| 477 | #MONO | CHCM |
| 478 | #MONO | RDW |
| 479 | #MONO | HDW |
| 480 | #MONO | HCDW |
| 481 | #MONO | #MACRO |
| 482 | #MONO | #HYPO |
| 483 | #MONO | #HYPE |
| 484 | #MONO | #MRBC |
| 485 | #MONO | #NRBC |
| 486 | #MONO | MHGB |
| 487 | #MONO | #NNRBC |
| 488 | #MONO | PLT |
| 489 | #MONO | MPC |
| 490 | #MONO | PDW |
| 491 | #MONO | PCT |
| 492 | #MONO | MPC |
| 493 | #MONO | #L-PLT |
| 494 | #MONO | PLT CLU |
| 495 | #MONO | PCDW |
| 496 | #EOS | #BASO |
| 497 | #EOS | #LUC |
| 498 | #EOS | KY |
| 499 | #EOS | #PERO SAT |
| 500 | #EOS | LUC |
| 501 | #EOS | LM |
| 502 | #EOS | PXDD |
| 503 | #EOS | PXYSIG |
| 504 | #EOS | PXXSIG |
| 505 | #EOS | PXY |
| 506 | #EOS | %BLASTS |
| 507 | #EOS | #BLST |
| 508 | #EOS | MNX |
| 509 | #EOS | MNY |
| 510 | #EOS | MNPMN |
| 511 | #EOS | NEUTX |
| 512 | #EOS | NEUTY |
| 513 | #EOS | LI |
| 514 | #EOS | PMR |
| 515 | #EOS | PMNX |
| 516 | #EOS | RBC |
| 517 | #EOS | HCT |
| 518 | #EOS | MCV |
| 519 | #EOS | MCH |
| 520 | #EOS | MCHC |
| 521 | #EOS | CHCM |
| 522 | #EOS | RDW |
| 523 | #EOS | HDW |
| 524 | #EOS | HCDW |
| 525 | #EOS | #MACRO |
| 526 | #EOS | #HYPO |
| 527 | #EOS | #HYPE |
| 528 | #EOS | #MRBC |
| 529 | #EOS | #NRBC |
| 530 | #EOS | MHGB |
| 531 | #EOS | #NNRBC |
| 532 | #EOS | PLT |
| 533 | #EOS | MPC |
| 534 | #EOS | PDW |
| 535 | #EOS | PCT |
| 536 | #EOS | MPC |
| 537 | #EOS | #L-PLT |
| 538 | #EOS | PLT CLU |
| 539 | #EOS | PCDW |
| 540 | #BASO | #LUC |
| 541 | #BASO | KY |
| 542 | #BASO | #PERO SAT |
| 543 | #BASO | LUC |
| 544 | #BASO | LM |
| 545 | #BASO | PXDD |
| 546 | #BASO | PXYSIG |
| 547 | #BASO | PXXSIG |
| 548 | #BASO | PXY |
| 549 | #BASO | %BLASTS |
| 550 | #BASO | #BLST |
| 551 | #BASO | MNX |
| 552 | #BASO | MNY |
| 553 | #BASO | MNPMN |
| 554 | #BASO | NEUTX |
| 555 | #BASO | NEUTY |
| 556 | #BASO | LI |
| 557 | #BASO | PMR |
| 558 | #BASO | PMNX |
| 559 | #BASO | RBC |
| 560 | #BASO | HCT |
| 561 | #BASO | MCV |
| 562 | #BASO | MCH |
| 563 | #BASO | MCHC |
| 564 | #BASO | CHCM |
| 565 | #BASO | RDW |
| 566 | #BASO | HDW |
| 567 | #BASO | HCDW |
| 568 | #BASO | #MACRO |
| 569 | #BASO | #HYPO |
| 570 | #BASO | #HYPE |
| 571 | #BASO | #MRBC |
| 572 | #BASO | #NRBC |
| 573 | #BASO | MHGB |
| 574 | #BASO | #NNRBC |
| 575 | #BASO | PLT |
| 576 | #BASO | MPC |
| 577 | #BASO | PDW |
| 578 | #BASO | PCT |
| 579 | #BASO | MPC |
| 580 | #BASO | #L-PLT |
| 581 | #BASO | PLT CLU |
| 582 | #BASO | PCDW |
| 583 | #LUC | KY |
| 584 | #LUC | #PERO SAT |
| 585 | #LUC | LUC |
| 586 | #LUC | LM |
| 587 | #LUC | PXDD |
| 588 | #LUC | PXYSIG |
| 589 | #LUC | PXXSIG |
| 590 | #LUC | PXY |
| 591 | #LUC | %BLASTS |
| 592 | #LUC | #BLST |
| 593 | #LUC | MNX |
| 594 | #LUC | MNY |
| 595 | #LUC | MNPMN |
| 596 | #LUC | NEUTX |
| 597 | #LUC | NEUTY |
| 598 | #LUC | LI |
| 599 | #LUC | PMR |
| 600 | #LUC | PMNX |
| 601 | #LUC | RBC |
| 602 | #LUC | HCT |
| 603 | #LUC | MCV |
| 604 | #LUC | MCH |
| 605 | #LUC | MCHC |
| 606 | #LUC | CHCM |
| 607 | #LUC | RDW |
| 608 | #LUC | HDW |
| 609 | #LUC | HCDW |
| 610 | #LUC | #MACRO |
| 611 | #LUC | #HYPO |
| 612 | #LUC | #HYPE |
| 613 | #LUC | #MRBC |
| 614 | #LUC | #NRBC |
| 615 | #LUC | MHGB |
| 616 | #LUC | #NNRBC |
| 617 | #LUC | PLT |
| 618 | #LUC | MPC |
| 619 | #LUC | PDW |
| 620 | #LUC | PCT |
| 621 | #LUC | MPC |
| 622 | #LUC | #L-PLT |
| 623 | #LUC | PLT CLU |
| 624 | #LUC | PCDW |
| 625 | KY | #PERO SAT |
| 626 | KY | LUC |
| 627 | KY | LM |
| 628 | KY | PXDD |
| 629 | KY | PXYSIG |
| 630 | KY | PXXSIG |
| 631 | KY | PXY |
| 632 | KY | %BLASTS |
| 633 | KY | #BLST |
| 634 | KY | MNX |
| 635 | KY | MNY |
| 636 | KY | MNPMN |
| 637 | KY | NEUTX |
| 638 | KY | NEUTY |
| 639 | KY | LI |
| 640 | KY | PMR |
| 641 | KY | PMNX |
| 642 | KY | RBC |
| 643 | KY | HCT |
| 644 | KY | MCV |
| 645 | KY | MCH |
| 646 | KY | MCHC |
| 647 | KY | CHCM |
| 648 | KY | RDW |
| 649 | KY | HDW |
| 650 | KY | HCDW |
| 651 | KY | #MACRO |
| 652 | KY | #HYPO |
| 653 | KY | #HYPE |
| 654 | KY | #MRBC |
| 655 | KY | #NRBC |
| 656 | KY | MHGB |
| 657 | KY | #NNRBC |
| 658 | KY | PLT |
| 659 | KY | MPC |
| 660 | KY | PDW |
| 661 | KY | PCT |
| 662 | KY | MPC |
| 663 | KY | #L-PLT |
| 664 | KY | PLT CLU |
| 665 | KY | PCDW |
| 666 | #PERO SAT | LUC |
| 667 | #PERO SAT | LM |
| 668 | #PERO SAT | PXDD |
| 669 | #PERO SAT | PXYSIG |
| 670 | #PERO SAT | PXXSIG |
| 671 | #PERO SAT | PXY |
| 672 | #PERO SAT | %BLASTS |
| 673 | #PERO SAT | #BLST |
| 674 | #PERO SAT | MNX |
| 675 | #PERO SAT | MNY |
| 676 | #PERO SAT | MNPMN |
| 677 | #PERO SAT | NEUTX |
| 678 | #PERO SAT | NEUTY |
| 679 | #PERO SAT | LI |
| 680 | #PERO SAT | PMR |
| 681 | #PERO SAT | PMNX |
| 682 | #PERO SAT | RBC |
| 683 | #PERO SAT | HCT |
| 684 | #PERO SAT | MCV |
| 685 | #PERO SAT | MCH |
| 686 | #PERO SAT | MCHC |
| 687 | #PERO SAT | CHCM |
| 688 | #PERO SAT | RDW |
| 689 | #PERO SAT | HDW |
| 690 | #PERO SAT | HCDW |
| 691 | #PERO SAT | #MACRO |
| 692 | #PERO SAT | #HYPO |
| 693 | #PERO SAT | #HYPE |
| 694 | #PERO SAT | #MRBC |
| 695 | #PERO SAT | #NRBC |
| 696 | #PERO SAT | MHGB |
| 697 | #PERO SAT | #NNRBC |
| 698 | #PERO SAT | PLT |
| 699 | #PERO SAT | MPC |
| 700 | #PERO SAT | PDW |
| 701 | #PERO SAT | PCT |
| 702 | #PERO SAT | MPC |
| 703 | #PERO SAT | #L-PLT |
| 704 | #PERO SAT | PLT CLU |
| 705 | #PERO | SATPCDW |
| 706 | LUC | LM |
| 707 | LUC | PXDD |
| 708 | LUC | PXYSIG |
| 709 | LUC | PXXSIG |
| 710 | LUC | PXY |
| 711 | LUC | %BLASTS |
| 712 | LUC | #BLST |
| 713 | LUC | MNX |
| 714 | LUC | MNY |
| 715 | LUC | MNPMN |
| 716 | LUC | NEUTX |
| 717 | LUC | NEUTY |
| 718 | LUC | LI |
| 719 | LUC | PMR |
| 720 | LUC | PMNX |
| 721 | LUC | RBC |
| 722 | LUC | HCT |
| 723 | LUC | MCV |
| 724 | LUC | MCH |
| 725 | LUC | MCHC |
| 726 | LUC | CHCM |
| 727 | LUC | RDW |
| 728 | LUC | HDW |
| 729 | LUC | HCDW |
| 730 | LUC | #MACRO |
| 731 | LUC | #HYPO |
| 732 | LUC | #HYPE |
| 733 | LUC | #MRBC |
| 734 | LUC | #NRBC |
| 735 | LUC | MHGB |
| 736 | LUC | #NNRBC |
| 737 | LUC | PLT |
| 738 | LUC | MPC |
| 739 | LUC | PDW |
| 740 | LUC | PCT |
| 741 | LUC | MPC |
| 742 | LUC | #L-PLT |
| 743 | LUC | PLT CLU |
| 744 | LUC | PCDW |
| 745 | LM | PXDD |
| 746 | LM | PXYSIG |
| 747 | LM | PXXSIG |
| 748 | LM | PXY |
| 749 | LM | %BLASTS |
| 750 | LM | #BLST |
| 751 | LM | MNX |
| 752 | LM | MNY |
| 753 | LM | MNPMN |
| 754 | LM | NEUTX |
| 755 | LM | NEUTY |
| 756 | LM | LI |
| 757 | LM | PMR |
| 758 | LM | PMNX |
| 759 | LM | RBC |
| 760 | LM | HCT |
| 761 | LM | MCV |
| 762 | LM | MCH |
| 763 | LM | MCHC |
| 764 | LM | CHCM |
| 765 | LM | RDW |
| 766 | LM | HDW |
| 767 | LM | HCDW |
| 768 | LM | #MACRO |
| 769 | LM | #HYPO |
| 770 | LM | #HYPE |
| 771 | LM | #MRBC |
| 772 | LM | #NRBC |
| 773 | LM | MHGB |
| 774 | LM | #NNRBC |
| 775 | LM | PLT |
| 776 | LM | MPC |
| 777 | LM | PDW |
| 778 | LM | PCT |
| 779 | LM | MPC |
| 780 | LM | #L-PLT |
| 781 | LM | PLT CLU |
| 782 | LM | PCDW |
| 783 | PXDD | PXYSIG |
| 784 | PXDD | PXXSIG |
| 785 | PXDD | PXY |
| 786 | PXDD | %BLASTS |
| 787 | PXDD | #BLST |
| 788 | PXDD | MNX |
| 789 | PXDD | MNY |
| 790 | PXDD | MNPMN |
| 791 | PXDD | NEUTX |
| 792 | PXDD | NEUTY |
| 793 | PXDD | LI |
| 794 | PXDD | PMR |
| 795 | PXDD | PMNX |
| 796 | PXDD | RBC |
| 797 | PXDD | HCT |
| 798 | PXDD | MCV |
| 799 | PXDD | MCH |
| 800 | PXDD | MCHC |
| 801 | PXDD | CHCM |
| 802 | PXDD | RDW |
| 803 | PXDD | HDW |
| 804 | PXDD | HCDW |
| 805 | PXDD | #MACRO |
| 806 | PXDD | #HYPO |
| 807 | PXDD | #HYPE |
| 808 | PXDD | #MRBC |
| 809 | PXDD | #NRBC |
| 810 | PXDD | MHGB |
| 811 | PXDD | #NNRBC |
| 812 | PXDD | PLT |
| 813 | PXDD | MPC |
| 814 | PXDD | PDW |
| 815 | PXDD | PCT |
| 816 | PXDD | MPC |
| 817 | PXDD | #L-PLT |
| 818 | PXDD | PLT CLU |
| 819 | PXDD | PCDW |
| 820 | PXYSIG | PXXSIG |
| 821 | PXYSIG | PXY |
| 822 | PXYSIG | %BLASTS |
| 823 | PXYSIG | #BLST |
| 824 | PXYSIG | MNX |
| 825 | PXYSIG | MNY |
| 826 | PXYSIG | MNPMN |
| 827 | PXYSIG | NEUTX |
| 828 | PXYSIG | NEUTY |
| 829 | PXYSIG | LI |
| 830 | PXYSIG | PMR |
| 831 | PXYSIG | PMNX |
| 832 | PXYSIG | RBC |
| 833 | PXYSIG | HCT |
| 834 | PXYSIG | MCV |
| 835 | PXYSIG | MCH |
| 836 | PXYSIG | MCHC |
| 837 | PXYSIG | CHCM |
| 838 | PXYSIG | RDW |
| 839 | PXYSIG | HDW |
| 840 | PXYSIG | HCDW |
| 841 | PXYSIG | #MACRO |
| 842 | PXYSIG | #HYPO |
| 843 | PXYSIG | #HYPE |
| 844 | PXYSIG | #MRBC |
| 845 | PXYSIG | #NRBC |
| 846 | PXYSIG | MHGB |
| 847 | PXYSIG | #NNRBC |
| 848 | PXYSIG | PLT |
| 849 | PXYSIG | MPC |
| 850 | PXYSIG | PDW |
| 851 | PXYSIG | PCT |
| 852 | PXYSIG | MPC |
| 853 | PXYSIG | #L-PLT |
| 854 | PXYSIG | PLT CLU |
| 855 | PXYSIG | PCDW |
| 856 | PXXSIG | PXY |
| 857 | PXXSIG | %BLASTS |
| 858 | PXXSIG | #BLST |
| 859 | PXXSIG | MNX |
| 860 | PXXSIG | MNY |
| 861 | PXXSIG | MNPMN |
| 862 | PXXSIG | NEUTX |
| 863 | PXXSIG | NEUTY |
| 864 | PXXSIG | LI |
| 865 | PXXSIG | PMR |
| 866 | PXXSIG | PMNX |
| 867 | PXXSIG | RBC |
| 868 | PXXSIG | HCT |
| 869 | PXXSIG | MCV |
| 870 | PXXSIG | MCH |
| 871 | PXXSIG | MCHC |
| 872 | PXXSIG | CHCM |
| 873 | PXXSIG | RDW |
| 874 | PXXSIG | HDW |
| 875 | PXXSIG | HCDW |
| 876 | PXXSIG | #MACRO |
| 877 | PXXSIG | #HYPO |
| 878 | PXXSIG | #HYPE |
| 879 | PXXSIG | #MRBC |
| 880 | PXXSIG | #NRBC |
| 881 | PXXSIG | MHGB |
| 882 | PXXSIG | #NNRBC |
| 883 | PXXSIG | PLT |
| 884 | PXXSIG | MPC |
| 885 | PXXSIG | PDW |
| 886 | PXXSIG | PCT |
| 887 | PXXSIG | MPC |
| 888 | PXXSIG | #L-PLT |
| 889 | PXXSIG | PLT CLU |
| 890 | PXXSIG | PCDW |
| 891 | PXY | %BLASTS |
| 892 | PXY | #BLST |
| 893 | PXY | MNX |
| 894 | PXY | MNY |
| 895 | PXY | MNPMN |
| 896 | PXY | NEUTX |
| 897 | PXY | NEUTY |
| 898 | PXY | LI |
| 899 | PXY | PMR |
| 900 | PXY | PMNX |
| 901 | PXY | RBC |
| 902 | PXY | HCT |
| 903 | PXY | MCV |
| 904 | PXY | MCH |
| 905 | PXY | MCHC |
| 906 | PXY | CHCM |
| 907 | PXY | RDW |
| 908 | PXY | HDW |
| 909 | PXY | HCDW |
| 910 | PXY | #MACRO |
| 911 | PXY | #HYPO |
| 912 | PXY | #HYPE |
| 913 | PXY | #MRBC |
| 914 | PXY | #NRBC |
| 915 | PXY | MHGB |
| 916 | PXY | #NNRBC |
| 917 | PXY | PLT |
| 918 | PXY | MPC |
| 919 | PXY | PDW |
| 920 | PXY | PCT |
| 921 | PXY | MPC |
| 922 | PXY | #L-PLT |
| 923 | PXY | PLT CLU |
| 924 | PXY | PCDW |
| 925 | %BLASTS | #BLST |
| 926 | %BLASTS | MNX |
| 927 | %BLASTS | MNY |
| 928 | %BLASTS | MNPMN |
| 929 | %BLASTS | NEUTX |
| 930 | %BLASTS | NEUTY |
| 931 | %BLASTS | LI |
| 932 | %BLASTS | PMR |
| 933 | %BLASTS | PMNX |
| 934 | %BLASTS | RBC |
| 935 | %BLASTS | HCT |
| 936 | %BLASTS | MCV |
| 937 | %BLASTS | MCH |
| 938 | %BLASTS | MCHC |
| 939 | %BLASTS | CHCM |
| 940 | %BLASTS | RDW |
| 941 | %BLASTS | HDW |
| 942 | %BLASTS | HCDW |
| 943 | %BLASTS | #MACRO |
| 944 | %BLASTS | #HYPO |
| 945 | %BLASTS | #HYPE |
| 946 | %BLASTS | #MRBC |
| 947 | %BLASTS | #NRBC |
| 948 | %BLASTS | MHGB |
| 949 | %BLASTS | #NNRBC |
| 950 | %BLASTS | PLT |
| 951 | %BLASTS | MPC |
| 952 | %BLASTS | PDW |
| 953 | %BLASTS | PCT |
| 954 | %BLASTS | MPC |
| 955 | %BLASTS | #L-PLT |
| 956 | %BLASTS | PLT CLU |
| 957 | %BLASTS | PCDW |
| 958 | #BLST | MNX |
| 959 | #BLST | MNY |
| 960 | #BLST | MNPMN |
| 961 | #BLST | NEUTX |
| 962 | #BLST | NEUTY |
| 963 | #BLST | LI |
| 964 | #BLST | PMR |
| 965 | #BLST | PMNX |
| 966 | #BLST | RBC |
| 967 | #BLST | HCT |
| 968 | #BLST | MCV |
| 969 | #BLST | MCH |
| 970 | #BLST | MCHC |
| 971 | #BLST | CHCM |
| 972 | #BLST | RDW |
| 973 | #BLST | HDW |
| 974 | #BLST | HCDW |
| 975 | #BLST | #MACRO |
| 976 | #BLST | #HYPO |
| 977 | #BLST | #HYPE |
| 978 | #BLST | #MRBC |
| 979 | #BLST | #NRBC |
| 980 | #BLST | MHGB |
| 981 | #BLST | #NNRBC |
| 982 | #BLST | PLT |
| 983 | #BLST | MPC |
| 984 | #BLST | PDW |
| 985 | #BLST | PCT |
| 986 | #BLST | MPC |
| 987 | #BLST | #L-PLT |
| 988 | #BLST | PLT CLU |
| 989 | #BLST | PCDW |
| 990 | MNX | MNY |
| 991 | MNX | MNPMN |
| 992 | MNX | NEUTX |
| 993 | MNX | NEUTY |
| 994 | MNX | LI |
| 995 | MNX | PMR |
| 996 | MNX | PMNX |
| 997 | MNX | RBC |
| 998 | MNX | HCT |
| 999 | MNX | MCV |
| 1000 | MNX | MCH |
| 1001 | MNX | MCHC |
| 1002 | MNX | CHCM |
| 1003 | MNX | RDW |
| 1004 | MNX | HDW |
| 1005 | MNX | HCDW |
| 1006 | MNX | #MACRO |
| 1007 | MNX | #HYPO |
| 1008 | MNX | #HYPE |
| 1009 | MNX | #MRBC |
| 1010 | MNX | #NRBC |
| 1011 | MNX | MHGB |
| 1012 | MNX | #NNRBC |
| 1013 | MNX | PLT |
| 1014 | MNX | MPC |
| 1015 | MNX | PDW |
| 1016 | MNX | PCT |
| 1017 | MNX | MPC |
| 1018 | MNX | #L-PLT |
| 1019 | MNX | PLT CLU |
| 1020 | MNX | PCDW |
| 1021 | MNY | MNPMN |
| 1022 | MNY | NEUTX |
| 1023 | MNY | NEUTY |
| 1024 | MNY | LI |
| 1025 | MNY | PMR |
| 1026 | MNY | PMNX |
| 1027 | MNY | RBC |
| 1028 | MNY | HCT |
| 1029 | MNY | MCV |
| 1030 | MNY | MCH |
| 1031 | MNY | MCHC |
| 1032 | MNY | CHCM |
| 1033 | MNY | RDW |
| 1034 | MNY | HDW |
| 1035 | MNY | HCDW |
| 1036 | MNY | #MACRO |
| 1037 | MNY | #HYPO |
| 1038 | MNY | #HYPE |
| 1039 | MNY | #MRBC |
| 1040 | MNY | #NRBC |
| 1041 | MNY | MHGB |
| 1042 | MNY | #NNRBC |
| 1043 | MNY | PLT |
| 1044 | MNY | MPC |
| 1045 | MNY | PDW |
| 1046 | MNY | PCT |
| 1047 | MNY | MPC |
| 1048 | MNY | #L-PLT |
| 1049 | MNY | PLT CLU |
| 1050 | MNY | PCDW |
| 1051 | MNPMN | NEUTX |
| 1052 | MNPMN | NEUTY |
| 1053 | MNPMN | LI |
| 1054 | MNPMN | PMR |
| 1055 | MNPMN | PMNX |
| 1056 | MNPMN | RBC |
| 1057 | MNPMN | HCT |
| 1058 | MNPMN | MCV |
| 1059 | MNPMN | MCH |
| 1060 | MNPMN | MCHC |
| 1061 | MNPMN | CHCM |
| 1062 | MNPMN | RDW |
| 1063 | MNPMN | HDW |
| 1064 | MNPMN | HCDW |
| 1065 | MNPMN | #MACRO |
| 1066 | MNPMN | #HYPO |
| 106 | 7MNPMN | #HYPE |
| 1068 | MNPMN | #MRBC |
| 1069 | MNPMN | #NRBC |
| 1070 | MNPMN | MHGB |
| 1071 | MNPMN | #NNRBC |
| 1072 | MNPMN | PLT |
| 1073 | MNPMN | MPC |
| 1074 | MNPMN | PDW |
| 1075 | MNPMN | PCT |
| 1076 | MNPMN | MPC |
| 1077 | MNPMN | #L-PLT |
| 1078 | MNPMN | PLT CLU |
| 1079 | MNPMN | PCDW |
| 1080 | NEUTX | NEUTY |
| 1081 | NEUTX | LI |
| 1082 | NEUTX | PMR |
| 1083 | NEUTX | PMNX |
| 1084 | NEUTX | RBC |
| 1085 | NEUTX | HCT |
| 1086 | NEUTX | MCV |
| 1087 | NEUTX | MCH |
| 1088 | NEUTX | MCHC |
| 1089 | NEUTX | CHCM |
| 1090 | NEUTX | RDW |
| 1091 | NEUTX | HDW |
| 1092 | NEUTX | HCDW |
| 1093 | NEUTX | #MACRO |
| 1094 | NEUTX | #HYPO |
| 1095 | NEUTX | #HYPE |
| 1096 | NEUTX | #MRBC |
| 1097 | NEUTX | #NRBC |
| 1098 | NEUTX | MHGB |
| 1099 | NEUTX | #NNRBC |
| 1100 | NEUTX | PLT |
| 1101 | NEUTX | MPC |
| 1102 | NEUTX | PDW |
| 1103 | NEUTX | PCT |
| 1104 | NEUTX | MPC |
| 1105 | NEUTX | #L-PLT |
| 1106 | NEUTX | PLT CLU |
| 1107 | NEUTX | PCDW |
| 1108 | NEUTY | LI |
| 1109 | NEUTY | PMR |
| 1110 | NEUTY | PMNX |
| 1111 | NEUTY | RBC |
| 1112 | NEUTY | HCT |
| 1113 | NEUTY | MCV |
| 1114 | NEUTY | MCH |
| 1115 | NEUTY | MCHC |
| 1116 | NEUTY | CHCM |
| 1117 | NEUTY | RDW |
| 1118 | NEUTY | HDW |
| 1119 | NEUTY | HCDW |
| 1120 | NEUTY | #MACRO |
| 1121 | NEUTY | #HYPO |
| 1122 | NEUTY | #HYPE |
| 1123 | NEUTY | #MRBC |
| 1124 | NEUTY | #NRBC |
| 1125 | NEUTY | MHGB |
| 1126 | NEUTY | #NNRBC |
| 1127 | NEUTY | PLT |
| 1128 | NEUTY | MPC |
| 1129 | NEUTY | PDW |
| 1130 | NEUTY | PCT |
| 1131 | NEUTY | MPC |
| 1132 | NEUTY | #L-PLT |
| 1133 | NEUTY | PLT CLU |
| 1134 | NEUTY | PCDW |
| 1135 | LI | PMR |
| 1136 | LI | PMNX |
| 1137 | LI | RBC |
| 1138 | LI | HCT |
| 1139 | LI | MCV |
| 1140 | LI | MCH |
| 1141 | LI | MCHC |
| 1142 | LI | CHCM |
| 1143 | LI | RDW |
| 1144 | LI | HDW |
| 1145 | LI | HCDW |
| 1146 | LI | #MACRO |
| 1147 | LI | #HYPO |
| 1148 | LI | #HYPE |
| 1149 | LI | #MRBC |
| 1150 | LI | #NRBC |
| 1151 | LI | MHGB |
| 1152 | LI | #NNRBC |
| 1153 | LI | PLT |
| 1154 | LI | MPC |
| 1155 | LI | PDW |
| 1156 | LI | PCT |
| 1157 | LI | MPC |
| 1158 | LI | #L-PLT |
| 1159 | LI | PLT CLU |
| 1160 | LI | PCDW |
| 1161 | PMR | PMNX |
| 1162 | PMR | RBC |
| 1163 | PMR | HCT |
| 1164 | PMR | MCV |
| 1165 | PMR | MCH |
| 1166 | PMR | MCHC |
| 1167 | PMR | CHCM |
| 1168 | PMR | RDW |
| 1169 | PMR | HDW |
| 1170 | PMR | HCDW |
| 1171 | PMR | #MACRO |
| 1172 | PMR | #HYPO |
| 1173 | PMR | #HYPE |
| 1174 | PMR | #MRBC |
| 1175 | PMR | #NRBC |
| 1176 | PMR | MHGB |
| 1177 | PMR | #NNRB |
| 1178 | PMR | PLT |
| 1179 | PMR | MPC |
| 1180 | PMR | PDW |
| 1181 | PMR | PCT |
| 1182 | PMR | MPC |
| 1183 | PMR | #L-PLT |
| 1184 | PMR | PLT CLU |
| 1185 | PMR | PCDW |
| 1186 | PMNX | RBC |
| 1187 | PMNX | HCT |
| 1188 | PMNX | MCV |
| 1189 | PMNX | MCH |
| 1190 | PMNX | MCHC |
| 1191 | PMNX | CHCM |
| 1192 | PMNX | RDW |
| 1193 | PMNX | HDW |
| 1194 | PMNX | HCDW |
| 1195 | PMNX | #MACRO |
| 1196 | PMNX | #HYPO |
| 1197 | PMNX | #HYPE |
| 1198 | PMNX | #MRBC |
| 1199 | PMNX | #NRBC |
| 1200 | PMNX | MHGB |
| 1201 | PMNX | #NNRBC |
| 1202 | PMNX | PLT |
| 1203 | PMNX | MPC |
| 1204 | PMNX | PDW |
| 1205 | PMNX | PCT |
| 1206 | PMNX | MPC |
| 1207 | PMNX | #L-PLT |
| 1208 | PMNX | PLT CLU |
| 1209 | PMNX | PCDW |
| 1210 | RBC | HCT |
| 1211 | RBC | MCV |
| 1212 | RBC | MCH |
| 1213 | RBC | MCHC |
| 1214 | RBC | CHCM |
| 1215 | RBC | RDW |
| 1216 | RBC | HDW |
| 1217 | RBC | HCDW |
| 1218 | RBC | #MACRO |
| 1219 | RBC | #HYPO |
| 1220 | RBC | #HYPE |
| 1221 | RBC | #MRBC |
| 1222 | RBC | #NRBC |
| 1223 | RBC | MHGB |
| 1224 | RBC | #NNRBC |
| 1225 | RBC | PLT |
| 1226 | RBC | MPC |
| 1227 | RBC | PDW |
| 1228 | RBC | PCT |
| 1229 | RBC | MPC |
| 1230 | RBC | #L-PLT |
| 1231 | RBC | PLT CLU |
| 1232 | RBC | PCDW |
| 1233 | HCT | MCV |
| 1234 | HCT | MCH |
| 1235 | HCT | MCHC |
| 1236 | HCT | CHCM |
| 1237 | HCT | RDW |
| 1238 | HCT | HDW |
| 1239 | HCT | HCDW |
| 1240 | HCT | #MACRO |
| 1241 | HCT | #HYPO |
| 1242 | HCT | #HYPE |
| 1243 | HCT | #MRBC |
| 1244 | HCT | #NRBC |
| 1245 | HCT | MHGB |
| 1246 | HCT | #NNRBC |
| 1247 | HCT | PLT |
| 1248 | HCT | MPC |
| 1249 | HCT | PDW |
| 1250 | HCT | PCT |
| 1251 | HCT | MPC |
| 1252 | HCT | #L-PLT |
| 1253 | HCT | PLT CLU |
| 1254 | HCT | PCDW |
| 1255 | MCV | MCH |
| 1256 | MCV | MCHC |
| 1257 | MCV | CHCM |
| 1258 | MCV | RDW |
| 1259 | MCV | HDW |
| 1260 | MCV | HCDW |
| 1261 | MCV | #MACRO |
| 1262 | MCV | #HYPO |
| 1263 | MCV | #HYPE |
| 1264 | MCV | #MRBC |
| 1265 | MCV | #NRBC |
| 1266 | MCV | MHGB |
| 1267 | MCV | #NNRBC |
| 1268 | MCV | PLT |
| 1269 | MCV | MPC |
| 1270 | MCV | PDW |
| 1271 | MCV | PCT |
| 1272 | MCV | MPC |
| 1273 | MCV | #L-PLT |
| 1274 | MCV | PLT CLU |
| 1275 | MCV | PCDW |
| 1276 | MCH | MCHC |
| 1277 | MCH | CHCM |
| 1278 | MCH | RDW |
| 1279 | MCH | HDW |
| 1280 | MCH | HCDW |
| 1281 | MCH | #MACRO |
| 1282 | MCH | #HYPO |
| 1283 | MCH | #HYPE |
| 1284 | MCH | #MRBC |
| 1285 | MCH | #NRBC |
| 1286 | MCH | MHGB |
| 1287 | MCH | #NNRBC |
| 1288 | MCH | PLT |
| 1289 | MCH | MPC |
| 1290 | MCH | PDW |
| 1291 | MCH | PCT |
| 1292 | MCH | MPC |
| 1293 | MCH | #L-PLT |
| 1294 | MCH | PLT CLU |
| 1295 | MCH | PCDW |
| 1296 | MCHC | CHCM |
| 1297 | MCHC | RDW |
| 1298 | MCHC | HDW |
| 1299 | MCHC | HCDW |
| 1300 | MCHC | #MACRO |
| 1301 | MCHC | #HYPO |
| 1302 | MCHC | #HYPE |
| 1303 | MCHC | #MRBC |
| 1304 | MCHC | #NRBC |
| 1305 | MCHC | MHGB |
| 1306 | MCHC | #NNRBC |
| 1307 | MCHC | PLT |
| 1308 | MCHC | MPC |
| 1309 | MCHC | PDW |
| 1310 | MCHC | PCT |
| 1311 | MCHC | MPC |
| 1312 | MCHC | #L-PLT |
| 1313 | MCHC | PLT CLU |
| 1314 | MCHC | PCDW |
| 1315 | CHCM | RDW |
| 1316 | CHCM | HDW |
| 1317 | CHCM | HCDW |
| 1318 | CHCM | #MACRO |
| 1319 | CHCM | #HYPO |
| 1320 | CHCM | #HYPE |
| 1321 | CHCM | #MRBC |
| 1322 | CHCM | #NRBC |
| 1323 | CHCM | MHGB |
| 1324 | CHCM | #NNRBC |
| 1325 | CHCM | PLT |
| 1326 | CHCM | MPC |
| 1327 | CHCM | PDW |
| 1328 | CHCM | PCT |
| 1329 | CHCM | MPC |
| 1330 | CHCM | #L-PLT |
| 1331 | CHCM | PLT CLU |
| 1332 | CHCM | PCDW |
| 1333 | RDW | HDW |
| 1334 | RDW | HCDW |
| 1335 | RDW | #MACRO |
| 1336 | RDW | #HYPO |
| 1337 | RDW | #HYPE |
| 1338 | RDW | #MRBC |
| 1339 | RDW | #NRBC |
| 1340 | RDW | MHGB |
| 1341 | RDW | #NNRBC |
| 1342 | RDW | PLT |
| 1343 | RDW | MPC |
| 1344 | RDW | PDW |
| 1345 | RDW | PCT |
| 1346 | RDW | MPC |
| 1347 | RDW | #L-PLT |
| 1348 | RDW | PLT CLU |
| 1349 | RDW | PCDW |
| 1350 | HDW | HCDW |
| 1351 | HDW | #MACRO |
| 1352 | HDW | #HYPO |
| 1353 | HDW | #HYPE |
| 1354 | HDW | #MRBC |
| 1355 | HDW | #NRBC |
| 1356 | HDW | MHGB |
| 1357 | HDW | #NNRBC |
| 1358 | HDW | PLT |
| 1359 | HDW | MPC |
| 1360 | HDW | PDW |
| 1361 | HDW | PCT |
| 1362 | HDW | MPC |
| 1363 | HDW | #L-PLT |
| 1364 | HDW | PLT CLU |
| 1365 | HDW | PCDW |
| 1366 | HCDW | #MACRO |
| 1367 | HCDW | #HYPO |
| 1368 | HCDW | #HYPE |
| 1369 | HCDW | #MRBC |
| 1370 | HCDW | #NRBC |
| 1371 | HCDW | MHGB |
| 1372 | HCDW | #NNRBC |
| 1373 | HCDW | PLT |
| 1374 | HCDW | MPC |
| 1375 | HCDW | PDW |
| 1376 | HCDW | PCT |
| 1377 | HCDW | MPC |
| 1378 | HCDW | #L-PLT |
| 1379 | HCDW | PLT CLU |
| 1380 | HCDW | PCDW |
| 1381 | #MACRO | #HYPO |
| 1382 | #MACRO | #HYPE |
| 1383 | #MACRO | #MRBC |
| 1384 | #MACRO | #NRBC |
| 1385 | #MACRO | MHGB |
| 1386 | #MACRO | #NNRBC |
| 1387 | #MACRO | PLT |
| 1388 | #MACRO | MPC |
| 1389 | #MACRO | PDW |
| 1390 | #MACRO | PCT |
| 1391 | #MACRO | MPC |
| 1392 | #MACRO | #L-PLT |
| 1393 | #MACRO | PLT CLU |
| 1394 | #MACRO | PCDW |
| 1395 | #HYPO | #HYPE |
| 1396 | #HYPO | #MRBC |
| 1397 | #HYPO | #NRBC |
| 1398 | #HYPO | MHGB |
| 1399 | #HYPO | #NNRBC |
| 1400 | #HYPO | PLT |
| 1401 | #HYPO | MPC |
| 1402 | #HYPO | PDW |
| 1403 | #HYPO | PCT |
| 1404 | #HYPO | MPC |
| 1405 | #HYPO | #L-PLT |
| 1406 | #HYPO | PLT CLU |
| 1407 | #HYPO | PCDW |
| 1408 | #HYPE | #MRBC |
| 1409 | #HYPE | #NRBC |
| 1410 | #HYPE | MHGB |
| 1411 | #HYPE | #NNRBC |
| 1412 | #HYPE | PLT |
| 1413 | #HYPE | MPC |
| 1414 | #HYPE | PDW |
| 1415 | #HYPE | PCT |
| 1416 | #HYPE | MPC |
| 1417 | #HYPE | #L-PLT |
| 1418 | #HYPE | PLT CLU |
| 1419 | #HYPE | PCDW |
| 1420 | #MRBC | #NRBC |
| 1421 | #MRBC | MHGB |
| 1422 | #MRBC | #NNRBC |
| 1423 | #MRBC | PLT |
| 1424 | #MRBC | MPC |
| 1425 | #MRBC | PDW |
| 1426 | #MRBC | PCT |
| 1427 | #MRBC | MPC |
| 1428 | #MRBC | #L-PLT |
| 1429 | #MRBC | PLT CLU |
| 1430 | #MRBC | PCDW |
| 1431 | #NRBC | MHGB |
| 1432 | #NRBC | #NNRBC |
| 1433 | #NRBC | PLT |
| 1434 | #NRBC | MPC |
| 1435 | #NRBC | PDW |
| 1436 | #NRBC | PCT |
| 1437 | #NRBC | MPC |
| 1438 | #NRBC | #L-PLT |
| 1439 | #NRBC | PLT CLU |
| 1440 | #NRBC | PCDW |
| 1441 | MHGB | #NNRBC |
| 1442 | MHGB | PLT |
| 1443 | MHGB | MPC |
| 1444 | MHGB | PDW |
| 1445 | MHGB | PCT |
| 1446 | MHGB | MPC |
| 1447 | MHGB | #L-PLT |
| 1448 | MHGB | PLT CLU |
| 1449 | MHGB | PCDW |
| 1450 | #NNRBC | PLT |
| 1451 | #NNRBC | MPC |
| 1452 | #NNRBC | PDW |
| 1453 | #NNRBC | PCT |
| 1454 | #NNRBC | MPC |
| 1455 | #NNRBC | #L-PLT |
| 1456 | #NNRBC | PLT CLU |
| 1457 | #NNRBC | PCDW |
| 1458 | PLT | MPC |
| 1459 | PLT | PDW |
| 1460 | PLT | PCT |
| 1461 | PLT | MPC |
| 1462 | PLT | #L-PLT |
| 1463 | PLT | PLT CLU |
| 1464 | PLT | PCDW |
| 1465 | MPC | PDW |
| 1466 | MPC | PCT |
| 1467 | MPC | MPC |
| 1468 | MPC | #L-PLT |
| 1469 | MPC | PLT CLU |
| 1470 | MPC | PCDW |
| 1471 | PDW | PCT |
| 1472 | PDW | MPC |
| 1473 | PDW | #L-PLT |
| 1474 | PDW | PLT CLU |
| 1475 | PDW | PCDW |
| 1476 | PCT | MPC |
| 1477 | PCT | #L-PLT |
| 1478 | PCT | PLT CLU |
| 1479 | PCT | PCDW |
| 1480 | MPC | #L-PLT |
| 1481 | MPC | PLT CLU |
| 1482 | MPC | PCDW |
| 1483 | #L-PLT | PLT CLU |
| 1484 | #L-PLT | PCDW |
| 1485 | PLT CLU | PCDW |

### II. Marker Analyzers

The markers of the present invention may be detected with any type of analyzer that is capable of detecting any of the markers from Table 50 in a sample from a subject. In certain embodiments, the analyzers are blood analyzers configured to detect at least one of the markers from Table 50. In preferred embodiments, the analyzers are hematology analyzers.

A hematology analyzer (a.k.a. haematology analyzer, hematology analyzer, haematology analyser) is an automated instrument (e.g. clinical instrument and/or laboratory instrument) which analyzes the various components (e.g. blood cells) of a blood sample. Typically, hematology analyzers are automated cell counters used to perform cell counting and separation tasks including: differentiation of individual blood cells, counting blood cells, separating blood cells in a sample based on cell-type, quantifying one or more specific types of blood cells, and/or quantifying the size of the blood cells in a sample. In some embodiments, hematology analyzers are automated coagulometers which measure the ability of blood to clot (e.g. partial thromboplastin times, prothrombin times, lupus anticoagulant screens, D dimer assays, factor assays, etc.), or automatic erythrocyte sedimentation rate (ESR) analyzers. In general, a hematology analyzer performing cell counting functions samples the blood, and quantifies, classifies, and describes cell populations using both electrical and optical techniques. A properly outfitted hematology analyzer (e.g. with peroxidase staining capability) is capable of providing values for Markers 1-55, using various analyses.

Electrical analysis by a hematology analyzer generally involves passing a dilute solution of a blood sample through an aperture across which an electrical current is flowing. The passage of cells through the current changes the impedance between the terminals (the Coulter principle). A lytic reagent is added to the blood solution to selectively lyse red blood cells (RBCs), leaving only white blood cells (WBCs), and platelets intact. Then the solution is passed through a second detector. This allows the counts of RBCs, WBCs, and platelets to be obtained. The platelet count is easily separated from the WBC count by the smaller impedance spikes they produce in the detector due to their lower cell volumes.

Optical detection by a hematology analyzer may be utilized to gain a differential count of the populations of white cell types. In general, a suspension of cells (e.g. dilute cell suspension) is passed through a flow cell, which passes cells one at a time through a capillary tube past a laser beam. The reflectance, transmission, and scattering of light from each cell are analyzed by software giving a numerical representation of the likely overall distribution of cell populations.

In some embodiments, RBCs are lysed to release hemoglobin. The heme group of the hemoglobin is oxidized from the ferrous to ferric state by an oxidizing agent (e.g. dimethyllaurylamine oxide) and subsequently combined with cyanide. Optical reading are then obtained colorimetrically (e.g. at 546 nm). In some embodiments, parameters including, but not limited to: hemoglobin content, mean corpuscular hemoglobin, and mean corpuscular hemoglobin concentration are measure via the above process.

In some embodiments, an RBC count is obtained by applying a sphereing reagent (e.g. sodium dodecyl sulfate (SDS) and glutaraldehyde) is added to a sample to isovolumetrically sphere RBCs and platelets, thereby eliminating shape variability in measurements. Absorption, low-angle scattering, and high-angle scattering are then measured and RBCs are classified by volume and hemoglobin concentration. A variety of parameters are calculated including, but not limited to: RBC count, mean corpuscular volume, hematocrit, mean corpuscular hemoglobin, mean corpuscular hemoglobin concentration, corpuscular hemoglobin concentration mean, corpuscular hemoglobin content, red cell volume distribution width, hemoglobin concentration width, percent of RBCs smaller than 60 fL, percent of RBCs larger than 120 fL, percent of RBCs with less than 28 g/dL hemoglobin, and percent of RBCs with more than 41 g/dL hemoglobin.

In some embodiments, reticulocyte counts are performed using a supravital and/or cationic dye (e.g. methylene blue, Oxazine 750, etc.) to stain the RBCs containing reticulin prior to counting. A detergent or surfactant may be employed to isovolumetrically sphere RBCs. Absorption and light-scatter measurements are taken and, based on cell maturation and cell size, cells are classified as mature RBCs; low-, medium-, or high-absorption reticulocytes; or platelets. A variety of parameters can be obtain from this analysis including, but not limited to: the percent reticulocytes, number of reticulocytes, mean cell volume (MCV) of reticulocytes, cellular hemoglobin content of reticulocytes, cell hemoglobin concentration mean reticulocytes, immature reticulocytes fraction high, and immature reticulocytes fraction medium and high.

In some embodiments, neutrophil granules are counted using a peroxidase method to classify WBCs. In some embodiments, hydrogen peroxide and a stabilizer (e.g. 4-chloro-1-naphthol) are added to a sample to generate precipitate (e.g. dark precipitate) at sites of peroxidase activity in the granules of WBCs. Based on the number of cellular granules and the degree of cell maturation, cells may be classified into groups including: myeloblasts, promyeloblasts, myelocytes, metamyelocytes, metamyelocytes, band cells, neutrophils, eosinophils, basophils, lymphoblasts, prolymphocytes, atypical lymphocytes, monoblasts, promonocytes, monocytes, or plasma cells. Using the peroxidase method, parameters are obtained including, but not limited to: WBC count perox, percent neutrophils, number of neutrophils, percent lymphocytes, number of lymphocytes, percent monocytes, number of monocytes, percent eosinophils, number of eosinophils, percent large unstained cells, number of large unstained cells, presence of atypical lymphocytes, presence of immature granulocytes, myeloperoxidase deficiency, presence of nucleated RBCs, and presence of clumped platelets.

In some embodiments, basophils are counted using a procedure in which acid (e.g. pthalic acid and/or hydrochloric acid) and a surfactant are applied to a sample to lyse RBCs, platelets, and all WBCs except basophils. Based on the nuclear configuration (based on high-angle light scattering) and cell size (based on low-angle light scattering), cells/nuclei are classified as blast cell nuclei, mononuclear WBCs, basophils, suspect basophils, or polymorphonuclear WBCs. Using the basophil method, parameters are obtained including, but not limited to: percent basophils, number of basophils, percent blasts, number of blasts, percent mononuclear cells, number of mononuclear cells, the present of blasts, and the presence of nonsegmented neutrophils (bands).

In some embodiments, any suitable hematology analyzer may find use with embodiments of the present invention. In some embodiments, an ADVIA 120, earlier models, newer models, or similar hematology analyzers find use in embodiments of the present invention (e.g. embodiments using in situ cytochemical peroxidase based staining procedures (e.g. PEROX, PEROX-CHRP, etc.)). In some embodiments, a hematology analyzer comprises a unified fluids circuit (UFC); and a light generation, light manipulation (e.g. focusing, bending, directing, filtering, splitting, etc.) absorption, and detection assembly comprising one or more of a lamp assembly (e.g. tungsten lamp), filters, photodiode, laserdiode, beam splitters, dark stops, mirrors, absorption detector, scatter detector, low-angle scatter detector, high-angle scatter detector, and/or additional components understood by those in the art. In some embodiments, a UFC provides: a pump assembly, pathways for fluids and air-flow, valves (e.g. shear valve), and reaction chambers. In some embodiments, a UFC comprises multiple reaction chambers including, but not limited to: a hemoglobin reaction chamber, basophil reaction chamber, RBC reaction chamber, reticulocyte reaction chamber, PEROX reaction chamber, etc.

### III. Generating Risk Profiles

The present invention is not limited by the mathematic methods that are employed to generate risk profiles for an individual patient, where such risk profiles may be used to predict risk of death of MI at, for example, one year. Examples of mathematical/statistical approaches useful for generation of individual risk profiles includes, using some or all of the markers disclosed herein include, but are not limited to:

1. The Logical Analysis of Data (LAD) method (34-36);

2. Linear discriminant analysis (LDA) and the related Fisher's linear discriminant (Fisher, R. A, 1936, Annal of Eugenics, 7:179-188, herein incorporated by reference in its entirety) are methods used in statistics, pattern recognition and machine learning to find a linear combination of markers which characterize or separate two or more classes of objects or events.

3. Quardratic discriminant analysis (QDA) (Sathyanarayana, Shashi, 2010, Wolfram Demonstrations Project, http://, followed by demonstrations.wolfram.com/PatternRecognition PrimerII) is closely related to LDA. QDA finds a quadratic combination of markers which best separates two or more classes of objects or events.

4. Flexible discriminant analysis (FDA) (Hastie et al., 1994, JASA, 1255-1270, herein incorporated by reference in its entirety) recasts LDA as a linear regression problem and substitutes linear combination by a non parametric one.

5. Penalized discriminant analysis (PDA) (Hastie et al., 1995, Annals of Statistics, 23(1):73-102, herein incorporated by reference in its entirety) is an extension of LDA. It is designed for situations in which there are many highly correlated predictors.

6. Mixture discriminant analysis (MDA) (Hastie wt al., 1996, JRSS-B, 155-176, herein incorporated by reference in its entirety) is a method for classification based on mixture models. It is an extension of LDA, and the mixture of normal distributions is used to obtain a density estimation for each class.

7. K-nearest-neighbors (KNN) (Cover et al., 1967, IEEE Transactions on Information Theory 13 (1): 21-27, herein incorporated by reference in its entirety) is a method for classifying objects based on closest training examples in the feature space. An object is classified by a majority vote of its neighbors, with the object being assigned to the class most common amongst its k nearest neighbors (k is a positive integer, typically small).

8. Support vector machine (SVM) (Meyer et al., 2003, Nuroocomputing 55(1-2): 169-186, herein incorporated by reference) finds a hyperplane separating the classes in the training set in a feature space. The goal in training a SVM is to find an optimal separating hyperplane that separates the two classes and maximizes the distance to the closest point from either class. Not only does this provide a unique solution to the separating hyperplane problem, but it also maximizes the margin between the two classes on the training data which leads to better classification performance on testing data.

9. Random Forest (RF) (Breiman, 2001, Machine learning, 45:5-32, herein incorporated by reference in its entirety) is a collection of identically distributed trees. Each tree is constructed using a tree classification algorithm. The RF is formed by taking bootstrap samples from the training set. For each bootstrap sample, a classification tree is formed, and the tree grows until all terminal nodes are pure. After the tree is grown, one drops a new case down each of the trees. The classification that receives the majority vote is the one that is assigned to the new observation. RF handles missing data very well and provides estimates of the relative importance of each of the peaks in the classification rule, which can be used to discover the most important biomarkers.

10. Multivariate Adaptive Regression Splines (MARS) (Friedman, J. H., 1991, Annals of Statistics, 19 (1): 1-67, herein incorporated by reference in its entirety) is an adaptive procedure for regression, and is well suited for data with a large number of elements. It can be viewed as a generalization of stepwise linear regression. The MARS method can be extended to handle classification problems.

11. Recursive Partitioning and Regression Trees (RPART) (Breiman et al., 1984, Classification and Regression Trees, New York: Chapman & Hall, herein incorporated by reference in its entirety) is an iterative process of splitting the data into increasingly homogeneous partitions until it is infeasible to continue based on a set of “stopping rules.”

12. Cox model (Cox, D. R., 1972, JRSS-B 34 (2): 187-220, herein incorporated by reference in its entirety) is a well-recognized statistical technique for exploring the relationship between the time to event of a subject and several explanatory variables. It allows us to estimate the hazard (or risk) of death, or other event of interest, for individuals, given their prognostic variables.

13. Random Survival Forest (RSF) (Ishwaran et al., 2008, The Annals of Applied Statistics, 2(3):841-860, herein incorporated by reference in its entirety) is an ensemble tree method for analysis of right-censored survival data. Random survival forest methodology extends Breiman's random forest method.

### IV. Biological Samples

Biological samples include, but are not necessarily limited to bodily fluids such as blood-related samples (e.g., whole blood, serum, plasma, and other blood-derived samples), urine, cerebral spinal fluid, bronchoalveolar lavage, and the like. Another example of a biological sample is a tissue sample. In preferred embodiments, the biological sample is blood.

A biological sample may be fresh or stored (e.g. blood or blood fraction stored in a blood bank). The biological sample may be a bodily fluid expressly obtained for the assays of this invention or a bodily fluid obtained for another purpose which can be sub-sampled for the assays of this invention.

In one embodiment, the biological sample is whole blood. Whole blood may be obtained from the subject using standard clinical procedures. In another embodiment, the biological sample is plasma. Plasma may be obtained from whole blood samples by centrifugation of anticoagulated blood. Such process provides a buffy coat of white cell components and a supernatant of the plasma. In another embodiment, the biological sample is serum. Serum may be obtained by centrifugation of whole blood samples that have been collected in tubes that are free of anti-coagulant. The blood is permitted to clot prior to centrifugation. The yellowish-reddish fluid that is obtained by centrifugation is the serum. In another embodiment, the sample is urine.

The sample may be pretreated as necessary by dilution in an appropriate buffer solution, heparinized, concentrated if desired, or fractionated by any number of methods including but not limited to ultracentrifugation, fractionation by fast performance liquid chromatography (FPLC), or precipitation of apolipoprotein B containing proteins with dextran sulfate or other methods. Any of a number of standard aqueous buffer solutions at physiological pH, such as phosphate, Tris, or the like, can be used.

### V. Subjects

In certain embodiments, the subject is any human or other animal to be tested for characterizing its risk of CVD (e.g. congestive heart failure, aortic aneurysm or aortic dissection). In certain embodiments, the subject does not otherwise have an elevated risk of an adverse cardiovascular event. Subjects having an elevated risk of experiencing a cardiovascular event include those with a family history of cardiovascular disease, elevated lipids, smokers, prior acute cardiovascular event, etc. (See, e.g., Harrison's Principles of Experimental Medicine, 15th Edition, McGraw-Hill, Inc., N.Y.—hereinafter “Harrison's”).

In certain embodiments the subject is apparently healthy. “Apparently healthy”, as used herein, describes a subject who does not have any signs or symptoms of CVD or has not previously been diagnosed as having any signs or symptoms indicating the presence of atherosclerosis, such as angina pectoris, history of a cardiovascular event such as a myocardial infarction or stroke, or evidence of atherosclerosis by diagnostic imaging methods including, but not limited to coronary angiography. Apparently healthy subjects also do not have any signs or symptoms of having heart failure or an aortic disorder.

In other embodiments, the subject already exhibits symptoms of cardiovascular disease. For example, the subject may exhibit symptoms of heart failure or an aortic disorder such as aortic dissection or aortic aneurysm. For subjects already experiencing cardiovascular disease, the values for the markers of the present invention can be used to predict the likelihood of further cardiovascular events or the outcome of ongoing cardiovascular disease.

In certain embodiments, the subject is a nonsmoker. “Nonsmoker” describes an individual who, at the time of the evaluation, is not a smoker. This includes individuals who have never smoked as well as individuals who have smoked but have not used tobacco products within the past year. In certain embodiments, the subject is a smoker.

In some embodiments, the subject is a nonhyperlipidemic subject. “Nonhyperlipidemic” describes a subject that is a nonhypercholesterolemic and/or a nonhypertriglyceridemic subject. A “nonhypercholesterolemic” subject is one that does not fit the current criteria established for a hypercholesterolemic subject. A nonhypertriglyceridemic subject is one that does not fit the current criteria established for a hypertriglyceridemic subject (See, e.g., Harrison's Principles of Experimental Medicine, 15th Edition, McGraw-Hill, Inc., N.Y.—hereinafter “Harrison's”). Hypercholesterolemic subjects and hypertriglyceridemic subjects are associated with increased incidence of premature coronary heart disease. A hypercholesterolemic subject has an LDL level of >160 mg/dL, or >130 mg/dL and at least two risk factors selected from the group consisting of male gender, family history of premature coronary heart disease, cigarette smoking (more than 10 per day), hypertension, low HDL (<35 mg/dL), diabetes mellitus, hyperinsulinemia, abdominal obesity, high lipoprotein (a), and personal history of cerebrovascular disease or occlusive peripheral vascular disease. A hypertriglyceridemic subject has a triglyceride (TG) level of >250 mg/dL. Thus, a nonhyperlipidemic subject is defined as one whose cholesterol and triglyceride levels are below the limits set as described above for both the hypercholesterolemic and hypertriglyceridemic subjects.

### VI. Threshold Values

In certain embodiments, values of the markers of the present invention in the biological sample obtained from the test subject may compared to a threshold value. A threshold value is a concentration or number of an analyte (e.g., particular cells type) that represents a known or representative amount of an analyte. For example, the control value can be based upon values of certain markers in comparable samples obtained from a reference cohort (e.g., see Examples 1-4). In certain embodiments, the reference cohort is the general population. In certain embodiments, the reference cohort is a select population of human subjects. In certain embodiments, the reference cohort is comprised of individuals who have not previously had any signs or symptoms indicating the presence of atherosclerosis, such as angina pectoris, history of a cardiovascular event such as a myocardial infarction or stroke, evidence of atherosclerosis by diagnostic imaging methods including, but not limited to coronary angiography. In certain embodiments, the reference cohort includes individuals, who if examined by a medical professional would be characterized as free of symptoms of disease (e.g., cardiovascular disease). In another example, the reference cohort may be individuals who are nonsmokers (i.e., individuals who do not smoke cigarettes or related items such as cigars). The threshold values selected may take into account the category into which the test subject falls. Appropriate categories can be selected with no more than routine experimentation by those of ordinary skill in the art. The threshold value is preferably measured using the same units used to measures one or more markers of the present invention.

The threshold value can take a variety of forms. The threshold value can be a single cut-off value, such as a median or mean. The control value can be established based upon comparative groups such as where the risk in one defined group is double the risk in another defined group. The threshold values can be divided equally (or unequally) into groups, such as a low risk group, a medium risk group and a high-risk group, or into quadrants, the lowest quadrant being individuals with the lowest risk the highest quadrant being individuals with the highest risk, and the test subject's risk of having CVD can be based upon which group his or her test value falls. Threshold values for markers in biological samples obtained, such as mean levels, median levels, or “cut-off” levels, are established by assaying a large sample of individuals in the general population or the select population and using a statistical model such as the predictive value method for selecting a positivity criterion or receiver operator characteristic curve that defines optimum specificity (highest true negative rate) and sensitivity (highest true positive rate) as described in Knapp, R. G., and Miller, M. C. (1992). Clinical Epidemiology and Biostatistics. William and Wilkins, Harual Publishing Co. Malvern, Pa., which is specifically incorporated herein by reference. A “cutoff” value can be determined for each risk predictor that is assayed.

Levels of particular markers in a subject's biological sample may be compared to a single threshold value or to a range of threshold values. If the level of the marker in the test subject's biological sample is greater than the threshold value or exceeds or is in the upper range of threshold values, the test subject may, depending on the marker, be at greater risk of developing or having CVD or experiencing a cardiovascular event within the ensuing year, two years, and/or three years than individuals with levels comparable to or below the threshold value or in the lower range of threshold values. In contrast, if levels of the marker in the test subject's biological sample is below the threshold value or is in the lower range of threshold values, the test subject, depending on the marker, be at a lower risk of developing or having CVD or experiencing a cardiovascular event within the ensuing year, two years, and/or three years than individuals whose levels are comparable to or above the threshold value or exceeding or in the upper range of threshold values. The extent of the difference between the test subject's marker levels and threshold value may also useful for characterizing the extent of the risk and thereby determining which individuals would most greatly benefit from certain aggressive therapies. In those cases, where the threshold value ranges are divided into a plurality of groups, such as the threshold value ranges for individuals at high risk, average risk, and low risk, the comparison involves determining into which group the test subject's level of the relevant marker falls.

### VII. Evaluation of Therapeutic Agents or Therapeutic Interventions

Also provided are methods for evaluating the effect of CVD therapeutic agents, or therapeutic interventions, on individuals who have been diagnosed as having or as being at risk of developing CVD. Such therapeutic agents include, but are not limited to, antibiotics, anti-inflammatory agents, insulin sensitizing agents, antihypertensive agents, anti-thrombotic agents, anti-platelet agents, fibrinolytic agents, lipid reducing agents, direct thrombin inhibitors, ACAT inhibitor, CDTP inhibitor thioglytizone, glycoprotein IIb/IIIa receptor inhibitors, agents directed at raising or altering HDL metabolism such as apoA-I milano or CETP inhibitors (e.g., torcetrapib), agents designed to act as artificial HDL, particular diets, exercise programs, and the use of cardiac related devices. Accordingly, a CVD therapeutic agent, as used herein, refers to a broader range of agents that can treat a range of cardiovascular-related conditions, and may encompass more compounds than the traditionally defined class of cardiovascular agents.

Evaluation of the efficacy of CVD therapeutic agents, or therapeutic interventions, can include obtaining a predetermined value of one or more markers in a biological sample, and determining the level of one or more markers in a corresponding biological fluid taken from the subject following administration of the therapeutic agent or use of the therapeutic intervention. A decrease in the level of one or more markers, depending the marker, in the sample taken after administration of the therapeutic as compared to the level of the selected risk markers in the sample taken before administration of the therapeutic agent (or intervention) may be indicative of a positive effect of the therapeutic agent on cardiovascular disease in the treated subject.

A predetermined value can be based on the levels of one or more markers in a biological sample taken from a subject prior to administration of a therapeutic agent or intervention. In another embodiment, the predetermined value is based on the levels of one or more markers taken from control subjects that are apparently healthy, as defined herein.

Embodiments of the methods described herein can also be useful for determining if and when therapeutic agents (or interventions) that are targeted at preventing CVD or for slowing the progression of CVD should and should not be prescribed for a individual. For example, individuals with marker values above a certain cutoff value, or that are in the higher tertile or quartile of a “normal range,” could be identified as those in need of more aggressive intervention with lipid lowering agents, insulin, life style changes, etc.

### EXAMPLES

The following examples are for purposes of illustration only and are not intended to limit the scope of the claims.

### Example 1

### Comprehensive Peroxidase-Based Hematologic Profiling for the Prediction of One-Year Myocardial Infarction and Death

This example describes methods and analyses used to screen a patient population for markers that predict cardiovascular disease.

Methods and Results:

Stable patients (N=7,369) undergoing elective cardiac evaluation at a tertiary care center were enrolled. A model (PEROX) that predicts incident one-year death and MI was derived from standard clinical data combined with information captured by a high throughput peroxidase-based hematology analyzer during performance of a complete blood count with differential. The PEROX model was developed using a random sampling of subjects in a Derivation Cohort (N=5,895) and then independently validated in a non-overlapping Validation Cohort (N=1,474). Twenty-three high-risk (observed in ≧10% of subjects with events) and 24 low-risk (observed in ≧10% of subjects without events) patterns were identified in the Derivation Cohort. Erythrocyte- and leukocyte (peroxidase)-derived parameters dominated the variables predicting risk of death, whereas, variables in MI risk patterns included traditional cardiac risk factors and elements from all blood cell lineages. Within the Validation Cohort, the PEROX model demonstrated superior prognostic accuracy (78%) for one-year risk of death or MI compared with traditional risk factors alone (67%). Furthermore, the PEROX model reclassifies 23.5% (p<0.001) of patients to different risk categories for death/MI when added to traditional risk factors.

This Example shows that comprehensive pattern recognition of high and low-risk clusters of clinical, biochemical, and hematological parameters provides incremental prognostic value in both primary and secondary prevention patients for near-term (one year) risks for death and MI.

Methods:

Study Sample: GeneBank is an Institutional Review Board approved prospective cohort study at the Cleveland Clinic with enrollment from 2002-2006. Patients were eligible for inclusion if they were undergoing elective diagnostic cardiac catheterization, were age 18 years or above, and were both stable and without active chest pain at time of enrollment. All subjects with positive cardiac troponin T test (≧0.03 ng/ml) on enrollment blood draw immediately prior to catheterization were excluded from the study. Indications for catheterization included: history of positive or equivocal stress test (46%), rule out cardiovascular disease in presence of cardiac risk factors (63%), prior to surgery or intervention (24%), recent but historical myocardial infarction (MI, 7%), prior coronary artery bypass or percutaneous intervention with recurrence of symptoms (37%), history of cardiomyopathy (3%) or remote history of acute coronary syndrome (0.9%). All subjects gave written informed consent approved by the Institutional Review Board.

Collection of Specimens and Clinical Data:

Patients were interviewed using a standardized demographics and clinical history questionnaire. Blood samples were taken from femoral artery at onset of catheterization procedure prior to administration of heparin and collected into an EDTA tube, stored either on ice or at 4° C. until transfer to laboratory (typically within 2 hours) for immediate hematology analyzer analysis and subsequent processing and storage of plasma at −80° C. Basic metabolic panel, fasting lipid profile, and high sensitivity Creactive protein (hsCRP) levels were measured on the Abbott Architect platform (Abbott Laboratories, Abbott Park Ill.) in a core laboratory. Samples were identified by barcode only, and all laboratory personnel remained blinded to clinical data. Follow-up telephone interviews were performed by research personnel to track patient outcomes at one year, with all events (death and MI) adjudicated and confirmed by source documentation.

Comprehensive Hematology Analyses:

Hematology analyses were performed using an Advia 120 hematology analyzer (Siemens, New York, N.Y.). This hematology analyzer functions as a flow cytometer, using in situ peroxidase cytochemical staining to generate a CBC (complete blood count) and differential based on flow cytometry analysis of whole anticoagulated blood. All hematology measurements used in this Example were generated automatically by the analyzer during routine performance of a CBC and differential and do not require any additional sample preparation or processing steps to be performed. However, additional steps were taken to ensure the data was saved and extracted appropriately, since not all measurements are routinely reported. All leukocyte-, erythrocyte-, and platelet-related parameters derived from both cytograms and absorbance data were extracted from instrument DAT files by blinded laboratory technicians.

All hematology parameters utilized demonstrated reproducible results (with standard deviation from mean≦30%) upon replicate both intra-day and inter-day (>10 times) analyses. An example of a leukocyte cytogram and a table listing all hematology analyzer elements recovered and utilized for analysis is described further below.

Statistical Analyses and Construction of the PEROX Score:

An initial 7,466 subjects were consented for hematology analyses. Of these, 7,369 (98.7%) were included in statistical analyses. The 97 subjects not included in statistical analyses were excluded because they either were lost to follow-up, subsequently asked to be withdrawn from the study, or the hematology lab data failed to meet quality control parameters (e.g. platelet clumping or hemolyzed sample). The initial dataset was stratified based on whether a patient experienced an adjudicated event (non-fatal MI or death) by one-year following enrollment. Randomization using a uniform distribution method was performed to randomly select 80% of patients (Derivation Cohort) for model building and the remaining 20% (Validation Cohort) was set aside for model testing and validation prior to statistical analyses. Mean and median differences were assessed with Student's t-test and Mann-Whitney, respectively. Univariate hazard ratios (HR) were generated for continuous variables or logarithmically transformed continuous variables (if not normally distributed) for the purpose of ranking, as noted in Tables 2A and B.

In order to establish an individual subject's risk, a score was developed (PEROX) by initially identifying binary variable pairs that form reproducible high-risk (observed in ≧10% of subjects with events) and low-risk (observed in ≧10% of subjects without events) patterns for death or MI at one-year using the logical analysis of data (LAD) method (34-36). Using this combinatorics and optimization-based mathematical method, a single calculated value for an individual's overall one-year risk for death or MI was derived from a weighted integer sum of high- and low-risk patterns present. Briefly, LAD was first used to identify binary variable pairs that form reproducible positive and negative predictive patterns for risk for death or MI at one year.

Variables were included based on clinical significance, perceived potential informativeness, reproducibility (for hematology parameters) as monitored in inter-day and intra-day replicates, as well as non-redundancy, as assessed by cluster analysis performed within leukocyte, erythrocyte, and platelet subgroups. Criteria for the development of the PEROX model included three equal proportions for each hematology parameter, two variables per pattern, and a minimal prevalence of 10% of the events for high-risk and 10% of non-events for low-risk patterns. Patterns were generated using LAD software (http:// followed by “pit.kamick.free.fr/lemaire/LAD/”), and tuned for both homogeneity and prevalence to obtain best accuracy on cross validation experiments. The weight for each positive pattern was (+1/number of high-risk patterns), while for each negative pattern was (−1/number of low-risk patterns). An overall risk score for a patient was calculated by the sum of positive and negative pattern weights. A maximum score of +1 would be calculated in a patient with only positive patterns whereas a minimum score of −1 would be present in a patient with only negative patterns. The original score range was adjusted from ±1 to a range of 0 to 100 by assuming 50 (rather than 0) as midpoint of equal variance. The PEROX score was thus calculated as: 50×[(1/23 possible high-risk patterns)×(# actual high-risk patterns)−(1/24 possible low-risk patterns)×(# low-risk patterns)]+50. The reproducibility of the PEROX score was assessed by examining multiple replicate samples from multiple subjects both within and between days, revealing intra-day and inter-day coefficients of variance of 5±0.4% (mean±S.D.) and 10±2%, respectively. A more detailed explanation of how the PEROX score was built and a complete list of all hematology analyzer variables used within the PEROX score (including an example calculation using patient data) are provided further below.

Validation of PEROX Score and Comparisons:

Kaplan-Meier survival curves for PEROX model tertiles were generated within the Validation Cohort for the one-year outcomes including death, non-fatal myocardial infarction (MI) or either outcome, and compared by logrank test. Cox proportional hazards regression was used for time-to-event analysis to calculate HR and 95% confidence intervals (95% CI) for one-year outcomes of death, MI or either outcome. Cubic splines (with 95% confidence intervals) were generated to examine the relationship between PEROX model and one-year outcomes from the Derivation cohort, superimposed with absolute one-year event rates observed in the Validation Cohort. Receiver operating characteristic (ROC) curves were plotted and area under the curve (AUC) were estimated for one-year outcomes for the Validation Cohort using risk scores assigned by the PEROX model along with traditional risk factors (including age, gender, smoking, LDL cholesterol, HDL cholesterol, systolic blood pressure and history of diabetes) and compared to risk models incorporating traditional risk factors alone. In order to obtain an unbiased estimate of AUC, re-sampling (250 bootstrap samples from the Validation Cohort) was performed. For each bootstrap sample, AUC values were calculated for traditional risk factors with and without PEROX. AUC were compared using a method of comparing correlated ROC curves to calculate p-values for each bootstrap sample (37). The Friedman's test blocked on replicate was also used to compare AUC of 250 bootstrap samples (38). In addition, the net reclassification improvement (NRI) was determined by assessing net improvement in risk classification (higher predicted risk in subjects with events at one year, lower predicted risk in subjects without events at one year) using a ratio of 6:3:1 for low, medium, and high-risk categories (39). Consistency of risk stratification was also evaluated by applying ROC analyses to models comprised of traditional risk factors alone or in combination with the PEROX risk score within the entire cohort, as well as within primary prevention and secondary prevention subgroups. Statistical analyses were performed using SAS 8.2 (SAS Institute Inc, Cary N.C.) and R 2.8.0 (Vienna, Austria), and p-values<0.05 were considered statistically significant.

### Results

Clinical and laboratory parameters used in development of the PEROX model are shown in Table 1, and were similar between Derivation and Validation Cohorts.

TABLE 1
Clinical and Laboratory Parameters

| | Derivation | Validation | | |
|---|---|---|---|---|
| | Cohort | Cohort | Death One-year | MI One-year |
| | (N = 5,895) | (N = 1,474) | HR (95% CI) | HR (95% CI) |
| Traditional Risk Factors | | | | |
| Age (years)^{†} | 64.1 ± 11.3 | 64.1 ± 10.9 | 1.88 (1.65-2.14)* | 1.14 (0.99-1.32) |
| Male - n (%)^{†} | 4,021 (68) | 1,024 (69) | 0.93 (0.73-1.18) | 1.21 (0.88-1.66) |
| History of Hypertension - n (%)^{†} | 4,335 (74) | 1,075 (73) | 1.67 (1.24-2.25)* | 1.53 (1.07-2.19)* |
| Current smoking - n (%)^{†} | 770 (13) | 162 (11)* | 0.90 (0.63-1.29) | 1.28 (0.87-1.89) |
| History of smoking - n (%) | 3,869 (66) | 995 (68) | 1.35 (1.04-1.74)* | 0.90 (0.67-1.20) |
| Diabetes mellitus - n (%)^{†} | 2,054 (35) | 544 (37) | 2.09 (1.66-2.62)* | 1.55 (1.17-2.06)* |
| History of CVD - n (%) | 4,056 (71) | 1017 (71) | 2.95 (1.85, 4.70)* | 2.41 (1.39, 4.19)* |
| Laboratory Measurements | | | | |
| Fasting blood glucose (mg/dl)^{†} | 111 ± 47 | 112 ± 43 | 1.23 (1.13-1.33)* | 1.27 (1.16-1.39)* |
| Creatinine (mg/dl)^{†} | 1.1 (0.8-1.1) | 1.1 (0.8-1.1) | 1.57 (1.48-1.67)* | 1.22 (1.09-1.37)* |
| Potassium (mmol/l)^{†} | 4.2 (4.0-4.5) | 4.2 (4.0-4.5) | 1.10 (1.04-1.17)* | 0.97 (0.84-1.12) |
| C-reactive protein (mg/dl)^{†} | 3.0 (1.7-5.9) | 3.0 (1.6-5.5) | 1.92 (1.71-2.16)* | 1.21 (1.05-1.40)* |
| Total cholesterol (mg/dl) | 176 ± 43 | 178 ± 43 | 0.71 (0.62-0.81)* | 0.93 (0.80-1.07) |
| LDL cholesterol (mg/dl) | 100 ± 36 | 101 ± 36 | 0.78 (0.69-0.89)* | 0.97 (0.84-1.13) |
| HDL cholesterol (mg/dl)^{†} | 46 ± 14 | 46 ± 14 | 0.84 (0.74-0.95)* | 0.71 (0.60-0.84)* |
| Triglycerides (mg/dl)^{†} | 160 ± 119 | 163 ± 120 | 0.82 (0.71-0.96)* | 1.07 (0.96-1.19) |
| Clinical Characteristics | | | | |
| Systolic blood pressure (mmHg)^{†} | 135 ± 21 | 136 ± 22* | 0.96 (0.85-1.07) | 1.17 (1.02-1.34)* |
| Diastolic blood pressure (mmHg) | 75 ± 12 | 75 ± 13 | 0.81 (0.73-0.90)* | 0.97 (0.85-1.12) |
| Body mass index (kg/m²)^{†} | 30 ± 6 | 30 ± 6 | 0.78 (0.68-0.89)* | 0.90 (0.78-1.05) |
| Aspirin use - n (%) | 4,270 (72) | 1,087 (73) | 0.64 (0.51-0.81)* | 0.93 (0.68-1.27) |
| Statin use - n (%) | 3,450 (59) | 869 (59) | 0.82 (0.65-1.03) | 0.70 (0.53-0.92)* |
| Events | | | | |
| One-year Death - n (%) | 242 (4) | 54 (4) | | |
| One-year MI - n (%) | 148 (3) | 44 (3) | | |

^{†}Indicates variable was present in PEROX risk score model. Data are shown as mean ± standard deviation for normally distributed continuous variables, median (interquartile range) for non-normally distributed continuous variables, or number in category (percent of total in category) for categorical variables. Hazard ratios were calculated per standard deviation (for normally distributed variables). For variables with non-normal distribution (creatinine, potassium, c-reactive protein), values were log transformed and hazard ratios calculated per log of standard deviation.
*p < 0.05
Abbreviations:
MI, myocardial infarction;
HR, hazard ratio;
CI, confidence interval.

One-year event rates for incident non-fatal MI or death, individually, and as a composite, did not significantly differ between the Derivation and Validation Cohorts (p=0.37 for MI; p=0.50 for death; p=1.00 for MI or death). Many traditional cardiac risk factors predicted one-year death or MI as expected, such as elevations in total cholesterol, LDL cholesterol, and triglycerides. Reduced diastolic blood pressure and body mass index were associated with decrease in risk, likely reflecting confounding by indication bias whereby patients with a higher prevalence of comorbidities are more likely to be taking medication or undergoing aggressive interventions.

Multiple statistically-significant hazard ratios were observed between various leukocyte, erythrocyte, and platelet parameters and incident one-year risks for non-fatal MI and death in univariate analyses, consistent with multiple prior individual reported associations with various hematological parameters (30-33).

Comprehensive Hematological Profile Patterns Identify Patient Risk for Myocardial Infarction or Death.

In the Derivation Cohort, 23 high-risk patterns (Table 2A) were identified in patients that were more likely to experience death (>3.6-fold risk) or MI (>1.4-fold risk) over the ensuing year.

TABLE 2A
High-risk Patterns in PEROX Model for One-year Death or Myocardial Infarction

| Death High Risk | Pattern | N | Death Rate | HR (95% CI) |
|---|---|---|---|---|
| 1 | Hgb content distribution width >3.93, | 815 | 13% | 4.94 (3.88-6.30) |
| | & RBC hgb concentration mean <35.07 | | | |
| 2 | Hypochromic RBC count >189, | 658 | 13% | 4.47 (3.48-5.73) |
| | & Hgb content distribution width >3.93 | | | |
| 3 | Mean corpuscular hgb concentration <34.38, | 466 | 14% | 4.46 (3.42-5.81) |
| | & Perox d/D <0.89 | | | |
| 4 | Hypochromic RBC count >189, | 588 | 13% | 4.37 (3.39-5.64) |
| | & Macrocytic RBC count >192 | | | |
| 5 | Mean corpuscular hgb concentration <33.00, | 422 | 14% | 4.37 (3.33-5.74) |
| | & Mononuclear central x channel <14.38 | | | |
| 6 | Age >67, | 515 | 13% | 4.08 (3.13-5.32) |
| | & Hematocrit <36.45 | | | |
| 7 | Mononuclear polymorphonuclear valley <18.50, | 474 | 13% | 3.85 (2.93-5.07) |
| | Peroxidase y sigma >9.48 | | | |
| 8 | Mononuclear central x channel <14.38, | 494 | 12% | 3.68 (2.80-4.85) |
| | & Peroxidase y mean >19.02 | | | |
| 9 | C-reactive protein >13.75, | 531 | 12% | 3.63 (2.77-4.76) |
| | & History of hypertension | | | |

| MI High Risk | Pattern | N | MI Rate | HR (95% CI) |
|---|---|---|---|---|
| 1 | Mean platelet concentration >27.89, | 332 | 5% | 2.17 (1.33-3.56) |
| | & Potassium <3.85 | | | |
| 2 | Triglycerides <130, | 464 | 5% | 1.94 (1.23-3.04) |
| | & Age >76 | | | |
| 3 | RBC distribution width >13.83, | 371 | 5% | 1.93 (1.18-3.17) |
| | & Lymphocyte count >1.75 | | | |
| 4 | Hypochromic RBC count >56, | 1,212 | 4% | 1.91 (1.37-2.68) |
| | & Diabetes | | | |
| 5 | Body mass index <24.7, | 446 | 4% | 1.91 (1.20-3.03) |
| | & Neutrophil count <3.58 | | | |
| 6 | Systolic blood pressure >150, | 1,163 | 4% | 1.89 (1.35-2.66) |
| | & History of hypertension | | | |
| 7 | Polymorphonuclear cluster x axis mode >29.87, | 729 | 4% | 1.80 (1.22-2.67) |
| | & RBC distribution width >13.22 | | | |
| 8 | Hgb distribution width >2.69, | 842 | 4% | 1.79 (1.23-2.61) |
| | & Peroxidase y sigma >8.59 | | | |
| 9 | Platelet concentration distribution width <5.39, | 870 | 4% | 1.79 (1.23-2.60) |
| | & RBC hgb concentration mean <34.69 | | | |
| 10 | Mean corpuscular hemoglobin >32.60, | 500 | 4% | 1.78 (1.13-2.81) |
| | & Male | | | |
| 11 | Lymphocyte count <0.96, | 387 | 4% | 1.73 (1.04-2.87) |
| | & Potassium >4.4 | | | |
| 12 | Platelet concentration distribution width >6.04, | 119 | 4% | 1.7 (0.71-4.06) |
| | & Monocyte count >0.46 | | | |
| 13 | Neutrophil cluster mean y <71.19, | 447 | 4% | 1.69 (1.04-2.74) |
| | & Current smoker | | | |
| 14 | Mean platelet concentration >23.19, | 178 | 3% | 1.36 (0.61-3.03) |
| | & Basophil count >0.12 | | | |

Shown above are high risk patterns present in the population, with N representing the number of patients in Derivation Cohort in each pattern. The event rate within each pattern and hazard ratio (95% confidence interval) are shown for each pattern based on univariate Cox models for ranking purposes. Units for each variable are shown in Table 1.

Unique discriminating patterns in those who died included variables derived from multiple erythrocyte- and leukocyte (peroxidase)-related parameters, as well as plasma levels of C-reactive protein. High-risk patterns for MI included multiple erythrocyte, leukocyte (peroxidase) and platelet parameters, traditional risk factors, and blood chemistries (Table 2A). Variables common to both high-risk death and MI patterns included age, hypertension, mean red blood cell hemoglobin concentration, hemoglobin concentration distribution width, hypochromic erythrocyte cell count, and perox Y sigma (a peroxidase-based measure of neutrophil size distribution). An additional 24 low-risk patterns (Table 2B) were observed in patients less likely to experience death (<0.34-fold risk) or MI (<0.57-fold risk).

TABLE 2B
Low-risk Patterns in PEROX Model for One-year Death or Myocardial Infarction

| Death Low Risk | Pattern | N | Death Rate | HR (95% CI) |
|---|---|---|---|---|
| 1 | RBC hgb concentration mean >35.07, | 1,443 | 1% | 0.18 (0.10-0.31) |
| | & Hematocrit >42.25 | | | |
| 2 | Macrocytic RBC count <192, | 2,283 | 1% | 0.22 (0.15-0.32) |
| | & Age <67 | | | |
| 3 | RBC hgb concentration mean >35.07, | 1,494 | 1% | 0.24 (0.15-0.38) |
| | & RBC count >4.42 | | | |
| 4 | Mean platelet concentration >27.52, | 1,651 | 1% | 0.24 (0.18-0.38) |
| | & Age <67 | | | |
| 5 | Peroxidase y sigma <8.10, | 1,982 | 1% | 0.26 (0.17-0.38) |
| | & Age <87 | | | |
| 6 | C-reactive protein <4.0, | 1,688 | 1% | 0.26 (0.17-0.40) |
| | & Hematocrit >42.25 | | | |
| 7 | Hematocrit >42.25, | 1,972 | 1% | 0.27 (0.18-0.40) |
| | & Perox d/D >0.89 | | | |
| 8 | Mononuclear polymorphonuclear valley >18.50, | 1,750 | 1% | 0.27 (0.18-0.41) |
| | & Age <67 | | | |
| 9 | RBC hgb concentration mean >35.07, | 1,436 | 1% | 0.30 (0.19-0.46) |
| | & White blood cell count <5.86 | | | |
| 10 | Neutrophil count <3.96, | 1,697 | 2% | 0.34 (0.23-0.49) |
| | & Age <67 | | | |

| MI Low Risk | Pattern | N | MI Rate | HR (95% CI) |
|---|---|---|---|---|
| 1 | No history of cardiovascular disease, | 919 | 1% | 0.31 (0.15-0.63) |
| | & RBC distribution width <13.22 | | | |
| 2 | Lymphocyte/Large unstained cell threshold <44.50, | 946 | 1% | 0.34 (0.17-0.66) |
| | & Blasts % <0.51 | | | |
| 3 | Systolic blood pressure <134, | 743 | 1% | 0.34 (0.16-0.73) |
| | & Basophil count <0.03 | | | |
| 4 | Platelet clumps >41, | 782 | 1% | 0.37 (0.18-0.76) |
| | & Fasting Blood Glucose <92.5 | | | |
| 5 | Hemoglobin distribution width <2.69, | 891 | 1% | 0.41 (0.22-0.77) |
| | & Hypochromic RBC count <14 | | | |
| 6 | Hypochromic RBC count <14, | 1,159 | 1% | 0.43 (0.25-0.74) |
| | & Neutrophil count <5.83 | | | |
| 7 | Mononuclear central x channel <12.70, | 841 | 1% | 0.44 (0.23-0.82) |
| | & Neutrophil y cluster mean >69.30 | | | |
| 8 | Mononuclear polymorphonuclear valley >14.50, | 910 | 1% | 0.44 (0.24-0.81) |
| | & Creatinine <0.75 | | | |
| 9 | No history of cardiovascular disease, | 756 | 1% | 0.44 (0.23-0.86) |
| | & Systolic blood pressure <134 | | | |
| 10 | Number of peroxidase saturated cells <0.01, | 781 | 1% | 0.47 (0.25-0.90) |
| | & Neutrophil count <4.69 | | | |
| 11 | High density lipoprotein cholesterol >59, | 830 | 1% | 0.49 (0.27-0.90) |
| | & Mean platelet concentration <28.56 | | | |
| 12 | Mononuclear central x channel <12.70, | 896 | 1% | 0.49 (0.27-0.88) |
| | & C-reactive protein <5.31 | | | |
| 13 | Mononuclear central x channel <12.70, | 961 | 1% | 0.54 (0.31-0.93) |
| | & Basophil count <0.07 | | | |
| 14 | No history of cardiovascular disease, | 1,261 | 2% | 0.57 (0.36-0.92) |
| | & Neutrophil cluster mean x <66.07 | | | |

Shown are low risk patterns present in the population, with N representing the number of patients in Derivation cohort in each pattern. The event rate within each pattern and hazard ratio (95% confidence interval) are shown for each pattern based on univariate Cox models for ranking purposes. Units for each variable are shown in Table 1.

Variables that were shared between low-risk patterns for both death and MI risk included C-reactive protein levels, absolute neutrophil count, mean platelet concentration (a flow cytometry determined index of platelet granule content), and monocyte/polymorphonuclear valley (a measure of separation among clusters of peroxidase-containing cell populations). In general, the low-risk patterns for incident one-year death and MI risk are dominated by multiple diverse hematology analyzer variables of all three blood cell types (erythrocyte, leukocyte, platelet) and age.

A composite PEROX model for prediction of incident one-year death or non-fatal MI risk was generated within the Derivation Cohort by summing individual high and low-risk patterns for death and MI individually. The reproducibility of the PEROX model was assessed by examining multiple replicate samples from multiple subjects both within and between days, revealing intra-day and inter-day coefficients of variance of 5±0.4% (mean±S.D.) and 10±2%, respectively. Stability of high- and low-risk patterns used for construction of the PEROX score, and model validation analyses with Somers' D rank correlation 40 and Hosmer-Lemeshow statistic 41 are provided further below.

The PEROX Model Predicts Incident One-Year Risks for Non-Fatal MI and Death.

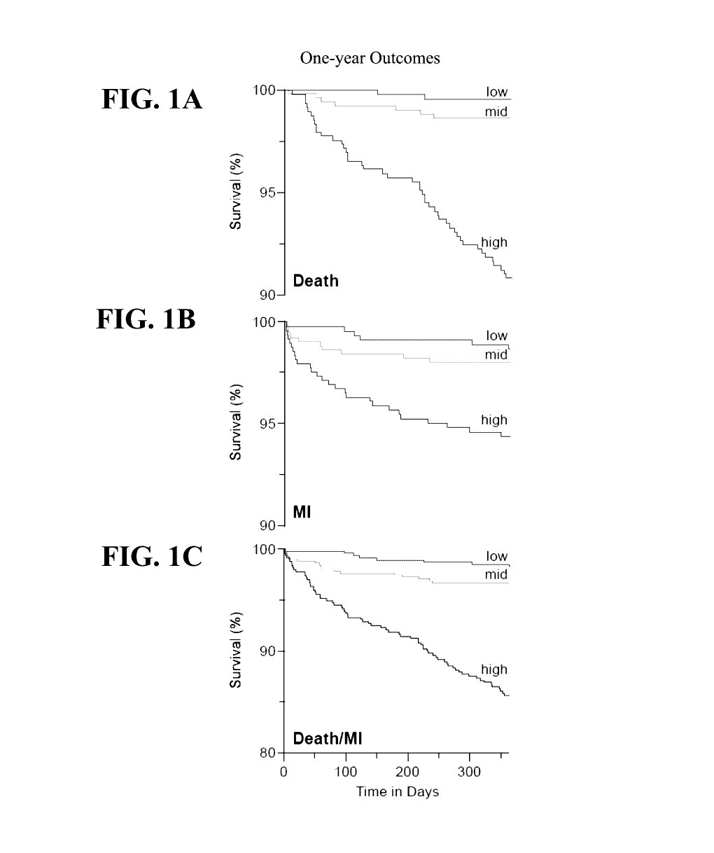

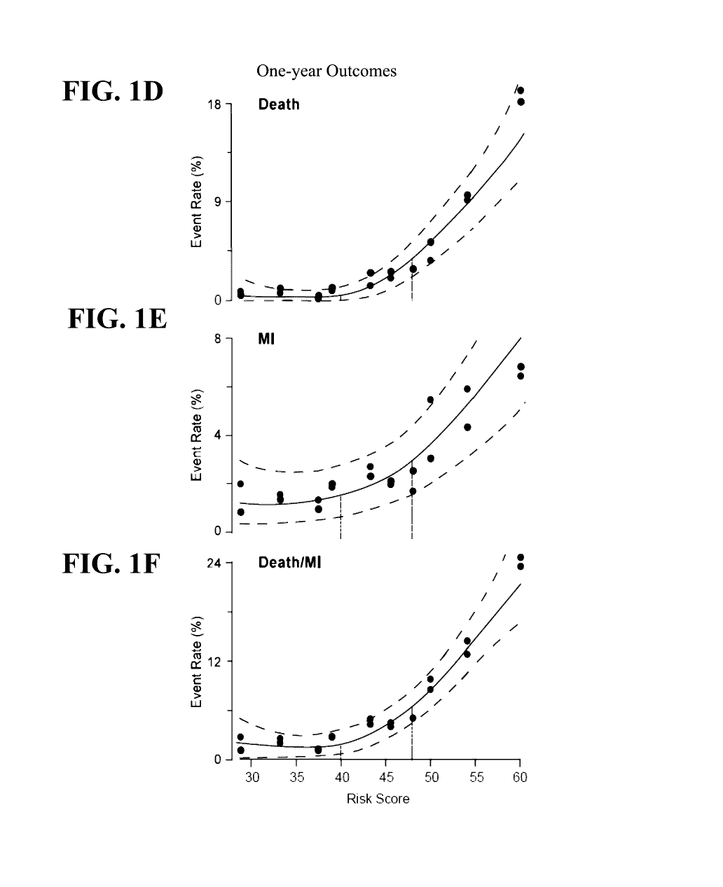

Within the Derivation Cohort, the PEROX model ROC curve analyses for the one-year endpoints of death, MI and the composite of death/MI demonstrated an area under the curve of 80%, 66% and 75%, respectively. For the composite endpoint, a ROC curve potential cut point was identified, virtually identical to the top tertile cut-point within the Derivation Cohort. Initial characterization of the performance of the PEROX score within the Validation Cohort included time-to-event analysis for death, MI or the composite of either event using risk score tertiles to stratify subjects into equivalent sized groups of low, medium and high risk (FIG. 1A-C). For each outcome monitored, increasing cumulative event rates were noted over time within increasing tertiles (log rank P<0.001 for each outcome). FIG. 1D-F demonstrates the relationship between predicted (and 95% confidence interval) absolute one year event rates estimated by PEROX score within the Validation Cohort. Also shown are actual event rates plotted in deciles of PEROX scores for both the Derivation and Validation Cohorts. Observed event rates from the Derivation Cohort were similar to those observed in the Validation Cohort (FIG. 1D-F), and strong tight positive associations were noted between increasing risk score and risk for experiencing non-fatal MI, death or the composite adverse outcome.

Relative Performance of the PEROX Model for Accurate Risk Assessment and Reclassification of Patients.

In additional analyses within the Validation Cohort, ROC curve analyses were performed comparing the accuracy of traditional cardiac risk factors alone versus with PEROX for the prediction of one-year death or MI. Traditional risk factors alone showed modest accuracy (AUC=67%) for one-year death or MI, while addition of the PEROX risk score to traditional risk factors significantly increased prognostic accuracy (AUC=78%, p<0.001). To further evaluate the validity of the PEROX score, re-sampling (250 bootstrap samples from the Validation Cohort, n=1,474) was performed and ROC analyses and accuracy for each bootstrap sample was calculated for prediction of one-year death or MI risk.

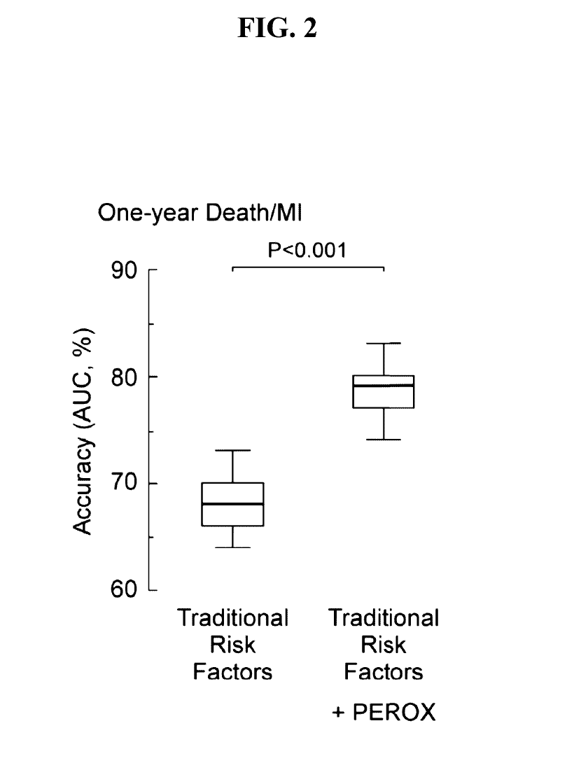

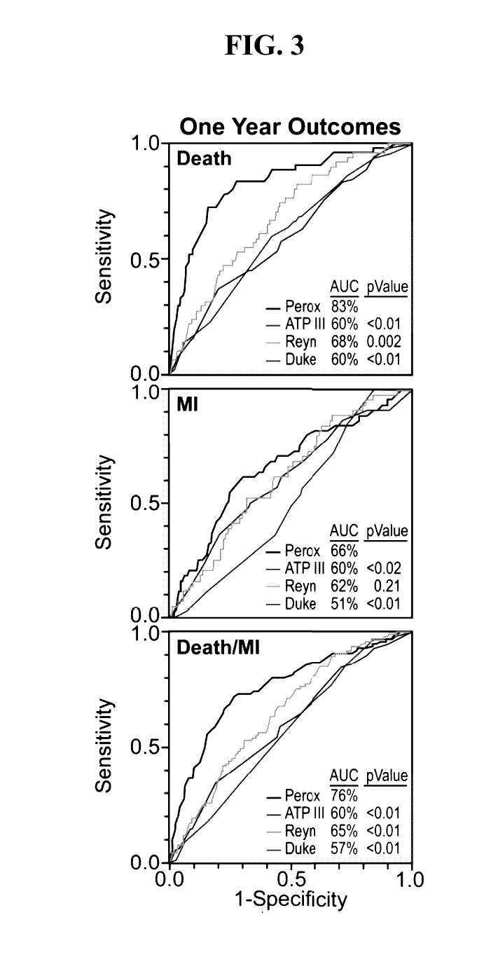

Compared with traditional risk factors alone, the PEROX score demonstrated superior prognostic accuracy among subjects within the independent Validation Cohort (FIG. 2). When PEROX risk score categories were defined by tertiles (in which approximately equal proportions of subjects within the entire cohort are stratified into each risk bin), the one-year event rate for death/MI among subjects stratified within high versus low PEROX risk groups was 14% versus 2%, a risk gradient of 7-fold. Results of Cox proportional hazards regression for time-to-event analyses within the Validation Cohort (N=1,434) are shown in Table 3, and reveal that the PEROX risk score significantly predicts major adverse cardiac endpoints of death, MI, or the composite endpoint even following adjustment for traditional risk factors.

TABLE 3
Unadjusted and adjusted hazard ratio (HR) of PEROX risk scores
for adverse cardiac events at one-year follow-up.

| | Hazard ratio with 95% CI | p-value |
|---|---|---|
| Death | | |
| Unadjusted | 3.68 (2.72, 4.96) | <0.001 |
| Adjusted | 3.74 (2.61, 5.36) | <0.001 |
| MI | | |
| Unadjusted | 1.77 (1.31, 2.38) | <0.001 |
| Adjusted | 2.00 (1.40, 2.87) | <0.001 |
| Death/MI | | |
| Unadjusted | 2.57 (2.06, 3.21) | <0.001 |
| Adjusted | 2.76 (2.14, 3.57) | <0.001 |

Multivariate Cox models were constructed within the Validation Cohort (N = 1,434) for the endpoints death, myocardial infarction (MI), or the composite endpoint death or MI using either the PEROX risk score alone or the PEROX risk score adjusted for traditional risk factors including age, gender, smoking, LDL cholesterol, HDL cholesterol, systolic blood pressure and history of diabetes. Hazard ratios (HR) shown correspond to 1 standard deviation increment. Numbers in parentheses represent 95 percent confidence intervals.

Subjects with a high (top tertile) PEROX risk category relative to low (bottom tertile) PEROX risk show a hazard ratio of 6.5 (95% confidence interval 4.9-8.6) for one-year death/MI. The clinical utility of the PEROX risk score was further compared to traditional risk factors in reclassifying patients into risk groups. As shown in Table 4, adding PEROX score significantly improves risk classification on one-year follow-up for death (NRI=19.4%, p<0.001), MI (NRI=15.6, p=0.002) or both events (NRI=23.5, p<0.001) compared to traditional risk factors alone.

TABLE 4
Reclassification Among Subjects who Experienced versus Did Not
Experienced Adverse Clinical Event on One-Year Follow-up

| | |
|---|---|
| Integrated | |
| Discrimination | Event-Specific |
| Improvement | Reclassification |

| | IDI (%) | p-value | NRI (%) | p-value |
|---|---|---|---|---|
| Death | | | | |
| Without PEROX | — | — | — | — |
| With PEROX | 0.316 | <0.001 | 0.194 | <0.001 |
| MI | | | | |
| Without PEROX | — | — | — | — |
| With PEROX | 0.140 | <0.001 | 0.156 | 0.002 |
| Death/MI | | | | |
| Without PEROX | — | — | — | — |
| With PEROX | 0.220 | <0.001 | 0.235 | <0.001 |

Both net reclassification improvement (NRI) and Integrated Discrimination Improvement (IDI) were used to quantify improvement in model performance.
P-values compare models with/without PEROX risk scores.
Both models were adjusted for traditional risk factors including age, gender, smoking, LDL, cholesterol HDL cholesterol, systolic blood pressure and history of diabetes mellitus.
Cutoff values for NRI estimation used a ratio of 6:3:1 for low, medium and high risk categories.
The risk of adverse cardiac events was estimated using the Cox model.

These findings are consistent among either primary or secondary prevention subjects (Table 5).

TABLE 5
Area under the curve (AUC) values of models with/without PEROX risk
scores for adverse cardiac events at one-year follow-up, stratified
according to primary versus secondary prevention status

| | Primary | Secondary |
|---|---|---|
| | prevention | prevention |
| | (n = 1,859) | (n = 5,510) |
| Death events | 40 events | 256 events |
| Without PEROX | 69 | 70 |
| With PEROX | 81 | 80 |
| p-value | 0.009 | <0.001 |
| MI events | 23 events | 169 events |
| Without PEROX | 58 | 62 |
| With PEROX | 71 | 68 |
| p-value | 0.072 | 0.007 |
| Death/MI events | 63 events | 416 events |
| Without PEROX | 64 | 65 |
| With PEROX | 78 | 75 |
| p-value | <0.001 | <0.001 |

Receiver operating characteristic (ROC) and AUCs (area under the curve) were calculated for one-year death, MI, and combined death or MI endpoints.
ROC curves for the models with/without PEROX were constructed and the corresponding AUC values were compared.
One-year predicted probabilities of an adverse cardiac event were estimated from the Cox model.
P values shown represent comparison of AUC values estimated from models with/without PEROX risk score among primary prevention or secondary prevention subjects within the whole cohort (n = 7,369).
Both models were adjusted for traditional risk factors including age, gender, smoking, LDL cholesterol, HDL cholesterol, systolic blood pressure and history of diabetes.

Table 6: C-statistics comparing one year prognostic accuracy of PEROX vs. alternative clinical risk scores among primary prevention and secondary prevention subjects.

TABLE 6

| | |
|---|---|
| Primary | Secondary |
| prevention | prevention |

| | | | |
|---|---|---|---|
| AUC | P value | AUC | P value |

Death

| | | | | |
|---|---|---|---|---|
| PEROX | 78 | | 81 | |
| ATP III | 58 | <0.001 | 57 | <0.001 |
| Reynolds | 60 | <0.001 | 65 | <0.001 |
| Duke | 50 | NA | 64 | <0.001 |

MI

| | | | | |
|---|---|---|---|---|
| PEROX | 69 | | 64 | |
| ATP III | 54 | 0.054 | 57 | 0.017 |
| Reynolds | 50 | 0.004 | 59 | 0.074 |
| Duke | NA | NA | 54 | 0.001 |

Death/MI

| | | | | |
|---|---|---|---|---|
| PEROX | 75 | | 74 | |
| ATP III | 57 | <0.001 | 57 | <0.001 |
| Reynolds | 56 | <0.001 | 63 | <0.001 |
| Duke | 50 | NA | 60 | <0.001 |

Receiver operating characteristic (ROC) curves and AUC (area under the curve) were calculated (250 bootstrap samples from Primary or Secondary prevention subjects within the Validation Cohort, n = 1474) for one-year death.
MI, and combined death or MI endpoints using risk scores assigned by the PEROX model, the Adult Treatment Panel III (ATP III), Reynolds Risk Score (Reynolds), and Duke angiographic scoring system (Duke) as described under Methods.
P values shown represent comparison of PEROX risk score AUC values relative to ATP III, Reynolds and Duke's angiographic risk scores among primary prevention or secondary prevention subjects.

TABLE 7
Cox proportional hazard model for Predicting
Death/MI at one year in the Validation Cohort

| | Hazard ratio with | |
|---|---|---|
| | 95% CI | P-value |
| PEROX | 2.58 (2.00-3.32) | <0.001 |
| ATP-III | 1.41 (1.14-1.75) | <0.001 |
| Reynolds | 1.33 (1.15-1.55) | <0.001 |
| Duke | 1.28 (1.03-1.59) | <0.001 |

Multivariate Cox Proportional Hazard model time to event (death or non-fatal myocardial infarction) analyses within the Validation Cohort (n = 1,434) for the PEROX, ATP-III, Reynolds and Duke Angiographic risk scores.
COX analyses variables were adjusted to +1 standard deviation increment: Confidence intervals were adjusted for multiplicity using Bonferroni correction.
Abbreviations:
PEROX, PEROX score;
MI, myocardial infarction;
ATP-III, Adult Treatment Panel-III score.

As the above analyses makes clear, the patterns generated by a combination of clinical information and alternative hematology measures can provide significant incremental value. In particular, review of the components contributing to the high- and low-risk patterns that contribute to the PEROX model reveals that a striking number of erythrocyte- and leukocyte related phenotypes, as well as a smaller number of platelet-related parameters, provide prognostic value in identifying individuals at both increased and decreased risk for near term adverse cardiac events. The present Example shows that alterations in multiple subtle phenotypes within leukocyte, erythrocyte and platelet lineages provide prognostic information relevant to cardiovascular health and atherothrombotic risk, consistent with the numerous mechanistic links to cardiovascular disease pathogenesis for each of these hematopoietic lineages.

Hematology analyzers are some of the most commonly used instruments within hospital laboratories. This Example shows that information already captured by these instruments during routine use (but not typically reported) can aide in the clinical assessment of a stable cardiology patient, dramatically improving the accuracy with which subjects can be risk classified at both the high- and low-risk ends of the spectrum.

Blood is a dynamic integrated sensor of the physiologic state. A hematology analyzer profile serves as a holistic assessment of a broad spectrum of phenotypes related to multiple diverse and mechanistically relevant cell types from which can be recognized patterns, like fingerprints, providing clinically useful information in the evaluation of cardiovascular risk in subjects.

The performance of the PEROX score in stable cardiac patients was remarkably accurate given the population examined was comprised of subjects receiving standard of care (i.e. medicated with predominantly normalized lipids and blood pressure) and the relatively short endpoint of one-year outcomes used. Another important finding in the present Example is how much hematology parameters, especially from erythrocyte and leukocyte lineages, contribute to the prognostic value of the PEROX model. This observation strongly underscores the growing appreciation that atherosclerosis is a systemic disease—with parameters in the blood combined with biochemical profiles of systemic inflammation being strongly linked to disease pathogenesis. While many of the patterns identified as low- and high-risk traits within subjects are of unclear biological meaning, a large number are comprised of elements with recognizable mechanistic connections to disease pathogenesis. As a group, all patterns reported appear to be robust, reproducible and present in multiple independent samplings of the independent Validation Cohort. The identification of reproducible high- and low-risk patterns amongst the clinical, laboratory and hematological parameters monitored further indicates the presence of underlying complex relationships between multiple hematologic parameters, clinical and metabolic parameters, and cardiovascular disease pathogenesis.

Much interest focuses on the idea that array-based phenotyping will play an ever increasing role in the future of preventive medicine, serving as a powerful method to improve risk classification of subjects, and ultimately, individualize tailored therapies. Rather than utilize research-based arrays (genomic, proteomic, metabolomic, expression array) that are no doubt powerful and extremely useful, it was decided instead to utilize a robust, high-throughput workhorse of clinical laboratory medicine that is already in broad clinical use—a hematology analyzer. The hematology analyzer selected is commonly available worldwide and has the added advantage of being a flow cytometer that uses in situ peroxidase cytochemical staining for identifying and quantifying leukocytes, an added phenotypic dimension relevant to disease pathogenesis.

While the precise risk score described above is only an exemplary embodiment. Other embodiments for calculating and reporting a risk score may be employed with the present invention. This Example demonstrates, for example, that in the outpatient cardiology clinic setting using only clinical information routinely available plus a drop of blood (˜150 μl), utilization of a broad phenotypic array based approach can permit rapid development of a precise and accurate risk score that provides markedly improved prognostic value of near-term relevance.

### Additional Data and Methods

### I. General Methods and Clinical Definitions

Hematology analyses were performed using an ADVIA 120 hematology analyzer (Siemens, New York, N.Y.), which uses in situ peroxidase cytochemical staining to generate a CBC and differential based on flow cytometry analysis of whole anticoagulated blood.

Additional white blood cell, red blood cell, and platelet related parameters derived from both cytograms and absorbance data were extracted from DAT files used in generating the CBC and differential. All hematology parameters selected for potential use in the PEROX risk score demonstrated reproducible results upon replicate (>10 times) analysis (i.e. those with a standard deviation from mean greater than 30% were excluded from inclusion in the derivation of the PEROX risk score). A blinded reviewer using established screening criteria sequentially assessed all cytograms prior to accepting specimen data. The reproducibility of the PEROX risk score was assessed by examining multiple replicate samples from multiple subjects both within and between days, revealing intra-day and inter-day coefficients of variance of 5±0.4% (mean±S.D.) and 10±2%, respectively.

The mathematical method logical analysis of data (Lauer et al., Circulation. Aug. 6 2002; 106(6):685-690; Crama et al., Annals of Operations Research. 1988 1988; 16(1):299-326; and Boros et al., Math Programming. 1997 1997; 79:163-19; all of which are herein incorporated by reference) was used to identify binary variable pairs that form reproducible positive and negative predictive patterns, and to build a model predictive of risk for death or MI at one-year. Variables were included based on clinical significance, perceived potential informativeness, reproducibility (for hematology parameters) as monitored in inter-day and intra-day replicates, as well as non-redundancy, as assessed by cluster analysis performed within leukocyte, erythrocyte, and platelet subgroups. Definitions for these variables are listed below.

Criteria for the development of the PEROX risk score model included three equal proportions for each hematology parameter variable, two variables per pattern, and a minimal prevalence of 10% of the events for high-risk and 10% of non-events for low-risk patterns. Patterns were generated using logical analysis of data software (http:// followed by “pit.kamick.free.fr/lemaire/LAD/”), and tuned for both homogeneity and prevalence to obtain best accuracy on cross validation experiments. The weight for each positive pattern was [+1/number of high-risk patterns], while for each negative pattern was [−1/number of negative patterns]. The overall risk score a patient was assigned is calculated by the sum of positive and negative pattern weights. A maximum score of +1 would be calculated in a patient with only positive patterns whereas a maximum score of −1 would be present in a patient with only negative patterns. The original score range was adjusted from ±1 to a range of 0 to 100 by assuming 50 (rather than 0) as midpoint of equal variance. The PEROX risk score was calculated: 50×[(1/23 possible high-risk patterns)×(# actual high-risk patterns)−(1/24 possible low-risk patterns)×(# low-risk patterns)]+50. An example calculation is provided further below.

Clinical definitions for Table 1 were defined as follows. Hypertension was defined as systolic blood pressure>140 mmHg, diastolic blood pressure>90 mmHg or taking calcium channel blocker or diuretic medications. Current smoking was defined as any smoking within the past month. History of cardiovascular disease was defined as history of cardiovascular disease, coronary artery bypass graft surgery, percutaneous coronary intervention, myocardial infarction, stroke, transient ischemic attack or sudden cardiac death. Estimated creatinine clearance was calculated using Cockcroft-Gault formula. Myocardial infarction was defined by positive cardiac enzymes, or ST changes present on electrocardiogram. Death was defined by Social Security Death Index query.

### II. Hematology Analysis and Extraction of Data Using Microsoft Excel Macro

Hematology analyses were performed using an Advia 120 hematology analyzer (Siemens, New York, N.Y.). This hematology analyzer functions as a flow cytometer, using in situ peroxidase cytochemical staining to generate a CBC and differential based on flow cytometry analysis of whole anticoagulated blood. An example of a leukocyte cytogram and a table listing all hematology analyzer elements recovered for analysis are shown below. All hematology data utilized was generated automatically by the analyzer during routine performance of a CBC and differential without any additional sample preparation or processing steps. However, additional steps should be taken to ensure the data is saved and extracted appropriately. Information on how to save and extract data is included here. Also, note that these procedures are obtainable from the instrument technical manual as part of the standard operating procedure for the machine. To improve reproducibility of hematology parameters, increased frequency of the calibrator (Cal-Chex H produced by Streck, Omaha, Nebr.) for the hematology analyzer was used (twice weekly and with reagent changes).

Data is saved by going to “Data options” tab on the ADVIA 120 main menu and selecting the “Data export box” (this automatically stores the hematology data in DAT files). In addition, unselect “unit set” and “unit label”. This allows for data to be collected out to additional significant digits. Data can be extracted by opening the DAT files and cutting and pasting into Microsoft Excel. Alternatively, one can use an Excel macro. To utilize the macro, the user should create two folders on the computer desktop. One should be named “export data” and the user should copy the DAT file that needs to be extracted into this folder. The other folder should be named “output data”. The user should open the macro and put the location of the export data and output data in the boxes “Export data” and “Output data”. For example if these folders are on the desktop, one would type in “c: my computer/my desktop/export data” in the “Export data” field. The user should then select “Extract data” and when prompted select the desired DAT file to be extracted. Data will then automatically be extracted with the output present as an excel file in the “Output data” folder.

### III. Sample of Peroxidase-Based Flow Cytometry Cytogram

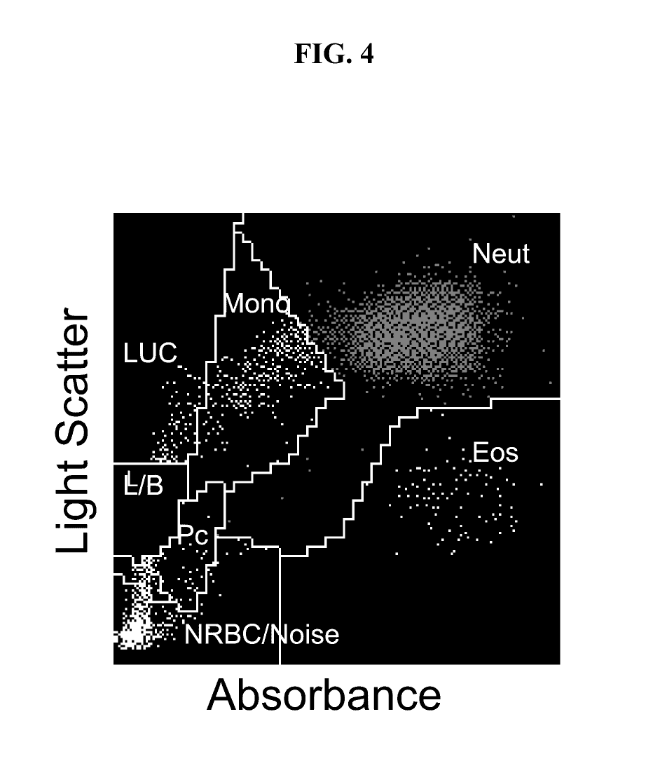

Shown in FIG. 4 is a sample of a peroxidase-based flow-cytometry cytogram from the ADVIA 120 (Siemens). Light scatter measures are on Y axis (surrogate of cellular size) and absorbance measurements are on X axis (surrogate of peroxidase activity). To generate a cell count and differential, populations within pre-specified gates (shown below) are counted. In particular, FIG. 4 shows an example of a Cytogram (˜50,000 cells) as it appears on the analyzer screen. Cell types are distinguished based on differences in peroxidase staining and associated absorbance and scatter measurements. Clusters are in different colors and abbreviations are included to help in distinguishing cell types. Abbreviations: Neutrophils (Neut), Monocytes (Mono), Large unstained cells (LUC), Eosinophils (Eos), Lymphocytes and basophils (L/B), Platelet clumps (Pc) and Nucleated RBCs and Noise (NRBC/Noise).

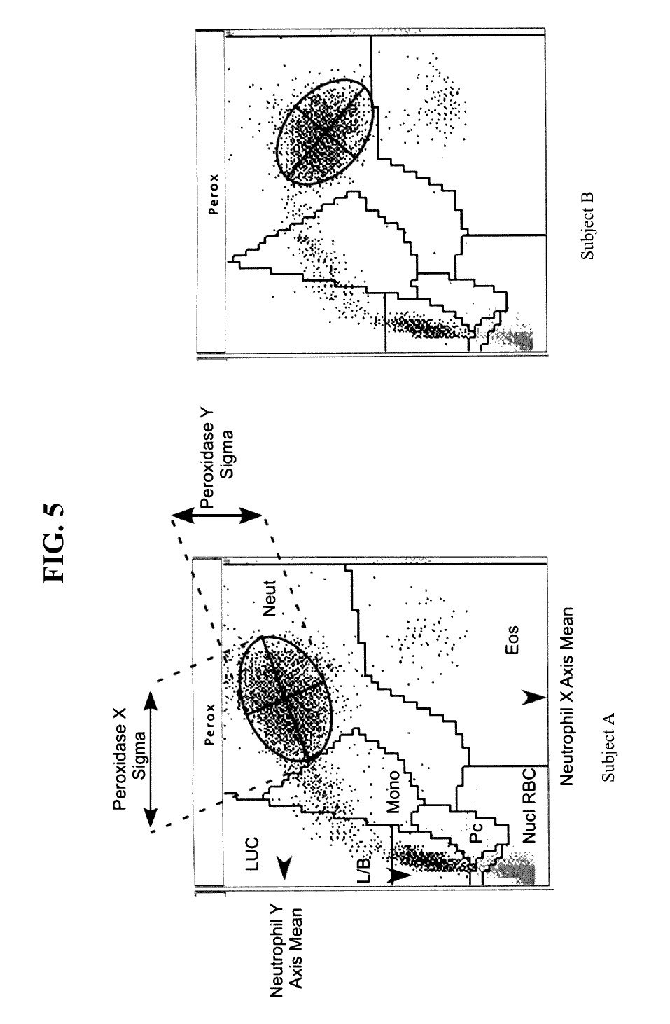

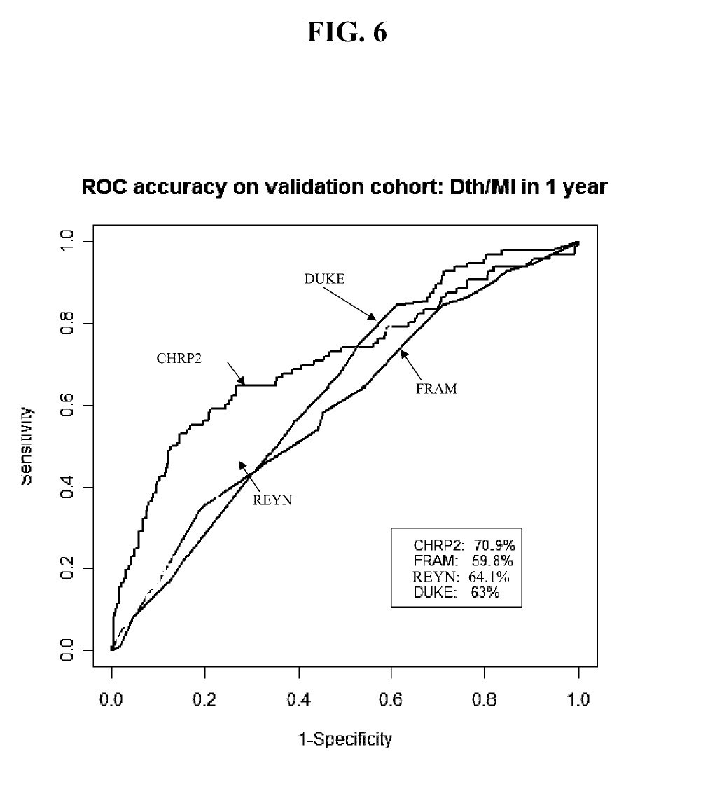

Shown, in FIG. 5, are two examples of cytograms from different subjects. Some of the hematology variables related to the neutrophil main cluster are shown. Subject A has low PEROX risk score. Subject B has a high PEROX risk score. While visual inspection of the cytograms reveals clear differences, the ultimate assignment into “low” (e.g. bottom tertile) vs. “high” (top tertile) risk categories is not possible by visual inspection, since the final PEROX risk score is dependent upon the weighted presence of multiple binary pairs of low and high risk patterns derived from clinical data, laboratory data and hematological parameters from erythrocyte, leukocyte and platelet lineages. In general, cellular clusters (and subclusters) can be defined mathematically by an ellipse, with major and minor axes, distribution widths along major and minor axes, location and angles relative to the X and Y axes, etc. In addition, positional relationships between various (sub)cellular clusters can also be quantified. In this manner, multiple specific quantifiable parameters derived from the leukocyte lineage are reproducibly defined in a given peroxidase (leukocyte) cytogram. Similar phenotypic characterization of erythrocyte (predominantly determined spectrophotometrically), and platelet (cytographic analysis) lineages are also routinely collected as part of a CBC and differential. The availability of this rich array of phenotypic data as part of a routine automated CBC and differential, combined with the fact that erythrocyte, leukocyte (peroxidase) and platelet related processes are mechanistically linked to atherothrombotic disease, was part of the stimulus for the hypothesis that cardiovascular risk information was available within a comprehensive hematology analysis.

The final PEROX score calculation uses only a subset of hematology analyzer elements that are generated during the course of a CBC and differential, in combination with clinical and laboratory data that would routinely be available at patient encounter in an outpatient setting. The table further below shows only those hematology elements that are used during calculation of the PEROX risk score. Also shown are the definition of the hematology elements, and the abbreviations used within the instrument DAT files.

### IV. Example Calculation of the PEROX Risk Score

A 62 year old stable, non-smoking, non-diabetic female with history of hypertension but no history of cardiovascular disease was seen. A CBC with differential was run. Results from a recent basic metabolic panel and fasting lipid profile are available. Blood pressure and body mass index were measured. Pertinent clinical and laboratory values are shown below in Table 8.

TABLE 8

| | Abbr. | Value |
|---|---|---|
| Clinical and Laboratory Data | | |
| Traditional Risk Factors | | |
| Age (years) | AGE | 62 |
| Male | MALE | No |
| History of Hypertension | HTN | Yes |
| Current smoker | SMOKE | No |
| Diabetes mellitus | DM | No |
| History cardiovascular disease | CAD | No |
| Laboratory Data | | |
| Fasting blood glucose (mg/dl) | GLUC | 95.2 |
| Creatinine (mg/dl) | CREAT | 0.83 |
| Potassium (mmol/l) | K | 4.0 |
| C-reactive protein (mg/dl) | CRP | 1.38 |
| High Density Lipoprotein cholesterol | HDL | 44 |
| (mg/dl) | | |
| Triglycerides (mg/dl) | TGS | 161 |
| Clinical Characteristics | | |
| Systolic blood pressure (mm Hg) | SBP | 125 |
| Body mass index (kg/m²) | BMI | 29.0 |
| Hematology Analyzer Data | | |
| White Blood Cell Related | | |
| White blood cell count (×10³/μl) | WBC | 7.34 |
| Neutrophil count (×10³/μl) | #NEUT | 4.53 |
| Lymphocyte count (×10³/μl) | #LYMPH | 2.10 |
| Monocyte count (×10³/μl) | #MONO | 0.37 |
| Eosinophil count (×10³/μl) | #EOS | 0.13 |
| Basophil count (×10³/μl) | #BASO | 0.02 |
| Number of peroxidase saturated cells | #PEROXSAT | 0.00 |
| (×10³/μl) | | |
| Neutrophil cluster mean x | NEUTX | 64.4 |
| Neutrophil cluster mean y | NEUTY | 74.8 |
| Ky | KY | 100 |
| Peroxidase x sigma | PXXSIG | 0.00 |
| Peroxidase y mean | PXY | 19.06 |
| Peroxidase y sigma | PXYSIG | 6.55 |
| Lobularity index | LI | 0.40 |
| Lymphocyte/large unstained cell threshold | LUC | 50 |
| Perox d/D | PXDD | 0.96 |
| Blasts (%) | % BLASTS | 1.8 |
| Polymorphonuclear ratio (%) | | 29.3 |
| Polymorphonuclear cluster x axis mode | PMNX | 64.4 |
| Mononuclear central x channel | MNX | 14.7 |
| Mononuclear central y channel | MNY | 13.3 |
| Mononuclear polymorphonuclear valley | MNPMN | 20 |
| Red Blood Cell Related | | |
| RBC count (×10⁶/μl) | RBC | 4.06 |
| Hematocrit (%) | HCT | 34.6 |
| Mean corpuscular hemoglobin (MCH; pg) | MCH | 30.9 |
| Mean corpuscular hemoglobin conc. | MCHC | 36.3 |
| (MCHC; g/dl) | | |
| RBC hemoglobin concentration mean | CHCM | 36.7 |
| (CHCM; g/dl) | | |
| RBC distribution width (RDW; %) | RDW | 14.1 |
| Hemoglobin distribution width (HDW; g/dl) | HDW | 2.69 |
| Hemoglobin content distribution width | HCDW | 3.50 |
| (CHDW; pg) | | |
| Normochromic/Normocytic RBC count | | 340 |
| (×10⁶/μl) | | |
| Macrocytic RBC count (×10⁶/μl) | #MACRO | 51 |
| Hypochromic RBC count (×10⁶/μl) | #HYPO | 0.0 |
| Platelet Related | | |
| Plateletcrit (PCT; %) | PCT | 0.20 |
| Mean platelet concentration (MPC; g/dl) | MPC | 28.9 |
| Platelet conc. distribution width(PCDW; g/dl) | PCDW | 5.1 |
| Large platelets (×10³/μl) | #-L-PLT | 4 |
| Platelet clumps (×10³/μl) | PLT CLU | 67 |

### Determining the PEROX Risk Score

With simple modifications to the hematology analyzer (ensuring data export for analysis) and allowing for data entry of clinical and laboratory parameters, calculation of the PEROX risk score can be done in automated fashion. Below is a longhand example.

### Step One—Determining Whether Criteria for Each High Risk and Low Risk Pattern are Met.

Elements used to calculate the PEROX risk score are used by determining in Yes/No fashion whether binary patterns associated with high vs. low risk are satisfied. Elements included in patterns combine a small set of clinical/laboratory data available (age, gender, history of hypertension, current smoking, DM, CVD, SBP, BMI and fasting blood glucose, triglycerides, HDL cholesterol, creatinine, CRP and potassium), combined with data measured during performance of a CBC and differential (not all of these values are reported but they are available within the hematology analyzer).

Table 9 below lists the high risk patterns for death and MI. The death high risk pattern #1 consists of a HCDW>3.93 and CHCM<35.07. The example subject has HCDW of 2.69 and CHCM of 36.7. Thus, this subject's data does not satisfy either criterion. Both criteria must be satisfied to have a pattern. This subject therefore does not possess the Death High Risk #1 pattern and is assigned a point value of zero for this pattern. If the subject did fulfill the criterion for the pattern, a point value of one would be assigned.

The above approach is used to fill in whether each High and Low Risk Patterns are satisfied. Table 9 below indicates whether criteria for each high risk pattern for death and MI are met in this example patient.

TABLE 9

| | | Subject | Pattern | Point |
|---|---|---|---|---|
| | Pattern | Values | Present | Value |
| Death | | | | |
| High Risk | | | | |
| 1 | Hemoglobin content distribution width >3.93, | HCDW = 3.50 | No | 0 |
| | & RBC hemoglobin concentration mean <35.07 | CHCM = 36.7 | | |
| 2 | Hypochromic RBC count >189, | #HYPO = 0 | No | 0 |
| | & Hemoglobin content distribution width >3.93 | HCDW = 3.50 | | |
| 3 | Mean corpuscular hemoglobin concentration <34.38, | MCHC = 36.3 | No | 0 |
| | & Perox d/D <0.89 | PXDD = 0.96 | | |
| 4 | Hypochromic RBC count >189, | #HYPO = 0 | No | 0 |
| | & Macrocytic RBC count >192 | #MACRO = 51 | | |
| 5 | Mean corpuscular hemoglobin concentration <33.00, | MCHC = 36.3 | No | 0 |
| | & Mononuclear central x channel <14.38 | MNX = 14.7 | | |
| 6 | Age >67, | AGE = 62 | No | 0 |
| | & Hematocrit <36.45 | HCT = 34.6 | | |
| 7 | Mononuclear polymorphonuclear valley <18.50, | MNPMN = 20 | No | 0 |
| | Peroxidase y sigma >9.48 | PXYSIG = 6.55 | | |
| 8 | Mononuclear central x channel <14.38, | MNX = 14.7 | No | 0 |
| | & Peroxidase y mean >19.02 | PXY = 19.06 | | |
| 9 | C-reactive protein >13.75, | CRP = 1.38 | No | 0 |
| | & History of hypertension | HTN = Yes | | |
| MI | | | | |
| High Risk | | | | |
| 1 | Mean platelet concentration >27.89, | MPC = 28.9 | No | 0 |
| | & Potassium <3.85 | K = 4.0 | | |
| 2 | Triglycerides <130, | TGS = 161 | No | 0 |
| | & Age >76 | AGE = 62 | | |
| 3 | RBC distribution width >13.83, | RDW = 14.1 | Yes | 1 |
| | & Lymphocyte count >1.75 | #LYMPH = 2.10 | | |
| 4 | Hypochromic RBC count >56, | #HYPO = 0 | No | 0 |
| | & Diabetes | DM = NO | | |
| 5 | Body mass index <24.7, | BMI = 29.0 | No | 0 |
| | & Neutrophil count <3.58 | #NEUT = 4.53 | | |
| 6 | Systolic blood pressure >150, | SBP = 125 | No | 0 |
| | & History of Hypertension | HTN = YES | | |
| 7 | Polymorphonuclear cluster x axis mode >29.87, | PMNX = 64.4 | Yes | 1 |
| | & RBC distribution width >13.22 | RDW = 14.1 | | |
| 8 | Hemoglobin distribution width >2.69, | HDW = 2.69 | No | 0 |
| | & Peroxidase y sigma >8.59 | PXYSIG = 6.55 | | |
| 9 | Platelet concentration distribution width <5.39, & | PCDW = 5.1 | No | 0 |
| | RBC hemoglobin concentration mean <34.69 | CHCM = 36.7 | | |
| 10 | Mean corpuscular hemoglobin >32.60, | MCH = 30.9 | No | 0 |
| | & Male | MALE = No | | |
| 11 | Lymphocyte count <0.96, | #LYMPH = 2.10 | No | 0 |
| | & Potassium >4.4 | K = 4.0 | | |
| 12 | Platelet concentration distribution width >6.04, | PCDW = 5.1 | No | 0 |
| | & Monocyte count >0.46 | #MONO = 0.37 | | |
| 13 | Neutrophil cluster mean y <71.19, | NEUT Y = 74.8 | No | 0 |
| | & Current smoker | SMOKE = No | | |
| 14 | Mean platelet concentration >23.19, | MPC = 28.9 | No | 0 |
| | & Basophil count >0.12 | #BASO = 0.02 | | |

Table 10 below indicates whether criteria for each low risk pattern for death and MI are met in this example patient.

TABLE 10

| | | Subject | Pattern | Point |
|---|---|---|---|---|
| | Pattern | Values | Present | Value |
| Death | | | | |
| Low Risk | | | | |
| 1 | RBC hemoglobin concentration mean >35.07, | CHCM = 36.7 | No | 0 |
| | & Hematocrit >42.25 | HCT = 34.6 | | |
| 2 | Macrocytic RBC count <192, | #MACRO = 51 | Yes | 1 |
| | & Age <67 | AGE = 62 | | |
| 3 | RBC hemoglobin concentration mean >35.07, | CHCM = 36.7 | No | 0 |
| | & RBC count >4.42 | RBC = 4.06 | | |
| 4 | Mean platelet concentration >27.52, | MPC = 28.9 | Yes | 1 |
| | & Age <67 | AGE = 62 | | |
| 5 | Peroxidase y sigma <8.10, | PXYSIG = 6.55 | Yes | 1 |
| | & Age <67 | AGE = 62 | | |
| 6 | C-reactive protein <4.0, | CRP = 1.38 | No | 0 |
| | & Hematocrit >42.25 | HCT = 34.6 | | |
| 7 | Hematocrit >42.25, | HCT = 34.6 | No | 0 |
| | & Perox d/D >0.89 | PXDD = 0.96 | | |
| 8 | Mononuclear polymorphonuclear valley >18.50, | MNPMN = 20 | Yes | 1 |
| | & Age <67 | AGE = 62 | | |
| 9 | RBC hemoglobin concentration mean >35.07, | CHCM = 36.7 | No | 0 |
| | & White blood cell count <5.86 | WBC = 7.34 | | |
| 10 | Neutrophil count <3.96, | #NEUT = 4.53 | No | 0 |
| | & Age <67 | AGE = 62 | | |
| MI | | | | |
| Low Risk | | | | |
| 1 | History of cardiovascular disease, | CAD = NO | No | 0 |
| | & RBC distribution width <13.22 | RDW = 14.1 | | |
| 2 | Lymphocyte/Large unstained cell threshold <44.50, | LUC = 50 | No | 0 |
| | & Blasts (%) <0.51 | % BLASTS = 1.8 | | |
| 3 | Systolic blood pressure <134, | SBP = 125 | Yes | 1 |
| | & Basophil count <0.03 | #BASO = 0.02 | | |
| 4 | Platelet clumps >41, | PLT CLU = 67 | No | 0 |
| | & Fasting blood glucose <92.5 | GLUC = 95.2 | | |
| 5 | Hgb distribution width <2.69, | HDW = 2.69 | No | 0 |
| | & Hypochromic RBC count <14 | #HYPO = 0.00 | | |
| 6 | Hypochromic RBC count <14, | #HYPO = 0.00 | Yes | 1 |
| | & Neutrophil count <5.83 | #NEUT = 4.53 | | |
| 7 | Mononuclear central x channel <12.70, | MNX = 14.7 | No | 0 |
| | & Neutrophil cluster mean y >69.30 | NEUTY = 74.8 | | |
| 8 | Mononuclear polymorphonuclear valley >14.50, | MNPMN = 20 | No | 0 |
| | & Creatinine <0.75 | CREAT = 0.83 | | |
| 9 | History of cardiovascular disease, | CAD = NO | No | 0 |
| | & Systolic blood pressure <134 | SBP = 125 | | |
| 10 | Number of peroxidase saturated cells <0.01, | #PEROX SAT = 0 | Yes | 1 |
| | & Neutrophil count <4.69 | #NEUT = 4.53 | | |
| 11 | High density lipoprotein cholesterol >59, | HDL = 44 | No | 0 |
| | & Mean platelet concentration <28.56 | MPC = 28.9 | | |
| 12 | Mononuclear central x channel <12.70, | MNX = 14.7 | No | 0 |
| | & C-reactive protein <5.31 | CRP = 1.38 | | |
| 13 | Mononuclear central x channel <12.70, | MNX = 14.7 | No | 0 |
| | & Basophil count <0.07 | #BASO = 0.02 | | |
| 14 | History of cardiovascular disease, | CAD = 0 | No | 0 |
| | & Neutrophil cluster mean x <66.07 | NEUTX = 64.4 | | |

Step Two—Counting the Number of High and Low Risk Patterns that are Satisfied.

The next step is to count how many positive and negative patterns are fulfilled. Each high risk pattern has a value of +1 and each low risk pattern has a value of −1.

In this example:
Number of high risk patterns: Subject has=2
Number of low risk patterns: Subject has=7

### Step Three—Calculating the Weighted Raw Score.

Subjects generally have combinations of both high and low risk patterns. Overall risk is calculated by a weighted sum of the number of high risk and low risk patterns. The weight for each positive pattern is [+1/number of high risk patterns satisfied], while for each negative pattern is [−1/number of low risk patterns satisfied]. Total possible number of high risk patterns is 23. Total possible number of low risk patterns is 24. Thus, if a subject had all 23 positive risk patterns and no low risk patterns they would have a maximal Raw Score of +1. If a subject had no high risk patterns and all low risk patterns, they would have a minimum Raw Score of −1. The Raw Score of a subject is calculated by the weighted sum of high risk and low risk patterns. In this example, we know:

$$\begin{matrix}Raw Score = ( 1 / 23 possible high \text{-} risk patterns ) \times \\ ( number of high \text{-} risk patterns satisfied ) + \\ ( - 1 / 24 possible low \text{-} risk patterns ) \times \\ ( number of low \text{-} risk patterns satisfied ) \\ = 1 / 23 \times 2 + - 1 / 24 \times 7 \\ = - 0.2047\end{matrix}$$

Note—the Raw Score can have a positive or negative value.

### Step Four—Calculating the Final PEROX Risk Score

The calculated Raw Score ranges from −1 to +1 with 0 as the midpoint. The PEROX Risk Score adjusts the range from ±1 to a range of 0 to 100 by assuming 50 (rather than 0) as the midpoint of the scale. This is achieved by multiplying the Raw Score by 50, and then adding 50.

$$\begin{matrix}PEROX Risk Score = ( 50 \times Raw Score ) + 50 \\ = ( 50 \times - 0.2047 ) + 50 \\ = 39.8\end{matrix}$$

FIG. 1F allows one to use the Perox Risk Score to estimate overall incident risk of death or MI over the ensuing one-year period. In this example, the subject's 1 yr event rate is approximately 2%.

### VI. PEROX Model Validation

The Somers' D rank correlation, Dxy, provides an estimate of the rank correlation of the observed binary response and a continuous variable. Thus, it can be used as an indicator of model fit for the PEROX model. Dxy in the PEROX model measures a correlation between the predicted PEROX score and observed binary response (event vs. non-event). The Dxy for both Derivation and Validation cohorts was calculated. A large difference in D×y values between these two cohorts indicates a large prediction error. As can be seen from the table below, there is no evidence of lack of fit since the differences are small for all three cases. Based upon these analyses, the PEROX risk score showed small overall prediction errors (e.g. 3.8% difference between Derivation and Validation Cohorts for one year Death or MI outcome).

TABLE 11
Model validation of the PEROX model using Dxy

| Dxy | Derivation | Validation | Difference (%) |
|---|---|---|---|
| Death | 0.607 | 0.676 | 11.4 |
| MI | 0.319 | 0.306 | 4.1 |
| Death/MI | 0.501 | 0.520 | 3.8 |

Hosmer-Lemeshow statistic is a goodness of fit measure for binary outcome models when the prediction is a probability. However the PEROX risk score is not a probability, hence the Hosmer-Lemeshow statistic cannot be directly applied to PEROX score. Therefore, the PEROX risk scores were converted on a probability scale through a logistic regression model. Then Hosmer-Lemeshow test was applied to examine the goodness of fit using PEROX score as a risk factor for event prediction. As can be seen from the results below, no evidence of lack of fit was observed since all p-values are significantly larger than 0.05.

TABLE 12
Model validation of the PEROX model
using Hosmer Lemeshow test

| | χ² | p-value |
|---|---|---|
| Death | 8.08 | 0.426 |
| MI | 2.73 | 0.950 |
| Death/MI | 11.68 | 0.166 |

To provide further realistic simulation, the method used for generating the PEROX risk score was cross-validated by using ten random 10-folding experiments within the learning dataset (Derivation Cohort). k-folding is a cross-validation technique in which the samples are randomly divided into k parts, 1 part is used as the test set and the remaining k−1 parts are used for training. The test set is permuted by leaving out a different test set each time. In this case, k=10 was used and the entire procedure was repeated 10 times, resulting in 100 experiments within the Derivation cohort. The data contains a relatively small proportion of deaths and MIs in 1 year. To ensure that there was a fair sampling of the Death and MI events in all the k-folds, random stratified sampling was performed (meaning that Death, MI, and controls were randomly divided into k parts separately within the Derivation cohort). Within each fold, separate LAD models were built for Death vs. controls and MI vs. controls. Cut-points were selected on the training data using 3 equal frequency cuts. The Death and MI models were combined and used to compute the PEROX score on the test set. Area under the ROC curve was computed on the test set. The summary results for the 100 experiments are presented in Table 13 below.

TABLE 13
Model validation of the PEROX model k [symbol] -folding technique

| | 25% | 50% | 75% |
|---|---|---|---|
| AUC | 0.68 | 0.72 | 0.75 |

TABLE 14
Univariate Cox Proportional Hazard Analysis for Prediction of One-Year Outcomes
Using Peroxidase-based Hematology Parameters Included in PEROX Model

| | Derivation | Validation | Death 1 Year | MI I Year |
|---|---|---|---|---|
| | Cohort | Cohort | HR (95% CI) | HR (95% CI) |
| White Blood Cell Related | | | | |
| White blood cell count (×10³/μl) | 6.50 ± 2.19 | 6.51 ± 2.22 | 1.31 (1.21-1.42) * | 1.04 (0.91-1.20) |
| Neutrophil count (×10³/μl) | 4.39 ± 1.97 | 4.42 ± 1.94 | 1.37 (1.26-1.48) * | 1.01 (0.88-1.16) |
| Lymphocyte count (×10³/μl) | 1.54 ± 0.76 | 1.52 ± 0.86 | 0.73 (0.62-0.86) * | 1.02 (0.89-1.16) |
| Monocyte count (×10³/μl) | 0.35 ± 0.18 | 0.35 ± 0.17 | 1.13 (1.09-1.16) * | 1.06 (0.96-1.16) |
| Eosinophil count (×10³/μl) | 0.21 ± 0.15 | 0.21 ± 0.18 | 1.11 (1.03-1.19) * | 1.05 (0.93-1.18) |
| Basophil count (×10³/μl) | 0.05 ± 0.03 | 0.05 ± 0.03 | 1.09 (0.98-1.21) | 1.07 (0.94-1.22) |
| Number of peroxidase saturated cells | 0.82 (0.30-1.53) | 0.80 (0.30-1.50) | 1.00 (0.89-1.12) | 1.06 (0.91-1.23) |
| (×10³/μl) | | | | |
| Neutrophil cluster mean x | 61.7 ± 6.0 | 61.7 ± 6.3 | 0.96 (0.86-1.06) | 0.97 (0.85-1.11) |
| Neutrophil cluster mean y | 70.0 ± 6.0 | 70.0 ± 6.4 | 1.01 (0.90-1.14) | 0.95 (0.84-1.07) |
| Ky | 97.36 ± 2.38 | 97.25 ± 2.41 | 0.97 (0.86-1.09) * | 0.90 (0.78-1.04) |
| Peroxidase x sigma | 0.01 ± 0.12 | 0.01 ± 0.12 | 1.10 (1.03-1.18) * | 1.06 (0.96-1.18) |
| Peroxidase y mean | 18.1 ± 0.7 | 18.1 ± 0.7 | 1.61 (1.46-1.77) * | 1.10 (0.96-1.27) |
| Peroxidase y sigma | 8.11 ± 1.07 | 8.12 ± 1.05 | 1.79 (1.61-1.99) * | 1.16 (1.01-1.33) * |
| Lobularity index | 1.9 (1.0-2.1) | 1.9 (1.0-2.1) | 0.92 (0.83-1.01) | 1.03 (0.89-1.20) |
| Lymphocyte/large unstained cell threshold | 45.0 ± 1.6 | 45.1 ± 1.6 | 1.16 (1.08-1.24) * | 1.07 (1.00-1.17) |
| Perox d/D | 0.9 (0.9-1.0) | 0.9 (0.9-1.0) | 0.91 (0.85-0.97) * | 1.16 (0.85-1.56) |
| Blasts (%) | 0.77 ± 0.49 | 0.77 ± 0.49 | 1.34 (1.22-1.47) * | 1.07 (0.93-1.23) |
| Polymorphonuclear ratio (%) | 1.0 (0.99-1.0) | 1.0 (0.99-1.0) | 0.77 (0.65-0.90) * | 0.99 (0.84-1.15) |
| Polymorphonuclear cluster x axis mode | 27.5 ± 3.6 | 27.4 ± 3.7 | 0.91 (0.82-1.02) | 1.08 (0.93-1.25) |
| Mononuclear central x channel | 14.1 (13.0-15.0) | 14.1 (13.0-15.0) | 0.80 (0.74-0.88) * | 1.12 (0.95-1.32) |
| Mononuclear central y channel | 14.5 ± 1.1 | 14.5 ± 1.1 | 0.79 (0.73-0.87) * | 1.04 (0.89-1.20) |
| Mononuclear polymorphonuclear valley | 18.0 (18.0-20.0) | 18.0 (18.0-20.0) | 0.69 (0.61-0.77) * | 1.06 (0.94-1.21) |
| Red Blood Cell Related | | | | |
| RBC count (×10⁶/μl) | 4.30 ± 0.52 | 4.33 ± 0.52 | 0.59 (0.53-0.66) * | 0.93 (0.81-1.08) |
| Hematocrit (%) | 40.9 ± 6.2 | 41.0 ± 4.2 | 0.51 (0.45-0.59) * | 0.78 (0.65-0.93) * |
| Mean corpuscular hgb (MCH; pg) | 30.4 ± 2.1 | 30.3 ± 2.0 | 0.83 (0.75-0.92) * | 1.03 (0.89-1.19) |
| Mean corpuscular hgb conc. (MCHC; g/dl) | 33.4 ± 5.7 | 33.4 ± 5.7 | 0.86 (0.80-0.92) * | 0.91 (0.82-1.01) |
| RBC hgb concentration mean (CHCM; g/dl) | 35.1 ± 1.3 | 35.2 ± 1.3 | 0.53 (0.49-0.59) * | 0.90 (0.78-1.04) |
| RBC distribution width (RDW; %) | 13.4 ± 1.2 | 13.4 ± 1.2 | 1.48 (1.42-1.55) * | 1.26 (1.14-1.40) * |
| Hgb distribution width (HDW; g/dl) | 2.7 ± 0.3 | 2.7 ± 0.3 | 1.52 (1.39-1.66) * | 1.26 (1.12-1.43) * |
| Hgb content distribution width (CHDW; pg) | 3.8 ± 0.4 | 3.8 ± 0.4 | 1.44 (1.37-1.51) * | 1.19 (1.07-1.33) * |
| Normochromic/Normocytic RBC count | 3.65 ± 0.39 | 3.66 ± 0.39 | 0.64 (0.60-0.68) * | 0.89 (0.78-1.01) |
| (×10⁶/μl) | | | | |
| Macrocytic RBC count (×10⁶/μl) | 0.01 (.01-.03) | 0.01 (.01-.03) | 1.76 (1.55-2.00) * | 1.03 (0.89-1.20) |
| Hypochromic RBC count (×10⁶/μl) | 0.006 (0.001-0.002) | 0.005 (0.001-0.002) | 1.12 (0.99-1.27) | 1.18 (1.00-1.38) |
| Platelet Related | | | | |
| Plateletcrit (PCT; %) | 0.18 ± 0.05 | 0.18 ± 0.06 | 1.15 (1.04-1.27) * | 0.99 (0.85-1.14) |
| Mean platelet concentration (MPC; g/dl) | 27.1 ± 1.7 | 27.0 ± 1.7 | 0.75 (0.68-0.83) * | 0.97 (0.84-1.12) |
| Platelet conc. distribution width | 5.6 ± 0.4 | 5.7 ± 0.4 | 0.95 (0.84-1.06) | 0.95 (0.83-1.01) |
| (PCDW; g/dl) | | | | |
| Large platelets (×10³/μl) | 4 (3-6) | 4 (3-6) | 1.10 (0.94-1.28) | 1.10 (0.91-1.34) |
| Platelet clumps (×10³/μl) | 41.5 ± 37.1 | 42.4 ± 36.1 | 1.00 (1.00-1.00) | 1.00 (1.00-1.00) |

All variables listed were present in the PEROX risk score model.
Data are shown as mean ± standard deviation for normally distributed continuous variables, or median (interquartile range) for non-normally distributed continuous variables.
Some variables have no unit of measure associated with them.
Median for peroxidase X sigma was zero, therefore, mean is shown.
Hazard ratios were calculated per standard deviation (for normally distributed variables).
For variables with non-normal distribution, values were log transformed and hazard ratios calculated per log of standard deviation.
Variable definitions are available in Supplemental Material.
Abbreviations:
MI, myocardial infarction;
HR, hazard ratio;
CI, confidence interval;
RBC, red blood cell;
Hgb, hemoglobin.

### Example 2

### Comprehensive Hematology Risk Profile (CHRP): Risk Predictor for One Year Myocardial Infarction and Death Using Data Generated by Conventional Hematology Analyzers During Performance of a Routine CBC with Differential

This example successfully tests the hypothesis that using only information generated from analysis of whole blood with a general hematology analyzer during the performance of a traditional CBC with differential, high and low risk patterns may be identified allowing for development of a Comprehensive Hematology Risk Profile (CHRP), a single laboratory value that accurately predicts incident risks for non-fatal MI and death in subjects.

Methods:

7,369 patients undergoing elective diagnostic cardiac evaluation at a tertiary care center were enrolled for the study. An extensive array of erythrocyte, leukocyte, and platelet related parameters were captured on whole blood analyzed from each subject at the time of performance of a CBC and differential. The patients were randomly divided into a Derivation (N=5,895) and a Validation Cohort (N=1,473). CHRP was developed using Logical Analysis of Data methodology. First, binary high-risk and low-risk patterns amongst collected erythrocyte, leukocyte and platelet data elements were identified for one year incident risk of non-fatal MI or death. Then, a comprehensive single prognostic risk value, CHRP, was developed by combining these high and low risk patterns to form a single prognostic score.

Results:

Using only parameters routinely available from whole blood analysis on a general hematology analyzer, 19 high-risk and 24 low-risk binary patterns were identified using the Derivation Cohort. These patterns were distilled down into a single, highly accurate prognostic value, the CHRP. Independent prospective testing of the CHRP within the Validation Cohort revealed superior prognostic accuracy (71%) for prediction of one-year risk of death or MI compared with traditional cardiovascular risk factors, laboratory tests, as well as clinically established risk scores including Adult Treatment Panel III (60%), Reynolds (65%), and Duke angiographic (57%) scoring systems. Superior prognostic accuracy for prediction of 1 year incident MI and death was also observed with CHRP in both primary and secondary prevention subgroups, diabetics and non-diabetics alike, and even amongst those with no evidence of significant coronary atherosclerotic burden (<50% stenosis in all major coronary vessels) at time of recent cardiac catheterization.

This example demonstrates that the use of a routine automated hematology analyzer for whole blood analysis generates a spectrum of data from which high and low risk patterns can be identified for predicting a subject's risk for experiencing major adverse cardiac events. A composite single value was built based upon these patterns, the Comprehensive Hematology Risk Profile (CHRP), which accurately predicts incident risks for non-fatal MI and death in subjects, and accurately classifies patients for both high and low near-term (one year) cardiovascular risks. Multivariate logistic regression analysis shows that the CHRP is a strong predictor of risk independent of traditional cardiac risk factors and laboratory markers in subjects. Moreover, CHRP provides strong prognostic value even within subjects who show no significant angiographic evidence of atherosclerosis on recent cardiac catheterization.

### Methods and Materials:

The same general methods and materials, including patient sample, described in Example 1 were used for this example.

TABLE 15
Clinical and Laboratory Parameters

| | Derivation | Validation | | |
|---|---|---|---|---|
| | Cohort | Cohort | Death 1 year | MI 1 year |
| | (N = 5,895) | (N = 1,474) | OR (95% CI) | OR (95% CI) |
| Traditional Risk Factors | | | | |
| Age (years) | 64.1 ± 11.3 | 64.1 ± 10.9 | 4.944 (3.316, 7.372)* | 1.296 (0.874, 1.923) |
| Male-n (%) | 4,021 (68) | 1,024 (69) | 0.960 (0.730, 1.263) | 1.222 (0.849, 1.759) |
| Hypertension-n (%) | 4,335 (74) | 1,075 (73) | 1.659 (1.183, 2.298)* | 1.261 (0.853, 1.885) |
| Current smoking-n (%) | 770 (13) | 162 (11)* | 0.866 (0.580, 1.294) | 1.232 (0.784, 1934) |
| History of smoking-n (%) | 3,869 (66) | 995 (68) | | |
| Diabetes mellitus-n (%) | 2,054 (35) | 544 (37) | 2.377 (1.828, 3.089)* | 1.427 (1.034, 1.998)* |
| Laboratory Measurements | | | | |
| Fasting blood glucose (mg/dl) | 111 ± 47 | 112 ± 43 | 1.700 (1.245, 2.321)* | 1.667 (1.088, 2.556)* |
| Creatinine (mg/dl) | 1.1 (0.8-1.1) | 1.1 (0.8-1.1) | 2.963 (2.132, 4.117)* | 1.789 (1.169, 2.738)* |
| Potassium (mmol/l) | 4.2 (4.0-4.5) | 4.2 (4.0-4.5) | | |
| C-reactive protein (mg/dl) | 3.0 (1.7-5.9) | 3.0 (1.6-5.5) | | |
| Total cholesterol (mg/dl) | 176 ± 43 | 178 ± 43 | 0.646 (0.475, 0.879)* | 0.839 (0.564, 1.247) |
| LDL cholesterol (mg/dl) | 100 ± 36 | 101 ± 36 | 0.646 (0.475, 0.879)* | 0.987 (0.666, 1.462) |
| HDL cholesterol (mg/dl) | 46 ± 14 | 46 ± 14 | 0.777 (0.569, 1.062) | 0.669 (0.431, 1.037) |
| Triglycerides (mg/dl) | 160 ± 119 | 163 ± 120 | 0.701 (0.506, 0.971)* | 1.032 (0,690, 1.545) |
| Clinical Characteristics | | | | |
| Systolic blood pressue (mm Hg) | 135 ± 21 | 136 ± 22* | | |
| Diastolic blood pressure (mm Hg) | 75 ± 12 | 75 ± 13 | | |
| Body mass index (kg/m²) | 30 ± 6 | 30 ± 6 | | |
| Aspirin use-n (%) | 4,270 (72) | 1,087 (73) | | |
| Statin use-n (%) | 3,450 (59) | 869 (59) | | |

Abbreviations: MI, myocardial infarction; OR, odds ratio: CI, confidence interval
Data are shown as median (interquartile range) for numerical variables, or number in category (percent of total in category). Odds ratios were calculated per standard deviation for continuous variables.
*p < 0.05

TABLE 16
Hematology Parameters for CHRP Risk Model

| | Derivation | Validation | Death in 1 year | MI in 1 year |
|---|---|---|---|---|
| | cohort | cohort | HR (95% CI)‡ | HR (95% CI)‡ |
| White blood cell related | | | | |
| White blood cell count (×10³/ml) | 6.1 (5.1-7.5) | 6.1 (5.0-7.5) | 1.64 (1.20-2.23) | 0.94 (0.64-1.37) |
| Neutrophils (%) | 63.9 (57.7-70.7) | 64.8 (58.1-71.2) | 2.27 (1.65-3.12) | 0.84 (0.56-1.25) |
| Lymphocytes (%) | 23.8 (18.1-29.6) | 23 (17.7-28.5) | 0.35 (0.26-0.49) | 1.07 (0.72-1.59) |
| Monocytes (%) | 5.3 (4.3-6.3) | 5.2 (4.3-6.4) | 1.52 (1.13-2.04) | 1.41 (0.95-2.10) |
| Eosinophils (%) | 3.0 (2.0-4.3) | 2.9 (1.9-4.1) | 0.85 (0.63-1.14) | 1.16 (0.77-1.75) |
| Basophils (%) | 0.6 (0.4-0.9) | 0.6 (0.4-0.9) | 0.70 (0.51-0.95) | 1.36 (0.90-2.05) |
| Large unstained cells (%) | 2.1 (1.6-2.7) | 2.1 (1.6-2.7) | 0.77 (0.56-1.04) | 1.12 (0.75-1.68) |
| Neutrophil count (×10³/ml) | 4.0 (3.1-5.2) | 4.0 (3.2-5.2) | 2.15 (1.56-2.95) | 1.00 (0.68-1.47) |
| Lymphocyte count (×10³/ml) | 1.5 (1.1-1.9) | 1.4 (1.1-1.8) | 0.45 (0.33-0.63) | 0.91 (0.61-1.36) |
| Monocyte count (×10³/ml) | 0.3 (0.3-0.4) | 0.3 (0.3-0.4) | 2.05 (1.50-2.80) | 1.19 (0.81-1.74) |
| Eosinophil count (×10³/ml) | 0.2 (0.1-0.3) | 0.2 (0.1-0.3) | 0.93 (0.70-1.25) | 1.05 (0.72-1.54) |
| Basophil count (×10³/ml) | 0 (0-0.1) | 0 (0-0.1) | 0.90 (0.66-1.23) | 1.25 (0.81-1.91) |
| Red blood cell related | | | | |
| RBC count (×10⁶/ml) | 4.3 (4.0-4.6) | 4.3 (4.0-4.7) | 0.32 (0.23-0.46) | 0.83 (0.56-1.23) |
| Hematocrit (%) | 41.2 (38.1-43.8) | 41.3 (38.4-43.9) | 0.32 (0.23-0.45) | 0.69 (0.46-1.02) |
| Mean Corpuscular volume (MCV) | 88.4 (85.5-91.4) | 88.4 (85.3-91.3) | 1.52 (1.11-2.07) | 1.14 (0.79-1.65) |
| Mean corpuscular hgb (MCH; pg) | 30.5 (29.4-31.6) | 30.5 (29.3-31.6) | 0.77 (0.58-1.03) | 1.20 (0.83-1.75) |
| Mean corpuscular hgb concentration | 34.4 (33.7-35.0) | 34.4 (33.6-35.1) | 0.24 (0.17-0.35) | 0.93 (0.62-1.39) |
| (MCHC; g/dl) | | | | |
| RBC hgb concentration mean | 35.2 (34.3-35.9) | 35.2 (34.4-36.0) | 0.24 (0.17-0.35) | 0.79 (0.54-1.15) |
| (CHCM; g/dl) | | | | |
| RBC distribution width (RDW; %) | 13.2 (12.7-13.8) | 13.1 (12.6-13.8) | 5.84 (3.96-8.62) | 1.95 (1.28-2.97) |
| Hgb distribution width (HDW; g/dl) | 2.6 (2.5-2.8) | 2.6 (2.5-2.8) | 2.74 (1.95-3.85) | 1.52 (1.03-2.23) |
| Hgb content distribution width | 3.8 (3.6-4.0) | 3.8 (3.6-4.0) | 4.23 (2.95-6.06) | 1.25 (0.84-1.86) |
| (CHDW; pg) | | | | |
| Macrocytic RBC count (×10⁶/ml) | 140 (65-296) | 133.5 (64-293) | 3.30 (2.31-4.73) | 1.31 (0.89-1.91) |
| Hypochromic RBC count (×10⁶/ml) | 56 (16-165) | 49 (15-148) | 2.36 (1.74-3.20) | 1.67 (1.12-2.49) |
| Hyperchromic RBC count (×10⁶/ml) | 685 (389-1217) | 722.5 (403-1247) | 0.42 (0.30-0.58) | 0.97 (0.65-1.43) |
| Microcytic RBC count (×10⁶/ml) | 236 (133-437) | 244 (134-444) | 1.90 (1.39-2.59) | 0.92 (0.63-1.34) |
| NRBC count | 42 (30-60) | 43 (30-61) | 1.48 (1.09-1.99) | 0.93 (0.63-1.38) |
| Measured HGB | 13.1 (12-14.1) | 13.2 (12.1-14.2) | 0.23 (0.16-0.33) | 0.79 (0.53-1.18) |
| Platelet related | | | | |
| Platelet count (PLT; %) | 224 (186-266) | 220 (183-264) | 0.95 (0.70-1.28) | 0.83 (0.57-1.23) |
| Mean platelet volume (MPV) | 7.8 (7.3-8.4) | 7.8 (7.4-8.4) | 1.49 (1.10-2.03) | 1.14 (0.77-1.69) |
| Platelet distribution width (PDW) | 55.6 (51.5-59.9) | 55.8 (51.6-60.3) | 1.31 (0.96-1.79) | 1.15 (0.77-1.72) |
| Plateletcrit (PCT; %) | 0.2 (0.2-0.2) | 0.2 (0.2-0.2) | 1.10 (0.81-1.48) | 0.77 (0.52-1.14) |
| Mean platelet concentration | 27.3 (26.2-28.2) | 27.3 (26.3-28.1) | 0.45 (0.33-0.62) | 0.94 (0.65-1.36) |
| (MPC; g/dl) | | | | |
| Large platelets (×10³/ml) | 4 (3-6) | 4 (3-6) | 1.31 (0.98-1.75) | 1.06 (0.72-1.56) |
| Flag for left shift >0^{∫} | 2331 (39.5) | 592 (40.2) | 1.57 (1.22-2.02) | 0.99 (0.71-1.38) |

Abbreviations:
MI, myocardial infarction;
HR, hazard ratio;
CI, confidence interval;
RBC, red blood cell;
Hgb, hemoglobin.
Data are shown as median (interquartile range). Some variables have no unit of measure associate with them.
Hazard ratios were calculated for tertile 3 vs. tertile 1.
‡Derivation Cohort only
^{∫}Dichotomous variable presented as number in category (percent of total in category).

TABLE 17a
High Risk Patterns for CHRP model for 1 year death or MI

| | Dth/MI in 1 year | Dth/MI in 1 year | MI in 1 year |
|---|---|---|---|
| | RR (95% CI) | RR (95% CI) | RR (95% CI) |
| Death (1 year) high risk patterns | | | |
| RBC distribution width >13.35 & | 3.43 (2.68-4.39) | 3.78 (2.9-4.94) | 1.55 (0.77-3.11) |
| Percent Eosinophils <38.5 | | | |
| Hematocrit <43.55 & | 2.45 (1.93-3.12) | 2.81 (2.17-3.65) | 0.98 (0.49-1.98) |
| Percent Lymphocytes <28.15 | | | |
| Mean corpuscular hgb concentration < | 2.21 (1.77-2.77) | 2.29 (1.8-2.91) | 1.49 (0.74-2.99) |
| 35.25 & | | | |
| Lymphocyte count <1.405 | | | |
| Mean corpuscular hgb concentration < | 2.08 (1.67-2.6) | 2.18 (1.73-2.75) | 1.05 (0.49-2.27) |
| 33.65 & | | | |
| Percent Lymphocytes >5.1 | | | |
| RBC count <4.135 & | 2.03 (1.62-2.54) | 2.17 (1.71-2.75) | 1.81 (0.9-3.63) |
| Percent Basophils <2.75 | | | |
| White blood cell count >6.715 | 1.88 (1.51-2.35) | 2.03 (1.61-2.57) | 1.24 (0.61-2.54) |
| Eosinophil count <0.08 or >0.37 & | 1.72 (1.36-2.18) | 1.84 (1.44-2.35) | 0.73 (0.28-1.89) |
| Monocyte count >0.265 | | | |
| MI (1 year) high risk patterns | | | |
| Platelet count <226.5 & | 2.1 (1.57-2.81) | 2.05 (1.09-3.83) | 2.34 (1.69-3.24) |
| Hematocrit <40.35 | | | |
| Monocyte count >0.365 & | 1.96 (1.49-2.59) | 1.87 (1.03-3.39) | 2.08 (1.52-2.86) |
| Percent Eosinophils >2.15 | | | |
| RBC distribution width >12.85 & | 2.12 (1.6-2.8) | 2.55 (1.43-4.53) | 2.03 (1.47-2.8) |
| Percent Monocytes >5.85 | | | |
| Platelet count <175.5 & | 2.05 (1.47-2.85) | 2.05 (1.01-4.17) | 2.02 (1.38-2.96) |
| RBC distribution width >12.85 | | | |
| Platelet count <226.5 & | 1.91 (1.38-2.66) | 2.39 (1.23-4.62) | 1.99 (1.37-2.89) |
| Monocyte count >0.365 | | | |
| RBC distribution width >14.25 & | 2.31 (1.72-3.11) | 3.07 (1.89-5.58) | 1.95 (1.36-2.8) |
| Neutrophil count >1.21 | | | |
| Percent Neutrophils >51.8 and <78.1 & | 1.68 (1.16-2.43) | 1.14 (0.46-2.85) | 1.95 (1.31-2.91) |
| Mean corpuscular hgb >32.35 | | | |
| Percent Lymphocytes <12.8 or >34.9 & | 2.09 (1.49-2.93) | 3.25 (1.72-6.14) | 1.92 (1.29-2.87) |
| Hematocrit <40.35 | | | |
| Percent Lymphocytes <23.75 & | 1.81 (1.35-2.42) | 1.34 (0.69-2.6) | 1.91 (1.37-2.66) |
| Percent Neutrophils <69.75 | | | |
| Hematocrit <40.35 & | 2.17 (1.63-2.89) | 3.47 (1.97-6.14) | 1.9 (1.35-2.67) |
| Percent Lymphocytes <23.75 | | | |
| Mean corpuscular hgb >32.35 & | 1.75 (1.22-2.52) | 1.4 (0.6-3.26) | 1.86 (1.23-2.79) |
| Percent Neutrophils >57.29 | | | |
| Eosinophil count >0.305 & | 1.81 (1.3-2.51) | 1.75 (0.86-3.56) | 1.8 (1.23-2.63) |
| Percent Monocytes >3.75 | | | |

Abreviations:
RR, Relative risk;
CI, Confidence interval.
Shown above are high risk patterns present in the population along with relative risk (95% confidence interval) are shown for each pattern in the subset of the derivation cohort on which they were generated (i.e. patients in the derivation cohort with Dth/MI = 1 or maximum stenosis <50%).
Units for each variable are shown in Tables 16.

TABLE 17b
Low Risk Patterns for CHRP model for 1 year death and MI

| | Death or MI | Death | MI |
|---|---|---|---|
| Death (1 year) low risk patterns | RR (95% CI) | RR (95% CI) | RR (95% CI) |
| RBC distribution width <15.05 & | 0.25 (0.2-0.31) | 0.22 (0.18-0.28) | 0.75 (0.32-1.72) |
| Percent Lymphocytes >13.45 | | | |
| RBC distribution width <15.05 & | 0.26 (0.21-0.32) | 0.23 (0.19-0.29) | 0.62 (0.28-1.38) |
| RBC count >3.625 | | | |
| Monocyte count <0.465 & | 0.31 (0.25-0.38) | 0.27 (0.22-0.34) | 0.89 (0.4-1.98) |
| Lymphocyte count >0.865 | | | |
| Hematocrit >39.15 & | 0.34 (0.27-0.42) | 0.29 (0.22-0.37) | 0.72 (0.36-1.46) |
| Percent Neutrophils <76.65 | | | |
| RBC distribution width <17.05 & | 0.42 (0.34-0.53) | 0.39 (0.3-0.49) | 0.58 (0.29-1.17) |
| RBC count >4.135 | | | |
| Hematocrit >34.95 & | 0.43 (0.34-0.54) | 0.4 (0.31-0.51) | 0.6 (0.3-1.2) |
| White blood cell count <6.715 | | | |
| RBC distribution width <13.35 & | 0.47 (0.36-0.62) | 0.45 (0.34-0.61) | 0.7 (0.32-1.5) |
| White blood cell count >5.285 | | | |
| Eosinophil count <0.375 & | 0.58 (0.44-0.76) | 0.53 (0.39-0.71) | 1.12 (0.54-2.32) |
| White blood cell count <5.285 | | | |
| Percent Basophils >0.3 and <1.2 | 0.56 (0.42-0.73) | 0.53 (0.4-0.71) | 0.81 (0.38-1.76) |
| & Percent Monocytes <6.25 | | | |

| | Death/MI in 1 year | Death in 1 year | MI in 1 year |
|---|---|---|---|
| MI-1 low risk patterns | R (95% CI) | RR (95% CI) | RR (95% CI) |
| Hematocrit >40.35 & | 0.51 (0.37-0.71) | 0.59 (0.31-1.14) | 0.46 (0.31-0.67) |
| White blood cell count <6.365 | | | |
| RBC distribution width <12.85 & | 0.42 (0.3-0.59) | 0.23 (0.1-0.55) | 0.48 (0.33-0.69) |
| Percent Neutrophils >32.88 | | | |
| Mean corpuscular hgb <32.35 & | 0.5 (0.38-0.67) | 0.45 (0.25-0.82) | 0.49 (0.35-0.67) |
| Hematocrit >40.35 | | | |
| Monocyte count <0.365 & | 0.43 (0.3-0.62) | 0.2 (0.07-0.54) | 0.49 (0.33-0.73) |
| Lymphocyte count >1.455 | | | |
| Percent Monocytes <5.85 & | 0.54 (0.4-0.74) | 0.54 (0.28-1.04) | 0.51 (0.35-0.73) |
| White blood cell count <6.365 | | | |
| Platelet count >226.5 & | 0.45 (0.32-0.65) | 0.23 (0.09-0.57) | 0.53 (0.36-0.77) |
| Monocyte count <0.365 | | | |
| Platelet count >226.5 & | 0.49 (0.34-0.71) | 0.28 (0.11-0.69) | 0.54 (0.36-0.8) |
| Percent Lymphocytes >23.75 | | | |
| Percent Monocytes <5.85 & | 0.5 (0.36-0.69) | 0.29 (0.13-0.65) | 0.56 (0.39-0.8) |
| Percent Lymphocytes >23.75 | | | |
| Lymphocyte count >1.455 & | 0.53 (0.36-0.77) | 0.41 (0.17-0.95) | 0.57 (0.37-0.86) |
| White blood cell count <6.365 | | | |
| Percent Lymphocytes >23.75 & | 0.52 (0.36-0.74) | 0.4 (0.18-0.88) | 0.58 (0.39-0.85) |
| Percent Neutrophils >57.29 | | | |
| RBC distribution width <14.25 & | 0.57 (0.41-0.8) | 0.59 (0.29-1.17) | 0.58 (0.39-0.85) |
| Mean corpuscular hgb <30.05 | | | |
| Measured hemoglobin >13.05 & | 0.57 (0.41-0.8) | 0.42 (0.2-0.9) | 0.59 (0.41-0.86) |
| Monocyte count <0.365 | | | |
| Platelet count >226.5 & | 0.59 (0.41-0.84) | 0.47 (0.21-1.03) | 0.59 (0.4-0.89) |
| White blood cell count <6.365 | | | |
| RBC distribution width <14.25 & | 0.56 (0.37-0.84) | 0.53 (0.23-1.25) | 0.6 (0.39-0.95) |
| Percent Lymphocytes >31.25 | | | |
| Hematocrit >44.05 & | 0.67 (0.45-0.99) | 0.7 (0.32-1.55) | 0.62 (0.39-0.99) |
| Percent Neutrophils >57.29 | | | |

Abreviations:
RR, Relative risk;
CI, Confidence interval.
Shown above are low risk patterns present in the population along with relative risk (95% confidence interval) are shown for each pattern in the subset of the derivation cohort on which they were generated (i.e. patients in the derivation cohort with Dth/MI = 1 or maximum stenosis <50%).
Unites for each variable are shown in Tables 16.

TABLE 18
Area under the ROC curve (%) for CHRP and
traditional cardiovascular risk parameters

| | DMI-1 | Dth-1 | MI-1 |
|---|---|---|---|
| CHRP | 70.9 | 78.3 | 60.9 |
| CHRP - primary prevention | 82.6 | 80.9 | 87.7 |
| CHRP - secondary prevention | 68.7 | 77.3 | 57.7 |
| Age | 62.7 | 68.2 | 54.7 |
| Male | 49.6 | 47.6 | 51.7 |
| Hypertension | 57.2 | 55.4 | 59.3 |
| Current smoking | 50.8 | 50.1 | 52.5 |
| Past smoking | 51.2 | 54.4 | 46.8 |
| Diabetes mellitus | 57.0 | 57.8 | 55.6 |
| Total cholesterol | 48.5 | 47.8 | 50.1 |
| Low density lipoprotein | 48.3 | 47.4 | 50.3 |
| High density lipoprotein | 45.2 | 49.2 | 39.6 |
| Triglycerides | 52.1 | 47.2 | 58.9 |
| Glucose | 55.9 | 52.8 | 58.6 |
| Creatinine | 64.5 | 67.9 | 57.9 |
| HemoglobinA1C | 50.5 | 47.5 | 54.4 |
| H/o cardiovascular disease | 59.2 | 58.9 | 59.1 |
| H/o myocardial infarction | 58.5 | 57.9 | 59.2 |
| H/o revascularisation | 58.0 | 57.6 | 58.0 |
| H/o stroke | 54.1 | 56.6 | 51.6 |
| Max stenosis ≧50 | 59.6 | 59.5 | 59.3 |

TABLE 19
Odds ratio of CHRP and traditional cardiovascular
risk measures for tertiles

| | 1st tertile | 2nd tertile | 3rd tertile |
|---|---|---|---|
| CHRP(2) | ≦38.17 | >38.17, ≦49.08 | >49.08 |
| Unadjusted | 1 | 1.51 (1.116, 2.06) | 5.030 (3.84, 6.58) |
| Adjusted | 1 | 1.36 (0.99, 1.87) | 3.90 (2.94, 5.19) |
| Age | ≦59.34 | >59.34, ≦70 | >70 |
| Unadjusted | 1 | 1.547 (1.19, 2.02) | 2.692 (2.11, 3.44) |
| Adjusted | 1 | 1.401 (1.06, 1.85) | 2.031 (1.55, 2.66) |
| Gender | 0 | 1 | |
| Unadjusted | 1 | 1.05 (0.86, 1.29) | |
| Adjusted | 1 | 1.15 (0.92, 1.43) | |
| Hypertension | 0 | 1 | |
| Unadjusted | 1 | 1.63 (1.29, 2.07) | |
| Adjusted | 1 | 1.16 (0.91, 1.49) | |
| Current Smoking | 0 | 1 | |
| Unadjusted | 1 | 1.03 (0.78, 1.36) | |
| Adjusted | 1 | 1.23 (0.90, 1.69) | |
| Past Smoking | 0 | 1 | |
| Unadjusted | 1 | 1.14 (0.93, 1.39) | |
| Adjusted | 1 | 1.00 (0.80, 1.24) | |
| LDL | ≦82 | >82, ≦110.8 | >110.8 |
| Unadjusted | 1 | 0.69 (0.55, 0.86) | 0.73 (0.59, 0.91) |
| Adjusted | 1 | 0.81 (0.64, 1.02) | 1.03 (0.81, 1.30) |
| HDL | ≦39 | >39, ≦49 | >49 |
| Unadjusted | 1 | 0.90 (0.72, 1.12) | 0.72 (0.57, 0.91) |
| Adjusted | 1 | 0.91 (0.72, 1.14) | 0.73 (0.57, 0.94) |
| Diabetes | 0 | 1 | |
| Unadjusted | 1 | 1.89 (1.56, 2.27) | |
| Adjusted | 1 | 1.47 (1.21, 1.79) | |

### Example Calculation of the CHRP Risk Score

A 74 year old non-smoking, non-diabetic female with history of cardiovascular disease but no history of hypertension was seen by her primary care physician because of intervening history of occasional chest discomfort with exertion over the past several months. A stress echo was performed and showed non-diagnostic eletrocardiographic changes that were unchanged from prior studies. The study was otherwise normal. A complete blood cell count with differential was run prior to elective diagnostic cardiac catheterization (Table 20).

TABLE 20

| Hematology Analyzer Data | Value |
|---|---|
| White blood cell related | |
| White blood cell count (×10³/ml) | 13.93 |
| Neutrophils (%) | 77.1 |
| Lymphocytes (%) | 14.8 |
| Monocytes (%) | 6.2 |
| Eosinophils (%) | 0.5 |
| Basophils (%) | 0.3 |
| Large unstained cells (%) | 1.1 |
| Neutrophil count (×10³/ml) | 10.7 |
| Lymphocyte count (×10³/ml) | 2.05 |
| Monocyte count (×10³/ml) | 0.86 |
| Eosinophil count (×10³/ml) | 0.07 |
| Basophil count (×10³/ml) | 0.04 |
| Red blood cell related | |
| RBC count (×10⁶/ml) | 3.58 |
| Hematocrit (%) | 30.2 |
| Mean Corpuscular volume (MCV) | 83.4 |
| Mean corpuscular hgb (MCH; pg) | 28.0 |
| Mean corpuscular hgb concentration | 33.5 |
| (MCHC; g/dl) | |
| RBC hgb concentration mean (CHCM; | 34.2 |
| g/dl) | |
| RBC distribution width (RDW; %) | 14.4 |
| Hgb distribution width (HDW; g/dl) | 2.72 |
| Hgb content distribution width (CHDW; pg) | 34.2 |
| Macrocytic RBC count (×10⁶/ml) | 43 |
| Hypochromic RBC count (×10⁶/ml) | 379 |
| Hyperchromic RBC count (×10⁶/ml) | 347 |
| Microcytic RBC count (×10⁶/ml) | 805 |
| NRBC (%) | 0 |
| Measured Hgb | 10 |
| Platelet related | |
| Platelet count (PLT; %) | 491 |
| Mean platelet volume (MPV) | 7.9 |
| Platelet distribution width (PDW) | 55.5 |
| Plateletcrit (PCT; %) | 0.39 |
| Mean platelet concentration (MPC; g/dl) | 25.8 |
| Large platelets (×10³/ml) | 8 |
| Flag for left shift | 0 |

### Determining the CHRP Risk Score

With simple modifications to the hematology analyzer, calculation of the CHRP risk score can be done in automated fashion and provided as a value just like all other hematology analyzed calculated elements. Below, however, is a longhand example.

### Step One—Determining Whether Criteria for Each High Risk and Low Risk Pattern are Met.

Elements used to calculate the CHRP risk score are used by determining in Yes/No fashion whether binary patterns associated with high vs. low risk are satisfied. Elements included in patterns combine only data measured during performance of a routine CBC and differential (some of the data elements are measured but not routinely reported within common hematology analyzers). Table 22 lists the high risk patterns for death and MI, while Table 23 lists the low risk patterns for death and MI. The death high risk pattern #1 consists of a RDW<13.35 and % Eos<38.5. The example subject has RDW of 14.4 and % Eos of 0.5 (Table 21). Thus, this subject's data satisfies both criterion. Both criteria must be satisfied to have a pattern. This subject therefore possesses the Death High Risk #1 pattern and is assigned a point value of one (1). If the subject did not fulfill the criterion for the pattern, a point value of zero (0) would be assigned.

TABLE 21

| | Subject | | Point |
|---|---|---|---|
| Death (1 year) high risk patterns | Values | Pattern | Value |
| RBC distribution width >13.35 | RDW = 14.4 | Yes | 1 |
| & Percent Eosinophils <38.5 | % EOS = 0.5 | | |

The above approach is used to fill in whether each High and Low Risk Patterns are satisfied.

TABLE 22
indicating whether criteria for each high risk pattern for death and MI are met

| | Subject | | Point |
|---|---|---|---|
| | Values | Pattern | Value |
| Death (1 year) high risk patterns | | | |
| RBC distribution width >13.35 & | RDW = 14.4 | Yes | 1 |
| Percent Eosinophils <38.5 | % EOS = 0.5 | | |
| Hematocrit <43.55 & | HCT = 30.2 | Yes | 1 |
| Percent Lymphocytes <28.15 | % Lymph = 14.8 | | |
| Mean corpuscular hgb concentration <35.25 & | MCHC = 33.5 | No | 0 |
| Lymphocyte count <1.405 | Lymph = 2.05 | | |
| Mean corpuscular hgb concentration <33.65 & | MCHC = 33.5 | Yes | 1 |
| Percent Lymphocytes >5.1 | % Lymph = 14.8 | | |
| RBC count <4.135 & | RBC = 3.58 | Yes | 1 |
| Percent Basophils <2.75 | % Baso = 0.3 | | |
| White blood cell count >6.715 | WBCP = 13.93 | Yes | 1 |
| Eosinophil count <0.08 or >0.37 & | Eos = 0.07 | Yes | 1 |
| Monocyte count >0.265 | Mono = 0.86 | | |
| MI (1 year) high risk patterns | | | |
| Platelet count <226.5 & | Plt = 491 | No | 0 |
| Hematocrit <40.35 | HCT = 30.2 | | |
| Monocyte count >0.365 & | Mono = 0.86 | No | 0 |
| Percent Eosinophils >2.15 | % Eos = 0.5 | | |
| RBC distribution width >12.85 & | RDW = 14.4 | Yes | 1 |
| Percent Monocytes >5.85 | % Mono = 6.2 | | |
| Platelet count <175.5 & | Plt = 491 | No | 0 |
| RBC distribution width >12.85 | RDW = 14.4 | | |
| Platelet count <226.5 & | Plt = 491 | No | 0 |
| Monocyte count >0.365 | Mono = 0.86 | | |
| RBC distribution width >14.25 & | RDW = 14.4 | Yes | 1 |
| Neutrophil count >1.21 | Neut = 10.7 | | |
| Percent Neutrophils >51.8 and <78.1 & | % Neut = 77.1 | No | 0 |
| Mean corpuscular hgb >32.35 | MCH = 28 | | |
| Percent Lymphocytes <12.8 or >34.9 & | % Lymph = 14.8 | No | 0 |
| Hematocrit <40.35 | HCT = 30.2 | | |
| Percent Lymphocytes <23.75 & | % Lymph = 14.8 | No | 0 |
| Percent Neutrophils <69.75 | % Neut = 77.1 | | |
| Hematocrit <40.35 & | HCT = 30.2 | Yes | 1 |
| Percent Lymphocytes <23.75 | % Lymph = 14.8 | | |
| Mean corpuscular hgb >32.35 & | MCH = 28 | No | 0 |
| Percent Neutrophils >57.29 | % Neut = 77.1 | | |
| Eosinophil count >0.305 & | Eos = 0.07 | No | 0 |
| Percent Monocytes >3.75 | % Mono = 6.2 | | |

TABLE 23
indicating whether criteria for each low
risk pattern for death and MI are met

| | Subject | | Point |
|---|---|---|---|
| | Values | Pattern | Value |
| Death (1 year) low risk patterns | | | |
| RBC distribution width <15.05 & | RDW = 14.4 | Yes | 1 |
| Percent Lymphocytes >13.45 | % Lymph = 14.8 | | |
| RBC distribution width <15.05 & | RDW = 14.4 | No | 0 |
| RBC count >3.625 | RBC = 3.58 | | |
| Monocyte count <0.465 & | Mono = 0.86 | No | 0 |
| Lymphocyte count >0.865 | Lymph = 2.05 | | |
| Hematocrit >39.15 & | HCT = 30.2 | No | 0 |
| Percent Neutrophils <76.65 | % Neut = 77.1 | | |
| RBC distribution width <17.05 & | RDW = 14.4 | No | 0 |
| RBC count >4.135 | RBC = 3.58 | | |
| Hematocrit >34.95 & | HCT = 30.2 | No | 0 |
| White blood cell count <6.715 | WBCP = 13.93 | | |
| RBC distribution width <13.35 & | RDW = 14.4 | No | 0 |
| White blood cell count >5.285 | WBCP = 13.93 | | |
| Eosinophil count <0.375 & | Eos = 0.07 | No | 0 |
| White blood cell count <5.285 | WBCP = 13.93 | | |
| Percent Basophils >0.3 and <1.2 | % Baso = 0.3 | No | 0 |
| & Percent Monocytes <6.25 | % Mono = 6.2 | | |
| MI-1 low risk patterns | | | |
| Hematocrit >40.35 & | HCT = 30.2 | No | 0 |
| White blood cell count <6.365 | WBCP = 13.93 | | |
| RBC distribution width <12.85 & | RDW = 14.4 | No | 0 |
| Percent Neutrophils >32.88 | % Neut = 77.1 | | |
| Mean corpuscular hgb <32.35 & | MCH = 28 | No | 0 |
| Hematocrit >40.35 | HCT = 30.2 | | |
| Monocyte count <0.365 & | Mono = 0.86 | No | 0 |
| Lymphocyte count >1.455 | Lymph = 2.05 | | |
| Percent Monocytes <5.85 & | % Mono = 6.2 | No | 0 |
| White blood cell count <6.365 | WBCP = 13.93 | | |
| Platelet count >226.5 & | Plt = 491 | No | 0 |
| Monocyte count <0.365 | Mono = 0.86 | | |
| Platelet count >226.5 & | Plt = 491 | No | 0 |
| Percent Lymphocytes >23.75 | % Lymph = 14.8 | | |
| Percent Monocytes <5.85 & | % Mono = 0.86 | No | 0 |
| Percent Lymphocytes >23.75 | % Lymph = 14.8 | | |
| Lymphocyte count >1.455 & | Lymph = 2.05 | No | 0 |
| White blood cell count <6.365 | WBCP = 13.93 | | |
| Percent Lymphocytes >23.75 & | % Lymph = 14.8 | No | 0 |
| Percent Neutrophils >57.29 | % Neut = 77.1 | | |
| RBC distribution width <14.25 & | RDW = 14.4 | No | 0 |
| Mean corpuscular hgb <30.05 | MCH = 28 | | |
| Measured hemoglobin >13.05 & | MCH = 28 | No | 0 |
| Monocyte count <0.365 | Mono = 0.86 | | |
| Platelet count >226.5 & | Plt = 491 | No | 0 |
| White blood cell count <6.365 | WBCP = 13.93 | | |
| RBC distribution width <14.25 & | RDW = 14.4 | No | 0 |
| Percent Lymphocytes >31.25 | % Lymph = 14.8 | | |
| Hematocrit >44.05 & | HCT = 30.2 | No | 0 |
| Percent Neutrophils >57.29 | % Neut = 77.1 | | |

Step Two—Counting the Number of High and Low Risk Patterns that are Satisfied.

The next step is to count how many positive and negative patterns are fulfilled. In this example:

Number of high risk patterns Subject has=9

Number of low risk patterns Subject has=1

### Step Three—Calculating the Weighted Raw Score.

Subjects generally have combinations of both high and low risk patterns. Overall risk is calculated as the difference in the average number of high risk patterns and the average number of low risk patterns fulfilled by the subject.

The number of high risk patterns is 19.

The number of low risk patterns is 24.

Average # high risk patterns satisfied by the subject=9/19

Average # low risk patterns satisfied by the subject=1/24

The Raw Score of a subject is calculated by the weighted sum of high risk and low risk patterns. In this example:
Raw Score=1/Total number of high risk patterns*Number of high risk patterns satisfied by subject−1/Total number of low risk patterns*Number of low risk patterns satisfied by subject=9/19−1/24=0.432
The calculated Raw Score ranges from −1 to +1 with 0 as the midpoint. A score of 0 is obtained if the patient satisfies none of the positive or negative patterns or if the patient satisfies equal proportions of positive and negative patterns.

### Step Four—Calculating the Final CHRP Value

The last step is to adjust the Raw Score (range from −1 to +1) to the CHRP (range of 0 to 100, assuming 50 as the midpoint of the scale) by multiplying the Raw Score by 50, and then adding 50.

$$\begin{matrix}CHRP = ( 50 \times Raw Score ) + 50 \\ = ( 50 \times - 0.432 ) + 50 \\ = 71.6\end{matrix}$$

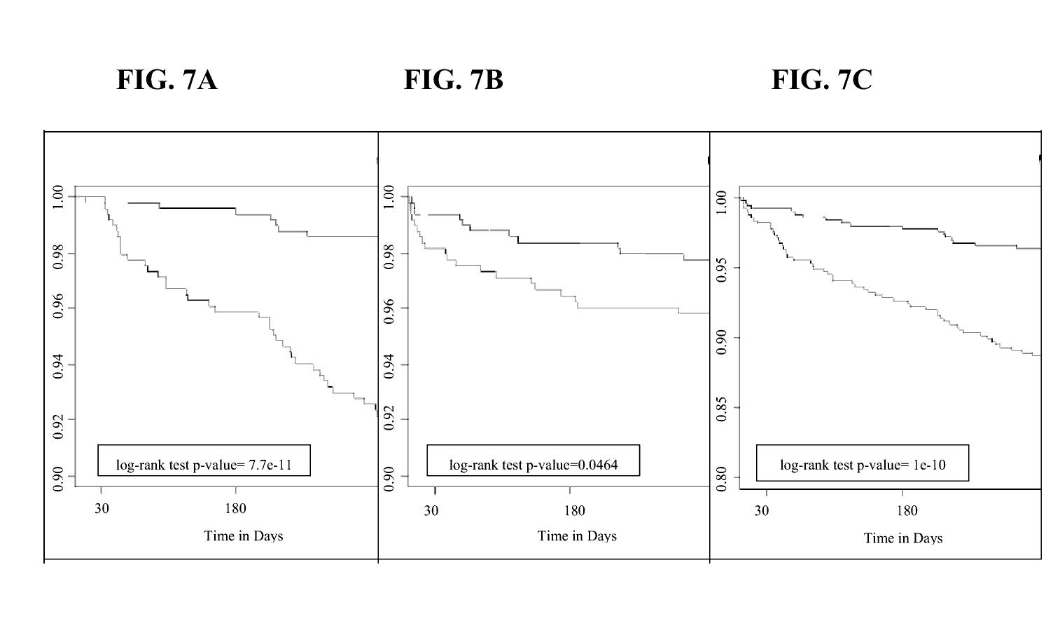

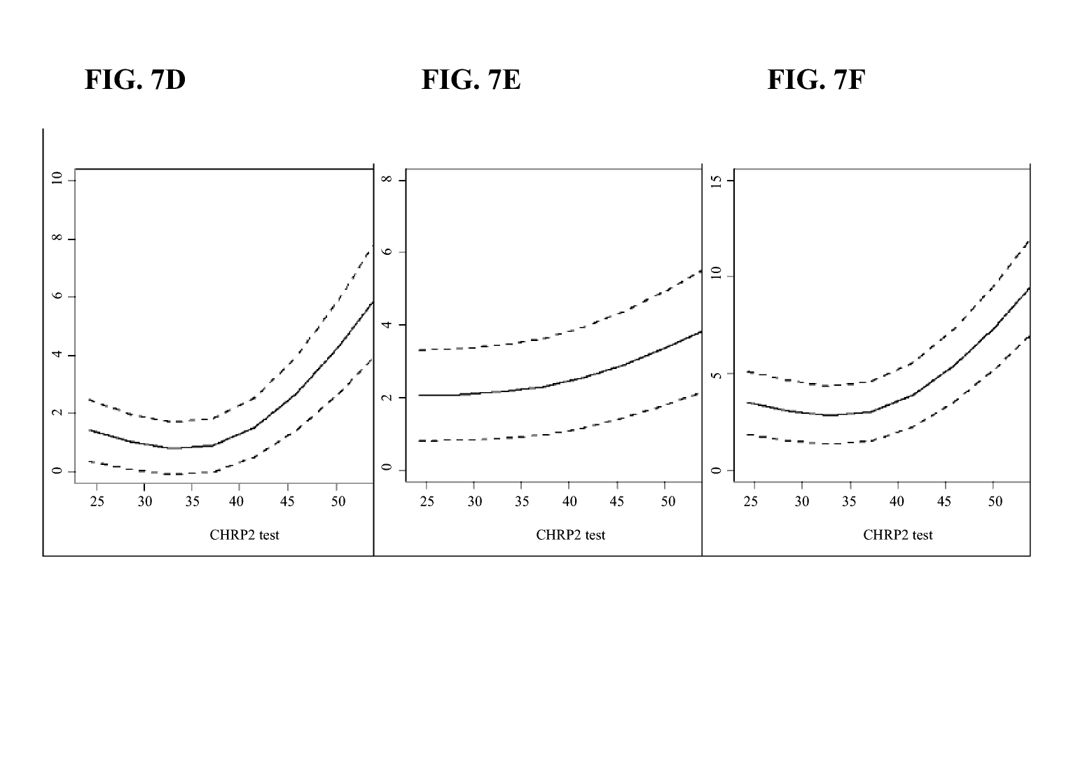

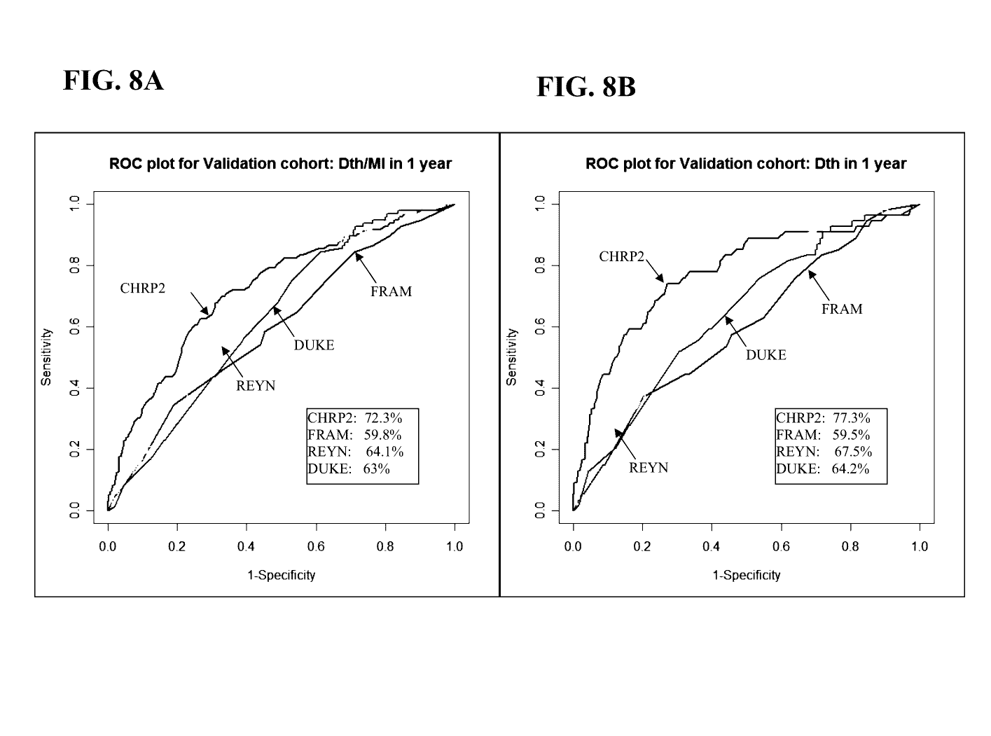

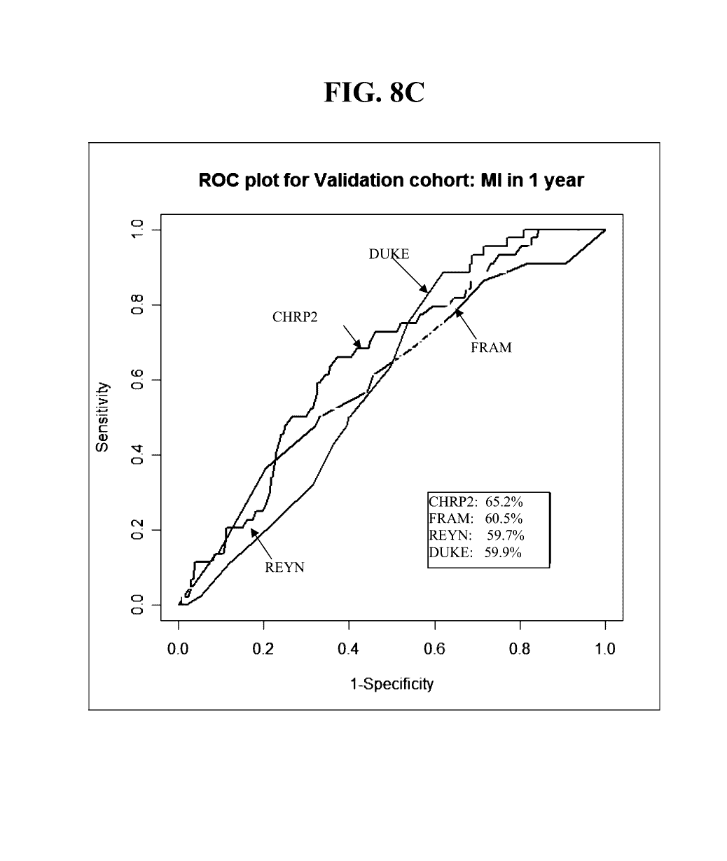

This subject falls into the high risk category. FIG. 7F allows one to use the CHRP Risk Score to estimate overall incident risk of death or MI over the ensuing 1 year period. In this example, the subject's 1 yr event rate is greater than 7%.

### Example 3

### CHRP (PEROX) Model

This Example successfully tests the hypothesis that using only information generated from analysis of whole blood with a hematology analyzer during the performance of a traditional CBC with differential including peroxidase based measurements, high and low risk patterns may be identified allowing for development of a Peroxidase-based Comprehensive Hematology Risk Profile (CHRP (PEROX)), a single laboratory value that accurately predicts incident risks for non-fatal MI and death in subjects.

Methods:

7,369 patients undergoing elective diagnostic cardiac evaluation at a tertiary care center were enrolled for the study. An extensive array of erythrocyte, leukocyte, and platelet related parameters were captured on whole blood analyzed from each subject at the time of performance of a CBC and differential. The patients were randomly divided into a Derivation (N=5,895) and a Validation Cohort (N=1,473). CHRP (PEROX) was developed using Logical Analysis of Data methodology. First, binary high-risk and low-risk patterns amongst collected erythrocyte, leukocyte and platelet data elements were identified for one year incident risk of non-fatal MI or death. Then, a comprehensive single prognostic risk value, CHRP (PEROX), was developed by combining these high and low risk patterns to form a single prognostic score.

Results:

Using only parameters routinely available from whole blood analysis on a peroxidase-based hematology analyzer, 25 high-risk and 34 low-risk binary patterns were identified using the Derivation Cohort. These patterns were distilled down into a single, highly accurate prognostic value, the CHRP (PEROX). Independent prospective testing of the CHRP (PEROX) within the Validation Cohort revealed superior prognostic accuracy (72%) for prediction of one-year risk of death or MI compared with traditional cardiovascular risk factors, laboratory tests, as well as clinically established risk scores including Adult Treatment Panel III (60%), Reynolds (64%), and Duke angiographic (63%) scoring systems. Superior prognostic accuracy for prediction of 1 year incident MI and death was also observed with CHRP in both primary and secondary prevention subgroups, diabetics and non-diabetics alike, and even amongst those with no evidence of significant coronary atherosclerotic burden (<50% stenosis in all major coronary vessels) at time of recent cardiac catheterization.

This Example shows that use of a routine automated hematology analyzer for whole blood analysis generates a spectrum of data from which high and low risk patterns can be identified for predicting a subject's risk for experiencing major adverse cardiac events. A composite single value was built based upon these patterns, the Peroxidase-based Comprehensive Hematology Risk Profile (CHRP (PEROX)), which accurately predicts incident risks for non-fatal MI and death in subjects, and accurately classifies patients for both high and low near-term (one year) cardiovascular risks. Multivariate logistic regression analysis shows that the CHRP (PEROX) is a strong predictor of risk independent of traditional cardiac risk factors and laboratory markers in subjects. Moreover, CHRP (PEROX) provides strong prognostic value even within subjects who show no significant angiographic evidence of atherosclerosis on recent cardiac catheterization.

TABLE 24
Clinical and laboratory parameters

| | Derivation | Validation | |
|---|---|---|---|
| | Cohort | Cohort | |
| | (N = 5,895) | (N = 1,474) | P-value |
| Traditional Risk Factors | | | |
| Age (years) | 64.1 ± 11.3 | 64.1 ± 10.9 | 0.95 |
| Male - n (%) | 4,021 (68) | 1,024 (69) | 0.35 |
| Hypertension - n (%) | 4,335 (74) | 1,075 (73) | 0.64 |
| Current smoking - n (%) | 770 (13) | 162 (11) | 0.03 |
| History of smoking - n (%) | 3,869 (66) | 995 (68) | 0.18 |
| Diabetes mellitus - n (%) | 2131 (36) | 577 (39) | 0.03 |
| Laboratory Measurements | | | |
| Fasting blood glucose (mg/dl) | 102 (91-123) | 104 (92-128) | 0.03^{†} |
| Creatinine (mg/dl) | 0.9 (0.8-1.1) | 0.9 (0.8-1.1) | 0.08^{†} |
| Potassium (mmol/l) | 4.2 (4.0-4.5) | 4.2 (4.0-4.5) | 0.44^{†} |
| C-reactive protein (mg/dl) | 2.7 (1.2-6.4) | 2.7 (1.1-5.9) | 0.10^{†} |
| Total cholesterol (mg/dl) | 170 ± 41 | 170 ± 41 | 0.50 |
| LDL cholesterol (mg/dl) | 99 ± 34 | 100 ± 33 | 0.33 |
| HDL cholesterol (mg/dl) | 40 ± 13 | 40 ± 14 | 0.50 |
| Triglycerides (mg/dl) | 122 (86-177) | 124 (87-181) | 0.46^{†} |
| Clinical Characteristics | | | |
| Systolic blood pressure | 135 ± 21 | 136 ± 22 | 0.02 |
| (mm Hg) | | | |
| Diastolic blood pressure | 75 ± 12 | 75 ± 13 | 0.30 |
| (mm Hg) | | | |
| Body mass index (kg/m²) | 30 ± 6 | 30 ± 6 | 0.84 |
| Aspirin use - n (%) | 4,270 (72) | 1,087 (73) | 0.31 |
| Statin use - n (%) | 3,450 (59) | 869 (59) | 0.76 |

Data are shown as median (interquartile range) for continuous variables, or number in category (percent of total in category).
^{†}Non-parametric test

TABLE 25
Hematology parameters for CHRP (PEROX) risk score model

| | Derivation | Validation | Death in 1 year | MI in 1 year |
|---|---|---|---|---|
| | cohort | cohort | HR (95% CI)‡ | HR (95% CI)‡ |
| White blood cell related | | | | |
| White blood cell count (×10³/ml) | 6.1 (5.1-7.5) | 6.1 (5.0-7.5) | 1.64 (1.20-2.23) | 0.94 (0.64-1.37) |
| Neutrophils (%) | 63.9 (57.7-70.7) | 64.8 (58.1-71.2) | 2.27 (1.65-3.12) | 0.84 (0.56-1.25) |
| Lymphocytes (%) | 23.8 (18.1-29.6) | 23 (17.7-28.5) | 0.35 (0.26-0.49) | 1.07 (0.72-1.59) |
| Monocytes (%) | 5.3 (4.3-6.3) | 5.2 (4.3-6.4) | 1.52 (1.13-2.04) | 1.41 (0.95-2.10) |
| Eosinophils (%) | 3.0 (2.0-4.3) | 2.9 (1.9-4.1) | 0.85 (0.63-1.14) | 1.16 (0.77-1.75) |
| Basophils (%) | 0.6 (0.4-0.9) | 0.6 (0.4-0.9) | 0.70 (0.51-0.95) | 1.36 (0.90-2.05) |
| Large unstained cells (%) | 2.1 (1.6-2.7) | 2.1 (1.6-2.7) | 0.77 (0.56-1.04) | 1.12 (0.75-1.68) |
| Neutrophil count (×10³/ml) | 4.0 (3.1-5.2) | 4.0 (3.2-5.2) | 2.15 (1.56-2.95) | 1.00 (0.68-1.47) |
| Lymphocyte count (×10³/ml) | 1.5 (1.1-1.9) | 1.4 (1.1-1.8) | 0.45 (0.33-0.63) | 0.91 (0.61-1.36) |
| Monocyte count (×10³/ml) | 0.3 (0.3-0.4) | 0.3 (0.3-0.4) | 2.05 (1.50-2.80) | 1.19 (0.81-1.74) |
| Eosinophil count (×10³/ml) | 0.2 (0.1-0.3) | 0.2 (0.1-0.3) | 0.93 (0.70-1.25) | 1.05 (0.72-1.54) |
| Basophil count (×10³/ml) | 0 (0-0.1) | 0 (0-0.1) | 0.90 (0.66-1.23) | 1.25 (0.81-1.91) |
| Large unstained cells count | | | | |
| Ky | | | | |
| High peroxidase staining cells count | | | | |
| Number of peroxidase saturated cells | | | | |
| (×10³/ml) | | | | |
| Lymphocyte/large unstained cell threshold | | | | |
| Lymphocytic mode | | | | |
| Perox d/D | | | | |
| Peroxidase y sigma | | | | |
| Blasts (%) | | | | |
| Blasts count | | | | |
| Mononuclear central y channel | | | | |
| Mononuclear polymorphonuclear valley | | | | |
| Red blood cell related | | | | |
| RBC count (×10⁶/ml) | 4.3 (4.0-4.6) | 4.3 (4.0-4.7) | 0.32 (0.23-0.46) | 0.83 (0.56-1.23) |
| Hematocrit (%) | 41.2 (38.1-43.8) | 41.3 (38.4-43.9) | 0.32 (0.23-0.45) | 0.69 (0.46-1.02) |
| Mean Corpuscular volume (MCV) | 88.4 (85.5-91.4) | 88.4 (85.3-91.3) | 1.52 (1.11-2.07) | 1.14 (0.79-1.65) |
| Mean corpuscular hgb (MCH; pg) | 30.5 (29.4-31.6) | 30.5 (29.3-31.6) | 0.77 (0.58-1.03) | 1.20 (0.83-1.75) |
| Mean corpuscular hgb concentration (MCHC; | 34.4 (33.7-35.0) | 34.4 (33.6-35.1) | 0.24 (0.17-0.35) | 0.93 (0.62-1.39) |
| g/dl) | | | | |
| RBC hgb concentration mean (CHCM; g/dl) | 35.2 (34.3-35.9) | 35.2 (34.4-36.0) | 0.24 (0.17-0.35) | 0.79 (0.54-1.15) |
| RBC distribution width (RDW; %) | 13.2 (12.7-13.8) | 13.1 (12.6-13.8) | 5.84 (3.96-8.62) | 1.95 (1.28-2.97) |
| Hgb distribution width (HDW; g/dl) | 2.6 (2.5-2.8) | 2.6 (2.5-2.8) | 2.74 (1.95-3.85) | 1.52 (1.03-2.23) |
| Hgb content distribution width (CHDW; pg) | 3.8 (3.6-4.0) | 3.8 (3.6-4.0) | 4.23 (2.95-6.06) | 1.25 (0.84-1.86) |
| Macrocytic RBC count (×10⁶/ml) | 140 (65-296) | 133.5 (64-293) | 3.30 (2.31-4.73) | 1.31 (0.89-1.91) |
| Hypochromic RBC count (×10⁶/ml) | 56 (16-165) | 49 (15-148) | 2.36 (1.74-3.20) | 1.67 (1.12-2.49) |
| Hyperchromic RBC count (×10⁶/ml) | 685 (389-1217) | 722.5 (403-1247) | 0.42 (0.30-0.58) | 0.97 (0.65-1.43) |
| Microcytic RBC count (×10⁶/ml) | 236 (133-437) | 244 (134-444) | 1.90 (1.39-2.59) | 0.92 (0.63-1.34) |
| NRBC count | 42 (30-60) | 43 (30-61) | 1.48 (1.09-1.99) | 0.93 (0.63-1.38) |
| Measured HGB | 13.1 (12-14.1) | 13.2 (12.1-14.2) | 0.23 (0.16-0.33) | 0.79 (0.53-1.18) |
| Platelet related | | | | |
| Platelet count (PLT; %) | 224 (186-266) | 220 (183-264) | 0.95 (0.70-1.28) | 0.83 (0.57-1.23) |
| Mean platelet volume (MPV) | 7.8 (7.3-8.4) | 7.8 (7.4-8.4) | 1.49 (1.10-2.03) | 1.14 (0.77-1.69) |
| Platelet distribution width (PDW) | 55.6 (51.5-59.9) | 55.8 (51.6-60.3) | 1.31 (0.96-1.79) | 1.15 (0.77-1.72) |
| Plateletcrit (PCT; %) | 0.2 (0.2-0.2) | 0.2 (0.2-0.2) | 1.10 (0.81-1.48) | 0.77 (0.52-1.14) |
| Mean platelet concentration (MPC; g/dl) | 27.3 (26.2-28.2) | 27.3 (26.3-28.1) | 0.45 (0.33-0.62) | 0.94 (0.65-1.36) |
| Large platelets (×10³/ml) | 4 (3-6) | 4 (3-6) | 1.31 (0.98-1.75) | 1.06 (0.72-1.56) |

Abbreviations:
MI, myocardial infarction;
HR, hazard ratio;
CI, confidence interval;
RBC, red blood cell;
Hgb, hemoglobin.
Data are shown as median (interquartile range). Some variables have no unit of measure associated with them.
Hazard ratios were calculated for tertile 3 vs. tertile 1.
‡Derivation Cohort only
∫Dichotomous variable presented as number in category (percent of total in category).

TABLE 26a
High Risk Patterns for CHRP (PEROX) test

| | Dth/MI in 1 year | Dth in 1 year | MI in 1 year |
|---|---|---|---|
| | RR | RR | RR |
| Dth-1 year high-risk patterns | | | |
| Hgb content distribution width >=3.66 & | 3.9 (3.03-5.04) | 4.6 (3.47-6.09) | 1.55 (0.77-3.11) |
| RBC hgb concentration mean <=35.7 | | | |
| Percent Lymphocytes <=20 & | 2.5 (2.01-3.12) | 2.94 (2.32-3.71) | 0.55 (0.23-1.33) |
| Percent Neutrophils >51.8 | | | |
| Hgb distribution width >2.76 & | 2.59 (2.07-3.25) | 2.83 (2.24-3.58) | 1.3 (0.54-3.14) |
| Mean Corpuscular volume >=86.5 | | | |
| Hematocrit <=39.2 & | 2.5 (2.01-3.1) | 2.74 (2.17-3.45) | 1.46 (0.7-3.02) |
| Percent Monocytes >=3.3 | | | |
| Mononuclear central y channel <=15.6 & | 2.35 (1.89-2.93) | 2.71 (2.15-3.41) | 0.75 (0.33-1.73) |
| Blasts count >5.4198 | | | |
| Mean platelet concentration <=26.7 & | 2.3 (1.84-2.87) | 2.42 (1.92-3.06) | 1.82 (0.86-3.82) |
| Hgb distribution width >2.52 | | | |
| Eosinophil count >0.37 & | 1.93 (1.39-2.67) | 2.15 (1.54-2.98) | 0.49 (0.07-3.54) |
| White blood cell count >=5.4 | | | |
| Hyperchromic RBC count <=239 & | 2.04 (1.57-2.64) | 2.14 (1.63-2.81) | 0.84 (0.26-2.73) |
| White blood cell count >4.244 | | | |
| MI-1 year high-risk patterns | | | |
| Large platelets <=2 & | 2.82 (1.95-4.07) | 1.71 (0.63-4.67) | 3.04 (2.01-4.6) |
| Peroxidase y sigma >8.53 | | | |
| Macrocytic RBC count <31.4 or >641 & | 2.43 (1.58-3.73) | 1.56 (0.5-4.92) | 2.78 (1.74-4.43) |
| Ky <=94 | | | |
| Microcytic RBC count <162 & | 2.11 (1.35-3.29) | 1.44 (0.46-4.56) | 2.57 (1.61-4.11) |
| Hgb distribution width >2.7598 | | | |
| Macrocytic RBC count <31.4 or >641 & | 2.2 (1.61-3.02) | 2.13 (1.08-4.23) | 2.54 (1.8-3.59) |
| Hematocrit <=39.2 | | | |
| Blasts count >5.4198 & | 2.1 (1.58-2.81) | 1.34 (0.67-2.67) | 2.53 (1.84-3.48) |
| Neutrophil count x high peroxidase | | | |
| staining count >0 | | | |
| Mean corpuscular hgb >=31.2 & | 2.45 (1.79-3.35) | 1.86 (0.88-3.92) | 2.5 (1.74-3.59) |
| Peroxidase y sigma >=8.53 | | | |
| NRBC <=34 & | 2 (1.44-2.78) | 0.97 (0.39-2.43) | 2.43 (1.71-3.47) |
| Plateletcrit <0.16 | | | |
| RBC count <3.64 or >4.96 & | 2.81 (1.98-3.99) | 4.91 (2.57-9.37) | 2.36 (1.52-3.67) |
| Lymphocytic mode >=35.5 | | | |
| Macrocytic RBC count <31.4 or >641 & | 2.45 (1.83-3.29) | 3.5 (1.94-6.29) | 2.34 (1.66-3.3) |
| Hypochromic RBC count >113 | | | |
| Percent Basophils*WBCP <1.68 or >8.21 & | 2.59 (1.79-3.75) | 2.95 (1.36-6.43) | 2.34 (1.49-3.67) |
| Percent Monocytes >=6 | | | |
| MPM <1.8 or >2.29 & | 2.17 (1.56-3.02) | 2.17 (1.07-4.42) | 2.24 (1.54-3.26) |
| Monocyte count >0.38 | | | |
| Mean platelet volume >=9.1 & | 1.89 (1.24-2.88) | 1.48 (0.54-4.04) | 2.2 (1.4-3.46) |
| High peroxidase staining cell count <5.72 | | | |
| Mean Platelet volume <7 or >9.1 & | 1.79 (1.14-2.82) | 0.8 (0.2-3.25) | 2.18 (1.35-3.51) |
| Percent Basophils*WBCP <1.68 or >8.21 | | | |
| Percent Lymphocytes <12.8 or >34.9 & | 2.5 (1.77-3.54) | 4.52 (2.4-8.49) | 2.18 (1.42-3.33) |
| Hematocrit <=39.2 | | | |
| RBC distribution width >=13.6 & | 2 (1.3-3.07) | 1.21 (0.38-3.84) | 2.16 (1.34-3.48) |
| Mononuclear polymorphonuclear valley >=21 | | | |
| NRBC <=53 & | 2.31 (1.56-3.4) | 3.39 (1.63-7.08) | 2.15 (1.35-3.42) |
| Percent Lymphocytes <=12.8 | | | |
| Hgb distribution width >=3.05 & | 2.15 (1.45-3.19) | 2.7 (1.24-5.9) | 2.14 (1.36-3.37) |
| Percent Large unstained cells <=2.5 | | | |

Abreviations:
RR, Relative risk;
CI, Confidence interval.

Table 26a provides high risk patterns present in the population along with relative risk (95% confidence interval) are shown for each pattern in the subset of the derivation cohort on which they were generated (i.e. patients in the derivation cohort with Dth/MI=1 or maximum stenosis<50%). Units for each variable are shown in Table 25.

TABLE 26b
Low Risk Patterns for CHRP (PEROX) test

| | Dth/MI in 1 year | Dth in 1 year | MI in 1 year |
|---|---|---|---|
| | RR | RR | RR |
| Dth-1 year low-risk patterns | | | |
| RBC distribution width <=13.6 & | 0.25 (0.2-0.31) | 0.22 (0.17-0.29) | 0.74 (0.36-1.52) |
| Mononuclear polymorphonuclear valley >=18 | | | |
| Hematocrit >=39.2 & | 0.28 (0.22-0.36) | 0.23 (0.18-0.3) | 0.78 (0.38-1.58) |
| Peroxidase y sigma <=9.49 | | | |
| Macrocytic RBC count <227 & | 0.33 (0.25-0.42) | 0.28 (0.21-0.37) | 0.78 (0.39-1.58) |
| Blasts count <5.4198 | | | |
| Percent Monocytes <=6 & | 0.34 (0.26-0.44) | 0.29 (0.21-0.38) | 0.95 (0.47-1.92) |
| Percent Lymphocytes >=20 | | | |
| Hypochromic RBC count <113 & | 0.32 (0.25-0.42) | 0.29 (0.22-0.38) | 0.63 (0.31-1.29) |
| White blood cell count <=6.96 | | | |
| Blasts count <3.15 & | 0.41 (0.3-0.56) | 0.34 (0.24-0.48) | 0.97 (0.46-2.04) |
| Percent Eosinophils >1.2 | | | |
| Microcytic RBC count <=349 & | 0.38 (0.28-0.5) | 0.35 (0.26-0.47) | 0.59 (0.27-1.27) |
| RBC count >=4.07 | | | |
| Mononuclear central y channel >=15.1 & | 0.42 (0.32-0.57) | 0.35 (0.25-0.49) | 1.01 (0.48-2.09) |
| Percent Lymphocytes >=12.8 | | | |
| Macrocytic RBC count <=86 & | 0.38 (0.27-0.53) | 0.36 (0.26-0.51) | 0.43 (0.16-1.1) |
| Percent Neutrophils >=51.8 | | | |
| Hgb distribution width <2.76 & | 0.42 (0.3-0.59) | 0.38 (0.26-0.55) | 0.69 (0.28-1.67) |
| White blood cell count <=5.4 | | | |
| Mononuclear polymorphonuclear valley <13.3 | 0.43 (0.31-0.59) | 0.38 (0.27-0.54) | 0.82 (0.37-1.81) |
| or >15.6 & | | | |
| Monocyte count <0.51 | | | |
| Platelet count >=251 & | 0.43 (0.3-0.62) | 0.4 (0.27-0.58) | 0.76 (0.31-1.83) |
| Monocyte count <0.38 | | | |
| Platelet count >=251 & | 0.44 (0.3-0.64) | 0.4 (0.26-0.6) | 0.69 (0.27-1.79) |
| Mean corpuscular hgb concentration >=33.9 | | | |
| Platelet distribution width <=52.9 & | 0.46 (0.33-0.65) | 0.4 (0.28-0.58) | 1.03 (0.46-2.28) |
| Blasts count <5.42 | | | |
| Lymphocyte count >1.21 & | 0.45 (0.32-0.63) | 0.4 (0.28-0.58) | 0.86 (0.37-1.98) |
| Percent Monocytes <4.6 | | | |
| MI-1 year low risk patterns | | | |
| Hypochromic RBC count <=27 & | 0.45 (0.29-0.72) | 0.82 (0.39-1.75) | 0.32 (0.18-0.59) |
| Ky >=98 | | | |
| RBC distribution width <=12.8 & | 0.31 (0.2-0.46) | 0.24 (0.09-0.6) | 0.33 (0.21-0.52) |
| Mean corpuscular hgb <=32.6 | | | |
| Hypochromic RBC count <=27 & | 0.39 (0.26-0.6) | 0.47 (0.21-1.04) | 0.35 (0.21-0.57) |
| Neutrophil count <4.71 | | | |
| MPM >1.8 and <2.29 & | 0.41 (0.26-0.63) | 0.67 (0.31-1.41) | 0.37 (0.22-0.62) |
| Peroxidase y sigma <=7.59 | | | |
| RBC distribution width <=12.8 & | 0.32 (0.21-0.49) | 0.11 (0.03-0.44) | 0.37 (0.24-0.59) |
| Neutrophil count <=4.71 | | | |
| Hypochromic RBC count <=27 & | 0.44 (0.3-0.64) | 0.65 (0.32-1.29) | 0.37 (0.24-0.59) |
| Monocyte count <0.38 | | | |
| RBC distribution width <=13.6 & | 0.48 (0.3-0.76) | 0.87 (0.41-1.85) | 0.37 (0.21-0.67) |
| Perox d/D >0.96 | | | |
| RBC distribution width <=12.8 & | 0.32 (0.21-0.5) | 0.11 (0.03-0.47) | 0.38 (0.23-0.6) |
| Lymphocyte count >1.21 | | | |
| Hypochromic RBC count <=27 & | 0.41 (0.27-0.62) | 0.39 (0.17-0.91) | 0.39 (0.25-0.63) |
| Percent Lymphocytes >=20 | | | |
| MPM >1.8 and <2.29 & | 0.53 (0.35-0.78) | 0.88 (0.44-1.76) | 0.4 (0.24-0.66) |
| Hypochromic RBC count <=27 | | | |
| Blasts count <3.15 & | 0.52 (0.34-0.79) | 0.84 (0.41-1.73) | 0.4 (0.24-0.68) |
| Eosinophil count >0.14 | | | |
| Blasts count <3.15 & | 0.47 (0.33-0.67) | 0.67 (0.34-1.31) | 0.4 (0.26-0.62) |
| Large unstained cell count >0.07 | | | |
| Percent blasts <0.5 & | 0.42 (0.28-0.63) | 0.39 (0.16-0.9) | 0.41 (0.26-0.65) |
| Percent Neutrophils <=78.1 | | | |
| Hgb content distribution width <=3.66 & | 0.39 (0.26-0.59) | 0.32 (0.13-0.79) | 0.41 (0.26-0.66) |
| Basophil count <0.05 | | | |
| Hgb distribution width <2.76 & | 0.45 (0.29-0.69) | 0.66 (0.31-1.4) | 0.42 (0.26-0.69) |
| Percent blasts <0.5 | | | |
| Flag for left shift <1 & | 0.47 (0.31-0.71) | 0.56 (0.25-1.25) | 0.42 (0.26-0.69) |
| Blasts count <3.15 | | | |
| Plateletcrit >0.16 & | 0.56 (0.38-0.82) | 0.94 (0.48-1.83) | 0.42 (0.26-0.69) |
| Lymphocyte/large unstained cell | | | |
| threshold <=44 | | | |
| Hgb content distribution width <=3.66 & | 0.43 (0.27-0.69) | 0.46 (0.18-1.15) | 0.44 (0.26-0.73) |
| Peroxidase y sigma <=7.59 | | | |
| Macrocytic RBC count >31.4 and <641 & | 0.62 (0.41-0.93) | 1.14 (0.57-2.27) | 0.44 (0.26-0.75) |
| Percent Basophils <0.5 | | | |

Abreviations:
RR, Relative risk;
CI, Confidence interval.

Table 26b shows low risk patterns present in the population along with relative risk (95% confidence interval) are shown for each pattern in the subset of the derivation cohort on which they were generated (i.e. patients in the derivation cohort with Dth/MI=1 or maximum stenosis <50%). Units for each variable are shown in Table 24.

### Formula for Computing CHRP (PEROX) Risk Score for Patient P:

50+50×(Average #high-risk patterns covering P−Average #low-risk patterns covering P].

TABLE 27
Area under the ROC curve (%) for CHRP (PEROX)
and traditional cardiovascular risk parameters

| | Dth/MI-1 | Dth-1 | MI-1 |
|---|---|---|---|
| CHRP(PEROX) | 72.3 | 77.3 | 65.2 |
| CHRP(PEROX) - primary prevention | 76.0 | 78.5 | 70.1 |
| CHRP(PEROX) - secondary prevention | 70.5 | 62.3 | 76.6 |
| Age | 62.7 | 68.2 | 54.7 |
| Male | 49.6 | 47.6 | 51.7 |
| Diabetis mellitus | 57.0 | 57.8 | 55.6 |
| Hypertension | 57.2 | 55.4 | 59.3 |
| Current smoking | 50.8 | 50.1 | 52.5 |
| Past smoking | 51.2 | 54.4 | 46.8 |
| Total cholesterol | 48.5 | 47.8 | 50.1 |
| Low density lipoprotein | 48.3 | 47.4 | 50.3 |
| High density lipoprotein | 45.2 | 49.2 | 39.6 |
| Triglycerides | 52.1 | 47.2 | 58.9 |
| Glucose | 55.9 | 52.8 | 58.6 |
| Creatinine | 64.5 | 67.9 | 57.9 |
| HemoglobinA1C | 50.5 | 47.5 | 54.4 |
| H/o cardiovascular disease | 59.2 | 58.9 | 59.1 |
| H/o myocardial infarction | 58.5 | 57.9 | 59.2 |
| H/o revascularisation | 58.0 | 57.6 | 58.0 |
| H/o stroke | 54.1 | 56.6 | 51.6 |
| Max stenosis ≧50 | 59.6 | 59.5 | 59.3 |

TABLE 28
Hazard ratio of CHRP (PEROX) and traditional
cardiovascular risk measures for tertiles

| | 1st tertile | 2nd tertile | 3rd tertile |
|---|---|---|---|
| CHRP (PEROX) | ≦37.94 | 38.23-49.09 | >49.17 |
| Unadjusted | 1 | 1.95 (1.43-2.68) | 6.34 (4.79-8.40) |
| Adjusted^{†} | 1 | 1.71 (1.24-2.36) | 4.98 (3.71-6.69) |
| Age | ≦59.34 | >59.34, ≦70 | >70 |
| Unadjusted | 1 | 1.53 (1.18-1.98) | 2.59 (2.04-3.28) |
| Adjusted^{†} | 1 | 1.36 (1.04-1.78) | 1.88 (1.45-2.43) |
| LDL | ≦82 | >82, ≦110.8 | >110.8 |
| Unadjusted | 1 | 0.67 (0.54-0.84) | 0.75 (0.61-0.93) |
| Adjusted^{†} | 1 | 0.81 (0.65-1.02) | 1.06 (0.85-1.33) |
| HDL | ≦39 | >39, ≦49 | >49 |
| Unadjusted | 1 | 0.84 (0.68-1.04) | 0.72 (0.58-0.91) |
| Adjusted^{†} | 1 | 0.91 (0.73-1.13) | 0.80 (0.64-1.01) |
| Gender | Female | Male | |
| Unadjusted | 1 | 1.05 (0.87-1.28) | |
| Adjusted^{†} | 1 | 0.94 (0.77-1.16) | |
| Hypertension | No | Yes | |
| Unadjusted | 1 | 1.60 (1.27-2.02) | |
| Adjusted^{†} | 1 | 1.17 (0.93-1.48) | |
| Current Smoking | No | Yes | |
| Unadjusted | 1 | 1.03 (0.79-1.35) | |
| Adjusted^{†} | 1 | 1.25 (0.93-1.68) | |
| Past Smoking | No | Yes | |
| Unadjusted | 1 | 1.13 (0.93-1.37) | |
| Adjusted^{†} | 1 | 0.95 (0.77-1.17) | |
| Diabetes | No | Yes | |
| Unadjusted | 1 | 1.79 (1.50-2.14) | |
| Adjusted^{†} | 1 | 1.40 (1.16-1.68) | |

^{†}Adjusted models contain CHRP(PEROX), age, LDL, HDL, gender, hypertension, current smoking, past smoking, and diabetes.

### Example Calculation of the CHRP (PEROX) Risk Score

A 74 year old non-smoking, non-diabetic female with history of cardiovascular disease but no history of hypertension was seen by her primary care physician because of intervening history of occasional chest discomfort with exertion over a number of months. A stress echo was performed and showed non-diagnostic eletrocardiographic changes that were unchanged from prior studies. The study was otherwise normal. A complete blood cell count with differential was run prior to elective diagnostic cardiac catheterization (Table 29).

TABLE 29

| Hematology Analyzer parameters | Value |
|---|---|
| White blood cell related | |
| White blood cell count (×10³/ml) | 13.93 |
| Neutrophils (%) | 77.1 |
| Lymphocytes (%) | 14.8 |
| Monocytes (%) | 6.2 |
| Eosinophils (%) | 0.5 |
| Basophils (%) | 0.3 |
| Large unstained cells (%) | 1.1 |
| Neutrophil count (×10³/ml) | 10.7 |
| Lymphocyte count (×10³/ml) | 2.05 |
| Monocyte count (×10³/ml) | 0.86 |
| Eosinophil count (×10³/ml) | 0.07 |
| Basophil count (×10³/ml) | 0.04 |
| Large unstained cells count | 0.15 |
| Ky | 98 |
| High peroxidase staining cells count | 6.27 |
| Number of peroxidase saturated cells (×10³/ml) | 25.1 |
| Lymphocyte/large unstained cell threshold | 48 |
| Lymphocytic mode | 36.5 |
| Perox d/D | 0.95 |
| Peroxidase y sigma | 8.74 |
| Blasts (%) | 0.8 |
| Blasts count | 11.1 |
| Mononuclear central y channel | 14.2 |
| Mononuclear polymorphonuclear valley | 17 |
| Red blood cell related | |
| RBC count (×10⁶/ml) | 3.58 |
| Hematocrit (%) | 30.2 |
| Mean Corpuscular volume (MCV) | 83.4 |
| Mean corpuscular hgb (MCH; pg) | 28.0 |
| Mean corpuscular hgb concentration (MCHC; | 33.5 |
| g/dl) | |
| RBC hgb concentration mean (CHCM; g/dl) | 34.2 |
| RBC distribution width (RDW; %) | 14.4 |
| Hgb distribution width (HDW; g/dl) | 2.72 |
| Hgb content distribution width (CHDW; pg) | 34.2 |
| Macrocytic RBC count (×10⁶/ml) | 43 |
| Hypochromic RBC count (×10⁶/ml) | 379 |
| Hyperchromic RBC count (×10⁶/ml) | 347 |
| Microcytic RBC count (×10⁶/ml) | 805 |
| NRBC (%) | 0 |
| Measured Hgb | 10 |
| Platelet related | |
| Platelet count (PLT; %) | 491 |
| Mean platelet volume (MPV) | 7.9 |
| Platelet distribution width (PDW) | 55.5 |
| Plateletcrit (PCT; %) | 0.39 |
| Mean platelet concentration (MPC; g/dl) | 25.8 |
| Large platelets (×10³/ml) | 8 |
| Flag for left shift | 0 |

### Determining the CHRP PEROX Risk Score

With simple modifications to the hematology analyzer, calculation of the CHRP PEROX risk score can be done in automated fashion and provided as a value just like all other hematology analyzed calculated elements. Below, however, is a longhand example.

### Step One—Determining Whether Criteria for Each High Risk and Low Risk Pattern are Met.

Elements used to calculate the CHRP PEROX risk score are used by determining in Yes/No fashion whether binary patterns associated with high vs. low risk are satisfied. Elements included in patterns combine only data measured during performance of a routine CBC and differential (some of the data elements are measured but not routinely reported within common hematology analyzers). Table 30 lists the high risk patterns for death and MI. The death high risk pattern #1 consists of a CHDW>=3.66 and CHCM<=35.7. The example subject has CHDW of 4.2 and CHCM of 34.2 (Table 30A). Thus, this subject's data satisfies both criterion. Both criteria must be satisfied to have a pattern. This subject therefore possesses the Death High Risk #1 pattern and is assigned a point value of one (1). If the subject did not fulfill the criterion for the pattern, a point value of zero (0) would be assigned.

TABLE 30A

| | Subject | | Point |
|---|---|---|---|
| Dth-1 year high-risk patterns | Value | Pattern | value |
| Hgb content distribution | CHDW = 4.2 | Yes | 1 |
| width >=3.66 & | CHCM = 34.2 | | |
| RBC hgb concentration | | | |
| mean <=35.7 | | | |

The above approach is used to fill in whether each High and Low Risk Patterns are satisfied.

TABLE 30B
indicating whether criteria for each high risk pattern for death and MI are met

| | Subject | | Point |
|---|---|---|---|
| | Value | Pattern | value |
| Dth-1 year high-risk patterns | | | |
| Hgb content distribution width >=3.66 & | CHDW = 4.2 | Yes | 1 |
| RBC hgb concentration mean <=35.7 | CHCM = 34.2 | | |
| Percent Lymphocytes <=20 & | % Lymph = 14.8 | Yes | 1 |
| Percent Neutrophils >51.8 | % Neut = 77.1 | | |
| Hgb distribution width >2.76 & | HDW = 2.72 | No | 0 |
| Mean Corpuscular volume >=86.5 | MCV = 83.4 | | |
| Hematocrit <=39.2 & | HCT = 30.2 | Yes | 1 |
| Percent Monocytes >=3.3 | % Mono = 6.2 | | |
| Mononuclear central y channel <=15.6 & | MNY = 14.2 | Yes | 1 |
| Blasts count >5.4198 | nblasts = 11.1 | | |
| Mean platelet concentration <=26.7 & | MPC = 25.8 | Yes | 1 |
| Hgb distribution width >2.52 | HDW = 2.72 | | |
| Eosinophil count >0.37 & | Eos = 0.07 | No | 0 |
| White blood cell count >=5.4 | WBCP = 13.93 | | |
| Hyperchromic RBC count <=239 & | Hyper = 347 | No | 0 |
| White blood cell count >4.244 | WBCP = 13.93 | | |
| MI-1 year high-risk patterns | | | |
| Large platelets <=2 & | Large_platelets = 8 | No | 0 |
| Peroxidase y sigma >8.53 | Pxy_sigma = 0 | | |
| Macrocytic RBC count <31.4 or >641 & | Macro = 43 | No | 0 |
| Ky <=94 | KY = 98 | | |
| Microcytic RBC count <162 & | Micro = 805 | No | 0 |
| Hgb distribution width >2.7598 | HDW = 2.72 | | |
| Macrocytic RBC count <31.4 or >641 & | Macro = 43 | No | 0 |
| Hematocrit <=39.2 | HCT = 30.2 | | |
| Blasts count >5.42 & | nblasts = 11.1 | Yes | 1 |
| Neutrophil count x high peroxidase staining | nperox_sat = 25.1 | | |
| count >0 | | | |
| Mean corpuscular hgb >=31.2 & | MCH = 28 | No | 0 |
| Peroxidase y sigma >=8.53 | Pxy_sigma = 0 | | |
| NRBC <=34 & | Nrbc = 87 | No | 0 |
| Plateletcrit <0.16 | PCT = 0.39 | | |
| RBC count <3.64 or >4.96 & | RBC = 3.58 | Yes | 1 |
| Lymphocytic mode >=35.5 | Lymph_mode = 36.5 | | |
| Macrocytic RBC count <31.4 or >641 & | Macro = 43 | No | 0 |
| Hypochromic RBC count >113 | Hypo = 379 | | |
| Percent Basophils*WBCP <1.68 or >8.21 & | Nbaso = 4.16 | No | 0 |
| Percent Monocytes >=6 | % Mono = 6.2 | | |
| MPM <1.8 or >2.29 & | MPM = 1.94 | No | 0 |
| Monocyte count >0.38 | Mono = 0.86 | | |
| Mean platelet volume >=9.1 & | MPV = 7.9 | No | 0 |
| High peroxidase staining cell count <5.72 | Nhpx = 25.1 | | |
| Mean Platelet volume <7 or >9.1 & | MPV = 7.9 | No | 0 |
| Percent Basophils*WBCP <1.68 or >8.21 | Nbaso_sat = 4.16 | | |
| Percent Lymphocytes <12.8 or >34.9 & | % Lymph = 14.8 | No | 0 |
| Hematocrit <=39.2 | HCT = 30.2 | | |
| RBC distribution width >=13.6 & | RDW = 14.4 | No | 0 |
| Mononuclear polymorphonuclear valley >=21 | MN_PMN_valley = 17 | | |
| NRBC <=53 & | Nrbc = 87 | No | 0 |
| Percent Lymphocytes <=12.8 | % Lymph = 14.8 | | |
| Hgb distribution width >=3.05 & | HDW = 2.72 | No | 0 |
| Percent Large unstained cells <=2.5 | % LUC = 1.1 | | |

TABLE 31
indicating whether criteria for each low risk pattern for death and MI are met

| | Subject | | Point |
|---|---|---|---|
| | Value | Pattern | value |
| Dth-1 year low-risk patterns | | | |
| RBC distribution width <=13.6 & | RDW = 14.4 | No | 0 |
| Mononuclear polymorphonuclear valley >=18 | MN_PMN_valley = 17 | | |
| Hematocrit >=39.2 & | HCT = 30.2 | No | 0 |
| Peroxidase y sigma <=9.49 | Pxy_sigma = 8.74 | | |
| Macrocytic RBC count <227 & | Macro = 43 | No | 0 |
| Blasts count <5.4198 | Nblasts = 11.1 | | |
| Percent Monocytes <=6 & | % Mono = 6.2 | No | 0 |
| Percent Lymphocytes >=20 | % Lymph = 14.8 | | |
| Hypochromic RBC count <113 & | Hypo = 379 | No | 0 |
| White blood cell count <=6.96 | WBCP = 13.93 | | |
| Blasts count <3.15 & | Nblasts = 11.1 | No | 0 |
| Percent Eosinophils >1.2 | % Eos = 0.5 | | |
| Microcytic RBC count <=349 & | Micro = 805 | No | 0 |
| RBC count >=4.07 | RBC = 3.58 | | |
| Mononuclear central y channel >=15.1 & | MNY = 14.2 | No | 0 |
| Percent Lymphocytes >=12.8 | % Lymph = 14.8 | | |
| Macrocytic RBC count <=86 & | Macro = 43 | Yes | 1 |
| Percent Neutrophils >=51.8 | % Neut = 77.1 | | |
| Hgb distribution width <2.76 & | HDW = 2.72 | No | 0 |
| White blood cell count <=5.4 | WBCP = 13.93 | | |
| Mononuclear polymorphonuclear valley <13.3 | MN_PMN_valley = 17 | No | 0 |
| or >15.6 & Monocyte count <0.51 | Mono = 0.86 | | |
| Platelet count >=251 & | PCT = 491 | No | 0 |
| Monocyte count <0.38 | Mono = 0.86 | | |
| Platelet count >=251 & | PCT = 491 | No | 0 |
| Mean corpuscular hgb concentration >=33.9 | MCHC = 33.5 | | |
| Platelet distribution width <=52.9 & | PDW = 55.5 | No | 0 |
| Blasts count <5.42 | Nblasts = 11.1 | | |
| Lymphocyte count >1.21 & | Lymph = 2.05 | No | 0 |
| Percent Monocytes <4.6 | % Mono = 6.2 | | |
| MI-1 year low-risk patterns | | | |
| Hypochromic RBC count <=27 & | Hypo = 379 | No | 0 |
| Ky >=98 | KY = 98 | | |
| RBC distribution width <=12.8 & | RDW = 14.4 | No | 0 |
| Mean corpuscular hgb <=32.6 | MCH = 28 | | |
| Hypochromic RBC count <=27 & | Hypo = 379 | No | 0 |
| Neutrophil count <4.71 | Neut = 10.7 | | |
| MPM >1.8 and <2.29 & | MPM = 1.94 | No | 0 |
| Peroxidase y sigma <=7.59 | Pxy_sigma = 8.74 | | |
| RBC distribution width <=12.8 & | RDW = 14.4 | No | 0 |
| Neutrophil count <=4.71 | Neut = 10.7 | | |
| Hypochromic RBC count <=27 & | Hypo = 379 | No | 0 |
| Monocyte count <0.38 | Mono = 0.86 | | |
| RBC distribution width <=13.6 & | RDW = 14.4 | No | 0 |
| Perox d/D >0.96 | Perox_d_D = 0.95 | | |
| RBC distribution width <=12.8 & | RDW = 14.4 | No | 0 |
| Lymphocyte count >1.21 | Lymph = 2.05 | | |
| Hypochromic RBC count <=27 & | Hypo = 379 | No | 0 |
| Percent Lymphocytes >=20 | % Lymph = 14.8 | | |
| MPM >1.8 and <2.29 & | MPM = 1.94 | No | 0 |
| Hypochromic RBC count <=27 | Hypo = 379 | | |
| Blasts count <3.15 & | Nblasts = 11.1 | No | 0 |
| Eosinophil count >0.14 | Eos = 0.5 | | |
| Blasts count <3.15 & | Nblasts = 11.1 | No | 0 |
| Large unstained cell count >0.07 | LUC = 0.15 | | |
| Percent blasts <0.5 & | % Blasts = 0.8 | No | 0 |
| Percent Neutrophils <=78.1 | % Neut = 77.1 | | |
| Hgb content distribution width <=3.66 & | CHDW = 4.2 | No | 0 |
| Basophil count <0.05 | Baso = 0.04 | | |
| Hgb distribution width <2.76 & | HDW = 2.72 | No | 0 |
| Percent blasts <0.5 | % blasts = 0.8 | | |
| Flag for left shift <1 & | F_leftshift = 0 | No | 0 |
| Blasts count <3.15 | Nblasts = 11.1 | | |
| Plateletcrit >0.16 & | PCT = 0.39 | No | 0 |
| Lymphocyte/large unstained cell | Lymph_LUC_thres = 48 | | |
| threshold <=44 | | | |
| Hgb content distribution width <=3.66 & | CHDW = 4.2 | No | 0 |
| Peroxidase y sigma <=7.59 | Pxy_sigma = 8.74 | | |
| Macrocytic RBC count >31.4 and <641 & | Macro = 43 | Yes | 1 |
| Percent Basophils <0.5 | % Baso = 0.3 | | |

Step Two—Counting the Number of High and Low Risk Patterns that are Satisfied.

The next step is to count how many positive and negative patterns are fulfilled. In this example:

Number of high risk patterns Subject has=7

Number of low risk patterns Subject has=2

### Step Three—Calculating the Weighted Raw Score.

Subjects almost always have combinations of both high and low risk patterns. Overall risk is calculated as the difference in the average number of high risk patterns and the average number of low risk patterns fulfilled by the subject.

The number of high risk patterns is 25.

The number of low risk patterns is 34.

Average # high risk patterns satisfied by the subject=7/25

Average # low risk patterns satisfied by the subject=2/34

The Raw Score of a subject is calculated by the weighted sum of high risk and low risk patterns. In this example:

Raw Score=1/Total number of high risk patterns*Number of high risk patterns satisfied by subject−1/Total number of low risk patterns*Number of low risk patterns satisfied by subject=7/25-2/34=0.221

The calculated Raw Score ranges from −1 to +1 with 0 as the midpoint. A score of 0 is set if the patient satisfies none of the positive or negative patterns or if the patient satisfies equal proportions of positive and negative patterns.

### Step Four—Calculating the Final CHRP Value

The last step is to adjust the Raw Score (range from −1 to +1) to the CHRP (range of 0 to 100, assuming 50 as the midpoint of the scale) by multiplying the Raw Score by 50, and then adding 50.

$$\begin{matrix}CHRP ( PEROX ) = ( 50 \times Raw Score ) + 50 \\ = ( 50 \times 0.221 ) + 50 \\ = 61.1\end{matrix}$$

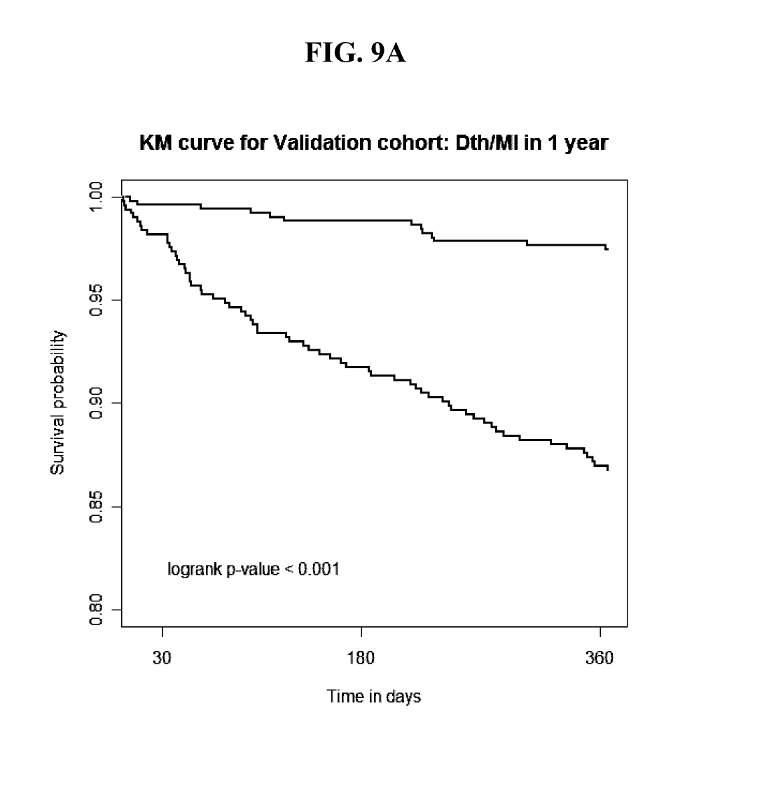

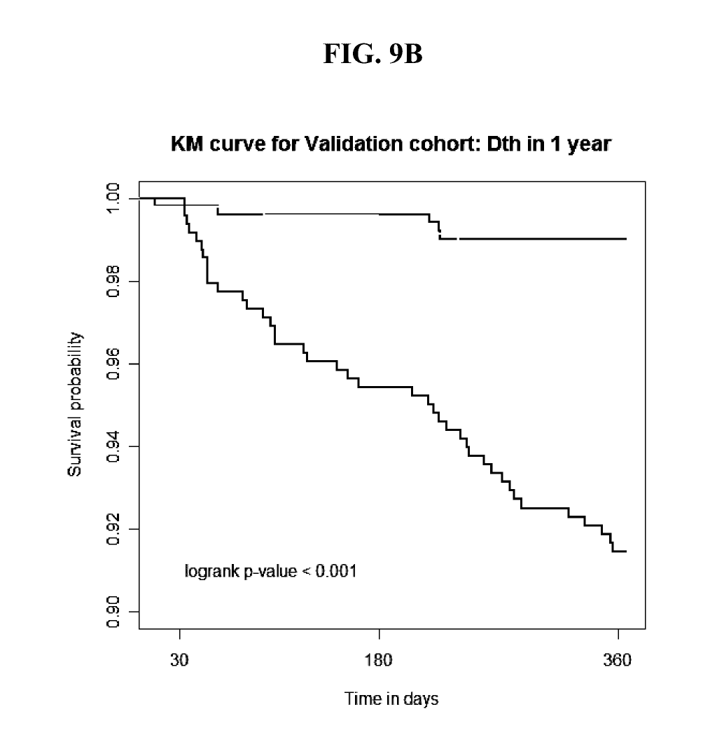

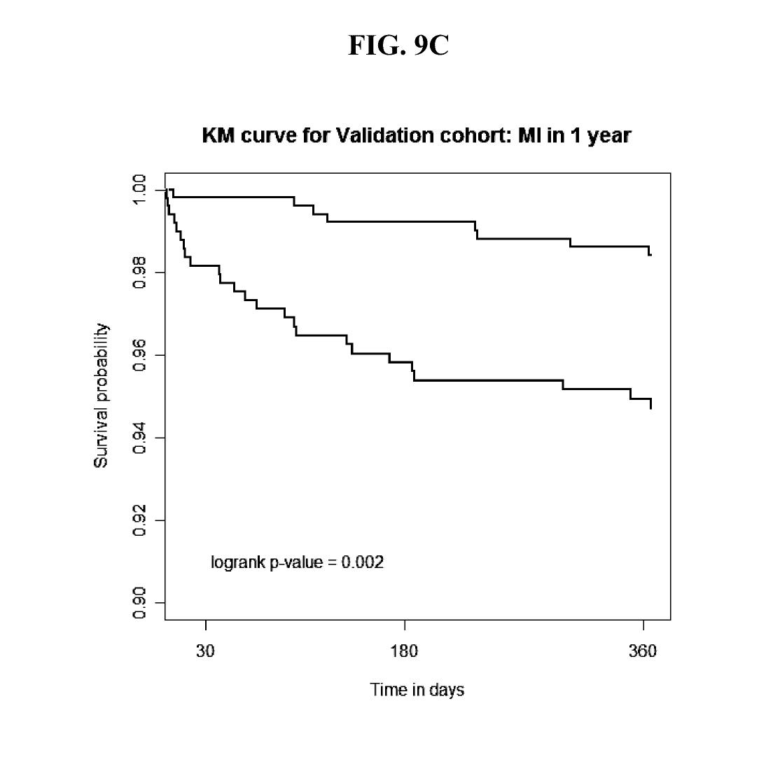

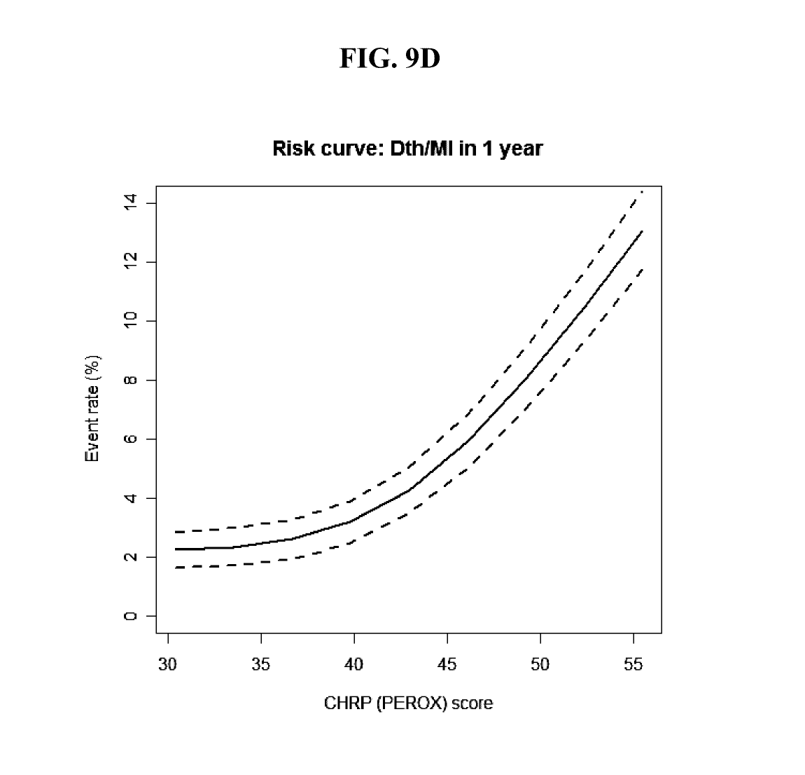

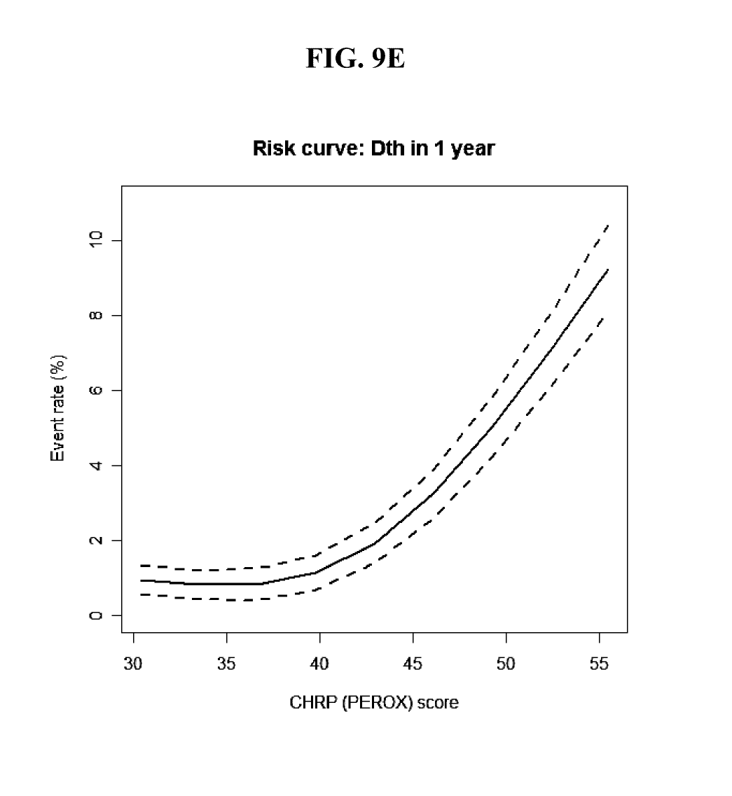

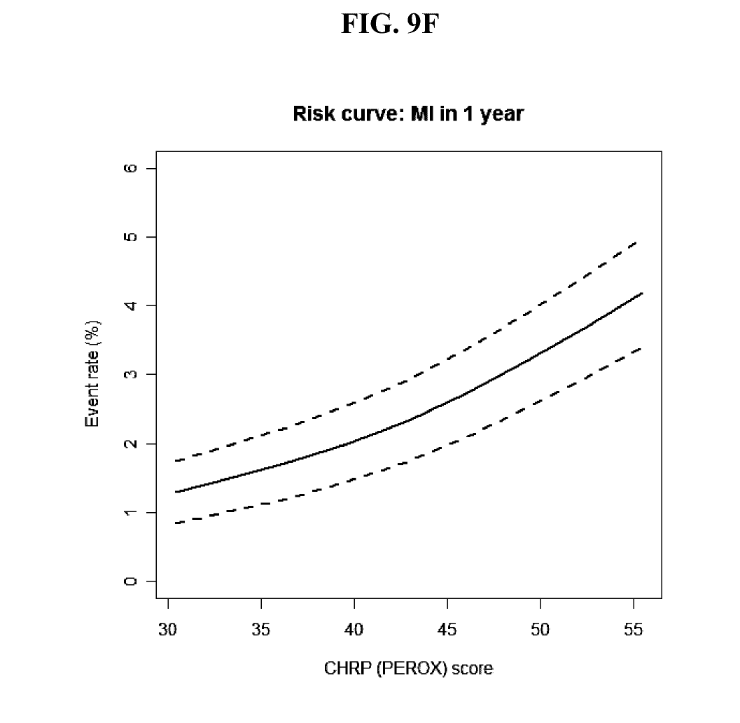

This subject falls into the high risk category. FIG. 9F allows one to use the CHRP Risk Score to estimate overall incident risk of death or MI over the ensuing 1 year period. In this example, the subject's 1 yr event rate is greater than 7%.

TABLE 32
Extensive list of variables that are potentially attainable from ADVIA 120 hematology analyzer.

| | | | | Hemoglobin | Platelet | | |
|---|---|---|---|---|---|---|---|
| Peroxidase Channel | Baso Channel | RBC Channel | RBC Channel | Abs | Channel | Flags | Subclusters |
| % lymph | baso % saturation | % hyper | # hypo norm | hgb | large plt | immature | % abnormal |
| | | | | | | granulocytes | cells |
| % mono | % blasts | % hypo | # hypo micro | delta hgb | mpc | left shift | x mean |
| % neut | % mn | % macro | caculated hgb | mch | mpm | atypical | y mean |
| | | | | | | lymphocytes | |
| % eos | % pmn | % micro | Ch | mchc | mpv | | kx |
| % luc | % pmn ratio | % micro/hypo ratio | Chcm | | pcdw | | ky |
| # lymph | % baso suspect | hyper count | Chdw | | pct | | cluster count |
| # mono | % baso | hypo count | Hct | | pdw | | cluster id |
| # neut | # baso | macro count | Hdw | | plt | | cell count |
| # eos | baso d/D | micro count | rbc scatter high max | | pltn | | area |
| # luc | lobularity index | % hyper macro | rbc scatter low min | | plbc | | weight |
| % hpx | baso mn/ | % hyper norm | rbc valid cells | | plty | | weight over |
| | pmn valley | | | | | | sigma |
| perox % sat | mnx | % hyper micro | Rbcx | | pmdw | | x bar |
| mpxi | mny | % norm macro | rbc x sigma | | rbc fragments | | y bar |
| neut x | pmnx | % norm norm | Rbcy | | rbc ghosts | | sigmax |
| neut y | baso wbc count | % norm micro | rbc y sigma | | | | sigmin |
| lymph mode | | % hypo macro | Rdw | | | | theta |
| lymph/luc threshold | | % hypo norm | rbc/plt average | | | | costheta |
| | | | pulse width | | | | |
| perox d/D | | % hypo micro | | | | | sinetheta |
| perox noise- | | # hyper macro | | | | | |
| lymph valley | | | | | | | |
| perox wbc count | | # hyper norm | | | | | |
| plt clumps | | # hyper mico | | | | | |
| kx | | # norm macro | | | | | |
| ky | | # norm norm | | | | | |
| valley count | | # norm micro | | | | | |
| # nrbc | | # hypo macro | | | | | |

Table 32 shows an extensive list of variables that are potentially attainable from ADVIA 120 (or either predecessor or successor model) hematology analyzer. There are −166 variables that known that are available and potentially informative from the ADVIA 120 hematology analyzer. Column headers indicate i) channel in which variable is determined (peroxidase, baso, rbc, platelet), ii) flags that are triggered by pre-set criteria, or iii) subcluster properties from analysis of specific cellular populations. Both channel and flag information are obtained from DAT files and extracted using a macro. Subcluster information can either be manually collected from cytogram printouts or extracted programatically.

Note that the parameters listed are a combination of raw and manipulated data. The data for the CHRP-PEROX was derived with data that was processed using Bayer 215 software. There are additional Bayer software programs (such as the newer SP3 software that differ in the griding matrix and some of the definitions) that can also be utilized. Separate from use of Bayer-proprietary software, the data that is present in the actual raw flow cytogram (RD files) can be processed using commercially available software (such as Flojo). To summarize, there are additional mathematical parameters that can be determined separately from the list of variables that are shown in the tables and that could be useful. Note also that reticulocyte parameters (104 potential variables) are not included here or in the CHRP-PEROX score as these analyses were not performed.

TABLE 33
List of variables CHRP-Perox might come from.

| | | | | Hemoglobin | Platelet | | |
|---|---|---|---|---|---|---|---|
| Peroxidase Channel | Baso Channel | RBC Channel | RBC Channel | Abs | Channel | Flags | Subclusters |
| % lymph | baso % | % hyper | # hypo norm | hgb | large plt | immature | % abnormal |
| | saturation | | | | | granulocytes | cells |
| % mono | % blasts | % hypo | # hypo micor | delta hgb | mpc | left shift | x mean |
| % neut | % mn | % macro | calculated hgb | mch | mpm | atypical | y mean |
| | | | | | | lymphocytes | |
| % eos | % pmn | % micro | Ch | mchc | mpv | | kx |
| % luc | % pmn ratio | % micro/hypo ratio | Chcm | | pcdw | | ky |
| # lymph | % baso suspect | hyper count | Chdw | | pct | | cluster count |
| # mono | % baso | hypo count | Hct | | pdw | | cluster id |
| # neut | # baso | macro count | Hdw | | plt | | cell count |
| # eos | baso d/D | micro count | rbc scattter high max | | pltn | | area |
| # luc | lobularity index | % hyper macro | rbc scatter low min | | plbc | | weight |
| % hpx | baso mn/pmn | % hyper norm | rbc valid cells | | plty | | weight |
| | valley | | | | | | over sigma |
| perox % sat | mnx | % hyper micro | Rbcx | | pmdw | | x bar |
| mpxi | mny | % norm macro | rbc x sigma | | rbc fragments | | y bar |
| neut x | pmnx | % norm norm | Rbcy | | rbc ghosts | | sigmax |
| neut y | baso wbc count | % norm micro | rbc y sigma | | | | sigmin |
| lymph mode | | % hypo macro | Rdw | | | | theta |
| lymph/luc threshold | | % hypo norm | rbc/plt average | | | | costheta |
| | | | pulse width | | | | |
| perox d/D | | % hypo micro | | | | | sinetheta |
| perox noise- | | # hyper macro | | | | | |
| lymph valley | | | | | | | |
| perox wbc count | | # hyper norm | | | | | |
| plt clumps | | # hyper micro | | | | | |
| kx | | # norm macro | | | | | |
| ky | | # norm norm | | | | | |
| valley count | | # norm micro | | | | | |
| # nrbc | | # hypo macro | | | | | |

Table 33 above shows a list of variables CHRP-Perox might come from. Streamlined version of Table 32 that excludes non-informative variables and includes variables of potential use in CHRP-Perox (i.e., box only using specifically a hematology analyzer that uses in situ cytochemical peroxidase based assay like ADVIA). Tables 34 and 35 are shortened versions of this table (Table 33).

TABLE 34
List of variables CHRP might come from that are common
to other hematology analyzers.

| Peroxidase Channel | Baso Channel | RBC Channel | Hemoglobin Abs | Platelet Channel | Flags |
|---|---|---|---|---|---|
| % lymph | % blasts | % hyper | measured hgb | large plt | immature granulocytes |
| % mono | % baso | % hypo | mch | mpv | left shift |
| % neut | # baso | % macro | mchc | pct | atypical lymphocytes |
| % eos | | % micro | | pdw | |
| % luc | | hyper count | | plt | |
| # lymph | | hypo count | | | |
| # mono | | macro count | | | |
| # neut | | micro count | | | |
| # eos | | hct | | | |
| # luc | | rdw | | | |
| valley count | | mcv | | | |
| | | rbc | | | |

Table 34 provides a list of variables CHRP might come from that are common to other hematology analyzers. Variables in CHRP-Perox (and CHRP) that can also be measured using other hematology analyzers.

TABLE 35
List of variables CHRP-Perox might come from that are unique to ADVIA 120

| Peroxidase Channel | Baso Channel | RBC Channel | RBC Channel | Hemoglobin Abs | Platelet Channel | Subclusters |
|---|---|---|---|---|---|---|
| % hpx | baso % saturation | % micro/hypo ratio | hdw | delta hgb | mpc | % abnormal cells |
| perox % sat | % mn | % hyper macro | rbc scatter high max | | pcdw | x mean |
| mpxi | % pmn | % hyper norm | rbc scatter low min | | mpm | y mean |
| neut x | % pmn ratio | % hyper micro | rbcx | | pmdw | kx |
| neut y | % baso suspect | % norm macro | rbc x sigma | | pltn | ky |
| lymph mode | baso d/D | % norm norm | rbcy | | pltx | cluster count |
| lymph/luc threshold | lobularity index | % norm micro | rbc y sigma | | plty | cluster id |
| perox d/D | baso mn/pmn valley | % hypo macro | | | rbc fragments | cell count |
| perox noise-lymph valley | mnx | % hypo norm | | | rbc ghosts | area |
| perox wbc count | mny | % hypo micro | | | | weight |
| plt clumps | pmnx | # hyper macro | | | | weight over sigma |
| kx | baso wbc count | # hyper norm | | | | x bar |
| ky | | # hyper micro | | | | y bar |
| | | # norm macro | | | | sigmax |
| | | # norm norm | | | | sigmin |
| | | # norm micro | | | | theta |
| | | # hypo macro | | | | costheta |
| | | # hypo norm | | | | sinetheta |
| | | # hypo micro | | | | |
| | | ch | | | | |
| | | chcm | | | | |
| | | chdw | | | | |
| | | caclulated hgb | | | | |

Table 35 provides a list of variables CHRP-Perox might come from that are unique to ADVIA 120. Variables in CHRP-Perox that are calculated by ADVIA 120 and that are not measured by other hematology analyzers.

TABLE 36
Key to Variable-name Abbreviations and Respective Calculations.

| | Abbreviation | Full Name | Definition |
|---|---|---|---|
| Peroxidase | % lymph | percent lymphocyte | percent of total wbcs |
| Channel | % mono | percent monocytes | percent of total wbcs |
| | % neut | percent neutrophils | percent of total wbcs |
| | % eos | percent eosinophils | percent of total wbcs |
| | % luc | percent large unstained cells | percent of total wbcs |
| | # lymph | number lymphocytes | number of total cells |
| | # mono | number monocytes | number of total cells |
| | # neut | number neutrophils | number of total cells |
| | # eos | number eosinophils | number of total cells |
| | # luc | number large unstained cells | number of total cells |
| | % hpx | percent high peroxidase staining cells | percent neuts to right of neut × * 1.4 |
| | perox % sat | percent peroxidase saturation | percent of total cells in last 3 channels perox cytogram |
| | mpxi | mean peroxidase index | [(×mean of sample neuts −66) * 100]/66 |
| | neut x | neutrophil x | mean channel value of neut cluster, x axis |
| | neut y | neutrophil y | mean channel value of neut cluster, y axis |
| | lymph mode | lymphocyte mode | y channel (scatter) that marks mode of lymph cluster |
| | lymph/luc threshold | lymphocyte/large unstained cell threshold | highest scatter of lymphs from noise/lymph histogram |
| | perox d/D | perox d/D | measure of valley between lymph/noise clusters |
| | perox noise-lymph valley | perox noise-lymphocyte valley | channel that marks valley between lymph/noise clusters |
| | perox wbc count | peroxidase-based wbc count | white blood cell count |
| | plt clumps | platelet clumps | number of platelet clumps |
| | kx | kx | how well neut & lymph clusters fit archetype |
| | ky | ky | how well neut & lymph clusters fit archetype |
| | valley count | valley count | number of cells in nrbc region of perox cytogram |
| Baso | baso % saturation | percent basophil saturation | percent of cells in baso saturaion area |
| Channel | % blasts | percent blastocytes | percent of cells in blast region |
| | % mn | percent mononuclear cells | percent of cells in mononuclear region |
| | % pmn | percent polymorphonuclear cells | percent of cells in polymorphonuclear region |
| | % pmn ratio | percent pmn ratio | percent pmn/[percentneut + percenteos] |
| | % baso suspect | percent basophil suspect | perecent of baso cells falling in suspect region |
| | % baso | percent basophils | perecent of total wbcs |
| | # baso | number basophils | number of total cells |
| | baso d/D | baso d/D | [Mn mode count − mn/pmn valley count]/mn mode count |
| | lobularity index | lobularity index | ratio of mode of pmn to mode of mn |
| | baso mn/pmn valley | basophil mononuclear | valley between mn and pmn clsuters |
| | | /polymorphonuclear valley | |
| | mnx | mnx | x channel value that marks center of initial located mn cluster |
| | mny | mny | y channel value that marks center of initial located mn cluster |
| | pmnx | pmnx | x channel value that is mode of pmn population |
| | baso wbc count | basophil wbc count | white blood cell count |
| RBC | % hyper | percent of hyperchromic rbcs | percent of total rbcs |
| Channel | % hypo | percent of hypochromic rbcs | percent of total rbcs |
| | % macro | percent of macrocytic rbcs | percent of total rbcs |
| | % micro | percent of microcytic rbcs | percent of total rbcs |
| | % micro/hypo ratio | percent of microcytic/hypochromic cells | percent of total rbcs |
| | hyper count | number of hyperchromic rbcs | number of cells |
| | hypo count | number of hypochromic rbcs | number of cells |
| | macro count | number of macrocytic rbcs | number of cells |
| | micro count | number of microcytic rbcs | number of cells |
| | % hyper macro | percent of hyperchromic/macrocytic rbcs | percent of total rbcs |
| | % hyper norm | percent of hyperchromic/normocytic rbcs | percent of total rbcs |
| | % hyper micro | percent of hyperchromic/microcytic rbcs | percent of total rbcs |
| | % norm macro | percent of normochromic/macrocytic rbcs | percent of total rbcs |
| | % norm norm | percent of normochromic/normocytic rbcs | percent of total rbcs |
| | % norm micro | percent of normochromic/microcytic rbcs | percent of total rbcs |
| | % hypo macro | percent of hypochromic/macrocytic rbcs | percent of total rbcs |
| | % hypo norm | percent of hypochromic/normocytic rbcs | percent of total rbcs |
| | % hypo micro | percent of hypochromic/microcytic rbcs | percent of total rbcs |
| | # hyper macro | number hyperchromic/macrocytic rbcs | number of cells |
| | # hyper norm | number hyperchromic/normocytic rbcs | number of cells |
| | # hyper micro | number hyperchromic/microcytic rbcs | number of cells |
| | # norm macro | number normochromic/macrocytic rbcs | number of cells |
| | # norm norm | number normochromic/normocytic rbcs | number of cells |
| | # norm micro | number normochromic/microcytic rbcs | number of cells |
| | # hypo macro | number hypochromic/macrocytic rbcs | number of cells |
| | # hypo norm | number hypochromic/normocytic rbcs | number of cells |
| | # hypo micro | number hypochromic/microcytic rbcs | number of cells |
| | caculated hgb | calculated hemoglobin | [chcm * mcv * rbc]/1000 |
| | ch | hemoglobin content | [hc * v]/100 |
| | chcm | cell hemoglobin concentration mean | |
| | chdw | hemoglobin content distribution width | standard deviation of ch histogram |
| | hct | hematocrit | percent of volume of blood consisting of rbcs |
| | hdw | hemoglobin distribution width | standard deviation of hemoglobin conentration histogram |
| | rbc scatter high max | rbc scatter high max | events in x channel bounding coincidence region |
| | rbc scatter low min | rbc scatter low min | events in y channel bounding coincidence region |
| | mcv | mean corpuscular volume | |
| | rbc | red blood cell count | number of red blood cells |
| | rbcx | rbcx | mean channel of rbc x-axis data |
| | rbc x sigma | rbc x sigma | standard deviation of rbc x-axis data |
| | rbcy | rbcy | mean channel of rbc y-axis data |
| | rbc y sigma | rbc y sigma | standard deviation of rbc y-axis data |
| | rdw | red cell distribution width | rbc volume SD/mcv * 100 |
| Hemoglobin | measured hgb | measured hemoglobin | determined using cyanide method algorithm |
| Abs | delta hgb | delta hemoglobin | difference between measured and calculated hemoglobin |
| | mch | mean corpuscular hemoglobin | hgb/rbc * 10 |
| | mchc | mean corpuscular hemoglobin concentration | 1000 * hgb/[rbc * mcv] |
| Platelet | large plt | large platelets | number of cells |
| Channel | mpc | mean platelet component concentration | derived from platelet histogram as name describes |
| | mpm | mean platelet dry mass | derived from platelet histogram as name describes |
| | mpv | mean platelet volume | derived from platelet histogram as name describes |
| | pcdw | platelet component concentration | derived from platelet histogram as name describes |
| | | distribution width | |
| | pct | plateletcrit | percent volume of blood that consists of platelets |
| | pdw | platelet distribution width | platelet volume standard deviation/mpv * 100 |
| | plt | platlet count | number of cells |
| | pltn | platelet mean n | mean of platelets counted |
| | pltx | platelet x | mean of all x-channel raw data |
| | plty | platelet y | mean of all y-channel raw data |
| | pmdw | platelet dry mass distribution width | standard deviation for cells identified as platelets |
| | rbc fragments | rbc fragments | number of cells |
| | rbc ghosts | rbc ghosts | number of cells |
| Flags | immature granulocytes | immature granulocytes | [(% neuts + % eos) − % pmn] >= 5% wbc |
| | left shift | left shift | |
| | atypical lymphocytes | atypical lymphocytes | % LUC >= 4.5% or % LUC >= (% blasts + 1.5%) |
| Subclusters | % abnormal cells | percent of abnormal cells | |
| | x mean | x mean | mean channel of x axis of raw data cluster |
| | y mean | y mean | mean channel of y axis of raw data cluster |
| | kx | kx | compares archetype and sample mean x for neut/lymph clusters |
| | ky | ky | compares archetype and sample mean y for neut/lymph clusters |
| | cluster count | cluster count | number of clusters in final cluster description list |
| | cluster id | cluster id | number associated with cluster |
| | cell count | cell count | number of cells within area of given cluster |
| | area | area | portion of data plane assigned to cluster by classifier |
| | weight | weight | number of cells in cluster divided by total number of cells |
| | weight over sigma | weight over sigma | ratio of cluster weight to product of clusters standard deviation |
| | x bar | x bar | location of cluster mean along x axis |
| | y bar | y bar | location of cluster mean along y axis |
| | sigmax | sigma max | standard deviation along major axis through cluster center |
| | sigmin | sigma min | standard deviation along minor axis through cluster center |
| | theta | theta | |
| | costheta | cosine theta | cosine of tilt of cluster from x axis |
| | sinetheta | sine theta | sine of tilt of cluster from y axis |

Table 36 provides a key to variable-name abbreviations and respective calculations.

### Example 4

### Further Data Analysis

This Example provides further, or alternative, data analysis of the data presented in Examples 1-3 above. In particular, this alternative analysis uses different cutoffs, or numbers, or patterns than discussed above.

### PEROX Results:

Table 37a provides hematology parameters significantly associated with Death or MI in 1 year. A hazard ration (HR) has been computed and the 95% confidence interval (CI) for tertile 3 vs. tertile 1 for the hematology parameters, and retained those parameters which are significantly associated with either Death or MI in 1 year.

TABLE 37a

| | Death in 1 year | MI in 1 year |
|---|---|---|
| | HR (95% CI)‡ | HR (95% CI)‡ |
| White blood cell related | | |
| White blood cell count (×10³/ml) | 1.64 (1.20-2.23) | 0.94 (0.64-1.37) |
| Neutrophils (%) | 2.27 (1.65-3.12) | 0.84 (0.56-1.25) |
| Monocytes (%) | 1.52 (1.13-2.04) | 1.41 (0.95-2.10) |
| Neutrophil count (×10³/ml) | 2.15 (1.56-2.95) | 1.00 (0.68-1.47) |
| Monocyte count (×10³/ml) | 2.05 (1.50-2.80) | 1.19 (0.81-1.74) |
| High peroxidase staining cells | 1.73 (1.31-2.29) | 0.79 (0.54-1.17) |
| count | | |
| Lymphocyte/large unstained cell | 1.41 (1.05-1.89) | 1.27 (0.86-1.87) |
| threshold | | |
| Lymphocytic mode | 1.42 (1.04-1.95) | 1.30 (0.85-1.99) |
| Perox d/D | 0.41 (0.30-0.56) | 0.99 (0.67-1.48) |
| Peroxidase y sigma | 2.70 (1.94-3.77) | 1.38 (0.94-2.04) |
| Blasts (%) | 1.93 (1.42-2.61) | 1.43 (0.97-2.11) |
| Blasts count | 2.28 (1.66-3.14) | 1.55 (1.03-2.33) |
| Mononuclear central y channel | 0.36 (0.26-0.51) | 1.08 (0.74-1.59) |
| Mononuclear polymorphonuclear | 0.50 (0.36-0.68) | 0.98 (0.68-1.41) |
| valley | | |
| Red blood cell related | | |
| RBC count (×10⁶/ml) | 0.32 (0.23-0.46) | 0.83 (0.56-1.23) |
| Hematocrit (%) | 0.32 (0.23-0.45) | 0.69 (0.46-1.02) |
| Mean Corpuscular volume (MCV) | 1.52 (1.11-2.07) | 1.14 (0.79-1.65) |
| Mean corpuscular hgb | 0.24 (0.17-0.35) | 0.93 (0.62-1.39) |
| concentration (MCHC; g/dl) | | |
| RBC hgb concentration mean | 0.24 (0.17-0.35) | 0.79 (0.54-1.15) |
| (CHCM; g/dl) | | |
| RBC distribution width (RDW; %) | 5.84 (3.96-8.62) | 1.95 (1.28-2.97) |
| Hgb distribution width | 2.74 (1.95-3.85) | 1.52 (1.03-2.23) |
| (HDW; g/dl) | | |
| Hgb content distribution width | 4.23 (2.95-6.06) | 1.25 (0.84-1.86) |
| (CHDW; pg) | | |
| Macrocytic RBC count (×10⁶/ml) | 3.30 (2.31-4.73) | 1.31 (0.89-1.91) |
| Hypochromic RBC count | 2.36 (1.74-3.20) | 1.67 (1.12-2.49) |
| (×10⁶/ml) | | |
| Hyperchromic RBC count | 0.42 (0.30-0.58) | 0.97 (0.65-1.43) |
| (×10⁶/ml) | | |
| Microcytic RBC count (×10⁶/ml) | 1.90 (1.39-2.59) | 0.92 (0.63-1.34) |
| NRBC count | 1.48 (1.09-1.99) | 0.93 (0.63-1.38) |
| Measured HGB | 0.23 (0.16-0.33) | 0.79 (0.53-1.18) |
| Platelet related | | |
| Mean platelet volume (MPV) | 1.49 (1.10-2.03) | 1.14 (0.77-1.69) |
| Mean platelet concentration | 0.45 (0.33-0.62) | 0.94 (0.65-1.36) |
| (MPC; g/dl) | | |

Table 37b provides hematology parameters not significantly associated with death or MI in 1 year. Not all hematology parameters examined are associated with incident risks for death or MI. Below is a list of examples of WBC, RBC and platelet related parameters that show no relationship with cardiovascular risks. This list shows that there is not an expectation that all hematology parameters are associated with cardiac disease risks. In fact, the vast majority do not show associations with incident MI or death risk, and only a partial listing of those that do not are shown here.

TABLE 37B

| | Death in 1 year | MI in 1 year |
|---|---|---|
| | HR (95% CI)‡ | HR (95% CI)‡ |
| White blood cell related | | |
| Eosinophils (%) | 0.85 (0.63-1.14) | 1.16 (0.77-1.75) |
| Large unstained cells (%) | 0.77 (0.56-1.04) | 1.12 (0.75-1.68) |
| Eosinophil count (×10³/ml) | 0.93 (0.70-1.25) | 1.05 (0.72-1.54) |
| Basophil count (×10³/ml) | 0.90 (0.66-1.23) | 1.25 (0.81-1.91) |
| Large unstained cells count | 1.11 (0.81-1.51) | 1.02 (0.68-1.52) |
| Ky | 1.03 (0.76-1.41) | 0.85 (0.57-1.26) |
| Number of peroxidase saturated | 1.24 (0.91-1.69) | 0.97 (0.64-1.45) |
| cells (×10³/ml) | | |
| Red blood cell related | | |
| Mean corpuscular hgb (MCH; pg) | 0.77 (0.58-1.03) | 1.20 (0.83-1.75) |
| Platelet related | | |
| Platelet count (PLT; %) | 0.95 (0.70-1.28) | 0.83 (0.57-1.23) |
| Platelet distribution width (PDW) | 1.31 (0.96-1.79) | 1.15 (0.77-1.72) |
| Plateletcrit (PCT; %) | 1.10 (0.81-1.48) | 0.77 (0.52-1.14) |
| Large platelets (×10³/ml) | 1.31 (0.98-1.75) | 1.06 (0.72-1.56) |

Abbreviations:
MI, myocardial infarction;
HR, hazard ratio;
CI, confidence interval;
RBC, red blood cell;
Hgb, hemoglobin.
Hazard ratios were calculated for tertile 3 vs. tertile 1.
‡Derivation Cohort only

Moreover, inspection of the hematology parameters listed in Table 37a (those elements that do show an association with either death or MI risk) often only show association with risk for either MI, or death individually, but not in both. Those with Hazard ratios (HR) that cross unity are not significant. Thus, a review of the RBC related parameters in Table 37a for example shows that RBC count, hematocrit, MCV, MCHC, and CHCM predict risk for death at 1 year but not MI (because for MI the 95% confidence interval for the HR crosses unity). Alternatively, RDW and HDW predict risks for MI and death both.

Collectively, the results in Tables 37a and 37b identify individual hematology analyzer elements that provide prognostic value for prediction of either death or MI risk.

Table 38 shows perturbing the cut-points for the patterns. In the analysis provided in the Examples above, three equal frequency cut-points (i.e., tertiles) were used to identify LAD patterns in the data associated with outcomes death or MI in 1 year. Each pattern is comprised of a binary pair of elements, whose cut points were based upon the above tertiles. However, it is readily conceivable that the cut points listed for the patterns are not the only ones that will work. Rather, there exist numerous possible cut point ranges, and one important thing is that binary pairs of the elements shown are discoveries because they show enhanced prognostic value for prediction of cardiovascular risks.

To illustrate that alternative cutoff values can be used within these binary pairs, and still provide prognostic value, in Table 38, the cut points have been perturbed to those being derived from quintile (i.e., 5 equal categories) based analyses, rather than tertile based for deriving cut-points. Using this quintiles based approach to derive LAD binary pairs, the relative risk (RR) has been computed and 95% confidence interval (CI) for death/MI in 1 year. For illustrative purposes only shown are analyses for Death High risk binary patterns, but the same can be done for death low risk, and MI high and low risk patterns.

Note that the binary patterns obtained after perturbation of the cut point values are also statistically significant. These results indicate that changes in the cut point values used within the binary patterns of high and low risk that are included within the PEROX risk score can still provide prognostic value, and do not yield significantly different patterns.

TABLE 38

| Death | | |
|---|---|---|
| High Risk | Pattern | RR (95% CI) |
| 1 | Hemoglobin content distribution | 2.98 (2.45-3.63) |
| | width >3.83, & Cell hgb concentration | |
| | mean <34.85 | |
| 2 | Hypochromic RBC count >219, & | 3.17 (2.59-3.88) |
| | Hemoglobin content distribution | |
| | width >3.83 | |
| 3 | Mean corpuscular hgb concentra- | 2.61 (2.10-3.24) |
| | tion <34.6, & Perox d/D <0.9 | |
| 4 | Hypochromic RBC count >219, | 2.87 (2.34-3.54) |
| | & Macrocytic RBC count >106 | |
| 5 | Mean corpuscular hgb concentra- | 2.48 (2.00-3.08) |
| | tion <33.4, & Monocyte cluster | |
| | X center <14.4 | |
| 6 | Age >67.83, & Hematocrit <37.3 | 2.74 (2.21-3.41) |
| 7 | Monocyte/polymorphonuclear | 1.69 (1.39-2.05) |
| | valley <18, Perox cluster Y | |
| | axis sigma >8.96 | |
| 8 | Monocyte cluster X center <14.4, | 2.14 (1.73-2.65) |
| | & Perox cluster Y axis mean >17.87 | |
| 9 | C-reactive protein >7.42, | 2.39 (1.94-2.93) |
| | & History of hypertension | |

Table 39 below shows varying the number of patterns selected in the LAD model for risk score computation. It has been shown that individual elements from the hematology analyzer are discovered to predict risk for death or MI, and thus have prognostic value (Table 37a). Then it was shown that binary patterns of elements generate LAD high and low risk patterns with improved prognostic value (Table 38), with the discovery of which elements synergistically pair to provide improved prognostic value being an important discover. If individual binary patterns have prognostic value, so too should combinations of binary patterns of high and low risk (even better in terms of prognostic value). To show this, N high-risk and N low-risk patterns were randomly selected and the area under the ROC curve (AUC) for Death/MI in 1 year was computed. This procedure was repeated 100 times. In Table 39 below, the mean AUC & 95% CI in the 100 bootstrap experiments is presented.

TABLE 39

| N | AUC (Mean & 95% CI) |
|---|---|
| 1 high-risk & | 59.9 (58.63-61.17) |
| 1 low-risk pattern | |
| 5 high-risk & | 70.5 (69.60-71.40) |
| 5 low-risk pattern | |
| 10 high-risk & | 75.6 (75.09-76.11) |
| 10 low-risk pattern | |
| 15 high-risk & | 76.9 (76.57-77.23) |
| 15 low-risk pattern | |

Selection of any 1 high risk, and any one low risk pattern, provided increased prognostic value as evidenced from the accuracy (reflected in the AUC) being significantly different than AUC=50. Moreover, as the number of binary high and low risk patterns used was increased, the accuracy of the model correspondingly increased—such that using any random sampling of 10 high risk binary patterns, and any random sampling of 10 low risk binary patterns, provided 75.6% accuracy in prediction of death or MI risk over the ensuing 1 year interval. Thus, modification of the PEROX risk score by using alternative smaller numbers of patterns of risk (as few as 1) still provides a risk score that has prognostic value.

Table 40 describes changing the weights in the formula for computing PEROX risk score. Numerous alternative weightings have been examined to assemble a cumulative risk score from the individual risk patterns, and find that all provide prognostic value. Equal weighting was given to the individual patterns of high and low risk in the original PEROX risk score since substantial differences with alternative weightings was not seen. This point is illustrated below.

Table 40 shows the results where the accuracy (AUC) for 1 year prediction of death or MI is calculated with patterns having either equal weights, or weights in proportion to the prevalence and prognostic value (relative risk (RR) based) of the patterns, in computing the PEROX score.

TABLE 40

| | PEROX score | PEROX score |
|---|---|---|
| | (equal weights) | (RR weights) |
| Dth1 | 82.84 | 82.56 |
| MI1 | 66.23 | 65.87 |
| DMI1 | 75.77 | 75.48 |

These results show similar prognostic value for PEROX score regardless of whether equal weightings or RR based weightings were used.

Table 41 shows PEROX score can predict other cardiovascular outcomes. The PEROX score was built for predicting death/MI in 1 year. In the table below, the AUC accuracy and relative risk (95% CI) for tertile 1 vs. tertile 3 for multiple alternative cardiovascular endpoints are presented.

TABLE 41

| | AUC | RR (95% C.I.) |
|---|---|---|
| Max Stenosis ≦50% | 68.34 | 1.53 (1.4-1.68) |
| Max Stenosis ≦70% | 65.30 | 1.5 (1.36-1.66) |
| Coronary Artery Disease | 70.10 | 1.49 (1.37-1.62) |
| Peripheral Artery Disease | 69.49 | 3.36 (2.62-4.31) |
| | AUC | RR (95% C.I.) |
| 30 days | | |
| Revasc | 56.37 | 1.38 (1.06-1.8) |
| Death/MI/Revasc | 56.46 | 1.4 (1.08-1.82) |
| 6 months | | |
| Death | 80.66 | 20.12 (2.72-148.99) |
| MI | 67.90 | 5.03 (1.74-14.54) |
| Revasc | 56.57 | 1.38 (1.11-1.73) |
| Death/MI | 73.36 | 7.67 (3.05-19.25) |
| Death/MI/Revasc | 58.98 | 1.58 (1.28-1.95) |
| MI/Revasc | 56.96 | 1.42 (1.15-1.77) |
| Stenosis <50% MI/Revasc | 68.09 | 1.51 (1.38-1.65) |
| 1 year | | |
| Death | 82.84 | 21.56 (5.26-88.36) |
| MI | 66.23 | 3.7 (1.63-8.4) |
| Revasc | 56.11 | 1.35 (1.09-1.67) |
| Death/MI | 75.77 | 7.45 (3.77-14.74) |
| MI/Revasc | 56.41 | 1.37 (1.12-1.68) |
| Stenosis <50% MI/Revasc | 68.28 | 1.52 (1.39-1.66) |
| 3 years | | |
| Death | 77.98 | 8.01 (4.35-14.78) |
| MI | 65.07 | 3.14 (1.62-6.09) |
| Revasc | 55.99 | 1.31 (1.09-1.59) |
| Death/MI | 74.33 | 5.27 (3.41-8.15) |
| Death/MI/Revasc | 62.88 | 1.73 (1.47-2.03) |

It is thus seen that application of the PEROX risk score to multiple alternative near term, and long term, cardiovascular endpoints provides significant prognostic value.

### Bootstrapping Data

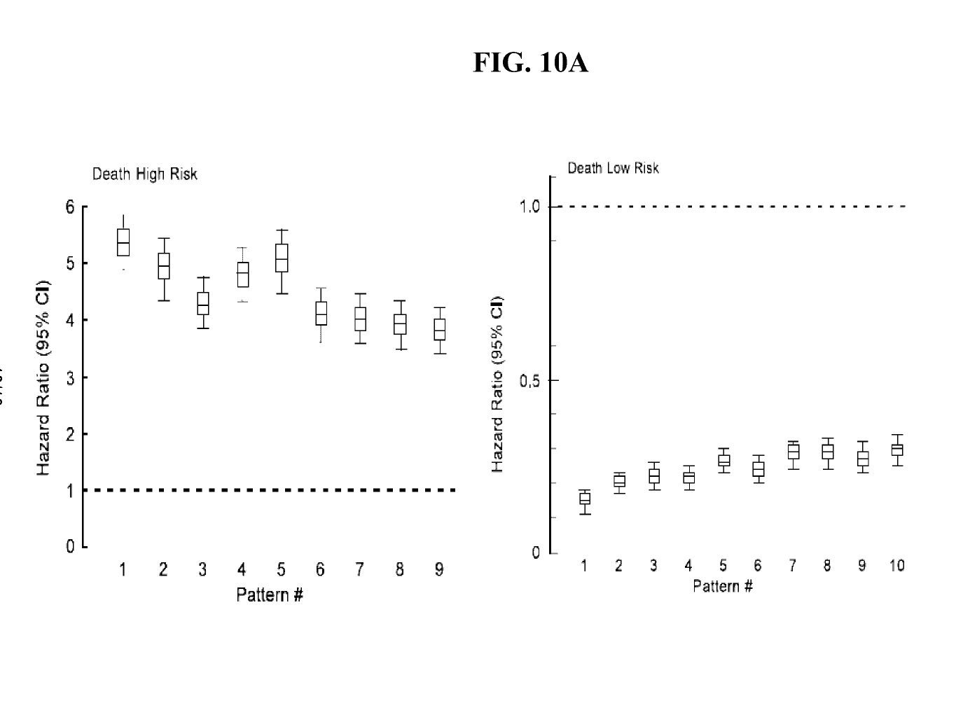

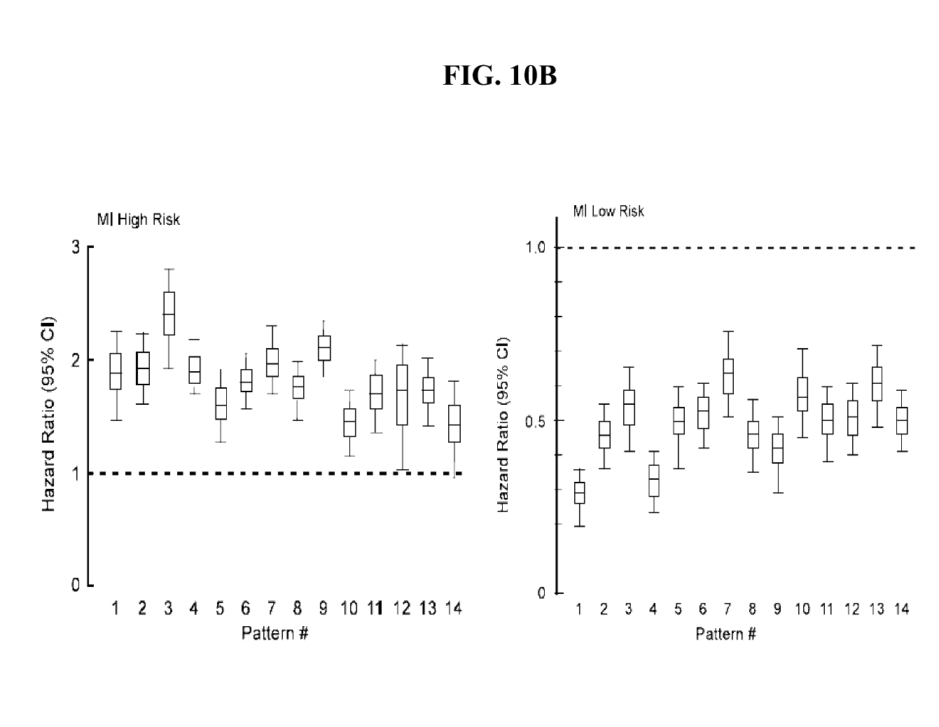

FIGS. 10A and B provide data illustrating that each of the high and low risk patterns for MI and death defined in the above results independently predicts risk. This data somewhat overlaps with the data in the Tables above, but also involves bootstrapping (see below). The results are shown in FIGS. 10A and B. To illustrate that the methodology employed to develop the PEROX risk score helps to define “stable” patterns, additional analyses were performed on the individual high and low risk patterns. The hazard ratios (HRs) were determined from 250 random bootstrap samples with a sample size of 5,895 from the derivation cohort, along with their 2.5th, 5th, 25th, 50th, 75th, 95th and 97th percentile estimates. The data shown in FIGS. 10A and B are the box whisker plots illustrating the distribution of HRs calculated from these independent bootstrap analyses. As can be seen, the high and low risk patterns are quite stable.

### CHRP (PEROX)

In these analyses, the focus is on the risk score using only those patterns available on the ADVIA, and no additional clinical information. The risk score calculated here we call CHRP (Comprehensive hematology risk profile)—PEROX (because it includes peroxidase based hematology analyzer data only available on the ADVIA or earlier versions of the Bayer technicon analyzer). Table 42 provides for Perturbing cut-points in the LAD patterns. In the analysis, three equal frequency cut-points were used to identify LAD patterns in the data associated with outcomes death or MI in 1 year. In the table below, the cut points were perturbed to the closest quintiles and the relative risk (RR) and 95% confidence interval (CI) for death in 1 year has been computed. The patterns obtained after perturbation of the cut point values are also statistically significant, demonstrating that changes in the cut point values of individual elements within the patterns can still provide prognostic value, and do not yield significantly different patterns.

TABLE 42

| | | Death in 1 year |
|---|---|---|
| | Dth-1 year high-risk patterns | RR (95% CI) |
| 1 | Hgb content distribution width >=3.7 & | 4.29 (3.33-5.52) |
| | RBC hgb concentration mean <=35.5 | |
| 2 | Percent Lymphocytes <=21.5 & | 2.81 (2.21-3.57) |
| | Percent Neutrophils >56.2 | |
| 3 | Hgb distribution width >2.7 & | 2.41 (1.89-3.06) |
| | Mean Corpuscular volume >=87.3 | |
| 4 | Hematocrit <=40.1 & | 2.72 (2.15-3.43) |
| | Percent Monocytes >=4 | |
| 5 | Mononuclear central y channel <=15.4 & | 2.67 (2.12-3.38) |
| | Blasts count >4.85 | |
| 6 | Mean platelet concentration <=27 & | 2.31 (1.82-2.91) |
| | Hgb distribution width >2.56 | |
| 7 | Eosinophil count >0.29 & | 1.8 (1.35-2.41) |
| | White blood cell count >=5.69 | |
| 8 | Hyperchromic RBC count <=340 & | 1.78 (1.38-2.29) |
| | White blood cell count >4.8 | |

Table 43 provides for varying the number of patterns selected in the LAD model for risk score computation. N high-risk and N low-risk patterns were randomly selected and the area under the ROC curve (AUC) for Death/MI in 1 year was computed. This procedure was repeated this 100 times. In the table below, the mean AUC & 95% CI in the 100 experiments are presented. All are highly significant with AUC markedly greater and statistically significantly greater than AUC=50. Thus, modification of the CHRP(PEROX) risk score by using alternative smaller numbers of patterns of risk (as few as 1) still provides a risk score that has prognostic value.

TABLE 43

| N | AUC (Mean & 95% CI) |
|---|---|
| 1 high-risk & | 57.4 (56.49-58.31) |
| 1 low-risk pattern | |
| 5 high-risk & | 66.1 (65.02-67.18) |
| 5 low-risk pattern | |
| 10 high-risk & | 68.8 (67.54-70.06) |
| 10 low-risk pattern | |
| 15 high-risk & | 70.7 (69.41-71.99) |
| 15 low-risk pattern | |

Table 44 provides for changing the weights in the formula for computing PEROX risk score. The relative risk (RR) associated with a pattern was used as the weight in computing the CHRP(PEROX) score, and the AUC accuracy for Death/MI in 1 year was computed. These results show similar prognostic value for CHRP(PEROX) score regardless of whether equal weightings or RR based weightings were used. Thus, the relative weights of the individual patterns of high and low risk used to calculate the CHRP(PEROX) can be changed and still provide prognostic value.

TABLE 44

| | PEROX score | PEROX score |
|---|---|---|
| | (equal weights) | (RR weights) |
| Dth1 | 77.30 | 76.58 |
| MI1 | 65.23 | 64.92 |
| DMI1 | 72.31 | 71.74 |

Table 45 shows that CHRP-PEROX score is predictive of other cardiovascular outcomes. The CHRP-PEROX score was built for predicting Death/MI in 1 year. In the table below, the AUC accuracy and relative risk (95% CI) was presented for tertile 1 vs. tertile 3 for multiple alternative cardiovascular endpoints.

TABLE 45

| | AUC | RR (95% CI) |
|---|---|---|
| Max stenosis <50% | 64.56 | 1.42 (1.3-1.54) |
| Max stenosis <70% | 62.89 | 1.43 (1.3-1.58) |
| CAD | 64.45 | 1.34 (1.25-1.45) |
| PAD | 65.19 | 2.56 (2.04-3.22) |
| | AUC | RR (95% CI) |
| 30 days | | |
| Revasc | 55.26 | 1.41 (1.08-1.82) |
| Death/MI/Revasc | 55.02 | 1.4 (1.08-1.8) |
| 6 months | | |
| Death | 78.67 | 10.78 (2.55-45.57) |
| MI | 67.54 | 4.9 (1.69-14.22) |
| Revasc | 55.67 | 1.4 (1.13-1.74) |
| Death/MI | 72.56 | 6.53 (2.79-15.26) |
| Death/MI/Revasc | 58.07 | 1.59 (1.3-1.94) |
| MI/Revasc | 56.1 | 1.44 (1.17-1.78) |
| Stenosis/MI/Revasc | 64.6 | 1.42 (1.3-1.54) |
| 1 year | | |
| Death | 77.3 | 8.03 (3.2-20.15) |
| MI | 65.23 | 3.06 (1.39-6.72) |
| Revasc | 55.36 | 1.38 (1.13-1.69) |
| Death/MI | 72.31 | 4.82 (2.69-8.64) |
| Stenosis/MI/Revasc | 64.7 | 1.42 (1.31-1.55) |
| MI/Revasc | 55.68 | 1.4 (1.15-1.71) |
| 3 year | | |
| Death | 74.46 | 7.3 (3.94-13.53) |
| MI | 63.94 | 3.03 (1.55-5.91) |
| Revasc | 55.82 | 1.43 (1.19-1.71) |
| Death/MI | 71.17 | 4.81 (3.09-7.47) |
| Death/MI/Revasc | 61.49 | 1.76 (1.5-2.06) |

It is thus seen that application of the CHRP(PEROX) to multiple alternative near term, and long term, cardiovascular endpoints provides significant prognostic value.

### CHRP Results:

Table 46 provides for perturbing cut points in the LAD patterns. In the analysis, three equal frequency cut points were used to identify LAD patterns in the data associated with outcomes death or MI in 1 year. In the table below, the cut points were perturbed to closest quintiles and the relative risk (RR) and 95% confidence interval (CI) for death in 1 year was computed. The patterns obtained after perturbation of the cut point values are also statistically significant, demonstrating that changes in the cut point values of individual elements within the patterns can still provide prognostic value, and do not yield significantly different patterns.

TABLE 46

| | | Death |
|---|---|---|
| | Death (1 year) high risk patterns | RR (95% CI) |
| 1 | RBC distribution width >13.4 & | 2.45 (1.94-3.1) |
| | Percent Eosinophils <4.6 | |
| 2 | Hematocrit <42.2 & | 3.47 (2.73-4.42) |
| | Percent Lymphocytes <25.78 | |
| 3 | Mean corpuscular hgb concentration <35.2 & | 2.31 (1.83-2.92) |
| | Lymphocyte count <1.3 | |
| 4 | Mean corpuscular hgb concentration <33.4 & | 1.31 (0.99-1.74) |
| | Percent Lymphocytes >16.6 | |
| 5 | RBC count <4.18 & Percent Basophils <0.9 | 1.93 (1.53-2.44) |
| 6 | White blood cell count >6.57 | 2.04 (1.61-2.58) |
| 7 | Eosinophil count <0.08 or >0.37 & | 1.79 (1.41-2.29) |
| | Monocyte count >0.24 | |

Table 47 provides for varying the number of patterns selected in the LAD model for CHRP risk score computation. N high-risk and N low-risk patterns were randomly selected and the area under the ROC curve (AUC) for Death/MI in 1 year was computed. This procedure was repeated 100 times. In the table below, the mean AUC & 95% CI in the 100 experiments are presented. All are highly significant with AUC markedly greater and statistically significantly greater than AUC=50. Thus, modification of the CHRP risk score by using alternative smaller numbers of patterns of risk (as few as 1) still provides a risk score that has prognostic value.

TABLE 47

| N | AUC (Mean & 95% CI) |
|---|---|
| 1 high-risk & | 59.3 (58.34-60.26) |
| 1 low-risk pattern | |
| 5 high-risk & | 67.1 (65.89-68.31) |
| 5 low-risk pattern | |
| 10 high-risk & | 69.1 (67.81-70.39) |
| 10 low-risk pattern | |
| 15 high-risk & | 70.0 (68.68-71.32) |
| 15 low-risk pattern | |

Table 48 provides for changing the weights in the formula for computing CHRP risk score. The relative risk (RR) associated was used with a pattern as the weight in computing the CHRP score, and the AUC accuracy for Death/MI in 1 year was computed. These results show similar prognostic value for CHRP score regardless of whether equal weightings or RR based weightings were used. Thus, the relative weights of the individual patterns of high and low risk used to calculate the CHRP can be changed and still provide prognostic value.

TABLE 48

| | PEROX score | PEROX score |
|---|---|---|
| | (equal weights) | (RR weights) |
| Dth1 | 77.52 | 77.61 |
| MI1 | 60.92 | 60.50 |
| DMI1 | 70.53 | 70.31 |

Table 49 indicates that CHRP score can predict other cardiovascular outcomes. The CHRP score was built for predicting death/MI in 1 year. In the table below, the AUC accuracy and relative risk (95% CI) for tertile 1 vs. tertile 3 for multiple alternative cardiovascular endpoints have been presented.

TABLE 49

| | AUC | RR (95% CI) |
|---|---|---|
| Max stenosis <50% | 58.88 | 1.24 (1.14-1.35) |
| Max stenosis <70% | 57.26 | 1.24 (1.13-1.37) |
| Coronary Artery Disease | 58.66 | 1.19 (1.1-1.28) |
| Peripheral Artery Disease | 66.28 | 2.83 (2.24-3.58) |
| | AUC | RR (95% CI) |
| 6 months | | |
| Death | 78.62 | 5.12 (1.76-14.86) |
| MI | 62.6 | 2.17 (0.95-4.99) |
| Revasc | 52.63 | 1.27 (1.02-1.59) |
| Death/MI | 69.91 | 3.07 (1.62-5.83) |
| Death/MI/Revasc | 55.44 | 1.44 (1.17-1.77) |
| Stenosis/MI/Revasc | 59.09 | 1.24 (1.15-1.35) |
| MI/Revasc | 53.36 | 1.32 (1.07-1.64) |
| 1 year | | |
| Death | 77.52 | 4.99 (2.36-10.56) |
| MI | 60.92 | 2.05 (1-4.17) |
| Revasc | 52.1 | 1.23 (1-1.52) |
| Death/MI | 70.53 | 3.23 (1.96-5.33) |
| Stenosis/MI/Revasc | 59.28 | 1.25 (1.15-1.35) |
| MI/Revasc | 52.78 | 1.28 (1.04-1.57) |
| Death | 73.18 | 4.14 (2.58-6.65) |
| MI | 59.92 | 1.85 (1.02-3.37) |
| Revasc | 51.5 | 1.16 (0.97-1.4) |
| Death/MI | 68.75 | 2.93 (2.05-4.19) |
| DMR3 | 57.43 | 1.45 (1.24-1.69) |
| 3 years | | |
| Death | 73.18 | 4.14 (2.58-6.65) |
| MI | 59.92 | 1.85 (1.02-3.37) |
| Revasc | 51.5 | 1.16 (0.97-1.4) |
| Death/MI | 68.75 | 2.93 (2.05-4.19) |
| Death/MI/Revasc | 57.43 | 1.45 (1.24-1.69) |

It is thus seen that application of the CHRP to multiple alternative near term, and long term, cardiovascular endpoints provides significant prognostic value.

### Example 5

### Generating Risk Profiles

This Example provides three exemplary ways that risk profiles can be generated for individual patients using three different mathematical models including random survival forest (RSF), the Cox model, and 3) Linear discriminant analysis (LDA). For all three of these, the markers from Table 16 were used and the following patient population was employed. 7,369 patients undergoing elective diagnostic cardiac evaluation at a tertiary care center were enrolled for the study. An extensive array of erythrocyte, leukocyte, and platelet related parameters (Table 16 of provisional application) were captured on whole blood analyzed from each subject at the time of elective cardiac evaluation. The patients were randomly divided into a Derivation (N=5,895) and a Validation Cohort (N=1,473). CHRP was developed using RSF analyses within the Derivation Cohort. Associations between individual markers and the combined outcome of death or MI at one year follow up were determined by using standard RSF methodology. The resultant CHRP formula to estimate risk was examined for its accuracy in the independent Validation Cohort.

Random Survival Forest (RSF)—

Table 52 below displays the prognostic value of CHRP generated using the RSF approach, as measured using AUC. The overall accuracy of the CHRP generated in this fashion was 83.3% for the composite endpoint of 1 year death or MI. When applied to just primary or secondary prevention subjects, comparable accuracies were observed (Table 52).

TABLE 52
AUC for CHRP calculated using Random Survival Forest

| | DMI1 | DTH1 | MI1 |
|---|---|---|---|
| Whole cohort | 83.3 | 87.9 | 74 |
| Primary prevention | 86.8 | 89 | 81.4 |
| Secondary prevention | 82.2 | 87.4 | 72 |

Cox Model—

Table 54 displays the prognostic value of CHRP generated using this approach, as measured using AUC. The overall accuracy of the CHRP generated in this fashion was 71.7% for the composite endpoint of 1 year death or MI. When applied to just primary or secondary prevention subjects, comparable accuracies were observed (Table 54).

TABLE 54
AUC for CHRP calculated using a Cox model

| | DMI1 | DTH1 | MI1 |
|---|---|---|---|
| Whole cohort (n = 7369) | 71.7 | 79.2 | 59 |
| Primary prevention (n = 1859) | 72.9 | 75.7 | 67 |
| Secondary prevention (n = 5510) | 70.7 | 79.2 | 56.6 |

Linear Discriminant Analysis (LDA)—

Table 55 displays the prognostic value of CHRP generated using this approach, as measured using AUC. The overall accuracy (as indicated by AUC) of the CHRP generated in this fashion was 53.1% for the composite endpoint of 1 year death or MI. When applied to just primary or secondary prevention subjects, comparable accuracies were observed (Table 55).

TABLE 55
AUC for CHRP calculated using linear
discriminant analysis (LDA)

| | DMI1 | DTH1 | MI1 |
|---|---|---|---|
| Whole cohort (n = 7369) | 53.1 | 54.6 | 50.4 |
| Primary prevention (n = 1859) | 52.9 | 54.7 | 49.6 |
| Secondary prevention (n = 5510) | 53.1 | 54.5 | 50.4 |

### REFERENCES

(all of which are herein incorporated by reference)
1. Naghavi M, Falk E, Hecht H S, Jamieson M J, Kaul S, Berman D, Fayad Z, Budoff M J, Rumberger J, Naqvi T Z, Shaw L J, Faergeman O, Cohn J, Bahr R, Koenig W, Demirovic J, Arking D, Herrera V L, Badimon J, Goldstein J A, Rudy Y, Airaksinen J, Schwartz R S, Riley W A, Mendes R A, Douglas P, Shah P K. From vulnerable plaque to vulnerable patient—Part III: Executive summary of the Screening for Heart Attack Prevention and Education (SHAPE) Task Force report. Am J Cardiol. 2006; 98:2H-15H.
2. Maisel A S, Bhalla V, Braunwald E. Cardiac biomarkers: a contemporary status report. Nat Clin Pract Cardiovasc Med. 2006; 3:24-34.
3. See R, Lindsey J B, Patel M J, Ayers C R, Khera A, McGuire D K, Grundy S M, de Lemos J A. Application of the screening for Heart Attack Prevention and Education Task Force recommendations to an urban population: observations from the Dallas Heart Study. Arch Intern Med. 2008; 168:1055-1062.
4. Wang T J, Gona P, Larson M G, Tofler G H, Levy D, Newton-Cheh C, Jacques P F, Rifai N, Selhub J, Robins S J, Benjamin E J, D'Agostino R B, Vasan R S. Multiple biomarkers for the prediction of first major cardiovascular events and death. N Engl J Med. 2006; 355:2631-2639.
5. Kathiresan S, Melander O, Anevski D, Guiducci C, Burtt N P, Roos C, Hirschhorn J N, Berglund G, Hedblad B, Groop L, Altshuler D M, Newton-Cheh C, Orho-Melander M. Polymorphisms associated with cholesterol and risk of cardiovascular events. N Engl J Med. 2008; 358:1240-1249.
6. Detrano R, Guerci A D, Carr J J, Bild D E, Burke G, Folsom A R, Liu K, Shea S, Szklo M, Bluemke D A, O'Leary D H, Tracy R, Watson K, Wong N D, Kronmal R A. Coronary calcium as a predictor of coronary events in four racial or ethnic groups. N Engl J Med. 2008; 358:1336-1345.
7. Gaziano T A, Young C R, Fitzmaurice G, Atwood S, Gaziano J M. Laboratory-based versus non-laboratory-based method for assessment of cardiovascular disease risk: the NHANES I Follow-up Study cohort. Lancet. 2008; 371:923-931.
8. Danesh J, Collins R, Appleby P, Peto R. Association of fibrinogen, C-reactive protein, albumin, or leukocyte count with coronary heart disease: meta-analyses of prospective studies. JAMA. 1998; 279:1477-1482.
9. Rana J S, Boekholdt S M, Ridker P M, Jukema J W, Luben R, Bingham S A, Day N E, Wareham N J, Kastelein J J, Khaw K T. Differential leucocyte count and the risk of future coronary artery disease in healthy men and women: the EPIC-Norfolk Prospective Population Study. J Intern Med. 2007; 262:678-689.
10. Packard R R, Libby P. Inflammation in atherosclerosis: from vascular biology to biomarker discovery and risk prediction. Clin Chem. 2008; 54:24-38.
11. Sugiyama S, Okada Y, Sukhova G K, Virmani R, Heinecke J W, Libby P. Macrophage myeloperoxidase regulation by granulocyte macrophage colony-stimulating factor in human atherosclerosis and implications in acute coronary syndromes. Am J Pathol. 2001; 158:879-891.
12. Nicholls S J, Hazen S L. Myeloperoxidase and cardiovascular disease. Arterioscler Thromb Vasc Biol. 2005; 25:1102-1111.
13. Podrez E A, Schmitt D, Hoff H F, Hazen S L. Myeloperoxidase-generated reactive nitrogen species convert LDL into an atherogenic form in vitro. J Clin Invest. 1999; 103:1547-1560.
14. Zhang R, Brennan M L, Shen Z, MacPherson J C, Schmitt D, Molenda C E, Hazen S L. Myeloperoxidase functions as a major enzymatic catalyst for initiation of lipid peroxidation at sites of inflammation. J Biol Chem. 2002; 277:46116-46122.
15. Zheng L, Settle M, Brubaker G, Schmitt D, Hazen S L, Smith J D, Kinter M. Localization of nitration and chlorination sites on apolipoprotein A-I catalyzed by myeloperoxidase in human atheroma and associated oxidative impairment in ABCA1-dependent cholesterol efflux from macrophages. J Biol Chem. 2005; 280:38-47.
16. Thukkani A K, McHowat J, Hsu F F, Brennan M L, Hazen S L, Ford D A. Identification of alpha-chloro fatty aldehydes and unsaturated lysophosphatidylcholine molecular species in human atherosclerotic lesions. Circulation. 2003; 108:3128-3133.
17. Weiss S J, Peppin G, Ortiz X, Ragsdale C, Test S T. Oxidative autoactivation of latent collagenase by human neutrophils. Science. 1985; 227:747-749.
18. Askari A T, Brennan M L, Zhou X, Drinko J, Morehead A, Thomas J D, Topol E J, Hazen S L, Penn M S. Myeloperoxidase and plasminogen activator inhibitor 1 play a central role in ventricular remodeling after myocardial infarction. J Exp Med. 2003; 197:615-624.
19. Abu-Soud H M, Hazen S L. Nitric oxide is a physiological substrate for mammalian peroxidases. J Biol Chem. 2000; 275:37524-37532.
20. Vita J A, Brennan M L, Gokce N, Mann S A, Goormastic M, Shishehbor M H, Penn M S, Keaney J F, Jr., Hazen S L. Serum myeloperoxidase levels independently predict endothelial dysfunction in humans. Circulation. 2004; 110:1134-1139.
21. Baldus S, Heitzer T, Eiserich J P, Lau D, Mollnau H, Ortak M, Petri S, Goldmann B, Duchstein H J, Berger J, Helmchen U, Freeman B A, Meinertz T, Munzel T. Myeloperoxidase enhances nitric oxide catabolism during myocardial ischemia and reperfusion. Free Radic Biol Med. 2004; 37:902-911.
22. Brennan M L, Penn M S, Van Lente F, Nambi V, Shishehbor M H, Aviles R J, Goormastic M, Pepoy M L, McErlean E S, Topol E J, Nissen S E, Hazen S L. Prognostic value of myeloperoxidase in patients with chest pain. N Engl J Med. 2003; 349:1595-1604.
23. Baldus S, Heeschen C, Meinertz T, Zeiher A M, Eiserich J P, Munzel T, Simoons M L, Hamm C W. Myeloperoxidase serum levels predict risk in patients with acute coronary syndromes. Circulation. 2003; 108:1440-1445.
24. Buffon A, Biasucci L M, Liuzzo G, D'Onofrio G, Crea F, Maseri A. Widespread coronary inflammation in unstable angina. N Engl J Med. 2002; 347:5-12.
25. Zhang R, Brennan M L, Fu X, Aviles R J, Pearce G L, Penn M S, Topol E J, Sprecher D L, Hazen S L. Association between myeloperoxidase levels and risk of coronary artery disease. JAMA. 2001; 286:2136-2142.
26. Schechter A N, Gladwin M T. Hemoglobin and the paracrine and endocrine functions of nitric oxide. N Engl J Med. 2003; 348:1483-1485.
27. Davi G, Patrono C. Platelet activation and atherothrombosis. N Engl J Med. 2007; 357:2482-2494.
28. Podrez E A, Byzova T V, Febbraio M, Salomon R G, Ma Y, Valiyaveettil M, Poliakov E, Sun M, Finton P J, Curtis B R, Chen J, Zhang R, Silverstein R L, Hazen S L. Platelet CD36 links hyperlipidemia, oxidant stress and a prothrombotic phenotype. Nat Med. 2007; 13:1086-1095.
29. Wang Z, Nicholls S J, Rodriguez E R, Kummu O, Horkko S, Barnard J, Reynolds W F, Topol E J, DiDonato J A, Hazen S L. Protein carbamylation links inflammation, smoking, uremia and atherogenesis. Nat Med. 2007; 13:1176-1184.
30. Tonelli M, Sacks F, Arnold M, Moye L, Davis B, Pfeffer M. Relation Between Red Blood Cell Distribution Width and Cardiovascular Event Rate in People With Coronary Disease. Circulation. 2008; 117:163-168.
3. Thompson S G, Kienast J, Pyke S D, Haverkate F, van de Loo J C. Hemostatic factors and the risk of myocardial infarction or sudden death in patients with angina pectoris. European Concerted Action on Thrombosis and Disabilities Angina Pectoris Study Group. N Engl J Med. 1995; 332:635-641.
32. Morange P E, Bickel C, Nicaud V, Schnabel R, Rupprecht H J, Peetz D, Lackner K J, Cambien F, Blankenberg S, Tiret L. Haemostatic factors and the risk of cardiovascular death in patients with coronary artery disease: the AtheroGene study. Arterioscler Thromb Vasc Biol. 2006; 26):2793-2799.
33. Danesh J, Collins R, Peto R, Lowe G D. Haematocrit, viscosity, erythrocyte sedimentation rate: meta-analyses of prospective studies of coronary heart disease. Eur Heart J. 2000; 21:515-520.
34. Lauer M S, Alexe S, Pothier Snader C E, Blackstone E H, Ishwaran H, Hammer P L. Use of the logical analysis of data method for assessing long-term mortality risk after exercise electrocardiography. Circulation. 2002; 106:685-690.
35. Crama Y H P, Ibaraki T. Cause-effect relationships and partially defined Boolean functions. Annals of Operations Research 1988; 16:299-326.
36. Boros E H P, Ibaraki T, et al. Logical analysis of numerical data. Math Programming. 1997; 79:163-190.
37. Hanley J A, McNeil B J. A method of comparing the areas under receiver operating characteristic curves derived from the same cases. Radiology. 1983; 148:839-843.
38. Hollander M, Wolfe D. Nonparametric Statistical Methods. New York: John Wiley & Sons; 1973.
39. Pencina M J, D'Agostino R B, Sr., D'Agostino R B, Jr., Vasan R S. Evaluating the added predictive ability of a new marker: from area under the ROC curve to reclassification and beyond. Stat Med. 2008; 27:157-172; discussion 207-112.
40. Potters L, Purrazzella R, Brustein S, Fearn P, Leibel S A, Kattan M W. A comprehensive and novel predictive modeling technique using detailed pathology factors in men with localized prostate carcinoma. Cancer. 2002; 95:1451-1456.
41. Hosmer D W, Hosmer T, Le Cessie S, Lemeshow S. A comparison of goodness-of-fit tests for the logistic regression model. Stat Med. 1997; 16:965-980.
42. Morrow D A, Sabatine M S, Brennan M L, de Lemos J A, Murphy S A, Ruff C T, Rifai N, Cannon C P, Hazen S L. Concurrent evaluation of novel cardiac biomarkers in acute coronary syndrome: myeloperoxidase and soluble CD40 ligand and the risk of recurrent ischaemic events in TACTICS-TIMI 18. Eur Heart J. 2008; 29:1096-1102.
43. Loscalzo J. The macrophage and fibrinolysis. Semin Thromb Hemost. 1996; 22:503-506.
44. Navab M, Ananthramaiah G M, Reddy S T, Van Lenten B J, Ansell B J, Fonarow G C, Vahabzadeh K, Hama S, Hough G, Kamranpour N, Berliner J A, Lusis A J, Fogelman A M. The oxidation hypothesis of atherogenesis: the role of oxidized phospholipids and HDL. J Lipid Res. 2004; 45:993-1007.
45. Naruko T, Ueda M, Haze K, van der Wal A C, van der Loos C M, Itoh A, Komatsu R, Ikura Y, Ogami M, Shimada Y, Ehara S, Yoshiyama M, Takeuchi K, Yoshikawa J, Becker A E. Neutrophil infiltration of culprit lesions in acute coronary syndromes. Circulation. 2002; 106:2894-2900.

Although only a few exemplary embodiments have been described in detail, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure.

We claim:

1. A method of characterizing a subject's risk of developing cardiovascular disease or experiencing a complication of cardiovascular disease, comprising:
a) determining the value of a first marker in a biological sample from said subject, wherein said first marker is selected from the group consisting of: Markers 1-19, 47, and 54-55 as defined in Table 50, and
b) comparing said value of said first marker to a first threshold value such that said subject's risk of developing cardiovascular disease or experiencing a complication of cardiovascular disease is at least partially characterized.

2. The method of claim 1, wherein said biological sample comprises blood.

3. The method of claim 1, wherein said complication is one or more of the following: non-fatal myocardial infarction, stroke, angina pectoris, transient ischemic attacks, congestive heart failure, aortic aneurysm, aortic dissection, and death.

4. The method of claim 1, wherein said method further comprises:
c) determining the value of a second marker in said biological sample, wherein said second marker is different from said first marker and is selected from the group consisting Markers 1-75 as defined in Table 50; and
d) comparing said value of said second marker to a second threshold value such that said subject's risk of developing cardiovascular disease or experiencing a complication of cardiovascular disease is further characterized.

5. The method of claim 4, wherein said method further comprises:
c) determining the value of a third marker in said biological sample, wherein said third marker is different from said first and second markers and is selected from the group consisting Markers 1-75 as defined in Table 50; and
d) comparing said value of said third marker to a third threshold value such that said subject's risk of developing cardiovascular disease or experiencing a complication of cardiovascular disease is further characterized.

6. The method of claim 1, wherein a hematology analyzer is employed to determine said value of said first marker.

7. The method of claim 1, wherein said comparing said value of said first marker to said first threshold value generates a first high-risk indicator, a first non-high/low-risk indicator, or a first low-risk indicator.

8. The method of claim 7, wherein said first high-risk indicator, said first non-high/low-risk indicator, or said first low-risk indicator is employed to generate an overall risk score for said subject.

9. A method of characterizing a subject's risk of developing cardiovascular disease or experiencing a complication of cardiovascular disease, comprising:
a) determining the value of a first marker in a biological sample from said subject, wherein said first marker is selected from the group consisting of: Markers 22, 24-26, 28, 30-31, 34-37, 39-45, 48, and 50-53 as defined in Table 50, and
b) comparing said value of said first marker to a first threshold value such that said subject's risk of developing cardiovascular disease or experiencing a complication of cardiovascular disease is at least partially characterized.

10. The method of claim 9, wherein said method further comprises:
c) determining the value of a second marker in said biological sample, wherein said second marker is different from said first marker and is selected from the group consisting Markers 1-75 as defined in Table 50; and
d) comparing said value of said second marker to a second threshold value such that said subject's risk of developing cardiovascular disease or experiencing a complication of cardiovascular disease is further characterized.

11. A system comprising:
a) a blood analyzer device; and
b) a computer program component comprising:
i) a computer readable medium;
ii) threshold value data on said computer readable medium comprising at least a first threshold value; and
iii) instructions on said computer readable medium adapted to enable a computer processor to perform operations comprising:
A) receiving subject data, wherein said subject data comprises the value of a first marker from a biological sample from said subject, wherein said first marker is selected from the group consisting of Markers 1-19, 47, and 54-55 as defined in Table 50;
B) comparing said value of said first marker to said first threshold value; and
C) generating first high-risk indicator data, first non-high/low-risk indicator data, or first low-risk indicator data based on said comparing.

12. The system of claim 11, wherein said system further comprises said computer processor, and wherein said computer program component is operably linked to said computer processor, and wherein said computer processor is operably linked to said blood analyzer device.

13. The system of claim 11, wherein said system further comprises a display component configured to display: i) said high-risk indicator data, first non-high/low risk indicator data, and/or first low-risk indicator data; and/or ii) a risk profile.

14. The system of claim 11, wherein said blood analyzer device comprises a hematology analyzer.

15. The system of claim 11, wherein said instruction are adapted to enable said computer processor to perform operations further comprising: iv) outputting said first high-risk indicator data, said first non-high/low risk indicator data, or said first low-risk indicator data.

16. The system of claim 11, wherein said instruction are adapted to enable said computer processor to perform operations further comprising: generating an overall risk score for said subject based on said first high-risk indicator data, said non-high/low risk indicator data, or said first low-risk indicator data.

17. The system of claim 11, wherein said threshold data further comprises a second threshold value; wherein said subject data further comprises the value of a second marker, wherein said second marker is different from said first marker and is selected from the group consisting Markers 1-75 as defined in Table 50; and wherein said instructions on said computer readable medium are further adapted to enable said computer processor to perform operations comprising: 1) comparing said value of said second marker to said second threshold value, and 2) generating second high-risk indicator data, second non-high/low-risk indicator data, or second low-risk indicator data based on said comparing.
